SEQUENCE LISTING (271 sequences)

SEQ ID NO: 1 - DNA, 23 bp, Artificial Sequence
  miR-135a-5p
uauggcuuuuuauuccuauguga

SEQ ID NO: 2 - DNA, 25 bp, Artificial Sequence
  miR-182-5p
uuuggcaaugguagaacucacaccg

SEQ ID NO: 3 - DNA, 22 bp, Artificial Sequence
  miR-542-5p
cucggggaucaucaugucacga

SEQ ID NO: 4 - DNA, 22 bp, Artificial Sequence
  miR-183-5p
uauggcacugguagaauucacu

SEQ ID NO: 5 - DNA, 22 bp, Artificial Sequence
  miR-296-3p
gaggguuggguggaggcucucc

SEQ ID NO: 6 - DNA, 23 bp, Artificial Sequence
  miR-96-5p
uuuggcacuagcacauuuuugcu

SEQ ID NO: 7 - DNA, 22 bp, Artificial Sequence
  miR-34a-5p
uggcagugucuuagcugguugu

SEQ ID NO: 8 - DNA, 22 bp, Artificial Sequence
  miR-22-3p
aagcugccaguugaagaacugu

SEQ ID NO: 9 - DNA, 22 bp, Artificial Sequence
  miR-199a-3p
acaguagucugcacauugguua

SEQ ID NO: 10 - DNA, 22 bp, Artificial Sequence
  miR-125b-5p
ucccugagacccuaacuuguga

SEQ ID NO: 11 - DNA, 22 bp, Artificial Sequence
  let-7c-5p
ugagguaguagguuguaugguu

SEQ ID NO: 12 - protein, 7 aa, Artificial Sequence
  Disintegrin and metalloproteinase domain- containing protein 10 (ADAM 10) (EC 3.4.24.81) (Kuzbanian protein homolog) (Mammalian disintegrin-metalloprotease) (CD antigen CD156c)
FSLCSIR

SEQ ID NO: 13 - protein, 10 aa, Artificial Sequence
  Disintegrin and metalloproteinase domain-containing protein 10 (ADAM 10) (EC 3.4.24.81) (Kuzbanian protein homolog) (Mammalian disintegrin-metalloprotease) (CD antigen CD156c)
LVDADGPLAR

SEQ ID NO: 14 - protein, 14 aa, Artificial Sequence
  Disintegrin and metalloproteinase domain-containing protein 10 (ADAM 10) (EC 3.4.24.81) (Kuzbanian protein homolog) (Mammalian disintegrin-metalloprotease) (CD antigen CD156c)
TITLQPGSPCNDFR

SEQ ID NO: 15 - protein, 10 aa, Artificial Sequence
  Aldo-keto reductase family 1 member B1 (EC 1.1.1.300) (EC 1.1.1.372) (EC 1.1.1.54) (Aldehyde reductase) (Aldo-keto reductase family 1 member B3) (Aldose reductase) (AR) (EC 1.1.1.21)
MPTLGLGTWK

SEQ ID NO: 16 - protein, 7 aa, Artificial Sequence
  Aldo-keto reductase family 1 member B1 (EC 1.1.1.300) (EC 1.1.1.372) (EC 1.1.1.54) (Aldehyde reductase) (Aldo-keto reductase family 1 member B3) (Aldose reductase) (AR) (EC 1.1.1.21)
NLVVIPK

SEQ ID NO: 17 - protein, 11 aa, Artificial Sequence
  Aldo-keto reductase family 1 member B1 (EC 1.1.1.300) (EC 1.1.1.372) (EC 1.1.1.54) (Aldehyde reductase) (Aldo-keto reductase family 1 member B3) (Aldose reductase) (AR) (EC 1.1.1.21)
SPPGQVTEAVK

SEQ ID NO: 18 - protein, 13 aa, Artificial Sequence
  Albumin
LGEYGFQNAILVR

SEQ ID NO: 19 - protein, 13 aa, Artificial Sequence
  Aldehyde dehydrogenase, mitochondrial (EC 1.2.1.3) (AHD-M1) (ALDH class 2) (ALDH-E2) (ALDHI)
GYFIQPTVFGDVK

SEQ ID NO: 20 - protein, 17 aa, Artificial Sequence
  Aldehyde dehydrogenase, mitochondrial (EC 1.2.1.3) (AHD-M1) (ALDH class 2) (ALDH-E2) (ALDHI)
VAEQTPLTALYVANLIK

SEQ ID NO: 21 - protein, 11 aa, Artificial Sequence
  Annexin A1 (Annexin I) (Annexin-1) (Calpactin II) (Calpactin-2) (Chromobindin-9) (Lipocortin I) (Phospholipase A2 inhibitory protein) (p35)
DITSDTSGDFR

SEQ ID NO: 22 - protein, 13 aa, Artificial Sequence
  Annexin A1 (Annexin I) (Annexin-1) (Calpactin II) (Calpactin-2) (Chromobindin-9) (Lipocortin I) (Phospholipase A2 inhibitory protein) (p35)
GVDEATIIDILTK

SEQ ID NO: 23 - protein, 11 aa, Artificial Sequence
  Annexin A1 (Annexin I) (Annexin-1) (Calpactin II) (Calpactin-2) (Chromobindin-9) (Lipocortin I) (Phospholipase A2 inhibitory protein) (p35)
TPAQFDADELR

SEQ ID NO: 24 - protein, 10 aa, Artificial Sequence
  Annexin A2 (Annexin II) (Annexin-2) (Calpactin I heavy chain) (Calpactin-1 heavy chain) (Chromobindin-8) (Lipocortin II) (Placental anticoagulant protein IV) (PAP-IV) (Protein I) (p36)
DALNIETAVK

SEQ ID NO: 25 - protein, 11 aa, Artificial Sequence
  Annexin A2 (Annexin II) (Annexin-2) (Calpactin I heavy chain) (Calpactin-1 heavy chain) (Chromobindin-8) (Lipocortin II) (Placental anticoagulant protein IV) (PAP-IV) (Protein I) (p36)
DIISDTSGDFR

SEQ ID NO: 26 - protein, 11 aa, Artificial Sequence
  Annexin A5 (Anchorin CII) (Annexin V) (Annexin-5) (Calphobindin I) (CBP-I) (Endonexin II) (Lipocortin V) (Placental anticoagulant protein 4) (PP4) (Placental anticoagulant protein I) (PAP-I) (Thromboplastin inhibitor) (Vascular anticoagulant-alpha)
NFATSLYSMIK

SEQ ID NO: 27 - protein, 9 aa, Artificial Sequence
  Annexin A5 (Anchorin CII) (Annexin V) (Annexin-5) (Calphobindin I) (CBP-I) (Endonexin II) (Lipocortin V) (Placental anticoagulant protein 4) (PP4) (Placental anticoagulant protein I) (PAP-I) (Thromboplastin inhibitor) (Vascular anticoagulant-alpha)
SEIDLFNIR

SEQ ID NO: 28 - protein, 9 aa, Artificial Sequence
  Annexin A5 (Anchorin CII) (Annexin V) (Annexin-5) (Calphobindin I) (CBP-I) (Endonexin II) (Lipocortin V) (Placental anticoagulant protein 4) (PP4) (Placental anticoagulant protein I) (PAP-I) (Thromboplastin inhibitor) (Vascular anticoagulant-alpha)
TPEELSAIK

SEQ ID NO: 29 - protein, 12 aa, Artificial Sequence
  Apolipoprotein B-100 (Apo B-100) [Cleaved into Apolipoprotein B-48 (Apo B-48)]
GFEPTLEALFGK

SEQ ID NO: 30 - protein, 10 aa, Artificial Sequence
  Apolipoprotein B-100 (Apo B-100) [Cleaved into Apolipoprotein B-48 (Apo B-48)]
QGFFPDSVNK

SEQ ID NO: 31 - protein, 9 aa, Artificial Sequence
  Apolipoprotein B-100 (Apo B-100) [Cleaved into Apolipoprotein B-48 (Apo B-48)]
YENYELTLK

SEQ ID NO: 32 - protein, 12 aa, Artificial Sequence
  Arrestin domain-containing protein 1 (Alpha-arrestin 1)
DTSPVVASLLQK

SEQ ID NO: 33 - protein, 8 aa, Artificial Sequence
  Arrestin domain-containing protein 1 (Alpha-arrestin 1)
LGAPLPFR

SEQ ID NO: 34 - protein, 11 aa, Artificial Sequence
  Arrestin domain-containing protein 1 (Alpha-arrestin 1)
TIAEVEGTGVK

SEQ ID NO: 35 - protein, 13 aa, Artificial Sequence
  Complement C1q tumor necrosis factor-related protein 3 (Collagenous repeat-containing sequence 26 kDa protein) (CORS26) (Secretory protein CORS26)
FSTFAGFLLFETK

SEQ ID NO: 36 - protein, 7 aa, Artificial Sequence
  Complement C1q tumor necrosis factor-related protein 3 (Collagenous repeat-containing sequence 26 kDa protein) (CORS26) (Secretory protein CORS26)
GDEVWLR

SEQ ID NO: 37 - protein, 12 aa, Artificial Sequence
  Complement C1q tumor necrosis factor-related protein 3 (Collagenous repeat-containing sequence 26 kDa protein) (CORS26) (Secretory protein CORS26)
MGNGALHGDHQR

SEQ ID NO: 38 - protein, 17 aa, Artificial Sequence
  Complement C4-B [Cleaved into Complement C4 beta chain; Complement C4 alpha chain; C4a anaphylatoxin; Complement C4 gamma chain]
GHIFVQTDQPIYNPGQR

SEQ ID NO: 39 - protein, 7 aa, Artificial Sequence
  Complement C4-B [Cleaved into Complement C4 beta chain; Complement C4 alpha chain; C4a anaphylatoxin; Complement C4 gamma chain]
VFALDQK

SEQ ID NO: 40 - protein, 16 aa, Artificial Sequence
  Calnexin
APVPTGEVYFADSFDR

SEQ ID NO: 41 - protein, 7 aa, Artificial Sequence
  Calnexin
GLVLMSR

SEQ ID NO: 42 - protein, 10 aa, Artificial Sequence
  Calnexin
GSLSGWILSK

SEQ ID NO: 43 - protein, 18 aa, Artificial Sequence
  CD63 antigen (CD antigen CD63)
ESTIHTQGCVETIAIWLR

SEQ ID NO: 44 - protein, 11 aa, Artificial Sequence
  CD63 antigen (CD antigen CD63)
SFQQQMQNYLK

SEQ ID NO: 45 - protein, 8 aa, Artificial Sequence
  CD81 antigen (26 kDa cell surface protein TAPA-1) (Target of the antiproliferative antibody 1) (CD antigen CD81)
IDELFSGK

SEQ ID NO: 46 - protein, 20 aa, Artificial Sequence
  CD81 antigen (26 kDa cell surface protein TAPA-1) (Target of the antiproliferative antibody 1) (CD antigen CD81)
QFYDQALQQAVMDDDANNAK

SEQ ID NO: 47 - protein, 7 aa, Artificial Sequence
  CD9 antigen (CD antigen CD9)
ELQEFYK

SEQ ID NO: 48 - protein, 11 aa, Artificial Sequence
  Calponin-3 (Calponin, acidic isoform)
DGIILCELINK

SEQ ID NO: 49 - protein, 13 aa, Artificial Sequence
  Calponin-3 (Calponin, acidic isoform)
GASQAGMLAPGTR

SEQ ID NO: 50 - protein, 9 aa, Artificial Sequence
  Calponin-3 (Calponin, acidic isoform)
GMSVYGLGR

SEQ ID NO: 51 - protein, 12 aa, Artificial Sequence
  CCR4-NOT transcription complex subunit 1 (CCR4-associated factor 1)
AVQELVHPVVDR

SEQ ID NO: 52 - protein, 11 aa, Artificial Sequence
  CCR4-NOT transcription complex subunit 1 (CCR4-associated factor 1)
IVEPPENIQEK

SEQ ID NO: 53 - protein, 9 aa, Artificial Sequence
  CCR4-NOT transcription complex subunit 1 (CCR4-associated factor 1)
NLILSAFPR

SEQ ID NO: 54 - protein, 18 aa, Artificial Sequence
  Collagen alpha-1(V) chain
FLGSNDEEMSYDNNPYIR

SEQ ID NO: 55 - protein, 11 aa, Artificial Sequence
  Collagen alpha-1(V) chain
RPLGTQQNPAR

SEQ ID NO: 56 - protein, 19 aa, Artificial Sequence
  Collagen alpha-1(V) chain
TGPIGPQGAPGKPGPDGLR

SEQ ID NO: 57 - protein, 7 aa, Artificial Sequence
  Coatomer subunit beta′ (Beta′-coat protein) (Beta′-COP) (p102)
IWDYQNK

SEQ ID NO: 58 - protein, 9 aa, Artificial Sequence
  Coatomer subunit beta′ (Beta′-coat protein) (Beta′-COP) (p102)
TYLPSQVSR

SEQ ID NO: 59 - protein, 7 aa, Artificial Sequence
  Coatomer subunit beta′ (Beta′-coat protein) (Beta′-COP) (p102)
VLPTIPK

SEQ ID NO: 60 - protein, 11 aa, Artificial Sequence
  Ceruloplasmin (EC 1.16.3.1) (Ferroxidase)
DIFTGLIGPMK

SEQ ID NO: 61 - protein, 15 aa, Artificial Sequence
  Ceruloplasmin (EC 1.16.3.1) (Ferroxidase)
NMATRPYSIHAHGVK

SEQ ID NO: 62 - protein, 13 aa, Artificial Sequence
  Alpha-crystallin B chain (Alpha(B)-crystallin) (P23)
APSWIDTGLSEMR

SEQ ID NO: 63 - protein, 11 aa, Artificial Sequence
  Alpha-crystallin B chain (Alpha(B)-crystallin) (P23)
RPFFPFHSPSR

SEQ ID NO: 64 - protein, 11 aa, Artificial Sequence
  Coxsackievirus and adenovirus receptor homolog (CAR) (mCAR)
APQSPTLAPAK

SEQ ID NO: 65 - protein, 8 aa, Artificial Sequence
  Coxsackievirus and adenovirus receptor homolog (CAR) (mCAR)
VAAPNLSR

SEQ ID NO: 66 - protein, 9 aa, Artificial Sequence
  Coxsackievirus and adenovirus receptor homolog (CAR) (mCAR)
VGSDQCMLR

SEQ ID NO: 67 - protein, 10 aa, Artificial Sequence
  Aspartate--tRNA ligase, cytoplasmic (EC 6.1.1.12) (Aspartyl-tRNA synthetase) (AspRS)
IHDPQLLTER

SEQ ID NO: 68 - protein, 10 aa, Artificial Sequence
  Aspartate--tRNA ligase, cytoplasmic (EC 6.1.1.12) (Aspartyl-tRNA synthetase) (AspRS)
IYVISLAEPR

SEQ ID NO: 69 - protein, 10 aa, Artificial Sequence
  Aspartate--tRNA ligase, cytoplasmic (EC 6.1.1.12) (Aspartyl-tRNA synthetase) (AspRS)
LQSGICHLFR

SEQ ID NO: 70 - protein, 14 aa, Artificial Sequence
  Dynactin subunit 2 (50 kDa dynein-associated polypeptide) (Dynactin complex 50 kDa subunit) (DCTN-50) (Growth cone membrane protein 23-48K) (GMP23-48K)
LLHEVQELTTEVEK

SEQ ID NO: 71 - protein, 9 aa, Artificial Sequence
  Dynactin subunit 2 (50 kDa dynein-associated polypeptide) (Dynactin complex 50 kDa subunit) (DCTN-50) (Growth cone membrane protein 23-48K) (GMP23-48K)
LLLQLEATK

SEQ ID NO: 72 - protein, 15 aa, Artificial Sequence
  Dynactin subunit 2 (50 kDa dynein-associated polypeptide) (Dynactin complex 50 kDa subunit) (DCTN-50) (Growth cone membrane protein 23-48K) (GMP23-48K)
VSALDLAVLDQVEAR

SEQ ID NO: 73 - protein, 12 aa, Artificial Sequence
  Spliceosome RNA helicase Ddx39b (EC 3.6.4.13) (56 kDa U2AF65-associated protein) (DEAD box protein UAP56) (HLA-B-associated transcript 1 protein)
NCPHIVVGTPGR

SEQ ID NO: 74 - protein, 19 aa, Artificial Sequence
  Spliceosome RNA helicase Ddx39b (EC 3.6.4.13) (56 kDa U2AF65-associated protein) (DEAD box protein UAP56) (HLA-B-associated transcript 1 protein)
VNIAFNYDMPEDSDTYLHR

SEQ ID NO: 75 - protein, 11 aa, Artificial Sequence
  ATP-dependent RNA helicase DDX3X (EC 3.6.4.13) (D1Pas1-related sequence 2) (DEAD box RNA helicase DEAD3) (mDEAD3) (DEAD box protein 3, X-chromosomal)
GCHLLVATPGR

SEQ ID NO: 76 - protein, 7 aa, Artificial Sequence
  ATP-dependent RNA helicase DDX3X (EC 3.6.4.13) (D1Pas1-related sequence 2) (DEAD box RNA helicase DEAD3) (mDEAD3) (DEAD box protein 3, X-chromosomal)
HAIPIIK

SEQ ID NO: 77 - protein, 12 aa, Artificial Sequence
  ATP-dependent RNA helicase DDX3X (EC 3.6.4.13) (D1Pas1-related sequence 2) (DEAD box RNA helicase DEAD3) (mDEAD3) (DEAD box protein 3, X-chromosomal)
SPILVATAVAAR

SEQ ID NO: 78 - protein, 8 aa, Artificial Sequence
  Probable ATP-dependent RNA helicase DDX5 (EC 3.6.4.13) (DEAD box RNA helicase DEAD1) (mDEAD1) (DEAD box protein 5) (RNA helicase p68)
LLQLVEDR

SEQ ID NO: 79 - protein, 11 aa, Artificial Sequence
  Probable ATP-dependent RNA helicase DDX5 (EC 3.6.4.13) (DEAD box RNA helicase DEAD1) (mDEAD1) (DEAD box protein 5) (RNA helicase p68)
NFYQEHPDLAR

SEQ ID NO: 80 - protein, 8 aa, Artificial Sequence
  Probable ATP-dependent RNA helicase DDX5 (EC 3.6.4.13) (DEAD box RNA helicase DEAD1) (mDEAD1) (DEAD box protein 5) (RNA helicase p68)
TIVFVETK

SEQ ID NO: 81 - protein, 10 aa, Artificial Sequence
  Pre-mRNA-splicing factor ATP-dependent RNA helicase DHX15 (EC 3.6.4.13) (DEAH box protein 15)
EAMNDPLLER

SEQ ID NO: 82 - protein, 13 aa, Artificial Sequence
  Pre-mRNA-splicing factor ATP-dependent RNA helicase DHX15 (EC 3.6.4.13) (DEAH box protein 15)
EVDDLGPEVGDIK

SEQ ID NO: 83 - protein, 12 aa, Artificial Sequence
  Pre-mRNA-splicing factor ATP-dependent RNA helicase DHX15 (EC 3.6.4.13) (DEAH box protein 15)
TLATDILMGVLK

SEQ ID NO: 84 - protein, 10 aa, Artificial Sequence
  ATP-dependent RNA helicase A (EC 3.6.4.13) (DEAH box protein 9) (mHEL-5) (Nuclear DNA helicase II) (NDH II) (RNA helicase A) (RHA)
DVVLAYPEVR

SEQ ID NO: 85 - protein, 9 aa, Artificial Sequence
  ATP-dependent RNA helicase A (EC 3.6.4.13) (DEAH box protein 9) (mHEL-5) (Nuclear DNA helicase II) (NDH II) (RNA helicase A) (RHA)
MSGEEAEIR

SEQ ID NO: 86 - protein, 17 aa, Artificial Sequence
  ATP-dependent RNA helicase A (EC 3.6.4.13) (DEAH box protein 9) (mHEL-5) (Nuclear DNA helicase II) (NDH II) (RNA helicase A) (RHA)
TTQVPQYILDDFIQNDR

SEQ ID NO: 87 - protein, 10 aa, Artificial Sequence
  Eukaryotic translation elongation factor 1 epsilon-1 (Elongation factor p18) (Multisynthase complex auxiliary component p18)
AMVQQWLEFR

SEQ ID NO: 88 - protein, 9 aa, Artificial Sequence
  Eukaryotic translation elongation factor 1 epsilon-1 (Elongation factor p18) (Multisynthase complex auxiliary component p18)
DLNSYLEDK

SEQ ID NO: 89 - protein, 12 aa, Artificial Sequence
  Eukaryotic translation elongation factor 1 epsilon-1 (Elongation factor p18) (Multisynthase complex auxiliary component p18)
WFCHIQHYPDIR

SEQ ID NO: 90 - protein, 13 aa, Artificial Sequence
  EGF-containing fibulin-like extracellular matrix protein 1 (Fibulin-3) (FIBL-3)
CVNHYGGYLCLPK

SEQ ID NO: 91 - protein, 13 aa, Artificial Sequence
  EGF-containing fibulin-like extracellular matrix protein 1 (Fibulin-3) (FIBL-3)
QTSPVSAMLVLVK

SEQ ID NO: 92 - protein, 11 aa, Artificial Sequence
  EGF-containing fibulin-like extracellular matrix protein 1 (Fibulin-3) (FIBL-3)
SGNENGEFYLR

SEQ ID NO: 93 - protein, 13 aa, Artificial Sequence
  Extended synaptotagmin-1 (E-Syt1) (Membrane-bound C2 domain-containing protein)
ALTLGALTLPLAR

SEQ ID NO: 94 - protein, 11 aa, Artificial Sequence
  Extended synaptotagmin-1 (E-Syt1) (Membrane-bound C2 domain-containing protein)
LLAETVAPAVR

SEQ ID NO: 95 - protein, 16 aa, Artificial Sequence
  Extended synaptotagmin-1 (E-Syt1) (Membrane-bound C2 domain-containing protein)
LLVPLVPDLQDVAQLR

SEQ ID NO: 96 - protein, 15 aa, Artificial Sequence
  Exocyst complex component 4 (Exocyst complex component Sec8)
FIQEIEHALGLGPAK

SEQ ID NO: 97 - protein, 13 aa, Artificial Sequence
  Exocyst complex component 4 (Exocyst complex component Sec8)
HVLNLLDEIENIK

SEQ ID NO: 98 - protein, 12 aa, Artificial Sequence
  Exocyst complex component 4 (Exocyst complex component Sec8)
STTQVADSAYQR

SEQ ID NO: 99 - protein, 17 aa, Artificial Sequence
  Fibrinogen alpha chain [Cleaved into Fibrinopeptide A; Fibrinogen alpha chain]
NNSPYEIENGVVWVPFR

SEQ ID NO: 100 - protein, 9 aa, Artificial Sequence
  Fibrinogen alpha chain [Cleaved into Fibrinopeptide A; Fibrinogen alpha chain]
VELEDWAGK

SEQ ID NO: 101 - protein, 8 aa, Artificial Sequence
  Fibrinogen gamma chain
IDGSLDFK

SEQ ID NO: 102 - protein, 9 aa, Artificial Sequence
  Flotillin-2 (Epidermal surface antigen) (ESA) (Membrane component chromosome 17 surface marker 1 homolog)
IQQIAEGEK

SEQ ID NO: 103 - protein, 12 aa, Artificial Sequence
  Flotillin-2 (Epidermal surface antigen) (ESA) (Membrane component chromosome 17 surface marker 1 homolog)
NVVLQTLEGHLR

SEQ ID NO: 104 - protein, 12 aa, Artificial Sequence
  Flotillin-2 (Epidermal surface antigen) (ESA) (Membrane component chromosome 17 surface marker 1 homolog)
NVVLQTLEGHLR

SEQ ID NO: 105 - protein, 10 aa, Artificial Sequence
  Ferritin heavy chain (Ferritin H subunit) (EC 1.16.3.1) [Cleaved into Ferritin heavy chain, N-terminally processed]
ELGDHVTNLR

SEQ ID NO: 106 - protein, 13 aa, Artificial Sequence
  Ferritin heavy chain (Ferritin H subunit) (EC 1.16.3.1) [Cleaved into Ferritin heavy chain, N-terminally processed]
QNYHQDAEAAINR

SEQ ID NO: 107 - protein, 14 aa, Artificial Sequence
  RNA-binding protein FUS (Protein pigpen)
CPNPTCENMNFSWR

SEQ ID NO: 108 - protein, 14 aa, Artificial Sequence
  RNA-binding protein FUS (Protein pigpen)
GEATVSFDDPPSAK

SEQ ID NO: 109 - protein, 12 aa, Artificial Sequence
  RNA-binding protein FUS (Protein pigpen)
TGQPMINLYTDR

SEQ ID NO: 110 - protein, 7 aa, Artificial Sequence
  Glutathione S-transferase omega-1 (GSTO-1) (EC 2.5.1.18) (Glutathione S-transferase omega 1-1) (GSTO 1-1) (Glutathione-dependent dehydroascorbate reductase) (EC 1.8.5.1) (Monomethylarsonic acid reductase) (MMA(V) reductase) (EC
FCPFAQR

SEQ ID NO: 111 - protein, 9 aa, Artificial Sequence
  Glutathione S-transferase omega-1 (GSTO-1) (EC 2.5.1.18) (Glutathione S-transferase omega 1-1) (GSTO 1-1) (Glutathione-dependent dehydroascorbate reductase) (EC 1.8.5.1) (Monomethylarsonic acid reductase) (MMA(V) reductase) (EC
HEVININLK

SEQ ID NO: 112 - protein, 10 aa, Artificial Sequence
  Glutathione S-transferase omega-1 (GSTO-1) (EC 2.5.1.18) (Glutathione S-transferase omega 1-1) (GSTO 1-1) (Glutathione-dependent dehydroascorbate reductase) (EC 1.8.5.1) (Monomethylarsonic acid reductase) (MMA(V) reductase) (EC
VPPLIASFVR

SEQ ID NO: 113 - protein, 10 aa, Artificial Sequence
  Hemicentin-1 (Fibulin-6) (FIBL-6)
ITGLSTIDFR

SEQ ID NO: 114 - protein, 12 aa, Artificial Sequence
  Hemicentin-1 (Fibulin-6) (FIBL-6)
LTHEVLQLIQQK

SEQ ID NO: 115 - protein, 7 aa, Artificial Sequence
  Hemicentin-1 (Fibulin-6) (FIBL-6)
QVTLECK

SEQ ID NO: 116 - protein, 16 aa, Artificial Sequence
  Heterogeneous nuclear ribonucleoprotein F (hnRNP F) [Cleaved into Heterogeneous nuclear ribonucleoprotein F, N-terminally processed]
HSGPNSADSANDGFVR

SEQ ID NO: 117 - protein, 13 aa, Artificial Sequence
  Heterogeneous nuclear ribonucleoprotein H (hnRNP H) [Cleaved into Heterogeneous nuclear ribonucleoprotein H, N-terminally processed]
GLPWSCSADEVQR

SEQ ID NO: 118 - protein, 9 aa, Artificial Sequence
  Heterogeneous nuclear ribonucleoprotein H (hnRNP H) [Cleaved into Heterogeneous nuclear ribonucleoprotein H, N-terminally processed]
IQNGAQGIR

SEQ ID NO: 119 - protein, 11 aa, Artificial Sequence
  Heterogeneous nuclear ribonucleoprotein H (hnRNP H) [Cleaved into Heterogeneous nuclear ribonucleoprotein H, N-terminally processed]
SNNVEMDWVLK

SEQ ID NO: 120 - protein, 14 aa, Artificial Sequence
  Heterogeneous nuclear ribonucleoprotein L (hnRNP L)
AITHLNNNFMFGQK

SEQ ID NO: 121 - protein, 9 aa, Artificial Sequence
  Heterogeneous nuclear ribonucleoprotein L (hnRNP L)
FSTPEQAAK

SEQ ID NO: 122 - protein, 10 aa, Artificial Sequence
  Heat shock protein HSP 90-alpha (EC 3.6.4.10) (Heat shock 86 kDa) (HSP 86) (HSP86) (Tumor-specific transplantation 86 kDa antigen) (TSTA)
ELHINLIPSK

SEQ ID NO: 123 - protein, 10 aa, Artificial Sequence
  Heat shock protein HSP 90-alpha (EC 3.6.4.10) (Heat shock 86 kDa) (HSP 86) (HSP86) (Tumor-specific transplantation 86 kDa antigen) (TSTA)
HIYFITGETK

SEQ ID NO: 124 - protein, 13 aa, Artificial Sequence
  Heat shock protein HSP 90-alpha (EC 3.6.4.10) (Heat shock 86 kDa) (HSP 86) (HSP86) (Tumor-specific transplantation 86 kDa antigen) (TSTA)
SLTNDWEEHLAVK

SEQ ID NO: 125 - protein, 7 aa, Artificial Sequence
  Heat shock protein HSP 90-beta (Heat shock 84 kDa) (HSP 84) (HSP84) (Tumor-specific transplantation 84 kDa antigen) (TSTA)
ALLFIPR

SEQ ID NO: 126 - protein, 10 aa, Artificial Sequence
  Heat shock protein HSP 90-beta (Heat shock 84 kDa) (HSP 84) (HSP84) (Tumor-specific transplantation 84 kDa antigen) (TSTA)
LGIHEDSTNR

SEQ ID NO: 127 - protein, 10 aa, Artificial Sequence
  Heat shock protein HSP 90-beta (Heat shock 84 kDa) (HSP 84) (HSP84) (Tumor-specific transplantation 84 kDa antigen) (TSTA)
SIYYITGESK

SEQ ID NO: 128 - protein, 11 aa, Artificial Sequence
  Isocitrate dehydrogenase [NADP], mitochondrial (IDH) (EC 1.1.1.42) (ICD-M) (IDP) (NADP(+)-specific ICDH) (Oxalosuccinate decarboxylase)
LIDDMVAQVLK

SEQ ID NO: 129 - protein, 13 aa, Artificial Sequence
  Isocitrate dehydrogenase [NADP], mitochondrial (IDH) (EC 1.1.1.42) (ICD-M) (IDP) (NADP(+)-specific ICDH) (Oxalosuccinate decarboxylase)
LVPGWTKPITIGR

SEQ ID NO: 130 - protein, 13 aa, Artificial Sequence
  Isocitrate dehydrogenase [NADP], mitochondrial (IDH) (EC 1.1.1.42) (ICD-M) (IDP) (NADP(+)-specific ICDH) (Oxalosuccinate decarboxylase)
TIEAEAAHGTVTR

SEQ ID NO: 131 - protein, 9 aa, Artificial Sequence
  Insulin-like growth factor 1 receptor (EC 2.7.10.1) (Insulin-like growth factor I receptor) (IGF-I receptor) (CD antigen CD221) [Cleaved into Insulin-like growth factor
DIYETDYYR

SEQ ID NO: 132 - protein, 12 aa, Artificial Sequence
  Insulin-like growth factor 1 receptor (EC 2.7.10.1) (Insulin-like growth factor I receptor) (IGF-I receptor) (CD antigen CD221) [Cleaved into Insulin-like growth factor
HSHALVSLSFLK

SEQ ID NO: 133 - protein, 8 aa, Artificial Sequence
  Insulin-like growth factor 1 receptor (EC 2.7.10.1) (Insulin-like growth factor I receptor) (IGF-I receptor) (CD antigen CD221) [Cleaved into Insulin-like growth factor 1 receptor alpha chain; Insulin-like growth factor 1 receptor beta chain]
IGDFGMTR

SEQ ID NO: 134 - protein, 8 aa, Artificial Sequence
  Inositol monophosphatase 1 (IMP 1) (IMPase 1) (EC 3.1.3.25) (D-galactose 1-phosphate phosphatase) (EC 3.1.3.94) (Inositol-1(or 4)-monophosphatase 1) (Lithium-sensitive myo-inositol monophosphatase A1)
IVLSNMEK

SEQ ID NO: 135 - protein, 11 aa, Artificial Sequence
  Inositol monophosphatase 1 (IMP 1) (IMPase 1) (EC 3.1.3.25) (D-galactose 1-phosphate phosphatase) (EC 3.1.3.94) (Inositol-1(or 4)-monophosphatase 1) (Lithium-sensitive myo-inositol monophosphatase A1)
SLLVTELGSSR

SEQ ID NO: 136 - protein, 10 aa, Artificial Sequence
  Importin-11 (Imp11) (Ran-binding protein 11) (RanBP11)
ALLTFYHVTK

SEQ ID NO: 137 - protein, 9 aa, Artificial Sequence
  Importin-11 (Imp11) (Ran-binding protein 11) (RanBP11)
QSEEHNMLR

SEQ ID NO: 138 - protein, 9 aa, Artificial Sequence
  Importin-11 (Imp11) (Ran-binding protein 11) (RanBP11)
SIGTDNVCR

SEQ ID NO: 139 - protein, 12 aa, Artificial Sequence
  Importin-7 (Imp7) (Ran-binding protein 7) (RanBP7)
AFAVGVQQVLLK

SEQ ID NO: 140 - protein, 9 aa, Artificial Sequence
  Importin-7 (Imp7) (Ran-binding protein 7) (RanBP7)
AIFQTIQNR

SEQ ID NO: 141 - protein, 8 aa, Artificial Sequence
  Importin-7 (Imp7) (Ran-binding protein 7) (RanBP7)
GTMDPALR

SEQ ID NO: 142 - protein, 12 aa, Artificial Sequence
  Integrin beta-3 (Platelet membrane glycoprotein IIIa) (GPIIIa) (CD antigen CD61)
EATSTFTNITYR

SEQ ID NO: 143 - protein, 14 aa, Artificial Sequence
  Integrin beta-3 (Platelet membrane glycoprotein IIIa) (GPIIIa) (CD antigen CD61)
NDASHLLVFTTDAK

SEQ ID NO: 144 - protein, 8 aa, Artificial Sequence
  Integrin beta-3 (Platelet membrane glycoprotein IIIa) (GPIIIa) (CD antigen CD61)
THIALDGR

SEQ ID NO: 145 - protein, 10 aa, Artificial Sequence
  Integral membrane protein 2B (Immature BRI2) (imBRI2) (Protein E25B) (Transmembrane protein BRI) (Bri) [Cleaved into BRI2, membrane form (Mature BRI2) (mBRI2); BRI2 intracellular domain (BRI2 ICD); BRI2C, soluble form; Bri23 peptide (Bri2-23) (ABri23)
VTFNSALAQK

SEQ ID NO: 146 - protein, 9 aa, Artificial Sequence
  Integral membrane protein 2B (Immature BRI2) (imBRI2) (Protein E25B) (Transmembrane protein BRI) (Bri) [Cleaved into BRI2, membrane form (Mature BRI2) (mBRI2); BRI2 intracellular domain (BRI2 ICD); BRI2C, soluble form; Bri23 peptide (Bri2-23) (ABri23)
YQTIEENIK

SEQ ID NO: 147 - protein, 11 aa, Artificial Sequence
  Inosine triphosphate pyrophosphatase (ITPase) (Inosine triphosphatase) (EC 3.6.1.9) (Non-canonical purine NTP pyrophosphatase) (Non-standard purine NTP pyrophosphatase) (Nucleoside-triphosphate diphosphatase) (Nucleoside-triphosphate
GQTSGQIVMPR

SEQ ID NO: 148 - protein, 14 aa, Artificial Sequence
  Importin subunit alpha-3 (Importin alpha Q1) (Qip1) (Karyopherin subunit alpha-4)
IEQLQNHENEDIYK

SEQ ID NO: 149 - protein, 11 aa, Artificial Sequence
  Importin subunit alpha-3 (Importin alpha Q1) (Qip1) (Karyopherin subunit alpha-4)
NVTWVMVNLCR

SEQ ID NO: 150 - protein, 13 aa, Artificial Sequence
  Importin subunit alpha-3 (Importin alpha Q1) (Qip1) (Karyopherin subunit alpha-4)
SGILPILVHCLER

SEQ ID NO: 151 - protein, 18 aa, Artificial Sequence
  Endothelial lipase (EC 3.1.1.3) (Endothelial cell-derived lipase) (EDL) (Phospholipase A1) (EC 3.1.1.32)
ITGLDPAGPMFEGVDINR

SEQ ID NO: 152 - protein, 11 aa, Artificial Sequence
  Endothelial lipase (EC 3.1.1.3) (Endothelial cell-derived lipase) (EDL) (Phospholipase A1) (EC 3.1.1.32)
NYLSQPSNPSR

SEQ ID NO: 153 - protein, 13 aa, Artificial Sequence
  Endothelial lipase (EC 3.1.1.3) (Endothelial cell-derived lipase) (EDL) (Phospholipase A1) (EC 3.1.1.32)
SSISPGQELWFHK

SEQ ID NO: 154 - protein, 13 aa, Artificial Sequence
  Prelamin-A/C [Cleaved into Lamin-A/C]
IDSLSAQLSQLQK

SEQ ID NO: 155 - protein, 9 aa, Artificial Sequence
  Prelamin-A/C [Cleaved into Lamin-A/C]
LADALQELR

SEQ ID NO: 156 - protein, 10 aa, Artificial Sequence
  Prelamin-A/C [Cleaved into Lamin-A/C]
SLETENAGLR

SEQ ID NO: 157 - protein, 10 aa, Artificial Sequence
  Latent-transforming growth factor beta-binding protein 1 (LTBP-1) (Transforming growth factor beta-1-binding protein 1) (TGF-beta1-BP-1)
CPLPGTAAFK

SEQ ID NO: 158 - protein, 14 aa, Artificial Sequence
  Latent-transforming growth factor beta-binding protein 1 (LTBP-1) (Transforming growth factor beta-1-binding protein 1) (TGF-beta1-BP-1)
HPPEASVQIHQVSR

SEQ ID NO: 159 - protein, 9 aa, Artificial Sequence
  Latent-transforming growth factor beta-binding protein 1 (LTBP-1) (Transforming growth factor beta-1-binding protein 1) (TGF-beta1-BP-1)
VVFTPSICK

SEQ ID NO: 160 - protein, 13 aa, Artificial Sequence
  DNA replication licensing factor MCM2 (EC 3.6.4.12) (Minichromosome maintenance protein 2 homolog) (Nuclear protein BM28)
ESMATGSIPITVR

SEQ ID NO: 161 - protein, 7 aa, Artificial Sequence
  DNA replication licensing factor MCM2 (EC 3.6.4.12) (Minichromosome maintenance protein 2 homolog) (Nuclear protein BM28)
HIESMIR

SEQ ID NO: 162 - protein, 12 aa, Artificial Sequence
  DNA replication licensing factor MCM2 (EC 3.6.4.12) (Minichromosome maintenance protein 2 homolog) (Nuclear protein BM28)
VAVGELTDEDVK

SEQ ID NO: 163 - protein, 17 aa, Artificial Sequence
  Midkine (MK) (Retanoic acid-responsive protein) (Retinoic acid-induced differentiation factor)
GSECSEWTWGPCTPSSK

SEQ ID NO: 164 - protein, 10 aa, Artificial Sequence
  Midkine (MK) (Retanoic acid-responsive protein) (Retinoic acid-induced differentiation factor)
YNAQCQETIR

SEQ ID NO: 165 - protein, 10 aa, Artificial Sequence
  S-methyl-5′-thioadenosine phosphorylase (EC 2.4.2.28) (5′-methylthioadenosine phosphorylase) (MTA phosphorylase) (MTAP) (MTAPase)
GTIVTIEGPR

SEQ ID NO: 166 - protein, 17 aa, Artificial Sequence
  S-methyl-5′-thioadenosine phosphorylase (EC 2.4.2.28) (5′-methylthioadenosine phosphorylase) (MTA phosphorylase) (MTAP) (MTAPase)
TSLRPQTFYDGSHCSAR

SEQ ID NO: 167 - protein, 17 aa, Artificial Sequence
  S-methyl-5′-thioadenosine phosphorylase (EC 2.4.2.28) (5′-methylthioadenosine phosphorylase) (MTA phosphorylase) (MTAP) (MTAPase)
YVDTPFGKPSDALILGK

SEQ ID NO: 168 - protein, 9 aa, Artificial Sequence
  Unconventional myosin-XVIIIa (Molecule associated with JAK3 N-terminus) (MAJN) (Myosin containing a PDZ domain) (Surfactant protein receptor SP-R210) (SP-R210)
IISNLFLGR

SEQ ID NO: 169 - protein, 9 aa, Artificial Sequence
  Unconventional myosin-XVIIIa (Molecule associated with JAK3 N-terminus) (MAJN) (Myosin containing a PDZ domain) (Surfactant protein receptor SP-R210) (SP-R210)
LTAELQDTK

SEQ ID NO: 170 - protein, 12 aa, Artificial Sequence
  Unconventional myosin-XVIIIa (Molecule associated with JAK3 N-terminus) (MAJN) (Myosin containing a PDZ domain) (Surfactant protein receptor SP-R210) (SP-R210)
VVHFAEPGAGTK

SEQ ID NO: 171 - protein, 11 aa, Artificial Sequence
  Nucleosome assembly protein 1-like 4
EAATVEELNPK

SEQ ID NO: 172 - protein, 15 aa, Artificial Sequence
  Nucleosome assembly protein 1-like 4
QVPNESFFNFFSPLK

SEQ ID NO: 173 - protein, 16 aa, Artificial Sequence
  Nuclear receptor-binding protein (HLS7-interacting protein kinase) (MLF1 adapter molecule)
AVFDNLIQLEHLNIVK

SEQ ID NO: 174 - protein, 15 aa, Artificial Sequence
  Nuclear receptor-binding protein (HLS7-interacting protein kinase) (MLF1 adapter molecule)
ELLFHPALFEVPSLK

SEQ ID NO: 175 - protein, 11 aa, Artificial Sequence
  Nuclear receptor-binding protein (HLS7-interacting protein kinase) (MLF1 adapter molecule)
LTSLLEETLNK

SEQ ID NO: 176 - protein, 10 aa, Artificial Sequence
  Ubiquitin thioesterase OTUB1 (EC 3.4.19.12) (Deubiquitinating enzyme OTUB1) (OTU domain-containing ubiquitin aldehyde-binding protein 1) (Otubain-1)
FFEHFIEGGR

SEQ ID NO: 177 - protein, 15 aa, Artificial Sequence
  Ubiquitin thioesterase OTUB1 (EC 3.4.19.12) (Deubiquitinating enzyme OTUB1) (OTU domain-containing ubiquitin aldehyde-binding protein 1) (Otubain-1)
IQQEIAVQNPLVSER

SEQ ID NO: 178 - protein, 9 aa, Artificial Sequence
  Ubiquitin thioesterase OTUB1 (EC 3.4.19.12) (Deubiquitinating enzyme OTUB1) (OTU domain-containing ubiquitin aldehyde-binding protein 1) (Otubain-1)
LLTSGYLQR

SEQ ID NO: 179 - protein, 8 aa, Artificial Sequence
  Programmed cell death 6-interacting protein (ALG-2-interacting protein 1) (ALG-2-interacting protein X) (E2F1-inducible protein) (Eig2)
ELPELLQR

SEQ ID NO: 180 - protein, 10 aa, Artificial Sequence
  Programmed cell death 6-interacting protein (ALG-2-interacting protein 1) (ALG-2-interacting protein X) (E2F1-inducible protein) (Eig2)
LALASLGYEK

SEQ ID NO: 181 - protein, 11 aa, Artificial Sequence
  Programmed cell death 6-interacting protein (ALG-2-interacting protein 1) (ALG-2-interacting protein X) (E2F1-inducible protein) (Eig2)
NIQVSHQEFSK

SEQ ID NO: 182 - protein, 14 aa, Artificial Sequence
  Protein disulfide-isomerase A4 (EC 5.3.4.1) (Endoplasmic reticulum resident protein 72) (ER protein 72) (ERp-72) (ERp72)
FAMEPEEFDSDTLR

SEQ ID NO: 183 - protein, 12 aa, Artificial Sequence
  Protein disulfide-isomerase A4 (EC 5.3.4.1) (Endoplasmic reticulum resident protein 72) (ER protein 72) (ERp-72) (ERp72)
IDATSASMLASK

SEQ ID NO: 184 - protein, 8 aa, Artificial Sequence
  Protein disulfide-isomerase A4 (EC 5.3.4.1) (Endoplasmic reticulum resident protein 72) (ER protein 72) (ERp-72) (ERp72)
SPPIPLAK

SEQ ID NO: 185 - protein, 9 aa, Artificial Sequence
  Proenkephalin-A [Cleaved into Synenkephalin; Met-enkephalin (Opioid growth factor) (OGF); PENK(114-133); PENK(143-184); Met-enkephalin-Arg-Ser-Leu; Leu-enkephalin; PENK(238-259); Met-enkephalin-Arg-Phe]
SPQLEDEAK

SEQ ID NO: 186 - protein, 12 aa, Artificial Sequence
  Proenkephalin-A [Cleaved into Synenkephalin; Met-enkephalin (Opioid growth factor) (OGF); PENK(114-133); PENK(143-184); Met-enkephalin-Arg-Ser-Leu; Leu-enkephalin; PENK(238-259); Met-enkephalin-Arg-Phe]
VGRPEWWMDYQK

SEQ ID NO: 187 - protein, 11 aa, Artificial Sequence
  Phospholipase A-2-activating protein (PLA2P) (PLAP)
ESLMSHAIELK

SEQ ID NO: 188 - protein, 17 aa, Artificial Sequence
  Phospholipase A-2-activating protein (PLA2P) (PLAP)
IGDVVGSSGANQQTSGK

SEQ ID NO: 189 - protein, 15 aa, Artificial Sequence
  Phospholipase A-2-activating protein (PLA2P) (PLAP)
ILPEQGLMLTGSADK

SEQ ID NO: 190 - protein, 10 aa, Artificial Sequence
  5′-3′ exonuclease PLD3 (EC 3.1.16.1) (Choline phosphatase 3) (Phosphatidylcholine-hydrolyzing phospholipase D3) (Phospholipase D3) (PLD 3) (Schwannoma-associated protein 9) (SAM-9)
ALLNVVDSAR

SEQ ID NO: 191 - protein, 8 aa, Artificial Sequence
  Prolyl endopeptidase (PE) (EC 3.4.21.26) (Post-proline cleaving enzyme)
ELPDVLER

SEQ ID NO: 192 - protein, 11 aa, Artificial Sequence
  Prolyl endopeptidase (PE) (EC 3.4.21.26) (Post-proline cleaving enzyme)
FIATLQYIVGR

SEQ ID NO: 193 - protein, 15 aa, Artificial Sequence
  Prolyl endopeptidase (PE) (EC 3.4.21.26) (Post-proline cleaving enzyme)
NILQLHDLTTGALLK

SEQ ID NO: 194 - protein, 9 aa, Artificial Sequence
  Pre-mRNA-processing-splicing factor 8 (Splicing factor Prp8)
ANPALYVLR

SEQ ID NO: 195 - protein, 8 aa, Artificial Sequence
  Pre-mRNA-processing-splicing factor 8 (Splicing factor Prp8)
IDLTLLNR

SEQ ID NO: 196 - protein, 10 aa, Artificial Sequence
  Pre-mRNA-processing-splicing factor 8 (Splicing factor Prp8)
TAEEVAALIR

SEQ ID NO: 197 - protein, 7 aa, Artificial Sequence
  26S proteasome non-ATPase regulatory subunit 1 (26S proteasome regulatory subunit RPN2) (26S proteasome regulatory subunit S1)
LEGIVNK

SEQ ID NO: 198 - protein, 10 aa, Artificial Sequence
  26S proteasome non-ATPase regulatory subunit 1 (26S proteasome regulatory subunit RPN2) (26S proteasome regulatory subunit S1)
QAIGIALETR

SEQ ID NO: 199 - protein, 8 aa, Artificial Sequence
  26S proteasome non-ATPase regulatory subunit 1 (26S proteasome regulatory subunit RPN2) (26S proteasome regulatory subunit S1)
QDVYDLLK

SEQ ID NO: 200 - protein, 7 aa, Artificial Sequence
  Transcriptional activator protein Pur-alpha (Purine-rich single-stranded DNA-binding protein alpha)
FGHTFCK

SEQ ID NO: 201 - protein, 8 aa, Artificial Sequence
  Transcriptional activator protein Pur-alpha (Purine-rich single-stranded DNA-binding protein alpha)
NSITVPYK

SEQ ID NO: 202 - protein, 9 aa, Artificial Sequence
  Transcriptional activator protein Pur-alpha (Purine-rich single-stranded DNA-binding protein alpha)
VSEVKPTYR

SEQ ID NO: 203 - protein, 10 aa, Artificial Sequence
  Glutamine--tRNA ligase (EC 6.1.1.18) (Glutaminyl-tRNA synthetase) (GlnRS)
AINFNFGYAK

SEQ ID NO: 204 - protein, 8 aa, Artificial Sequence
  Glutamine--tRNA ligase (EC 6.1.1.18) (Glutaminyl-tRNA synthetase) (GlnRS)
LFTLTALR

SEQ ID NO: 205 - protein, 11 aa, Artificial Sequence
  Glutamine--tRNA ligase (EC 6.1.1.18) (Glutaminyl-tRNA synthetase) (GlnRS)
QHLEITGGQVR

SEQ ID NO: 206 - protein, 11 aa, Artificial Sequence
  Ras-related protein Rab-6A (Rab-6)
LVFLGEQSVGK

SEQ ID NO: 207 - protein, 7 aa, Artificial Sequence
  Ras-related protein Rab-6A (Rab-6)
TMYLEDR

SEQ ID NO: 208 - protein, 14 aa, Artificial Sequence
  Ras-related protein Rab-6A (Rab-6)
VAAALPGMESTQDR

SEQ ID NO: 209 - protein, 16 aa, Artificial Sequence
  Histone-binding protein RBBP4 (Chromatin assembly factor 1 subunit C) (CAF-1 subunit C) (Chromatin assembly factor I p48 subunit) (CAF-I 48 kDa subunit) (CAF-I p48) (Nucleosome-remodeling factor subunit RBAP48)
HPSKPDPSGECNPDLR

SEQ ID NO: 210 - protein, 13 aa, Artificial Sequence
  60S ribosomal protein L22 (Heparin-binding protein HBp15)
AGNLGGGVVTIER

SEQ ID NO: 211 - protein, 11 aa, Artificial Sequence
  60S ribosomal protein L22 (Heparin-binding protein HBp15)
ITVTSEVPFSK

SEQ ID NO: 212 - protein, 11 aa, Artificial Sequence
  Protein S100-A11 (Calgizzarin) (Endothelial monocyte-activating polypeptide) (EMAP) (Protein S100-C) (S100 calcium-binding protein A11)
CIESLIAVFQK

SEQ ID NO: 213 - protein, 16 aa, Artificial Sequence
  Protein S100-A11 (Calgizzarin) (Endothelial monocyte-activating polypeptide) (EMAP) (Protein S100-C) (S100 calcium-binding protein A11)
TEFLSFMNTELAAFTK

SEQ ID NO: 214 - protein, 18 aa, Artificial Sequence
  Lysosome membrane protein 2 (85 kDa lysosomal membrane sialoglycoprotein) (LGP85) (Lysosome membrane protein II) (LIMP II) (Scavenger receptor class B member 2)
NGAPIIMSFPHFYQADEK

SEQ ID NO: 215 - protein, 11 aa, Artificial Sequence
  Protein transport protein Sec23A (SEC23-related protein A)
AETEEGPDVLR

SEQ ID NO: 216 - protein, 17 aa, Artificial Sequence
  Protein transport protein Sec23A (SEC23-related protein A)
HLLQAPVDDAQEILHSR

SEQ ID NO: 217 - protein, 14 aa, Artificial Sequence
  Protein transport protein Sec23A (SEC23-related protein A)
MVQVHELGCEGISK

SEQ ID NO: 218 - protein, 7 aa, Artificial Sequence
  Protein transport protein Sec31A (SEC31-like protein 1) (SEC31-related protein A)
FASSPLR

SEQ ID NO: 219 - protein, 8 aa, Artificial Sequence
  Protein transport protein Sec31A (SEC31-like protein 1) (SEC31-related protein A)
LITAVVMK

SEQ ID NO: 220 - protein, 14 aa, Artificial Sequence
  Protein transport protein Sec31A (SEC31-like protein 1) (SEC31-related protein A)
QVQHILASASPSGR

SEQ ID NO: 221 - protein, 17 aa, Artificial Sequence
  Serine hydroxymethyltransferase, mitochondrial (SHMT) (EC 2.1.2.1) (Glycine hydroxymethyltransferase) (Serine methylase)
AHLLADMAHISGLVAAK

SEQ ID NO: 222 - protein, 11 aa, Artificial Sequence
  Serine hydroxymethyltransferase, mitochondrial (SHMT) (EC 2.1.2.1) (Glycine hydroxymethyltransferase) (Serine methylase)
LIIAGTSAYAR

SEQ ID NO: 223 - protein, 7 aa, Artificial Sequence
  Serine hydroxymethyltransferase, mitochondrial (SHMT) (EC 2.1.2.1) (Glycine hydroxymethyltransferase) (Serine methylase)
VIPSPFK

SEQ ID NO: 224 - protein, 17 aa, Artificial Sequence
  Choline transporter-like protein 1 (Solute carrier family 44 member 1) (CD antigen CD92)
FAEALITFVSDNSVLHR

SEQ ID NO: 225 - protein, 14 aa, Artificial Sequence
  Choline transporter-like protein 1 (Solute carrier family 44 member 1) (CD antigen CD92)
LPVPASAPIPFFHR

SEQ ID NO: 226 - protein, 13 aa, Artificial Sequence
  Choline transporter-like protein 1 (Solute carrier family 44 member 1) (CD antigen CD92)
NLPFTPILASVNR

SEQ ID NO: 227 - protein, 19 aa, Artificial Sequence
  Osteopontin (2AR) (Bone sialoprotein 1) (Calcium oxalate crystal growth inhibitor protein) (Early T-lymphocyte activation 1 protein) (Minopontin) (Secreted phosphoprotein 1) (SPP-1)
ESQESADQSDVIDSQASSK

SEQ ID NO: 228 - protein, 9 aa, Artificial Sequence
  Osteopontin (2AR) (Bone sialoprotein 1) (Calcium oxalate crystal growth inhibitor protein) (Early T-lymphocyte activation 1 protein) (Minopontin) (Secreted phosphoprotein 1) (SPP-1)
GDSLAYGLR

SEQ ID NO: 229 - protein, 20 aa, Artificial Sequence
  Osteopontin (2AR) (Bone sialoprotein 1) (Calcium oxalate crystal growth inhibitor protein) (Early T-lymphocyte activation 1 protein) (Minopontin) (Secreted phosphoprotein 1) (SPP-1)
LYSLHPDPIATWLVPDPSQK

SEQ ID NO: 230 - protein, 11 aa, Artificial Sequence
  Spectrin alpha chain, non-erythrocytic 1 (Alpha-II spectrin) (Fodrin alpha chain)
DLSSVQTLLTK

SEQ ID NO: 231 - protein, 13 aa, Artificial Sequence
  Spectrin alpha chain, non-erythrocytic 1 (Alpha-II spectrin) (Fodrin alpha chain)
LDENSAFLQFNWK

SEQ ID NO: 232 - protein, 10 aa, Artificial Sequence
  Spectrin alpha chain, non-erythrocytic 1 (Alpha-II spectrin) (Fodrin alpha chain)
SSEEIESAFR

SEQ ID NO: 233 - protein, 9 aa, Artificial Sequence
  Spectrin beta chain, non-erythrocytic 1 (Beta-II spectrin) (Embryonic liver fodrin) (Fodrin beta chain)
VAVVNQIAR

SEQ ID NO: 234 - protein, 10 aa, Artificial Sequence
  Spectrin beta chain, non-erythrocytic 1 (Beta-II spectrin) (Embryonic liver fodrin) (Fodrin beta chain)
VLDNAIETEK

SEQ ID NO: 235 - protein, 9 aa, Artificial Sequence
  Spectrin beta chain, non-erythrocytic 1 (Beta-II spectrin) (Embryonic liver fodrin) (Fodrin beta chain)
VQAVVAVAR

SEQ ID NO: 236 - protein, 9 aa, Artificial Sequence
  Serine/arginine-rich splicing factor 7 (Splicing factor, arginine/serine-rich 7)
AFSYYGPLR

SEQ ID NO: 237 - protein, 15 aa, Artificial Sequence
  Serine/arginine-rich splicing factor 7 (Splicing factor, arginine/serine-rich 7)
RPFDPNDRCYECGEK

SEQ ID NO: 238 - protein, 9 aa, Artificial Sequence
  Serine/arginine-rich splicing factor 7 (Splicing factor, arginine/serine-rich 7)
VELSTGMPR

SEQ ID NO: 239 - protein, 7 aa, Artificial Sequence
  Lupus La protein homolog (La autoantigen homolog) (La ribonucleoprotein)
EGIILFK

SEQ ID NO: 240 - protein, 16 aa, Artificial Sequence
  Lupus La protein homolog (La autoantigen homolog) (La ribonucleoprotein)
ICHQIEYYFGDFNLPR

SEQ ID NO: 241 - protein, 10 aa, Artificial Sequence
  Lupus La protein homolog (La autoantigen homolog) (La ribonucleoprotein)
NANNGNLLLR

SEQ ID NO: 242 - protein, 8 aa, Artificial Sequence
  Single-stranded DNA-binding protein, mitochondrial (Mt-SSB) (MtSSB)
DVAYQYVK

SEQ ID NO: 243 - protein, 15 aa, Artificial Sequence
  Single-stranded DNA-binding protein, mitochondrial (Mt-SSB) (MtSSB)
NPVTIFSLATNEMWR

SEQ ID NO: 244 - protein, 15 aa, Artificial Sequence
  Single-stranded DNA-binding protein, mitochondrial (Mt-SSB) (MtSSB)
SGDSEVYQMGDVSQK

SEQ ID NO: 245 - protein, 10 aa, Artificial Sequence
  Sushi, von Willebrand factor type A, EGF and pentraxin domain-containing protein 1 (Polydom)
FAAGSVVSFK

SEQ ID NO: 246 - protein, 11 aa, Artificial Sequence
  Sushi, von Willebrand factor type A, EGF and pentraxin domain-containing protein 1 (Polydom)
GAFQQAAQILR

SEQ ID NO: 247 - protein, 9 aa, Artificial Sequence
  Sushi, von Willebrand factor type A, EGF and pentraxin domain-containing protein 1 (Polydom)
VAIVTFSSK

SEQ ID NO: 248 - protein, 8 aa, Artificial Sequence
  Threonine--tRNA ligase 1, cytoplasmic (EC 6.1.1.3) (Threonine--tRNA ligase, cytoplasmic) (Threonyl-tRNA synthetase) (ThrRS) (Threonyl-tRNA synthetase 1)
AILGSVER

SEQ ID NO: 249 - protein, 12 aa, Artificial Sequence
  Threonine--tRNA ligase 1, cytoplasmic (EC 6.1.1.3) (Threonine--tRNA ligase, cytoplasmic) (Threonyl-tRNA synthetase) (ThrRS) (Threonyl-tRNA synthetase 1)
NELSGALTGLTR

SEQ ID NO: 250 - protein, 10 aa, Artificial Sequence
  Threonine--tRNA ligase 1, cytoplasmic (EC 6.1.1.3) (Threonine--tRNA ligase, cytoplasmic) (Threonyl-tRNA synthetase) (ThrRS) (Threonyl-tRNA synthetase 1)
VNTPTTTVYR

SEQ ID NO: 251 - protein, 14 aa, Artificial Sequence
  Transformer-2 protein homolog beta (TRA-2 beta) (TRA2-beta) (Silica-induced gene 41 protein) (SIG-41) (Splicing factor, arginine/serine-rich 10) (Transformer-2 protein homolog B)
GFAFVYFENVDDAK

SEQ ID NO: 252 - protein, 16 aa, Artificial Sequence
  Transformer-2 protein homolog beta (TRA-2 beta) (TRA2-beta) (Silica-induced gene 41 protein) (SIG-41) (Splicing factor, arginine/serine-rich 10) (Transformer-2 protein homolog B)
YGPIADVSIVYDQQSR

SEQ ID NO: 253 - protein, 10 aa, Artificial Sequence
  Tumor susceptibility gene 101 protein (ESCRT-I complex subunit TSG101)
ASLISAVSDK

SEQ ID NO: 254 - protein, 10 aa, Artificial Sequence
  Tumor susceptibility gene 101 protein (ESCRT-I complex subunit TSG101)
DGTISEDTIR

SEQ ID NO: 255 - protein, 12 aa, Artificial Sequence
  Tumor susceptibility gene 101 protein (ESCRT-I complex subunit TSG101)
ELVNLTGTIPVR

SEQ ID NO: 256 - protein, 17 aa, Artificial Sequence
  NEDD8-activating enzyme E1 catalytic subunit (EC 6.2.1.64) (NEDD8-activating enzyme E1C) (Ubiquitin-activating enzyme E1C) (Ubiquitin-like modifier-activating enzyme 3) (Ubiquitin-activating enzyme 3)
QIHVIDMDTIDVSNLNR

SEQ ID NO: 257 - protein, 11 aa, Artificial Sequence
  NEDD8-activating enzyme E1 catalytic subunit (EC 6.2.1.64) (NEDD8-activating enzyme E1C) (Ubiquitin-activating enzyme E1C) (Ubiquitin-like modifier-activating enzyme 3) (Ubiquitin-activating enzyme 3)
SPAITATLEGK

SEQ ID NO: 258 - protein, 7 aa, Artificial Sequence
  Ubiquitin-conjugating enzyme E2 N (EC 2.3.2.23) (Bendless-like ubiquitin-conjugating enzyme) (E2 ubiquitin-conjugating enzyme N) (Ubc13) (Ubiquitin carrier protein N) (Ubiquitin-protein ligase N)
ICLDILK

SEQ ID NO: 259 - protein, 15 aa, Artificial Sequence
  Ubiquitin-conjugating enzyme E2 N (EC 2.3.2.23) (Bendless-like ubiquitin-conjugating enzyme) (E2 ubiquitin-conjugating enzyme N) (Ubc13) (Ubiquitin carrier protein N) (Ubiquitin-protein ligase N)
LELFLPEEYPMAAPK

SEQ ID NO: 260 - protein, 11 aa, Artificial Sequence
  Ubiquitin-conjugating enzyme E2 N (EC 2.3.2.23) (Bendless-like ubiquitin-conjugating enzyme) (E2 ubiquitin-conjugating enzyme N) (Ubc13) (Ubiquitin carrier protein N) (Ubiquitin-protein ligase N)
TNEAQAIETAR

SEQ ID NO: 261 - protein, 10 aa, Artificial Sequence
  Regulator of nonsense transcripts 1 (EC 3.6.4.-) (ATP-dependent helicase RENT1) (Nonsense mRNA reducing factor 1) (NORF1) (Up-frameshift suppressor 1 homolog) (mUpf1)
AGLSQSLFER

SEQ ID NO: 262 - protein, 14 aa, Artificial Sequence
  Regulator of nonsense transcripts 1 (EC 3.6.4.-) (ATP-dependent helicase RENT1) (Nonsense mRNA reducing factor 1) (NORF1) (Up-frameshift suppressor 1 homolog) (mUpf1)
GNTSGSHIVNHLVR

SEQ ID NO: 263 - protein, 11 aa, Artificial Sequence
  Regulator of nonsense transcripts 1 (EC 3.6.4.-) (ATP-dependent helicase RENT1) (Nonsense mRNA reducing factor 1) (NORF1) (Up-frameshift suppressor 1 homolog) (mUpf1)
NVFLLGFIPAK

SEQ ID NO: 264 - protein, 13 aa, Artificial Sequence
  General vesicular transport factor p115 (Protein USO1 homolog) (Transcytosis-associated protein) (TAP) (Vesicle-docking protein)
TLEQHDNIVTHYK

SEQ ID NO: 265 - protein, 10 aa, Artificial Sequence
  General vesicular transport factor p115 (Protein USO1 homolog) (Transcytosis-associated protein) (TAP) (Vesicle-docking protein)
TNNSNQNFFK

SEQ ID NO: 266 - protein, 17 aa, Artificial Sequence
  General vesicular transport factor p115 (Protein USO1 homolog) (Transcytosis-associated protein) (TAP) (Vesicle-docking protein)
VLVSPTNPPGATSSCQK

SEQ ID NO: 267 - protein, 11 aa, Artificial Sequence
  Vesicle-associated membrane protein-associated protein A (VAMP-A) (VAMP-associated protein A) (VAP-A) (33 kDa VAMP-associated protein) (VAP-33)
EAKPDELMDSK

SEQ ID NO: 268 - protein, 14 aa, Artificial Sequence
  Vesicle-associated membrane protein-associated protein A (VAMP-A) (VAMP-associated protein A) (VAP-A) (33 kDa VAMP-associated protein) (VAP-33)
HEQILVLDPPSDLK

SEQ ID NO: 269 - protein, 16 aa, Artificial Sequence
  Vesicle-associated membrane protein-associated protein A (VAMP-A) (VAMP-associated protein A) (VAP-A) (33 kDa VAMP-associated protein) (VAP-33)
VAHSDKPGSTSAVSFR

SEQ ID NO: 270 - DNA, 24 bp, Artificial Sequence
  miR-182-5p
uuuggcaaugguagaacucacacu

SEQ ID NO: 271 - DNA, 23 bp, Artificial Sequence
  miR-542-5p
ucggggaucaucaugucacgaga

(19) United States
(12) Patent Application Publication
(10) Pub. No.: US 2023/0332232 A1
(43) Pub. Date: Oct. 19, 2023

(54) COMPOSITIONS AND METHODS FOR DIAGNOSING AND TREATING A DYSTONIA

(71) Applicant: Duke University, Durham, NC (US)

(72) Inventors: Nicole Calakos, Durham, NC (US); Zachary Caffall, Durham, NC (US); Connor King, Durham, NC (US)

(73) Assignee: Duke University, Durham, NC (US)

(21) Appl. No.: 18/023,933

(22) PCT Filed: Sep. 14, 2021
(86) PCT No.: PCT/US21/50296

§ 371 Date: Feb. 28, 2023

Related U.S. Application Data
(60) Provisional application No. 63/077,754, filed on Sep. 14, 2020.
(60) Provisional application No. 63/155,046, filed on Mar. 1, 2021.

(51) Int. Cl. C12Q 1/6883; G01N 33/68; A61K 31/427

(52) U.S. Cl. CPC C12Q 1/6883; G01N 33/6893; A61K 31/427; C12Q 2600/178; C12Q 2600/158; G01N 2800/2835; G01N 2800/52

(57) ABSTRACT

Disclosed herein are compositions, kits, and methods for identifying protein or miRNA biomarkers for dystonia, for treating a subject having a dystonia, for predicting penetrance of a dystonia in a subject, and for predicting responsiveness to a dystonia treatment.

### I. CROSS-REFERENCE TO RELATED APPLICATIONS

This application is a national phase application of International Application No. PCT/US21/50296 filed 14 Sep. 2021, which claims the benefit of priority to U.S. Provisional Application Ser. No. 63/077,754 filed 14 Sep. 2020 and U.S. Provisional Patent Application No. 63/155,046 filed 1 Mar. 2021, the contents of each of which are incorporated by reference herein in their entirety.

### II. REFERENCE TO THE SEQUENCE LISTING

The Sequence Listing submitted 14 Sep. 2021 as a text file named “21_2024_WO_Sequence_Listing”, created on 14 Sep. 2021 and having a size of 68 kilobytes is hereby incorporated by reference pursuant to 37 C.F.R. § 1.52(e)(5).

### III. BACKGROUND

Dystonias are centrally driven movement disorders characterized by sustained involuntary postures and/or slow twisting movements that lead to motor disability and pain. Presentations range from focal dystonias, affecting single limbs or other body parts, to generalized dystonias where most of the body is involved in abnormal posturing and/or slow uncontrolled twisting movements. Once symptoms manifest, they typically endure throughout an individual's lifetime leading to a notable burden of disability and pain. In all its forms, dystonia is the third most common movement disorder after Parkinson's disease and essential tremor and can arise in many clinical settings—from sporadic and inherited forms to those that occur in association with traumatic brain injury, stroke, neurodegenerative diseases, metabolic disorders, or antipsychotic medication use.

The mainstays for oral medication treatment are anticholinergic drugs, benzodiazepines, and muscle relaxants. These medications typically reduce the intensity of, but do not eliminate, dystonia symptoms. The narrow therapeutic window that further limits the utility of these medications. While pallidal deep brain stimulation surgery has been shown to be beneficial for some subsets of patients with dystonia, including those with DYT1 dystonia, this is a highly invasive treatment available only at tertiary care centers. None of these treatments are disease-modifying. Thus, there is a major unmet need for the efficient diagnosis of a dystonia and for effective, affordable, and easily accessible dystonia treatments.

### IV. BRIEF DESCRIPTION OF THE FIGURES

FIGS. 1A-1C shows the isolation and evaluation of extracellular vesicles from conditioned media. FIG. 1A shows MEF culture media incubated for 24 hours prior to harvest and EV enrichment through ultracentrifugation. FIG. 1B shows a representative Western blot demonstrating enrichment of the EV marker (TSG101) and depletion of the endoplasmic reticulum (ER) marker (CANX or Calnexin) for Cohort 1. FIG. 1C shows a representative Western blot demonstrating enrichment of TSG101 and CANX for Cohort 2.

FIGS. 2A-2B shows the proteomic results demonstrating a strong correlation between independent cohort LC/MS/MS runs. FIG. 2A shows the EVs for Cohort 1 vs Cohort 2 isolated from WT while FIG. 2B shows the EVs for Cohort 1 vs. Cohort 2 isolated from DYT1 mice.

FIGS. 3A-3D shows that DYT1-genotype dependent shifts in EV proteome. FIG. 3A and FIG. 3B show volcano plots showing the Fold Change (FC) of DYT1(ΔE)/WT and uncorrected p-value results for all proteins detected in proteomic analysis of Cohort 1 (1059 proteins) and Cohort 2 (1974 proteins), respectively. FIG. 3C shows a Venn diagram demonstrating the overlap of detected proteins between Cohort1 (1059 proteins) and Cohort 2 (1974 proteins). The 51 proteins each had a combined p-value <0.01. FIG. 3D shows a comparison of a Log 2 fold change from Cohort 1 and Cohort 2. Symbol color represents Fisher's combined probability test for Cohort 1 and Cohort 2.

FIG. 4 shows the bioinformatic pathway analysis for 202 of the 1008 overlapping proteins in Cohort 1 and Cohort 2 (FIG. 3C) with mean FC>2 and a Fisher's combined p value<0.01. This analysis yielded the identified several pathways including pathways previously associated with DYT1.

FIG. 5A shows the hierarchical clustering of all proteins differentially expressed between WT cells and the DYT1 cells in Cohort 1, wherein green represents up-regulated expression of proteins in DYT1 cells and red represents down-regulated expression of proteins in DYT1 cells as compared to WT cells. FIG. 5B shows that the dysregulation of proteins in DYT1 cells was normalized by RTV in Cohort 2.

FIGS. 6A-6B show the RTV effect on EV composition in DYT1 cells (FIG. 6B) compared to WT cells (FIG. 6A) for the 363 proteins in the DYT1 Genotype Dependent Difference subset.

FIGS. 7A-7B show that ISRIB biases the distribution of EV biomarkers in WT cells (FIG. 7B) towards that of the DYT1 cells (FIG. 7A) for the 363 proteins in the DYT1 Genotype Dependent Difference.

FIGS. 8A-8B show the differential EV derived total RNA-seq (FIG. 8A) and microRNA-seq (FIG. 8B) analysis in DYT1 cells and WT cells (e.g., DYT1(ΔE)/WT).

FIGS. 9A-9B show the RTV effect on EV composition in DYT1 cells (FIG. 9B) compared to WT cells (FIG. 9A) for 279 miRNAs.

FIG. 10A shows the effect on 279 miRNAs in WT cells treated with ISRIB compared to non-treated WT cells while FIG. 10B shows the effect on the same 279 miRNAs in DYT1 cells treated with SAL compared to non-treated DYT1 cells.

FIGS. 11A-11B show that there was a strong correlation between the RNAseq analysis and RT-qPCR analysis for the RTV effect in DYT1 cells vs. WT cells (FIG. 11B) compared to non-treated DYT1 cells vs. WT cells (FIG. 11A).

FIG. 12 shows that although the specific disruptions in the eIF2a pathway are distinct among these three dystonias, they converge upon a common consequence of reduced eIF2a pathway signaling.

### V. BRIEF SUMMARY

Disclosed herein is a method of identifying a dystonia biomarker in a subject, the method comprising obtaining a biosample from a subject having a dystonia; obtaining a biosample from a subject not having a dystonia; determining the expression level of one or more proteins in both biosamples; identifying those proteins that are differentially expressed in the biosample obtained from the subject having a dystonia when compared to the biosample from the subject not having a dystonia; wherein those differentially expressed proteins are biomarkers of a dystonia.

Disclosed herein is a method of identifying a dystonia biomarker in a subject, the method comprising obtaining a biosample from a subject having a dystonia; determining the expression level of one or more proteins in the biosample; identifying those proteins that are differentially expressed in the dystonia biosample when compared to that of a reference biosample; wherein those differentially expressed proteins are biomarkers of a dystonia.

Disclosed herein is a method of treating a subject having a dystonia, the method comprising obtaining a biosample from a subject after treatment; determining the expression level of one or more proteins in the post-treatment biosample, wherein: if the post-treatment expression level represents an improvement over a pre-treatment expression level of the one or more proteins, or if the post-treatment expression level is within an acceptable range of a reference expression level, then continuing to administer the treatment.

Disclosed herein is a method of predicting penetrance of a dystonia in a subject comprising obtaining a biosample from a subject; determining the expression level of one or more proteins in the biosample; identifying those proteins that are differentially expressed in the biosample when compared to a reference biosample; wherein the degree of differential expression predicts the likelihood of penetrance.

Disclosed herein is a method of predicting responsiveness to a treatment, the method comprising obtaining a biosample from a subject having a dystonia; determining the expression level of one or more proteins in the biosample to create a proteomic profile; comparing the subject's proteomic profile to a proteomic profile of a treatment-responsive subject; and if the proteomic profiles are similar, then predicting that the subject having a dystonia will be responsive to the treatment, and if the proteomic profiles are dissimilar, then predicting that the subject having a dystonia will not be responsive to the treatment.

Disclosed herein is a method of identifying a dystonia biomarker in a subject comprising obtaining a biosample from a subject having a dystonia; obtaining a biosample from a subject not having a dystonia; determining the expression level of one or more miRNAs in both biosamples; identifying those miRNAs that are differentially expressed in the biosample obtained from the subject having a dystonia when compared to the biosample from the subject not having a dystonia; wherein those differentially expressed miRNAs are biomarkers of a dystonia.

Disclosed herein is a method of identifying a dystonia biomarker in a subject comprising obtaining a biosample from a subject having a dystonia; determining the expression level of one or more miRNAs in the biosample; identifying those miRNAs that are differentially expressed in the dystonia biosample when compared to that of a reference biosample; wherein those differentially expressed miRNAs are biomarkers of a dystonia.

Disclosed herein is a method of treating a subject having a dystonia comprising obtaining a biosample from a subject after treatment; determining the expression level of one or more miRNAs in the post-treatment biosample, wherein if the post-treatment expression level represents an improvement over a pre-treatment expression level of the one or more miRNAs, or if the post-treatment expression level is within an acceptable range of a reference expression level, then continuing to administer the treatment.

Disclosed herein is a method of predicting penetrance of a dystonia in a subject comprising obtaining a biosample from a subject; determining the expression level of one or more miRNAs in the biosample; identifying those miRNAs that are differentially expressed in the biosample when compared to a reference biosample; wherein the degree of differential expression predicts the likelihood of penetrance.

Disclosed herein is a method of predicting responsiveness to a treatment comprising obtaining a biosample from a subject having a dystonia; determining the expression level of one or more miRNAs in the biosample to create a miRNA profile; comparing the subject's miRNA profile to a miRNA profile of a treatment-responsive subject; and if the profiles are similar, then predicting that the subject having a dystonia will be responsive to the treatment, and if the profiles are dissimilar, then predicting that the subject having a dystonia will not be responsive to the treatment.

### VI. DETAILED DESCRIPTION

The present disclosure describes formulations, compounded compositions, kits, capsules, containers, and/or methods thereof. It is to be understood that the inventive aspects of which are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### A. Relevant Definitions

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

This disclosure describes inventive concepts with reference to specific examples. However, the intent is to cover all modifications, equivalents, and alternatives of the inventive concepts that are consistent with this disclosure.

As used in the specification and the appended claims, the singular forms “a”, “an”, and “the” include plural referents unless the context clearly dictates otherwise.

The phrase “consisting essentially of” limits the scope of a claim to the recited components in a composition or the recited steps in a method as well as those that do not materially affect the basic and novel characteristic or characteristics of the claimed composition or claimed method. The phrase “consisting of” excludes any component, step, or element that is not recited in the claim. The phrase “comprising” is synonymous with “including”, “containing”, or “characterized by”, and is inclusive or open-ended. “Comprising” does not exclude additional, unrecited components or steps.

As used herein, when referring to any numerical value, the term “about” means a value falling within a range that is +10% of the stated value.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5, and are present in such ratio regardless of whether additional components are contained in the compound.

As used herein, the terms “optional” or “optionally” means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

As used herein, the term “subject” refers to the target of administration, e.g., a human being. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, or rodent. The term does not denote a particular age or sex, and thus, adult and child subjects, as well as fetuses, whether male or female, are intended to be covered. In an aspect, a subject can be a human patient. In an aspect, a subject can have a dystonia, be suspected of having a dystonia, or be at risk of developing and/or acquiring a dystonia.

As used herein, the term “diagnosed” means having been subjected to an examination by a person of skill, for example, a physician, and found to have a condition that can be diagnosed or treated by one or more of the disclosed agents, disclosed therapeutic agents, disclosed pharmaceutical formulations, or a combination thereof, or by one or more of the disclosed methods. For example, “diagnosed with a dystonia” means having been subjected to an examination by a person of skill, for example, a physician, and found to have a condition that can be treated by one or more of the disclosed agents, disclosed therapeutic agents, disclosed pharmaceutical formulations or a combination thereof, or by one or more of the disclosed methods. For example, “suspected of having a dystonia” can mean having been subjected to an examination by a person of skill, for example, a physician, and found to have a condition that can likely be treated by one or more of the disclosed agents, disclosed therapeutic agents, disclosed pharmaceutical formulations or a combination thereof, or by one or more of the disclosed methods. In an aspect, an examination can be physical, can involve various tests (e.g., blood tests, genotyping, biopsies, etc.) and assays (e.g., enzymatic assay), or a combination thereof.

A “patient” can refer to a subject that has been diagnosed with or is suspected of having a dystonia. In an aspect, a patient can refer to a subject that has been diagnosed with or is suspected of having a dystonia such as for example, DYT1, and is seeking treatment or receiving treatment for a dystonia (such as DYT1).

As used herein, the phrase “identified to be in need of treatment for a disorder,” or the like, refers to selection of a subject based upon need for treatment of the disorder. For example, a subject can be identified as having a need for treatment of a disorder (e.g., such as a dystonia) based upon an earlier diagnosis by a person of skill and thereafter subjected to treatment for the disorder (e.g., a dystonia). In an aspect, the identification can be performed by a person different from the person making the diagnosis. In an aspect, the administration can be performed by one who performed the diagnosis.

As used herein, the term “movement disorder” includes neurological diseases or disorders that involve the motor and movement systems, resulting in a range of abnormalities that affect the speed, quality, and ease of movement. Movement disorders are often caused by or related to abnormalities in brain structure and/or function. Movement disorders include, but are not limited to (i) tremors: including, but not limited to, the tremor associated with Parkinson's Disease, physiologic tremor, benign familial tremor, cerebellar tremor, rubral tremor, toxic tremor, metabolic tremor, and senile tremor; (ii) chorea, including, but not limited to, chorea associated with Huntington's Disease, Wilson's Disease, ataxia telangiectasia, infection, drug ingestion, or metabolic, vascular or endocrine etiology (e.g., chorea gravidarum or thyrotoxicosis); (iii) ballism (defined herein as abruptly beginning, repetitive, wide, flinging movements affecting predominantly the proximal limb and girdle muscles); (iv) athetosis (defined herein as relatively slow, twisting, writhing, snake-like movements and postures involving the trunk, neck, face and extremities); (v) dystonia (defined herein as a movement disorder consisting of twisting, turning tonic skeletal muscle contractions, most, but not all of which are initiated distally); (vi) paroxysmal choreoathetosis and tonic spasm; (vii) tics (defined herein as sudden, behaviorally related, irregular, stereotyped, repetitive movements of variable complexity); (viii) tardive dyskinesia; (ix) akathesia, (x) muscle rigidity, defined herein as resistance of a muscle to stretch; (xi) postural instability; (xii) bradykinesia; (xiii) difficulty in initiating movements; (xiv) muscle cramps; (xv) dyskinesias and (xvi) myoclonus. In an aspect, a movement disorder comprises a dystonia.

As used herein, the terms “neurological diseases” or “neurological disorders” are used interchangeably and refer to a host of undesirable conditions affecting neurons in the brain of a subject. These diseases include but are not limited to the following: Alzheimer's disease, Parkinson's disease, Huntington's disease, Pick's disease, Kuf's disease, Lewy body disease, neurofibrillary tangles, Rosenthal fibers, Mallory's hyaline, senile dementia, myasthenia gravis, Gilles de la Tourette's syndrome, multiple sclerosis (MS), amyotrophic lateral sclerosis (ALS), progressive supranuclear palsy (PSP), epilepsy, Creutzfeldt-Jakob disease, deafness-dytonia syndrome, Leigh syndrome, Leber hereditary optic neuropathy (LHON), parkinsonism, dystonia, motor neuron disease, neuropathy-ataxia and retinitis pimentosa (NARP), maternal inherited Leigh syndrome (MILS), Friedreich ataxia, hereditary spastic paraplegia, Mohr-Tranebjaerg syndrome, Wilson disease, sporatic Alzheimer's disease, sporadic amyotrophic lateral sclerosis, sporadic Parkinson's disease, autonomic function disorders, hypertension, sleep disorders, neuropsychiatric disorders, depression, schizophrenia, schizoaffective disorder, korsakoff's psychosis, mania, anxiety disorders, phobic disorder, learning or memory disorders, amnesia or age-related memory loss, attention deficit disorder, dysthymic disorder, major depressive disorder, obsessive-compulsive disorder, psychoactive substance use disorders, panic disorder, bipolar affective disorder, severe bipolar affective (mood) disorder (BP-1), migraines, hyperactivity and movement disorders.

In an aspect, any disclosed method can be applied in the context of one or more neurological diseases or disorders. For example, a disclosed method of identifying a dystonia biomarker can be applied instead with a subject having Alzheimer's disease, depression, or anxiety, or any other disclosed neurological disease or disorder. The same applies to the other methods disclosed herein, including but not limited to, methods of treating a subject having a neurological disease or disorder, methods of predicting penetrance and/or severity of a neurological disease or disorder, and/or methods of predicting responsiveness to a treatment.

As used herein, the term “agent which prevents or reduces symptoms of the neurological disorder” or “agent used for the treatment of a neurological disorder” refers to those drugs that are used for the treatment of one or more of the disclosed neurological diseases and disorders. Examples of such agents include, but are not limited to, the following: anticholinergics, such as trihexyphenidyl (Artane®), benztropine (Cogentin®), ethopropazine (Parsitan®); benzodiazepines, such as diazepam (Valium®), clonazepam (Klonopin®), lorazepam (Ativan®); baclofen (Lioresal®), dopaminergic agents such as levodopa (Sinemet®) and bromocriptine (Parlodel®); tetrabenazine (Xenazine®), dopamine-depleting agents, ritonavir, lopinavir, and the like. In an aspect, the agent comprises ritonavir.

Other agents that treat, prevent, inhibit, and/or ameliorate symptoms and/or complications of a neurological disorder and/or a neurodegenerative disease include the following: Acamprosate tablets (Campral EC), Adrenaline (epinephrine) (Emerade, EpiPen, Jext), Agomelatine tablets (Valdoxan), Almotriptan (Almogran), Amantadine, Amisulpride (Solian), Amitriptyline (Elavil), Apomorphine (APO-go, Dacepton), Aripiprazole (Abilify), Aripiprazole long-acting injection (Abilify Maintena), Asenapine tablets (Sycrest), Atomoxetine (Strattera), Baclofen (Lyflex, Lioresal), Botulinum toxin type A (Botox), Bromocriptine (Parlodel), Buccal midazolam (Buccolam, Epistatus), Buprenorphine (BuTrans, Hapoctasin, Temgesic, Tephine, Transtec), Buspirone, Cabergoline tablets (Dostinex, Cabaser), Carbamazepine (Curatil, Tegretol), Chlordiazepoxide (Librium), Chlorpromazine, Citalopram (Cipramil, Celexa), Clobazam (Frisium, Perizam, Tapclob, Zacco), Clomethiazole, Clomipramine, Clonazepam, Clozapine (Clozaril, Denzapine, Zaponex), Co-beneldopa (Madopar), Co-careldopa (Sinemet), Dantrolene (Dantrium), Dexamfetamine (Amfexa), Diazepam (Diazemuls, Stesolid), Divalproex sodium (Depakote), Donepezil (Aricept), Doxepin capsules, Duloxetine (Cymbalta, Depalta, Duciltia), Eletriptan (Relpax), Entacapone (Comtess), Escitalopram (Cipralex), Eslicarbazepine (Zebinix), Ethosuximide, Fingolimod capsules (Gilenya), Fluoxetine (Olena, Prozac, Prozep), Flupentixol long-acting injection (Depixol, Psytixol), Flupentixol tablets (Depixol, Fluanxol), Fluphenazine long-acting injection (Modecate), Fluvoxamine tablets (Faverin), Frovatriptan for migraine (Migard), Gabapentin (Neurontin), Galantamine (Acumor, Consion, Elmino, Gaalin, Galsya, Galzemic, Gatalin, Gazylan, Lotprosin, Luventa, Reminyl), Haloperidol (Haldol, Serenace), Haloperidol long-acting injection (Haldol Decanoate), Hydromorphone (Palladone), Imipramine tablets and liquid medicine, Lacosamide (Vimpat), Lamotrigine (Lamictal), Levetiracetam for epilepsy (Keppra, Desitrend), Levomepromazine tablets (Nozinan), Lisdexamfetamine (Elvanse), Lithium tablets and liquid medicine (Camcolit, Liskonum, Priadel, Li-Liquid), Lofepramine, Loprazolam, Lorazepam, Lormetazepam tablets, Lurasidone (Latuda), Melatonin tablets (Circadin, Slenyto), Memantine (Ebixa, Nemtadine), Methylphenidate (Concerta, Equasym, Medikinet, Ritalin, Tranquilyn), Mianserin, Midodrine (Bramox), Mirtazapine (Zispin SolTab), Moclobemide (Manerix), Modafinil tablets (Provigil), Morphine (Morphgesic, Oramorph, Zomorph), Naratriptan (Naramig), Neostigmine, Nitrazepam (Mogadon), Nortriptyline tablets, Olanzapine (Zalasta, Zyprexa), Olanzapine long-acting injection (Zypadhera), Orlistat capsules (Alli, Beacita, Orlos, Xenical), Orphenadrine, Oxazepam, Oxcarbazepine (Trileptal), Oxycodone (Abtard, Longtec, OxyContin, OxyNorm, Shortec), Paliperidone (Invega), Paliperidone long-acting injection (Xeplion, Trevicta), Paroxetine (Seroxat), Perampanel (Fycompa), Pergolide, Pericyazine, Phenobarbital, Phenytoin (Epanutin), Piracetam (Nootropil), Pizotifen tablets, Pramipexole tablets (Mirapexin, Oprymea, Pipexus, Glepark), Pregabalin (Alzain, Axalid, Lecaent, Lyrica), Primidone, Prochlorperazine (Buccastem, Stemetil), Procyclidine (Kemadrin), Pyridostigmine (Mestinon), Quetiapine (Seroquel), Rasagiline (Azilect), Reboxetine tablets (Edronax), Risperidone (Risperdal), Risperidone long-acting injection (Risperdal Consta), Rivastigmine (Alzest, Exelon, Nimvastid), Rizatriptan for migraine (Maxalt), Ropinirole tablets (Requip, Adartrel), Rotigotine patches (Neupro), Rufinamide for epilepsy (Inovelon), Selegiline (Eldepryl), Sertraline (Lustral, Zoloft), Sodium oxybate (Xyrem), Sodium valproate (Epilim, Episenta, Epival, Convulex), Sulpiride, Sumatriptan (Imigran), Temazepam, Tetrabenazine tablets (Tardiben, Xenazine), Tiagabine (Gabitril), Tizanidine, Tolcapone (Tasmar), Topiramate (Topamax), Topiramate (Topamax), Trazodone (Molipaxin), Trihexyphenidyl, Trimipramine, Valproate semisodium (Belvo, Depakote, Syonell), Venlafaxine (Efexor XL, Effexor XR), Vigabatrin (Sabril, Kigabeq), Vortioxetine (Brintellix), Zolmitriptan (Zomig), Zolpidem tablets (Stilnoct), Zonisamide (Zonegran, Desizon), Zopiclone tablets (Zimovane), and Zuclopenthixol (Clopixol). In an aspect, any one or combination of these agents can be a therapeutic agent used in a disclosed method.

As used herein, “inhibit,” “inhibiting”, and “inhibition” mean to diminish or decrease an activity, level, response, condition, severity, disease, or other biological parameter. This can include, but is not limited to, the complete ablation of the activity, level, response, condition, severity, disease, or other biological parameter. This can also include, for example, a 10% inhibition or reduction in the activity, level, response, condition, severity, disease, or other biological parameter as compared to the native or control level (e.g., a subject not having a dystonia). Thus, in an aspect, the inhibition or reduction can be a 10%, 20%, 30%, 40%, 50%, 60%, 70%, 80%, 90%, 100%, or any amount of reduction in between as compared to native or control levels. In an aspect, the inhibition or reduction can be 10-20%, 20-30%, 30-40%, 40-50%, 50-60%, 60-70%, 70-80%, 80-90%, or 90-100% as compared to native or control levels. In an aspect, the inhibition or reduction can be 0-25%, 25-50%, 50-75%, or 75-100% as compared to native or control levels. In an aspect, a native or control level can be a pre-disease or pre-disorder level.

The words “treat” or “treating” or “treatment” include palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder (such as a dystonia). In an aspect, the terms cover any treatment of a subject, including a mammal (e.g., a human), and includes: (i) preventing the undesired physiological change, disease, pathological condition, or disorder from occurring in a subject that can be predisposed to the disease but has not yet been diagnosed as having it; (ii) inhibiting the physiological change, disease, pathological condition, or disorder, i.e., arresting its development; or (iii) relieving the physiological change, disease, pathological condition, or disorder, i.e., causing regression of the disease. For example, in an aspect, treating a dystonia (such as DYT1) can reduce the severity of an established dystonia in a subject by 1%-100% as compared to a control (such as, for example, an individual not having a dystonia). In an aspect, treating can refer to a 1%, 2%, 3%, 4%, 5%, 6%, 7%, 8%, 9%, 10%, 20%, 30%, 40%, 50%, 60%, 70%, 80%, 90%, or 100% reduction in the severity of a dystonia. For example, treating a dystonia can reduce one or more symptoms of a dystonia in a subject by 1%-100% as compared to a control (such as, for example, an individual not having a dystonia). In an aspect, treating can refer to 1%, 2%, 3%, 4%, 5%, 6%, 7%, 8%, 9%, 10%, 20%, 30%, 40%, 50%, 60%, 70%, 80%, 90%, 100% reduction of one or more symptoms of an established dystonia (such as DYT1). It is understood that treatment does not necessarily refer to a cure or complete ablation or eradication of a dystonia. However, in an aspect, treatment can refer to a cure or complete ablation or eradication of a dystonia.

As used herein, a “biomarker” refers to a defined characteristic that is measured as an indicator of normal biological processes, pathogenic processes, or response to an exposure of intervention. In an aspect, a biomarker can be diagnostic (i.e., detects or classifies a pathological condition), prognostic (i.e., predicts the probability of disease occurrence or progression), pharmacodynamic/responsive (i.e., identifies a change in response to a therapeutic intervention), predictive (i.e., predicts how an individual or subject might respond to a particular intervention or event). In an aspect, a biomarker can be diagnostic, prognostic, pharmacodynamic/responsive, and/or predictive at the same time. In an aspect, a biomarker can be diagnostic, prognostic, pharmacodynamic/responsive, and/or predictive at different times (e.g., first a biomarker can be diagnostic and then later, the same biomarker can be prognostic, pharmacodynamic/responsive, and/or predictive). A biomarker can be an objective measure that can be linked to a clinical outcome assessment. A biomarker can be used by the skilled person to make a clinical decision based on its context of use.

A “diagnostic biomarker” refers to a biomarker that distinguishes between subjects with a particular disease/ailment/condition and those who do not have the disease/ailment/condition.

A “prognostic biomarker” provides information on the likely course of disease/ailment/condition in an individual. A prognostic biomarker can inform the skilled person about the aggressiveness of the disease/ailment/condition and/or the expectation of how a particular subject would fare in the absence of therapeutic intervention. Typically, a prognostic biomarker can identify a patient who is probabilistically at either higher risk for adverse disease-related events or a faster rate of decline in his health status.

A “predictive biomarker” is linked to treatment it provides a forecast of the potential for a subject to respond in some identified manner (which may be favorable or unfavorable) to one or more specific treatments.

A “response biomarker” is a dynamic assessment that shows a biological response has occurred in a subject after having received a therapeutic intervention.

Parallel reaction monitoring (PRM) is an ion monitoring technique based on high-resolution and high-precision mass spectrometry. The principle of this technique is comparable to selected reaction monitoring (SRM/MRM), but it is more convenient in assay development for absolute quantification of proteins and peptides. It is most suitable for quantification of multiple proteins in complex sample with an attomole-level detection. Parallel reaction monitoring (PRM) is an increasingly popular alternative to SRM for targeted proteomics. PRM's strengths over SRM are that it monitors all product ions in a single spectrum, thus eliminating the need to select interference-free product ions prior to data acquisition, and that it is most frequently performed on high-resolution instruments, such as quadrupole-orbitrap and quadrupole-time of flight instruments.

As used herein, the term means “increased risk” is used to mean that a subject has an increased chance of developing or acquiring a dystonia when compared a subject known not to have a dystonia (e.g., a control subject). The increased risk may be relative or absolute and may be expressed qualitatively or quantitatively. For example, an increased risk can be expressed as simply determining a subject's proteomic and/or miRNA profile and placing the subject in an “increased risk” category, based upon previous population studies. Alternatively, a numerical expression of the subject's increased risk can be determined based upon the proteomic and/or miRNA profile. As used herein, examples of expressions of an increased risk of developing or acquiring a dystonia can include but are not limited to, odds, probability, odds ratio, p-values, attributable risk, relative frequency, positive predictive value, negative predictive value, and relative risk.

For example, the correlation between a subject's proteomic and/or miRNA profile and the likelihood of developing or acquiring a dystonia can be measured by an odds ratio (OR) and by the relative risk (RR). If P(R⁺) is the probability of developing or acquiring a dystonia for subjects with the risk profile (R) and P(R⁻) is the probability of developing memory impairment for individuals without the risk profile, then the relative risk is the ratio of the two probabilities: RR=P(R⁺)/P(R⁻).

In case-control studies, however, direct measures of the relative risk often cannot be obtained because of sampling design. The odds ratio allows for an approximation of the relative risk for low-incidence diseases and can be calculated: OR=(F⁺/(1−F⁺))/(F⁻/(1−F⁻)), where F⁺ is the frequency of a risk profile in cases studies and F⁻ is the frequency of risk profile in controls. F⁺ and F⁻ can be calculated using the proteomic and/or miRNA profile frequencies of the study.

The attributable risk (AR) can also be used to express an increased risk. The AR describes the proportion of individuals in a population exhibiting memory impairment due to a specific member of the proteomic and/or miRNArisk profile. AR may also be important in quantifying the role of individual components (specific member) in disease etiology and in terms of the public health impact of the individual marker. The public health relevance of the AR measurement lies in estimating the proportion of cases of memory impairment in the population that could be prevented if the profile or individual component were absent. AR may be determined as follows: AR=P_{E}(RR−1)/(P_{E}(RR−1)+1), where AR is the risk attributable to a profile or individual component of the profile, and P_{E} is the frequency of exposure to a profile or individual component of the profile within the population at large. RR is the relative risk, which can be approximated with the odds ratio when the profile or individual component of the profile under study has a relatively low incidence in the general population.

In an aspect, the increased risk of a subject can be determined from p-values that are derived from association studies. Specifically, associations with specific profiles can be performed using regression analysis by regressing the proteomic and/or miRNA profile with developing or acquiring a dystonia. In addition, the regression may or may not be corrected or adjusted for one or more factors. The factors for which the analyses may be adjusted include, but are not limited to age, sex, weight, ethnicity, geographic location, fasting state, state of pregnancy or post-pregnancy, menstrual cycle, general health of the subject, alcohol or drug consumption, caffeine or nicotine intake and circadian rhythms, and the subject's p-EIF2α dysfunction and/or ISR dysfunction to name a few.

Increased risk can also be determined from p-values that are derived using logistic regression. Binomial (or binary) logistic regression is a form of regression which is used when the dependent is a dichotomy and the independents are of any type. Logistic regression can be used to predict a dependent variable on the basis of continuous and/or categorical independents and to determine the percent of variance in the dependent variable explained by the independents; to rank the relative importance of independents; to assess interaction effects; and to understand the impact of covariate control variables. Logistic regression applies maximum likelihood estimation after transforming the dependent into a “logit” variable (the natural log of the odds of the dependent occurring or not). In this way, logistic regression estimates the probability of a certain event occurring. These analyses are conducted with the program SAS. SAS (“statistical analysis software”) is a general purpose package (similar to Stata and SPSS). Ready-to-use procedures handle a wide range of statistical analyses, including but not limited to, analysis of variance, regression, categorical data analysis, multivariate analysis, survival analysis, psychometric analysis, cluster analysis, and nonparametric analysis.

As used herein, a “Z-score” refers to a standard score that is a very useful statistic because it (a) allows one to calculate the probability of a score occurring within the normal distribution and (b) enables one to compare two scores that are from different normal distributions. The standard score does this by converting (in other words, standardizing) scores in a normal distribution to Z-scores in what becomes a standard normal distribution. A Z-score is a measure of how many standard deviations below or above the population mean a raw score is. A Z-score can be placed on a normal distribution curve. Z-scores range from −3 standard deviations (which would fall to the far left of the normal distribution curve) up to +3 standard deviations (which would fall to the far right of the normal distribution curve).

As used herein, “Cohen's D” or “standardized mean difference” refers to one of the most common ways to measure effect size. An effect size is how large an effect is. For example, medication A has a larger effect than medication B. While a p-value can tell you if there is an effect, it won't tell you how large that effect is. Cohen's D specifically measures the effect size of the difference between two means. The formula for Cohen's D (for equally sized groups) is: d=(M1−M2)/sₚₒₒₗₑₐ, where M1=mean of group 1, M2=mean of group 2, spooled=pooled standard deviations for the two groups. The formula is: √[(s12+s22)/2].

As used herein, the phrase “proteomic profile” means the combination of proteins found in a subject's biosample, which includes but is not limited to isolated EVs. The proteomic profile is a collection of measurements, such as but not limited to a quantity or concentration, for individual proteins taken from a subject's biosample. Techniques to determine the levels of individual components of the proteomic profile from biosamples are well known to the skilled technician and include, but are not limited to, mass spectrometry, ultra-performance liquid chromatography (UPLC), high-performance liquid chromatography (HPLC), mass spectrometery in conjunction with UPLC, LC/MS/MS, ELISA, and Western blots.

As used herein, the phrase “miRNA profile” means the combination of miRNAs found in a subject's biosample, which includes but is not limited to isolated EVs. The miRNA profile is a collection of measurements, such as but not limited to a quantity or concentration, for individual miRNAs taken from a subject's biosample. Techniques to determine the levels of individual components of the miRNA profile from biosamples are well known to the skilled technician and include, but are not limited to, RNAseq and RT-qPCR.

The assessment of the levels of the individual components of the proteomic and/or miRNA profile can be expressed as absolute or relative values and may or may not be expressed in relation to another component, a standard, an internal standard, or another molecule of compound known to be in the sample. If the levels are assessed as relative to a standard or internal standard, then the standard can be added to the test sample prior to, during, or after sample processing.

As used herein, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, preventing a dystonia is intended. The words “prevent” and “preventing” and “prevention” also refer to prophylactic or preventative measures for protecting or precluding a subject (e.g., an individual) not having a given dystonia or dystonia-related complication from progressing to that complication.

As used herein, the terms “administering” and “administration” refer to any method of providing one or more of the disclosed therapeutic agents, disclosed pharmaceutical formulations, or a combination thereof to a subject. Such methods are well known to those skilled in the art and include, but are not limited to, the following routes: oral administration, transdermal administration, administration by inhalation, nasal administration, topical administration, in utero administration, intrahepatic administration, intravaginal administration, ophthalmic administration, intraaural administration, otic administration, intracerebral administration, rectal administration, sublingual administration, buccal administration, and parenteral administration, including injectable such as intravenous administration, intra-CSF administration, intra-arterial administration, intramuscular administration, and subcutaneous administration. Administration can also include hepatic intra-arterial administration or administration through the hepatic portal vein (HPV). Administration of a disclosed therapeutic agent, a disclosed pharmaceutical composition, or a combination thereof can comprise administration directly into the CNS (e.g., intraparenchymal, intracerebroventriular, inthrathecal cisternal, intrathecal (lumbar), deep gray matter delivery, convection-enhanced delivery to deep gray matter) or the PNS. Administration can be continuous or intermittent.

In an aspect, a “therapeutic agent” can be a “biologically active agent” or “biologic active agent” or “bioactive agent”, which refers to an agent that is capable of providing a local or systemic biological, physiological, or therapeutic effect in the biological system to which it is applied. For example, the bioactive agent can act to control infection or inflammation, enhance cell growth and tissue regeneration, control tumor growth, act as an analgesic, promote anti-cell attachment, and enhance bone growth, among other functions. Other suitable bioactive agents can include anti-viral agents, vaccines, hormones, antibodies (including active antibody fragments sFv, Fv, and Fab fragments), aptamers, peptide mimetics, functional nucleic acids, therapeutic proteins, peptides, or nucleic acids. Other bioactive agents include prodrugs, which are agents that are not biologically active when administered but, upon administration to a subject are converted to bioactive agents through metabolism or some other mechanism. Additionally, any of the compositions of the invention can contain combinations of two or more bioactive agents. It is understood that a biologically active agent can be used in connection with administration to various subjects, for example, to humans (i.e., medical administration) or to animals (i.e., veterinary administration). As used herein, the recitation of a biologically active agent inherently encompasses the pharmaceutically acceptable salts thereof.

As used herein, the term “pharmaceutically active agent” includes a “drug” or a “vaccine” and means a molecule, group of molecules, complex or substance administered to an organism for diagnostic, therapeutic, preventative medical, or veterinary purposes. This term includes externally and internally administered topical, localized and systemic human and animal pharmaceuticals, treatments, remedies, nutraceuticals, cosmeceuticals, biologicals, devices, diagnostics and contraceptives, including preparations useful in clinical and veterinary screening, prevention, prophylaxis, healing, wellness, detection, imaging, diagnosis, therapy, surgery, monitoring, cosmetics, prosthetics, forensics and the like. This term may also be used in reference to agriceutical, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. This term can also specifically include nucleic acids and compounds comprising nucleic acids that produce a bioactive effect, for example deoxyribonucleic acid (DNA) or ribonucleic acid (RNA). Pharmaceutically active agents include the herein disclosed categories and specific examples. It is not intended that the category be limited by the specific examples. Those of ordinary skill in the art will recognize also numerous other compounds that fall within the categories and that are useful according to the invention. Examples include a radiosensitizer, the combination of a radiosensitizer and a chemotherapeutic, a steroid, a xanthine, a beta-2-agonist bronchodilator, an anti-inflammatory agent, an analgesic agent, a calcium antagonist, an angiotensin-converting enzyme inhibitors, a beta-blocker, a centrally active alpha-agonist, an alpha−1-antagonist, carbonic anhydrase inhibitors, prostaglandin analogs, a combination of an alpha agonist and a beta blocker, a combination of a carbonic anhydrase inhibitor and a beta blocker, an anticholinergic/antispasmodic agent, a vasopressin analogue, an antiarrhythmic agent, an antiparkinsonian agent, an antiangina/antihypertensive agent, an anticoagulant agent, an antiplatelet agent, a sedative, an ansiolytic agent, a peptidic agent, a biopolymeric agent, an antineoplastic agent, a laxative, an antidiarrheal agent, an antimicrobial agent, an antifungal agent, or a vaccine. In a further aspect, the pharmaceutically active agent can be coumarin, albumin, bromolidine, steroids such as betamethasone, dexamethasone, methylprednisolone, prednisolone, prednisone, triamcinolone, budesonide, hydrocortisone, and pharmaceutically acceptable hydrocortisone derivatives; xanthines such as theophylline and doxophylline; beta-2-agonist bronchodilators such as salbutamol, fenterol, clenbuterol, bambuterol, salmeterol, fenoterol; antiinflammatory agents, including antiasthmatic anti-inflammatory agents, antiarthritis antiinflammatory agents, and non-steroidal antiinflammatory agents, examples of which include but are not limited to sulfides, mesalamine, budesonide, salazopyrin, diclofenac, pharmaceutically acceptable diclofenac salts, nimesulide, naproxene, acetominophen, ibuprofen, ketoprofen and piroxicam; analgesic agents such as salicylates; calcium channel blockers such as nifedipine, amlodipine, and nicardipine; angiotensin-converting enzyme inhibitors such as captopril, benazepril hydrochloride, fosinopril sodium, trandolapril, ramipril, lisinopril, enalapril, quinapril hydrochloride, and moexipril hydrochloride; beta-blockers (i.e., beta adrenergic blocking agents) such as sotalol hydrochloride, timolol maleate, timol hemihydrate, levobunolol hydrochloride, esmolol hydrochloride, carteolol, propanolol hydrochloride, betaxolol hydrochloride, penbutolol sulfate, metoprolol tartrate, metoprolol succinate, acebutolol hydrochloride, atenolol, pindolol, and bisoprolol fumarate; centrally active alpha-2-agonists (i.e., alpha adrenergic receptor agonist) such as clonidine, brimonidine tartrate, and apraclonidine hydrochloride; alpha-1-antagonists such as doxazosin and prazosin; anticholinergic/antispasmodic agents such as dicyclomine hydrochloride, scopolamine hydrobromide, glycopyrrolate, clidinium bromide, flavoxate, and oxybutynin; vasopressin analogues such as vasopressin and desmopressin; prostaglandin analogs such as latanoprost, travoprost, and bimatoprost; cholinergics (i.e., acetylcholine receptor agonists) such as pilocarpine hydrochloride and carbachol; glutamate receptor agonists such as the N-methyl D-aspartate receptor agonist memantine; anti-Vascular endothelial growth factor (VEGF) aptamers such as pegaptanib; anti-VEGF antibodies (including but not limited to anti-VEGF-A antibodies) such as ranibizumab and bevacizumab; carbonic anhydrase inhibitors such as methazolamide, brinzolamide, dorzolamide hydrochloride, and acetazolamide; antiarrhythmic agents such as quinidine, lidocaine, tocainide hydrochloride, mexiletine hydrochloride, digoxin, verapamil hydrochloride, propafenone hydrochloride, flecaimide acetate, procainamide hydrochloride, moricizine hydrochloride, and diisopyramide phosphate; antiparkinsonian agents, such as dopamine, L-Dopa/Carbidopa, selegiline, dihydroergocryptine, pergolide, lisuride, apomorphine, and bromocryptine; antiangina agents and antihypertensive agents such as isosorbide mononitrate, isosorbide dinitrate, propranolol, atenolol and verapamil; anticoagulant and antiplatelet agents such as coumadin, warfarin, acetylsalicylic acid, and ticlopidine; sedatives such as benzodiazapines and barbiturates; ansiolytic agents such as lorazepam, bromazepam, and diazepam; peptidic and biopolymeric agents such as calcitonin, leuprolide and other LHRH agonists, hirudin, cyclosporin, insulin, somatostatin, protirelin, interferon, desmopressin, somatotropin, thymopentin, pidotimod, erythropoietin, interleukins, melatonin, granulocyte/macrophage-CSF, and heparin; antineoplastic agents such as etoposide, etoposide phosphate, cyclophosphamide, methotrexate, 5-fluorouracil, vincristine, doxorubicin, cisplatin, hydroxyurea, leucovorin calcium, tamoxifen, flutamide, asparaginase, altretamine, mitotane, and procarbazine hydrochloride; laxatives such as senna concentrate, casanthranol, bisacodyl, and sodium picosulphate; antidiarrheal agents such as difenoxine hydrochloride, loperamide hydrochloride, furazolidone, diphenoxylate hydrochloride, and microorganisms; vaccines such as bacterial and viral vaccines; antimicrobial agents such as penicillins, cephalosporins, and macrolides, antifungal agents such as imidazolic and triazolic derivatives; and nucleic acids such as DNA sequences encoding for biological proteins, and antisense oligonucleotides. It is understood that a pharmaceutically active agent can be used in connection with administration to various subjects, for example, to humans (i.e., medical administration) or to animals (i.e., veterinary administration). As used herein, the recitation of a pharmaceutically active agent inherently encompasses the pharmaceutically acceptable salts thereof.

In an aspect, a “therapeutic agent” can be any agent that effects a desired clinical outcome in a subject having a dystonia, suspected of having a dystonia, and/or likely to develop or acquire a dystonia. In an aspect, a disclosed therapeutic agent can be an oligonucleotide therapeutic agent. A disclosed oligonucleotide therapeutic agent can comprise a single-stranded or double-stranded DNA, iRNA, shRNA, siRNA, mRNA, non-coding RNA (ncRNA), an antisense molecule, miRNA, a morpholino, a peptide-nucleic acid (PNA), or an analog or conjugate thereof. In an aspect, a disclosed oligonucleotide therapeutic agent can be an ASO or an RNAi. In an aspect, a disclosed oligonucleotide therapeutic agent can comprise one or more modifications at any position applicable.

In an aspect, the skilled person can determine an efficacious dose, an efficacious schedule, and an efficacious route of administration for one or more of the disclosed agents, disclosed therapeutic agents, disclosed pharmaceutical formulations or a combination thereof (such as an agent that modulates ISR dysfunction and/or eIFα2 dysfunction) so as to treat or prevent a dystonia (such as DYT1). In an aspect, the skilled person can also alter, change, or modify an aspect of an administering step to improve efficacy of one or more of the disclosed agents, disclosed therapeutic agents, disclosed pharmaceutical formulations, or a combination thereof. In an aspect, the skilled person can determine an efficacious dose, an efficacious schedule, and an efficacious route of administration for any disclosed agent. These disclosed agents include but are not limited to anticholinergic drugs, benzodiazepines, muscle relaxants, agents that modulate the expression level of one or more disclosed differentially expressed proteins, agents that target eIF2α signaling, ritonavir, nelfinavir, lopinavir, saquinavir, deshydroxy-lopinavir, cobicistat, deshydroxy-ritonavir, or any combination thereof.

As used herein, “modifying the method” can comprise modifying or changing one or more features or aspects of one or more steps of a disclosed method. For example, in an aspect, a method can be altered by changing the amount of one or more of the disclosed agents, disclosed therapeutic agents, disclosed pharmaceutical formulations or a combination thereof administered to a subject, or by changing the frequency of administration of one or more of the disclosed agents, disclosed therapeutic agents, disclosed pharmaceutical formulations or a combination thereof, or by changing the duration of time one or more of the disclosed agents, disclosed therapeutic agents, disclosed pharmaceutical formulations or a combination thereof are administered to a subject.

As used herein, “concurrently” means (1) simultaneously in time, or (2) at different times during the course of a common treatment schedule.

The term “contacting” as used herein refers to bringing one or more of the disclosed therapeutic agents, disclosed pharmaceutical formulations, or a combination thereof together with a target area or intended target area in such a manner that the one or more of the disclosed agents, disclosed therapeutic agents, disclosed pharmaceutical formulations or a combination thereof exert an effect on the intended target or targeted area either directly or indirectly. A target area or intended target area can be one or more of a subject's organs (e.g., lungs, heart, liver, kidney, brain, etc.). In an aspect, a target area or intended target area can be any cell or any organ infected by a dystonia (such as DYT1). In an aspect, a target area or intended target area can be the brain or various neuron populations.

As used herein, “determining” can refer to measuring or ascertaining the presence and severity of a dystonia, such as, for example, DYT1. Methods and techniques used to determine the presence and/or severity of a dystonia are typically known to the medical arts. For example, the art is familiar with the ways to identify and/or diagnose the presence, severity, or both of a dystonia (such as, for example, a DYT1).

In an aspect, “determining” can also refer to measuring or ascertaining the level of one or more proteins or peptides in a biosample, or measuring or ascertaining the level or one or more RNAs or miRNAs in a biosample. Methods and techniques for determining the level of proteins/peptides and RNAs/miRNAs are known to the art and are disclosed herein.

The level of differential expression of proteins and/or miRNAs in a biosample when compared to a reference biosample (or any other biosample) can vary. For example, the level of any one or more differentially expressed proteins and/or miRNAs in a biosample can be at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 times or fold lower than that of a reference biosample. Or, the levels of any one or more differentially expressed proteins and/or miRNAs can be at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 times or fold higher than that of a reference biosample. In an aspect, the number of “times” the level of one or more differentially expressed proteins and/or miRNAs is lower or higher than that of a reference level can be a relative or an absolute number of times. Or, in an aspect, the level of the proteins and/or miRNAs can be normalized to a standard and these normalized levels can then be compared to one another to determine whether the differentially expressed proteins and/or miRNAs is lower or higher.

As used herein, “effective amount” and “amount effective” can refer to an amount that is sufficient to achieve the desired result such as, for example, the treatment and/or prevention of a dystonia (e.g., DYT1) or a suspected dystonia. As used herein, the terms “effective amount” and “amount effective” can refer to an amount that is sufficient to achieve the desired an effect on an undesired condition (e.g., a dystonia). For example, a “therapeutically effective amount” refers to an amount that is sufficient to achieve the desired therapeutic result or to have an effect on undesired symptoms, but is generally insufficient to cause adverse side effects. In an aspect, “therapeutically effective amount” means an amount of a disclosed pharmaceutical formulation, a disclosed agent, and/or disclosed therapeutic agent that (i) treats the particular disease, condition, or disorder (e.g., a dystonia such as DYT1), (ii) attenuates, ameliorates, or eliminates one or more symptoms of the particular disease, condition, or disorder (e.g., a dystonia), or (iii) delays the onset of one or more symptoms of the particular disease, condition, or disorder described herein (e.g., a dystonia). The specific therapeutically effective dose level for any particular patient will depend upon a variety of factors including the disorder being treated and the severity of the disorder; the disclosed therapeutic agents or disclosed pharmaceutical formulations employed; the disclosed methods employed; the age, body weight, general health, sex and diet of the patient; the time of administration; the route of administration; the rate of excretion of the disclosed isolated therapeutic agents or disclosed pharmaceutical formulations employed; the duration of the treatment; drugs used in combination or coincidental with the disclosed therapeutic agents or disclosed pharmaceutical formulations employed, and other like factors well known in the medical arts. For example, it is well within the skill of the art to start doses of the disclosed therapeutic agents or disclosed pharmaceutical formulations at levels lower than those required to achieve the desired therapeutic effect and to gradually increase the dosage until the desired effect is achieved. If desired, then the effective daily dose can be divided into multiple doses for purposes of administration. Consequently, a single dose of the disclosed therapeutic agents or disclosed pharmaceutical formulations can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition, such as, for example, a dystonia.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents, or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. In an aspect, a pharmaceutical carrier employed can be a solid, liquid, or gas. In an aspect, examples of solid carriers can include lactose, terra alba, sucrose, talc, gelatin, agar, pectin, acacia, magnesium stearate, and stearic acid. In an aspect, examples of liquid carriers can include sugar syrup, peanut oil, olive oil, and water. In an aspect, examples of gaseous carriers can include carbon dioxide and nitrogen. In preparing a disclosed composition for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “excipient” refers to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, for reference, Remington's Pharmaceutical Sciences, (1990) Mack Publishing Co., Easton, Pa., which is hereby incorporated by reference in its entirety.

As used herein, “small molecule” can refer to any organic or inorganic material that is not a polymer. Small molecules exclude large macromolecules, such as large proteins (e.g., proteins with molecular weights over 2,000, 3,000, 4,000, 5,000, 6,000, 7,000, 8,000, 9,000, or 10,000), large nucleic acids (e.g., nucleic acids with molecular weights of over 2,000, 3,000, 4,000, 5,000, 6,000, 7,000, 8,000, 9,000, or 10,000), or large polysaccharides (e.g., polysaccharides with a molecular weight of over 2,000, 3,000, 4,000, 5,000, 6,000, 7,000, 8,000, 9,000, or 10,000). In an aspect, a “small molecule”, for example, can be a drug that can enter cells easily because it has a low molecular weight. In an aspect, a disclosed small molecule can penetrate the blood-brain-barrier (BBB).

As known to the art, “microRNAs” or “miRNAs” are small non-coding RNAs that regulate the expression of protein coding RNAs. The binding of an antisense compound to a microRNA prevents that microRNA from binding to its messenger RNA targets, and thus interferes with the function of the microRNA. MicroRNA mimics can enhance native microRNA function. miRNAs are generally about 17 to about 25 nucleotide bases (nt) in length in their biologically active form. In an aspect, a disclosed miRNA can regulate gene expression post transcriptionally by decreasing target mRNA translation. In an aspect, a disclosed miRNA can function as a negative regulator. In an aspect, a disclosed miRNA is about 17 to about 25, about 17 to about 24, about 17 to about 23, about 17 to about 22, about 17 to about 21, about 17 to about 20, about 17 to about 19, about 18 to about 25, about 18 to about 24, about 18 to about 23, about 18 to about 22, about 18 to about 21, about 18 to about 20, about 19 to about 25, about 19 to about 24, about 19 to about 23, about 19 to about 22, about 19 to about 21, about 20 to about 25, about 20 to about 24, about 20 to about 23, about 20 to about 22, about 21 to about 25, about 21 to about 24, about 21 to about 23, about 22 to about 25, about 22 to about 24, or about 22 nucleotides in length. Generally, there are three forms of miRNAs: primary miRNAs (pri-miRNAs), premature miRNAs (pre-miRNAs), and mature miRNAs, all of which are within the scope of the present disclosure.

As used herein, “exosomes” refer to small membrane vesicles found in cell culture supernatants and in different biological fluids. Exosomes form in a particular population of endosomes, called multivesicular bodies (MVBs), by inward budding into the lumen of the compartment. Upon fusion of MVBs with the plasma membrane, these internal vesicles are secreted. Exosomes possess a defined set of membrane and cytosolic proteins. Exosomes are implicated in multiple biological processes.

As used herein, “extracellular vesicles” or “EVs” are known to facilitate intercellular communication in diverse cellular processes such as immune responses and coagulation. EVs can be broadly classified into exosomes, microvesicles (MVs) and apoptotic bodies according to their cellular origin as shown below.

| | Exosomes | Microvesicles | Apoptotic Bodies |
|---|---|---|---|
| Origin | Endocytic pathway | Plasma membrane | Plasma membrane |
| Size | 40-120 nm | 50-1,000 nm | 500-2,000 nm |
| Function | Intercellular | Intercellular | Facilitate |
| | communication | communication | phagocytosis |
| Markers | Alix, Tsg101, | Integrins, selectins, | Annexin V, |
| | tetraspanins (CD81, | CD40 | phosphatidylserine |
| | CD63, CD9), flotillin | | |
| Contents | Proteins and nucleic | Proteins and nucleic | Nuclear fractions, |
| | acids (mRNA, miRNA | acids (mRNA, miRNA | cell organelles |
| | and other non-coding | and other non-coding | |
| | RNAs) | RNAs) | |

As used herein, the term “package insert” is used to refer to instructions customarily included in commercial packages of therapeutic products, that contain information about the indications, usage, dosage, administration, contraindications and/or warnings concerning the use of such therapeutic products.

As used herein, the term “in combination” in the context of the administration of one or more of the disclosed agents, disclosed therapeutic agents, disclosed pharmaceutical formulations or a combination thereof includes the use of more than one therapy (e.g., additional therapeutic agents). Administration “in combination with” one or more additional therapeutic agents includes simultaneous (e.g., concurrent) and consecutive administration in any order. The use of the term “in combination” does not restrict the order in which therapies are administered to a subject. By way of non-limiting example, a first therapy (e.g., one or more of the disclosed agents, disclosed therapeutic agents, disclosed pharmaceutical formulations, or a combination thereof) may be administered prior to (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 8 weeks, 8 weeks, 9 weeks, 10 weeks, 11 weeks, or 12 weeks), concurrently, or after (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 7 weeks, 8 weeks, 9 weeks, 10 weeks, 11 weeks, or 12 weeks or longer) the administration of a second therapy (e.g., one or more of the disclosed agents, disclosed therapeutic agents, disclosed pharmaceutical formulations, or a combination thereof or one or more additional therapeutic agents) to a subject having or diagnosed with a dystonia (such as DYT1).

Disclosed are the components to be used to prepare the disclosed agents, disclosed therapeutic agents, and/or the disclosed pharmaceutical formulations as well the disclosed agents, disclosed therapeutic agents, and/or the disclosed pharmaceutical formulations used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific aspects or combination of aspects of the disclosed methods.

### B. Dystonias

A plethora of heterogeneous movement disorders is grouped under the umbrella term “dystonia”. The clinical presentation ranges from isolated dystonia to multi-systemic disorders where dystonia is only a co-occurring sign. In the past, definitions, nomenclature, and classifications have been repeatedly refined, adapted, and extended to reflect novel findings and increasing knowledge about the clinical, etiologic, and scientific background of dystonia. Currently, dystonia is suggested to be classified according to 2 axes. The first axis offers precise categories for the clinical presentation grouped into age at onset, body distribution, temporal pattern, and associated features. The second, etiologic, axis discriminates pathological findings, as well as inheritance patterns, mode of acquisition, or unknown causality. Furthermore, the recent recommendations regarding terminology and nomenclature of inherited forms of dystonia and related syndromes are illustrated in this article. Thus, dystonias are a group of chronic movement-disabling disorders for which highly effective oral medications or disease-modifying therapies are lacking. The most effective treatments require invasive procedures such as deep brain stimulation.

Presently, at least 24 genetic loci have been associated with isolated or combined heritable dystonias (Balint B., et al. (2015) Eur. J. Neurol. 22:610-617). While some cellular processes are implicated by those dystonia-associated genes for which there are known functions, the cellular mechanisms for dystonia remain largely unknown (Bragg D C, et al. (2011) Neurobiol. Dis. 42:136-147). The first identified gene was named TORIA by Ozelius and colleagues and is associated with early-onset torsion dystonia (Ozelius L J, et al. (1997). Nat. Genet. 17:40-48). TORIA encodes the protein torsinA. All torsinA-related dystonia cases found so far are due to the deletion of one of a pair of glutamate residues (E302/303) toward the C terminus of the encoded protein. TorsinA is part of the large AAA+ family of ATPases and the glutamate deletion is near to the ATP binding region. An interesting aspect of the genetics of torsinA dystonia is that there is greatly reduced penetrance. About one third of patients who carry the causal DE302/303 mutation develop dystonia, while the rest remaining asymptomatic. There also appears to be a time-dependent window for susceptibility. Generally, mutation carriers who are asymptomatic in their early 20s remain so throughout life, although there may be exceptions (Bressman S B, et al. (2000) Curr Treat Options Neurol. 2(3):275-285; Bressman S B. (2000) Clin Neuropharmacol. 23(5):239-251; Bressman S B, et al. (2000) Neurology. 54(9):1746-52). This implies that there is a critical timing for the expression of symptoms and indicates that dystonia is a developmental disease.

Inherited forms of dystonia require a confirmed genetic origin and can again be subdivided into multiple groups according to the pattern of inheritance. There are several forms of autosomal dominant dystonia such as DYT-TOR1A (Ozelius L J, et al. (1997). Nat. Genet. 17:40-48), DYT/PARK-GCH1 (Ichinose H, et al. (1994) Nat Genet. 8(3):236-242; Segawa M, et al. (2003) Ann Neurol. 54 Suppl 6:S32-S45), DYT-THAP1 (Fuchs T, et al. (2009) Nat Genet. 41(3):286-288), DYT-SGCE (Zimprich A, et al. (2001) Nat Genet. 29(1):66-69), and DYT/PARK-ATP1A3 (de Carvalho A P, et al. (2004). Neuron. 43(2):169-175). Autosomal recessive forms of dystonia include as DYT-ATP7B, also known as Wilson disease (Bull P C, et al. (1993) Genomics. 16(3):593-598), NBIA/DYT-PANK2 or pantothenate kinase-associated neurodegeneration (PKAN) (Zhou Y, et al. (2001) Neuropharmacology. 41(5):601-608), and NBIA/DYT/PARKa-PLA2G6 or PLA2G6-associated neurodegeneration (PLAN) (Morgan N V, et al. (2006) Nat Genet. 38(7):752-754). Also, multiple metabolic disorders can be found in this category. X-linked recessive dystonias include DYT/PARK-TAF1 (Makino S, et al. (2007) Am J Hum Genet. 80(3):393-406), DYT/CHOR-HPRT or Lesch-Nyhan syndrome (Gibbs R A, et al. (1987) Science. 236(4799):303-305), and DYT-TIMM8A, also known as Mohr-Tranebjaerg syndrome (Tranebjaerg L, et al. (2000) Adv Otorhinolaryngol. 56:176-180). Inherited forms with mutations in the mitochondrial genome are, for example, Leigh syndrome or DYT-mt-ND6 (Leber optic atrophy and dystonia) (Kim C E, et al. (2010). Proc. Natl. Acad. Sci. USA. 107:9861-9866). Notably, a large proportion of the recessive forms (autosomal and X-linked) as well as the mitochondrial forms are classified as complex dystonia forms, whereas all isolated dystonias with a known genetic causality are inherited in an autosomal dominant fashion (Klein C, et al. (2017) GeneReviews).

Several causal factors for the acquisition of dystonia have been documented so far. These factors include perinatal brain injury (e.g., dystonic cerebral palsy, delayed onset dystonia), infection/inflammation (e.g., viral encephalitis, encephalitis lethargica, subacute sclerosing panencephalitis, human immunodeficiency virus (HIV) infection, autoimmune causes, tuberculosis, syphilis), drugs (levodopa and dopamine agonists, neuroleptics like dopamine receptor blocking drugs, anticonvulsants, and calcium channel blockers), toxic (e.g., manganese, cobalt, carbon disulfide, cyanide, methanol, disulfiram, and 3-nitropropionic acid), vascular (ischemia, hemorrhage, and arteriovenous malformation including aneurysm), neoplastic (e.g., brain tumor, and paraneoplastic encephalitis), brain injury (e.g., head trauma, brain surgery including stereotactic ablations, and electrical injury).

For example, DYT1 dystonia is a rare, early-onset, generalized form of dystonia. DYT1 is caused by an in-frame trinucleotide deletion in the TOR1A gene, leading to loss of a glutamic acid residue (DE) from the AAA+ ATPase Torsin1a (Ozelius L J, et al. (1997). Nat. Genet. 17:40-48). Both the normal function of Torsin1a and significance of the mutant protein for disease pathogenesis have been intensively studied and at least five cellular processes have been suggested, including roles in nuclear transport, synaptic vesicle cycling, lipid metabolism, and endoplasmic reticulum (ER) stress (Burdette et al. (2010) Cell Stress Chaper. 15:605-617; Chen P, et al. (2010). Hum. Mol. Genet. 19:3502-3515; Goodchild R E, et al. (2005) Neuron. 48:923-932; Granata A, et al. (2011). EMBO J. 30, 181-193; Granata A, et al. (2008). J. Biol. Chem. 283:7568-7579; Grillet M, et al. (2016). Dev. Cell. 38:235-247; Jokhi V, et al. (2013). Cell Rep. 3:988-995; Nery F C, et al. (2011). Nat. Commun. 2:393).

Normally, wild-type (WT) Torsin1a cycles between the outer nuclear envelope (NE) and ER lumen in an ATP-dependent fashion, with the bulk of the protein detected in the ER (Goodchild R E, et al. (2004) Proc. Natl. Acad. Sci. U.S.A. 101:847-852; Naismith T V, et al. (2004). Proc. Natl. Acad. Sci. USA. 101:7612-7617). In contrast, when ΔE Torsin1a is the major species, as in overexpression experiments or homozygous knockin mouse models, it predominantly co-localizes with nuclear envelope markers and disrupts the normal subcellular NE membrane structure in a manner that is suggestive of a membrane-trafficking defect (Goodchild R E, et al. (2004) Proc. Natl. Acad. Sci. U.S.A. 101:847-852; Jokhi V, et al. (2013). Cell Rep. 3:988-995; Naismith T V, et al. (2004). Proc. Natl. Acad. Sci. USA. 101:7612-7617). At the light microscopic level, ΔE Torsin1a distribution appears as an abnormal punctate pattern. The ΔE TorsinA mislocalizing propensity is also observed independent of whether the protein is expressed as a fusion protein or not. Lastly, ΔE Torsin1a has been associated with activation of the unfolded protein response (UPR) (Bragg D C, et al. (2011) Neurobiol. Dis. 42:136-147; Chen P, et al. (2010). Hum. Mol. Genet. 19:3502-3515; Hewett J W, et al. (2007). Proc. Natl. Acad. Sci. USA. 104:7271-7276; Nery F C, et al. (2011). Nat. Commun. 2:393), and in DYT1 patient-derived fibroblasts, the chemical chaperone, phenylbutyric acid (PBA) reduces indicators of UPR activation (Cao S, et al. (2010). Dis. Model. Mech. 3:386-396). PBA significantly reduced punctate pathology. Thus, Torsin1a localization phenotypes predict known Torsin1a biology.

### C. Integrated Stress Response

The integrated stress response (ISR) is an elaborate signaling pathway present in eukaryotic cells, which is activated in response to a range of physiological changes and different pathological conditions. Such stresses commonly include cell extrinsic factors such as hypoxia, amino acid deprivation, glucose deprivation, and viral infection. However, cell intrinsic stresses such as endoplasmic reticulum (ER) stress, caused by the accumulation of unfolded proteins in the ER, can also activate the ISR. Furthermore, in the context of cancer biology, the ISR can be triggered by oncogene activation. The common point of convergence for all the stress stimuli that activate ISR is phosphorylation of the alpha subunit of eukaryotic translation initiation factor 2 (eIF2a) on serine 51. In mammalian cells, this is catalyzed by a family of four serine/threonine (S/T) eIF2a kinases that are activated by distinct stress stimuli. eIF2a phosphorylation causes a reduction in global protein synthesis while allowing the translation of selected genes including activating transcription factor 4 (ATF4), aiding cell survival and recovery. However, if the cellular stress is severe, either in intensity or in duration, it will overwhelm the capacity of the adaptive response to resolve it and additional components become activated to execute cell death.

Dephosphorylation of eIF2a signals termination of the ISR and return to normal protein synthesis. It is likely that the duration and level of eIF2a phosphorylation, as well as ATF4 regulation and its interactions with other proteins, determine the ultimate ISR outcome resulting from different environmental and physiological stresses. The initiation of the ISR relies on four evolutionarily related eIF2α kinases that each senses specific insults and signals by phosphorylating eIF2α. On the other hand, dephosphorylation of eIF2α and subsequent restoration of the translational capacity is emerging as a key event that controls the complete recovery from stress and ISR termination. Two cellular cofactors of the protein phosphatase-1 (PP1c) can specifically reverse the phosphorylation of eIF2α. The first one is GADD34, which is induced by the ISR to specifically direct PP1c to dephosphorylate eIF2α, allowing termination of the response and restoration of the homeostatic pace of translation. The second one is the protein CReP (Constitutive Repressor of eIF2α Phosphorylation), also known as PPPR151B, that is expressed ubiquitously in unstressed cells and was identified as a key factor maintaining low levels of eIF2α phosphorylation.

The “eukaryotic translation initiation factor 2” or “eIF2” refers to a heterotrimeric GTPase composed of a, b, and c subunits, which can bind GTP and methionine initiator tRNA to form a ternary complex. In conjunction with translation machinery, ternary complex scans along the 5′ untranslated region of mRNAs to detect the translation start site. Once the AUG start codon is decoded, GTP is hydrolyzed and eIF2-GDP is released as a binary complex from the ribosome. Exchange of GDP for GTP enables a new round of translation initiation. This occurs with the aid of a dedicated nucleotide exchange factor, translation initiation factor 2B (eIF2B), which is a decameric nucleotide exchange factor composed of two copies of subunits α, β, γ, δ, and ε.

The eIF2a kinases act as early responders to disturbances in cellular homeostasis. There are four members of the family: PKR-like ER kinase (PERK), double-stranded RNA-dependent protein kinase (PKR), heme-regulated eIF2a kinase (HRI), and general control nonderepressible 2 (GCN2). All four eIF2a kinases share extensive homology in their kinase catalytic domains, but possess distinct regulatory domain. Each eIF2a kinase dimerizes and autophosphorylates for full activation. However, each kinase responds to distinct environmental and physiological stresses, which reflects their unique regulatory mechanisms. But, when ER stress, viral infection, and other cellular stress signals activate PERK, PKR, HRI, and GCN2 kinases, they converge on phosphorylation of eIF2a, the core of ISR. Upon ISR induced phosphorylation, eIF2 is converted from substrate to competitive inhibitor of eIF2B, arresting general protein synthesis and upregulating translation of a select few mRNAs containing upstream open reading frames. These mRNAs encode stress-responsive factors such as the transcription factor ATF4. This leads to global attenuation of Cap-dependent translation while concomitantly initiates the preferential translation of ISR-specific mRNAs, such as ATF4.

ATF4 is a basic leucine zipper (bZIP) transcription factor that belongs to the activating transcription factor/cyclic AMP response element binding protein (ATF/CREB family) [100-102]. ATF4 has several dimerization partners that influence its regulation of gene transcription and can guide cellular outcome. In fact, ATF4 is a key deciding factor in cellular fate in response to ISR activation. It is regulated at the transcriptional, translational, and post-translational level, and moreover, its ability to interact with other transcription factors provides a further level of regulation. For example, ATF4 forms homo- and heterodimers that bind to DNA targets to control the expression of genes involved in cellular adaptation. Termination of the ISR is regulated by the constitutively expressed CReP and stress-inducible phosphatase GADD34 that dephosphorylate eIF2a.

Although multiple stresses converge on eIF2a phosphorylation to activate the ISR, the cellular outcome is not always the same. The effect of ISR activation depends not only on the nature of the stress, its duration and severity, but also on the extent of eIF2a phosphorylation and translation of ATF4 mRNA and other bZIP transcription factors. It is commonly accepted that a short-lived ISR is an adaptive, pro-survival response aiming at resolving stress and restoring homeostasis, while a prolonged ISR can signal toward cell death induction. Therefore, this dual effect of eIF2a phosphorylation raises an important question concerning how the switch between pro-survival and pro-death signaling by ISR is regulated, as well as whether a threshold of cell stress signals exists that favors the activation of cell death proteins.

The ISR, together with other cellular adaptation pathways, functions as an important part of the cellular defense strategy in response to stress. It does this mainly through altering global protein synthesis and through the regulation of genes that promote pro-survival signaling such as through the activation of autophagy, or that counteract pathways that lead to cell death such as apoptosis or proteotoxicity (impairment of cell function due to the effects of misfolded proteins). Notably, there is also a cross-talk between the ISR and other pro-survival pathways such as the UPR, phosphatidylinositol-3 kinase (PI3K) signaling, autophagy, and the ubiquitin-proteasome system.

One of the major effects of the ISR is on protein synthesis. The initial repression of global mRNA translation plays a very important role in promoting cell survival in the face of different stresses that activate the ISR. The accumulation of unfolded proteins in the ER induces a condition of ER stress, which is relieved by the reduced level of incoming proteins when global protein synthesis is inhibited. PKR activation of the ISR during viral infection helps to reduce the translation of viral mRNAs, thus protecting the cells. Under conditions of amino acid depletion, activation of the ISR by GCN2 reduces the need for amino acids for protein synthesis, thus alleviating this stress. Activation of the ISR by HRI under conditions of low heme lessens the need for heme by attenuating the translation of globin mRNAs, thus reducing the stress and promoting survival. It is important, however, to note that timely termination of the ISR also plays a key role in promoting long-term cell survival, by re-starting synthesis of essential proteins. This is achieved through dephosphorylation of eIF2a by the phosphatase GADD34, which is induced by ATF4 and its downstream targets CHOP and ATF3. Through the activation of macroautophagy, commonly known as autophagy, the ISR can regulate cell survival and cell death path.

In vitro human genome-wide screening and human genetics revealed weakened phosphor-eIF2α signaling in a variety of dystonias. For example, Prkra is gene discovered in familial DYT16 (Camargos S, et al. (2008). Lancet Neurol. 7:207-215), which is also associated with sporadic dystonia (Dos Santos C O, et al. (2018) Parkinsonism Relat. Disord. 48:93-96). THAP1 is a gene discovered in familial DYT6 (Fuchs T, et al. (2009) Nat Genet. 41(3):286-288), which also shows dysregulated eIF2a in mouse model (Zakirova Z, et al. (2018) PLoS Genet. 14(1):e1007169). Eif2ak1 and Eif2ak2 are two new genetic syndromes reported with features that include prominent dystonia (Mao D, et al. (2020) Am. J. Hum. Genet. 106:570-583 (2020)). EIF2B shows vanishing white matter disease symptoms include dystonia, torticollis (Klingelhoefer L, et al. (2014) Clin Med (Lond). 14(5):520-524).

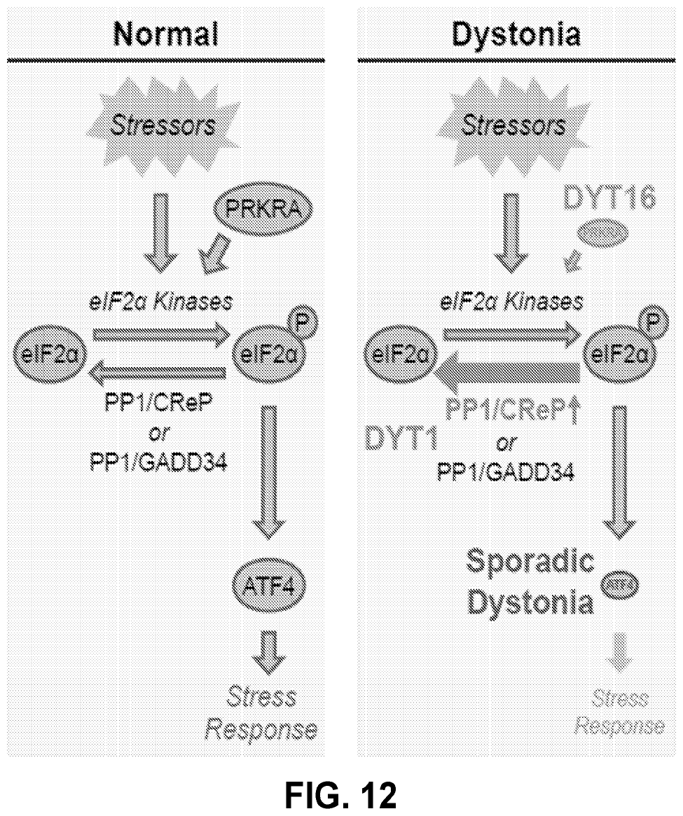

Although the specific disruptions in the eIF2a pathway are distinct among these three dystonias, they converge upon a common consequence of reduced eIF2a pathway signaling (FIG. 12). In DYT1 patient-derived fibroblasts, there was an attenuated ISR alongside high basal levels of a negative feedback regulator of eIF2a phosphorylation, the CReP/PPP1R15B phosphatase. In sporadic cervical dystonia patients, there was significant enrichment of rare ATF4 missense variants that reduce transcriptional activity. Transcriptional regulation by ATF4 is a major downstream effector of the ISR (Pakos-Zebrucka K, et al. (2016). EMBO Rep. 17:1374-1395). In DYT16, it is noteworthy that the most common PRKRA mutation is associated with both initially decreased and delayed excessive stress-induced phosphorylation of eIF2a among other effects such as promoting apoptosis.

Integrated stress response inhibitor (ISRIB) is a small drug-like that targets eIF2B. In vitro studies determined that ISRIB activates and stabilizes a decameric eIF2B complex. Specifically, ISRIB renders cells insensitive to eIF2a phosphorylation and thus inhibits the ISR downstream of eIF2a phosphorylation resulting in the attenuation of ATF4 synthesis. ISRIB restores the translational capacity and thus impairs the adaptation of cells to chronic ER stress. Additionally, ISRIB has been shown to prevent the formation of stress granules caused by eIF2a phosphorylation. In rodents, ISRIB is effective in a number of disease models in that treatment with the molecule can reverse cognitive deficits following traumatic brain injury, protect against prion-induced neurodegeneration, and prevent metastasis of a subset of cancers. ISRIB was shown to alter the normal subcellular localization of WT TorsinA in a way that was more similar to the distribution of dE TorsinA in DYT1.

Salubrinal is an agent that prolongs ISR activation by inhibiting or prevent eIF2α dephosphorylation by inhibiting the protein complex GADD34/protein phosphatase 1 (PP1), which consists of the general cellular serine/threonine phosphatase PP1 and the non-enzymatic cofactor GADD34. Salubrinal also inhibits CReP-PP1 complexes that dephosphorylate eIF2α. Salubrinal has a MW of 479.8 and a molecular formula of C₂₁H₁₇Cl₃N₄OS. Salubrinal is also known as PubChem ID 5717801. Salubrinal acts by slowing down protein synthesis, allowing increased time for protein folding within the ER, and thus protecting the cells from the deleterious effects of proteotoxicity.

Sal 003 is a cell-permeable inhibitor of cellular phosphatase complexes that dephosphorylate eukaryotic translation initiation factor 2 subunit α (eIF2α). Sal-003 is an analog of salubrinal with improved aqueous solubility. Sal-003 has a molecular weight of 463.21 and a molecular formula of C₁₈H₁₅Cl₄N₃OS. Sal 003 has the PubChem ID No. 5717737.

Guanabenz inhibits the stress-induced phosphatase GADD34 that causes the dephosphorylation of eIF2α. Guanabenz is not a selective GADD34 inhibitor though. Guanabenz has a MW of 231.08 and a molecular formula of C₈H₈Cl₂N₄. Guanabenz is also known as PubChem ID 5353646. Salubrinal acts by slowing down protein synthesis, allowing increased time for protein folding within the ER, and thus protecting the cells from the deleterious effects of proteotoxicity. Sephin 1, which is a guanabenz derivative, is a safe and selective GADD34-specific inhibitor.

Pharmacological approaches to targeting ISR signaling include (i) stimulating eIF2a phosphorylation through chemical activators of eIF2a kinases such as histidinol, asparaginase, halofuginone, arginine deiminase, BTdCPU, BEPP monohydrochloride, and CCT020312, or preventing eIF2a phosphorylation using indirubin-30-monoxime, SP600125, and SyK to inhibit GSK2, (iii) blocking PERK activation using GSK2606414 and GSK2656157, (iv) modulating PKR using C16 and 2-aminopurine, (v) inhibiting HRI using aminopyrazolindane, (vi) prolonging ISR using salubrinal, (vii) blocking GADD34 using guanabenz and Sephin1, (viii) decreasing CReP expression thereby affecting CReP-PP1 complex binding to eIF2a using nelfinavir [231] decreases CReP expression and affects CReP-PP1 complex, and (ix) reversing the consequences of eIF2a phosphorylation using ISRIB. Another approach to regulate ISR involves the modulation of ATF4 post-translational modifications. For example, phosphorylation of ATF4 at S251 can be blocked by SL0101, an RSK2 kinase inhibitor while the inhibition of phosphorylation of ATF4 at S254 can be achieved by inhibiting PKA with H-89. Further, nuclear to cytoplasmic shuttling of ATF4 followed by its phosphorylation-dependent proteasomal degradation can be induced by the synthesized RPL41 peptide.

### D. Compositions for Treating and/or Preventing a Dystonia

Disclosed herein is a pharmaceutical formulation comprising one or more disclosed agents in a pharmaceutically acceptable carrier. In an aspect, a disclosed pharmaceutical formulation can comprise one or more agents that modulate the expression level of one or more disclosed differentially expressed proteins such as, for example, increasing or decreasing the expression level. In an aspect, a disclosed pharmaceutical formulation can comprise one or more agents that target eIF2α signaling, such as, for example, contributing to the phosphorylation or de-phosphorylation of eIF2α. In an aspect, a disclosed pharmaceutical formulation can comprise ritonavir, nelfinavir, lopinavir, saquinavir, deshydroxy-lopinavir, cobicistat, deshydroxy-ritonavir, or any combination thereof. In an aspect, a disclosed pharmaceutical formulation can comprise ritonavir. In an aspect, a disclosed pharmaceutical formulation can comprise guanabenz, salubrinal, ISRIB, Sephin 1, or any combination thereof. In an aspect, a disclosed pharmaceutical formulation can comprise an agent that targets ISR signaling such as (i) stimulating eIF2a phosphorylation through chemical activators of eIF2a kinases such as histidinol, asparaginase, halofuginone, arginine deiminase, BTdCPU, BEPP monohydrochloride, and CCT020312, or preventing eIF2a phosphorylation using indirubin-30-monoxime, SP600125, and SyK to inhibit GSK2, (iii) blocking PERK activation using GSK2606414 and GSK2656157, (iv) modulating PKR using C16 and 2-aminopurine, (v) inhibiting HRI using aminopyrazolindane, (vi) prolonging ISR using salubrinal, (vii) blocking GADD34 using guanabenz and Sephin1, (viii) decreasing CReP expression thereby affecting CReP-PP1 complex binding to eIF2a using nelfinavir [231] decreases CReP expression and affects CReP-PP1 complex, and (ix) reversing the consequences of eIF2a phosphorylation using ISRIB. In an aspect, a disclosed pharmaceutical formulation can comprise an agent that targets ISR signaling such as modulation of ATF4 post-translational modifications including (i) phosphorylation of ATF4 at S251 can be blocked by SL0101, an RSK2 kinase inhibitor, (ii) inhibition of phosphorylation of ATF4 at S254 can be achieved by inhibiting PKA with H-89, and (iii) nuclear to cytoplasmic shuttling of ATF4 followed by its phosphorylation-dependent proteasomal degradation can be induced by the synthesized RPL41 peptide.

### E. Methods of Identifying Dystonia Biomarker

### 1. Protein Biomarkers

Disclosed herein is a method of identifying a dystonia biomarker in a subject, the method comprising obtaining a biosample from a subject having a dystonia; obtaining a biosample from a subject not having a dystonia; determining the expression level of one or more proteins in both biosamples; identifying those proteins that are differentially expressed in the biosample obtained from the subject having a dystonia when compared to the biosample from the subject not having a dystonia; wherein those differentially expressed proteins are biomarkers of a dystonia.

In an aspect, determining the level of one or more proteins in one or both samples can comprise using liquid chromatography with tandem mass spectrometry (LC-MS-MS), parallel reaction monitoring (PRM), or multiple reaction monitoring (MRM).

In an aspect, a biosample can comprise tissues, cells, biopsies, blood, lymph, CFS, serum, plasma, urine, saliva, mucus, tears, or a combination thereof.

A biosample can comprise extracellular vesicles or extracellular vesicles collected from cultured patient-derived cells. In an aspect, cultured patient-derived cells can comprise primary cells, immortalized cells, iPSC cells, or any combination thereof.

In an aspect, one or more disclosed proteins can be those proteins in Table 4.

In an aspect, one or more disclosed proteins can comprise isocitrate dehydrogenase [NADP] (mitochondrial), aldehyde dehydrogenase (mitochondrial), inosine triphosphate pyrophosphatase, eukaryotic translation EF1 epsilon-1, histone-binding protein RBBP4, integral membrane protein 2B, spliceosome RNA helicase Ddx39b, NEDD8-activating enzyme E1 catalytic subunit, exocyst complex component 4, importin subunit alpha-3, dynactin subunit 2, importin-11, protein transport protein Sec23A, glutamine-tRNA ligase, ferritin heavy chain, phospholipase A-2-activating protein, CCR4-NOT transcription complex subunit 1, single-stranded DNA-binding protein, mitochondrial, aspartate-tRNA ligase, cytoplasmic, heterogeneous nuclear ribonucleoprotein F, histone H3.1, serine hydroxymethyltransferase, mitochondrial, osteopontin, pre-mRNA-processing-splicing factor 8, ubiquitin thioesterase OTUB1, endothelial lipase, threonine-tRNA ligase, cytoplasmic, coatomer subunit beta′, heterogeneous nuclear ribonucleoprotein L, Regulator of nonsense transcripts 1, ATP-dependent RNA helicase A, prolyl endopeptidase, aldose reductase, protein transport protein Sec31A, EGF-containing fibulin-like ECM protein 1, S-methyl-5′-thioadenosine phosphorylase, probable ATP-dependent RNA helicase DDX5, 26S proteasome non-ATPase regulatory subunit 1, DNA replication licensing factor MCM2, prelamin-A/C, Pre-mRNA-splicing factor ATP-dependent RNA helicase DHX15, spectrin beta chain, non-erythrocytic 1, latent-transforming growth factor beta-binding protein 1, spectrin alpha chain, non-erythrocytic 1, collagen alpha-1(V) chain, ATP-dependent RNA helicase DDX3X, and any combination thereof.

In an aspect, one or more disclosed proteins can be isocitrate dehydrogenase [NADP](mitochondrial), aldehyde dehydrogenase (mitochondrial), inosine triphosphate pyrophosphatase, eukaryotic translation EF1 epsilon-1, histone-binding protein RBBP4, integral membrane protein 2B, spliceosome RNA helicase Ddx39b, NEDD8-activating enzyme E1 catalytic subunit, exocyst complex component 4, importin subunit alpha-3, dynactin subunit 2, importin-11, protein transport protein Sec23A, glutamine-tRNA ligase, ferritin heavy chain, phospholipase A-2-activating protein, CCR4-NOT transcription complex subunit 1, single-stranded DNA-binding protein, mitochondrial, aspartate-tRNA ligase, cytoplasmic, heterogeneous nuclear ribonucleoprotein F, histone H3.1, serine hydroxymethyltransferase, mitochondrial, osteopontin, pre-mRNA-processing-splicing factor 8, ubiquitin thioesterase OTUB1, and any combination thereof. In an aspect, one or more disclosed proteins can comprise 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, or 26 or more of the disclosed proteins.

In an aspect, one or more disclosed proteins can be associated with dysfunction in phosphor-eIF2α signaling. In an aspect, one or more disclosed proteins can be associated with dysfunction in the integrated stress response.

In an aspect, a dystonia can be focal dystonia, blepharospasm, cervical dystonia, oromandibular dystonia, task-specific or occupational dystonia, spasmodic dysphonia, generalized dystonia, segmental dystonia, DYT1-related dystonia, DYT6-related dystonia, DYT28-related dystonia, dopa-responsive dystonia, myoclonic dystonia, X-linked dystonia-Parkinsonism, rapid-onset dystonia-Parkinsonism, paroxysmal dystonia choreoathetosis, paroxysmal kinesigenic dystonia, paroxysmal nonkinesigenic dyskinesia, paroxysmal exertion-induced dyskinesia, primary dystonia, acquired dystonia, tardive dyskinesia, or tardive dystonia.

In an aspect, a disclosed method can comprise treating a subject having a dystonia.

In an aspect, treating a subject can comprise administering one or more agents that modulate the expression level of one or more differentially expressed proteins.

In an aspect, treating a subject can comprise administering one or more agents that target eIF2α signaling, such as, for example, contributing to the phosphorylation or de-phosphorylation of eIF2α. In an aspect, a disclosed agent can be ritonavir, nelfinavir, lopinavir, saquinavir, deshydroxy-lopinavir, cobicistat, deshydroxy-ritonavir, or any combination thereof. In an aspect, a disclosed agent can be ritonavir.

In an aspect, modulating the expression level can comprise increasing the expression level, decreasing the expression level, or both. In an aspect, the expression level of one or more of the differentially expressed proteins can be increased or decreased.

In an aspect, a disclosed method can comprise repeating one or more steps of the disclosed method. In an aspect, a disclosed method can comprise modifying an administering step.

In an aspect, disclosed differential expression can comprise at least a 2-fold change, at least at 5-fold change, at least a 7-fold change, at least a 10-fold change, or more than a 10-fold change between the two biosamples. In an aspect, disclosed differential expression can comprise at least a +1, +2, +3, −1, −2, or −3 Z-score. In an aspect, disclosed differential expression can comprise at least a Cohen's d effect size of at least 0.2, at least 0.5, at least 0.8, or greater than 0.8. In an aspect, disclosed differential expression can comprise at least a Cohen's d effect size of at least 1, at least 2, at least 3, or greater than 3.

In an aspect, a disclosed method can comprise generating a proteomic profile for one or both biosamples. Individual components of the proteomic profile include but are not limited to those members shown in Table 4. For the purposes of the present invention the proteomic profile comprises from least two to all 46 of the proteins listed in Table 4.

In an aspect, a disclosed method can comprise administering one or more additional therapeutic agents. Additional therapeutic agents can comprise any disclosed therapeutic agents. A therapeutic agent can be any that effects a desired clinical outcome in a subject having a dystonia, suspected of having a dystonia, and/or likely to develop or acquire a dystonia. In an aspect, a disclosed therapeutic agent can be an oligonucleotide therapeutic agent. A disclosed oligonucleotide therapeutic agent can comprise a single-stranded or double-stranded DNA, iRNA, shRNA, siRNA, mRNA, non-coding RNA (ncRNA), an antisense molecule, miRNA, a morpholino, a peptide-nucleic acid (PNA), or an analog or conjugate thereof. In an aspect, a disclosed oligonucleotide therapeutic agent can be an ASO or an RNAi. In an aspect, a disclosed oligonucleotide therapeutic agent can comprise one or more modifications at any position applicable. In an aspect, a disclosed therapeutic agent can comprise an isolated nucleic acid molecule encoding a protein that is deficient or absent in the subject. In an aspect, a disclosed therapeutic agent can be a biologically active agent, a pharmaceutically active agent, an anti-bacterial agent, an anti-fungal agent, a corticosteroid, an analgesic, an immunostimulant, an immune-based product, or any combination thereof.

Disclosed herein is a method of identifying a dystonia biomarker in a subject, the method comprising obtaining a biosample from a subject having a dystonia; determining the expression level of one or more proteins in the biosample; identifying those proteins that are differentially expressed in the dystonia biosample when compared to that of a reference biosample; wherein those differentially expressed proteins are biomarkers of a dystonia.

In an aspect, a reference biosample can comprise a biosample from a subject not having a dystonia or an aggregate of biosamples from subjects not having a dystonia. In an aspect, a reference biosample can comprise a biosample from the subject prior to the onset of a dystonia.

In an aspect, determining the level of one or more proteins in a biosample can comprise using liquid chromatography with tandem mass spectrometry (LC-MS-MS), parallel reaction monitoring (PRM), or multiple reaction monitoring (MRM).

In an aspect, a biosample can comprise tissues, cells, biopsies, blood, lymph, CFS, serum, plasma, urine, saliva, mucus, tears, or a combination thereof. In an aspect, a disclosed biosample can comprise extracellular vesicles or extracellular vesicles collected from cultured patient-derived cells. In an aspect, cultured patient-derived cells can comprise primary cells, immortalized cells, iPSC cells, or any combination thereof.

In an aspect, a disclosed method can comprise obtaining a reference biosample. In an aspect, obtaining a reference biosample can comprise obtaining a biosample from a subject not having a dystonia and determining the expression level of one or more proteins in the biosample. In an aspect, obtaining a reference biosample can comprise obtaining a biosample from subjects not having a dystonia and determining the expression level of one or more proteins in the biosamples.

In an aspect, one or more disclosed proteins can be those proteins in Table 4.

In an aspect, one or more disclosed proteins can comprise isocitrate dehydrogenase [NADP] (mitochondrial), aldehyde dehydrogenase (mitochondrial), inosine triphosphate pyrophosphatase, eukaryotic translation EF1 epsilon-1, histone-binding protein RBBP4, integral membrane protein 2B, spliceosome RNA helicase Ddx39b, NEDD8-activating enzyme E1 catalytic subunit, exocyst complex component 4, importin subunit alpha-3, dynactin subunit 2, importin-11, protein transport protein Sec23A, glutamine-tRNA ligase, ferritin heavy chain, phospholipase A-2-activating protein, CCR4-NOT transcription complex subunit 1, single-stranded DNA-binding protein, mitochondrial, aspartate-tRNA ligase, cytoplasmic, heterogeneous nuclear ribonucleoprotein F, histone H3.1, serine hydroxymethyltransferase, mitochondrial, osteopontin, pre-mRNA-processing-splicing factor 8, ubiquitin thioesterase OTUB1, endothelial lipase, threonine-tRNA ligase, cytoplasmic, coatomer subunit beta′, heterogeneous nuclear ribonucleoprotein L, Regulator of nonsense transcripts 1, ATP-dependent RNA helicase A, prolyl endopeptidase, aldose reductase, protein transport protein Sec31A, EGF-containing fibulin-like ECM protein 1, S-methyl-5′-thioadenosine phosphorylase, probable ATP-dependent RNA helicase DDX5, 26S proteasome non-ATPase regulatory subunit 1, DNA replication licensing factor MCM2, prelamin-A/C, Pre-mRNA-splicing factor ATP-dependent RNA helicase DHX15, spectrin beta chain, non-erythrocytic 1, latent-transforming growth factor beta-binding protein 1, spectrin alpha chain, non-erythrocytic 1, collagen alpha-1(V) chain, ATP-dependent RNA helicase DDX3X, and any combination thereof.

In an aspect, one or more disclosed proteins can be isocitrate dehydrogenase [NADP](mitochondrial), aldehyde dehydrogenase (mitochondrial), inosine triphosphate pyrophosphatase, eukaryotic translation EF1 epsilon-1, histone-binding protein RBBP4, integral membrane protein 2B, spliceosome RNA helicase Ddx39b, NEDD8-activating enzyme E1 catalytic subunit, exocyst complex component 4, importin subunit alpha-3, dynactin subunit 2, importin-11, protein transport protein Sec23A, glutamine-tRNA ligase, ferritin heavy chain, phospholipase A-2-activating protein, CCR4-NOT transcription complex subunit 1, single-stranded DNA-binding protein, mitochondrial, aspartate-tRNA ligase, cytoplasmic, heterogeneous nuclear ribonucleoprotein F, histone H3.1, serine hydroxymethyltransferase, mitochondrial, osteopontin, pre-mRNA-processing-splicing factor 8, ubiquitin thioesterase OTUB1, and any combination thereof. In an aspect, one or more disclosed proteins can comprise 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, or 26 or more of the disclosed proteins.

In an aspect, one or more disclosed proteins can be associated with dysfunction in phosphor-eIF2α signaling. In an aspect, one or more disclosed proteins can be associated with dysfunction in the integrated stress response.

In an aspect, a dystonia can be focal dystonia, blepharospasm, cervical dystonia, oromandibular dystonia, task-specific or occupational dystonia, spasmodic dysphonia, generalized dystonia, segmental dystonia, DYT1-related dystonia, DYT6-related dystonia, DYT28-related dystonia, dopa-responsive dystonia, myoclonic dystonia, X-linked dystonia-Parkinsonism, rapid-onset dystonia-Parkinsonism, paroxysmal dystonia choreoathetosis, paroxysmal kinesigenic dystonia, paroxysmal nonkinesigenic dyskinesia, paroxysmal exertion-induced dyskinesia, primary dystonia, acquired dystonia, tardive dyskinesia, or tardive dystonia.

In an aspect, a disclosed method can comprise treating a subject having a dystonia. In an aspect, treating a subject can comprise administering one or more agents that modulate the expression level of one or more differentially expressed proteins. In an aspect, modulating the expression level can comprise increasing the expression level, decreasing the expression level, or both. In an aspect, the expression level of one or more of the differentially expressed proteins can be increased or decreased.

In an aspect, treating a subject can comprise administering one or more agents that target eIF2α signaling, such as, for example, contributing to the phosphorylation or de-phosphorylation of eIF2α. In an aspect, a disclosed agent can be ritonavir, nelfinavir, lopinavir, saquinavir, deshydroxy-lopinavir, cobicistat, deshydroxy-ritonavir, or any combination thereof. In an aspect, a disclosed agent can be ritonavir.

In an aspect, a disclosed method can comprise repeating one or more steps of the disclosed method. In an aspect, a disclosed method can comprise modifying an administering step.

In an aspect, disclosed differential expression can comprise at least a 2-fold change, at least at 5-fold change, at least a 7-fold change, at least a 10-fold change, or more than a 10-fold change between the two biosamples. In an aspect, disclosed differential expression can comprise at least a +1, +2, +3, −1, −2, or −3 Z-score. In an aspect, disclosed differential expression can comprise at least a Cohen's d effect size of at least 0.2, at least 0.5, at least 0.8, or greater than 0.8. In an aspect, disclosed differential expression can comprise at least a Cohen's d effect size of at least 1, at least 2, at least 3, or greater than 3.

In an aspect, a disclosed method can comprise generating a proteomic profile for one or both biosamples. Individual components of the proteomic profile include but are not limited to those members shown in Table 4. For the purposes of the present invention the proteomic profile comprises from least two to all 46 of the proteins listed in Table 4.

In an aspect, a disclosed method can comprise administering one or more additional therapeutic agents. One or more additional therapeutic agents can comprise any therapeutic agents disclosed herein.

2. miRNA Biomarkers

Disclosed herein is a method of identifying a dystonia biomarker in a subject comprising obtaining a biosample from a subject having a dystonia; obtaining a biosample from a subject not having a dystonia; determining the expression level of one or more miRNAs in both biosamples; identifying those miRNAs that are differentially expressed in the biosample obtained from the subject having a dystonia when compared to the biosample from the subject not having a dystonia; wherein those differentially expressed miRNAs are biomarkers of a dystonia.

In an aspect, a disclosed method can comprise obtaining a reference biosample. In an aspect, obtaining a reference biosample can comprise obtaining a biosample from a subject not having a dystonia and determining the expression level of one or more miRNAs in the biosample. In an aspect, obtaining a reference biosample can comprise obtaining a biosample from subjects not having a dystonia and determining the expression level of one or more miRNAs in the biosamples.

In an aspect, a reference biosample can comprise a biosample from a subject not having a dystonia or an aggregate of biosamples from subjects not having a dystonia. In an aspect, a reference biosample can comprise a biosample from the subject prior to the onset of a dystonia. In an aspect, determining the level of one or more miRNAs in a biosample can comprise using RNASeq or RT-qPCR. In an aspect, determining the level of one or more miRNAs can comprise a commercial assay (e.g., NanoStrin nCounter® assays), isothermal amplification-based assays, oligonucleotide-templated reactions, nanobead-based systems, and microfluidic-based assays for miRNA capture from biosamples and detection, and any combination thereof.

In an aspect, a biosample can comprise tissues, cells, biopsies, blood, lymph, CFS, serum, plasma, urine, saliva, mucus, tears, or a combination thereof. A biosample can comprise extracellular vesicles or extracellular vesicles collected from cultured patient-derived cells. In an aspect, cultured patient-derived cells can comprise primary cells, immortalized cells, iPSC cells, or any combination thereof.

In an aspect, one or more disclosed miRNAs can be those miRNAs in Table 7.

In an aspect, one or more disclosed miRNAs can comprise miR-135a-5p, miR-182-5p, miR-542-5p, miR-298-5p, miR-183-5p, miR-296-3p, miR-96-5p, miR-344d-3p, miR-5121, miR-140-3p, miR-344-3p, miR-187-3p, miR-130b-3p, miR-125b-1-3p, miR-34a-5p, miR-532-5p, miR-148a-3p, miR-3535, miR-362-5p, miR-192-5p, miR-34c-5p, miR-1291, miR-30b-5p, miR-362-3p, miR-671-5p, miR-31-5p, miR-22-3p, miR-199b-3p, miR-199a-3p, miR-30e-5p, miR-30c-5p, miR-30a-5p, miR-93-5p, miR-19b-3p, let-7i-5p, miR-103-3p, let-7f-5p, miR-26b-5p, and any combination thereof.

In an aspect, one or more disclosed miRNAs can be miR-135a-5p, miR-182-5p, miR-542-5p, miR-183-5p, miR-296-3p, miR-96-5p, miR-34a-5p, miR-22-3p, miR-199a-3p, and any combination thereof. In an aspect, one or more disclosed miRNAs can be miR-135a-5p, miR-183-5p, miR-296-3p, miR-96-5p, miR-34a-5p, miR-22-3p, miR-199a-3p, and any combination thereof.

In an aspect, one or more disclosed miRNAs can comprise 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, or 26 or more of the disclosed miRNAs.

In an aspect, one or more disclosed miRNAs can be associated with dysfunction in phosphor-eIF2α signaling. In an aspect, one or more disclosed miRNAs can be associated with dysfunction in the integrated stress response.

In an aspect, a dystonia can be focal dystonia, blepharospasm, cervical dystonia, oromandibular dystonia, task-specific or occupational dystonia, spasmodic dysphonia, generalized dystonia, segmental dystonia, DYT1-related dystonia, DYT6-related dystonia, DYT28-related dystonia, dopa-responsive dystonia, myoclonic dystonia, X-linked dystonia-Parkinsonism, rapid-onset dystonia-Parkinsonism, paroxysmal dystonia choreoathetosis, paroxysmal kinesigenic dystonia, paroxysmal nonkinesigenic dyskinesia, paroxysmal exertion-induced dyskinesia, primary dystonia, acquired dystonia, tardive dyskinesia, or tardive dystonia.

In an aspect, a disclosed method can comprise treating a subject having a dystonia.

In an aspect, treating a subject can comprise administering one or more agents that modulate the expression level of one or more differentially expressed miRNAs In an aspect, modulating the expression level can comprise increasing the expression level, decreasing the expression level, or both. In an aspect, the expression level of one or more of the differentially expressed miRNAs can be increased or decreased.

In an aspect, a disclosed method can comprise treating a subject. In an aspect, treating a subject can comprise administering one or more agents that target eIF2α signaling, such as, for example, contributing to the phosphorylation or de-phosphorylation of eIF2α.

In an aspect, a disclosed agent can be ritonavir, nelfinavir, lopinavir, saquinavir, deshydroxy-lopinavir, cobicistat, deshydroxy-ritonavir, or any combination thereof. In an aspect, a disclosed agent can be ritonavir.

In an aspect, a disclosed method can comprise repeating one or more steps of the disclosed method. In an aspect, a disclosed method can comprise modifying an administering step.

In an aspect, disclosed differential expression can comprise at least a 2-fold change, at least at 5-fold change, at least a 7-fold change, at least a 10-fold change, or more than a 10-fold change between the two biosamples. In an aspect, disclosed differential expression can comprise at least a +1, +2, +3, −1, −2, or −3 Z-score. In an aspect, disclosed differential expression can comprise at least a Cohen's d effect size of at least 0.2, at least 0.5, at least 0.8, or greater than 0.8. In an aspect, disclosed differential expression can comprise at least a Cohen's d effect size of at least 1, at least 2, at least 3, or greater than 3.

In an aspect, a disclosed method can comprise administering one or more additional therapeutic agents. One or more additional therapeutic agents can comprise any therapeutic agents disclosed herein.

Disclosed herein is a method of identifying a dystonia biomarker in a subject comprising obtaining a biosample from a subject having a dystonia; determining the expression level of one or more miRNAs in the biosample; identifying those miRNAs that are differentially expressed in the dystonia biosample when compared to that of a reference biosample; wherein those differentially expressed miRNAs are biomarkers of a dystonia.

In an aspect, a disclosed method can comprise obtaining a reference biosample. In an aspect, obtaining a reference biosample can comprise obtaining a biosample from a subject not having a dystonia and determining the expression level of one or more miRNAs in the biosample.

In an aspect, obtaining a reference biosample can comprise obtaining a biosample from subjects not having a dystonia and determining the expression level of one or more miRNAs in the biosamples.

In an aspect, a reference biosample can comprise a biosample from a subject not having a dystonia or an aggregate of biosamples from subjects not having a dystonia. In an aspect, a reference biosample can comprise a biosample from the subject prior to the onset of a dystonia.

In an aspect, determining the level of one or more miRNAs in a biosample can comprise using RNASeq or RT-qPCR. In an aspect, determining the level of one or more miRNAs can comprise a commercial assay (e.g., NanoStrin nCounter® assays), isothermal amplification-based assays, oligonucleotide-templated reactions, nanobead-based systems, and microfluidic-based assays for miRNA capture from biosamples and detection, and any combination thereof.

In an aspect, a biosample can comprise tissues, cells, biopsies, blood, lymph, CFS, serum, plasma, urine, saliva, mucus, tears, or a combination thereof. A biosample can comprise extracellular vesicles or extracellular vesicles collected from cultured patient-derived cells. In an aspect, cultured patient-derived cells can comprise primary cells, immortalized cells, iPSC cells, or any combination thereof.

In an aspect, one or more disclosed miRNAs can be those miRNAs in Table 7.

In an aspect, one or more disclosed miRNAs can comprise miR-135a-5p, miR-182-5p, miR-542-5p, miR-298-5p, miR-183-5p, miR-296-3p, miR-96-5p, miR-344d-3p, miR-5121, miR-140-3p, miR-344-3p, miR-187-3p, miR-130b-3p, miR-125b-1-3p, miR-34a-5p, miR-532-5p, miR-148a-3p, miR-3535, miR-362-5p, miR-192-5p, miR-34c-5p, miR-1291, miR-30b-5p, miR-362-3p, miR-671-5p, miR-31-5p, miR-22-3p, miR-199b-3p, miR-199a-3p, miR-30e-5p, miR-30c-5p, miR-30a-5p, miR-93-5p, miR-19b-3p, let-7i-5p, miR-103-3p, let-7f-5p, miR-26b-5p, and any combination thereof.

In an aspect, one or more disclosed miRNAs can be miR-135a-5p, miR-182-5p, miR-542-5p, miR-183-5p, miR-296-3p, miR-96-5p, miR-34a-5p, miR-22-3p, miR-199a-3p, and any combination thereof.

In an aspect, one or more disclosed miRNAs can be associated with dysfunction in phosphor-eIF2α signaling. In an aspect, one or more disclosed miRNAs can be associated with dysfunction in the integrated stress response.

In an aspect, a dystonia can be focal dystonia, blepharospasm, cervical dystonia, oromandibular dystonia, task-specific or occupational dystonia, spasmodic dysphonia, generalized dystonia, segmental dystonia, DYT1-related dystonia, DYT6-related dystonia, DYT28-related dystonia, dopa-responsive dystonia, myoclonic dystonia, X-linked dystonia-Parkinsonism, rapid-onset dystonia-Parkinsonism, paroxysmal dystonia choreoathetosis, paroxysmal kinesigenic dystonia, paroxysmal nonkinesigenic dyskinesia, paroxysmal exertion-induced dyskinesia, primary dystonia, acquired dystonia, tardive dyskinesia, or tardive dystonia.

In an aspect, a disclosed method can comprise treating a subject having a dystonia. In an aspect, treating a subject can comprise administering one or more agents that modulate the expression level of one or more differentially expressed miRNAs. In an aspect, modulating the expression level can comprise increasing the expression level, decreasing the expression level, or both. In an aspect, the expression level of one or more of the differentially expressed miRNAs can be increased or decreased.

In an aspect, a disclosed method can comprise treating a subject. In an aspect, treating a subject can comprise administering one or more agents that target eIF2α signaling, such as, for example, contributing to the phosphorylation or de-phosphorylation of eIF2α.

In an aspect, a disclosed agent can be ritonavir, nelfinavir, lopinavir, saquinavir, deshydroxy-lopinavir, cobicistat, deshydroxy-ritonavir, or any combination thereof. In an aspect, a disclosed agent can be ritonavir.

In an aspect, a disclosed method can comprise repeating one or more steps of the disclosed method. In an aspect, a disclosed method can comprise modifying an administering step.

In an aspect, disclosed differential expression can comprise at least a 2-fold change, at least at 5-fold change, at least a 7-fold change, at least a 10-fold change, or more than a 10-fold change between the two biosamples. In an aspect, disclosed differential expression can comprise at least a +1, +2, +3, −1, −2, or −3 Z-score. In an aspect, disclosed differential expression can comprise at least a Cohen's d effect size of at least 0.2, at least 0.5, at least 0.8, or greater than 0.8. In an aspect, disclosed differential expression can comprise at least a Cohen's d effect size of at least 1, at least 2, at least 3, or greater than 3.

In an aspect, a disclosed method can comprise administering one or more additional therapeutic agents. One or more additional therapeutic agents can comprise any therapeutic agents disclosed herein.

### F. Methods of Treating a Subject Having A Dystonia

### 1. Protein Biomarkers

Disclosed herein is a method of treating a subject having a dystonia, the method comprising obtaining a biosample from a subject after treatment; determining the expression level of one or more proteins in the post-treatment biosample, wherein: if the post-treatment expression level represents an improvement over a pre-treatment expression level of the one or more proteins, or if the post-treatment expression level is within an acceptable range of a reference expression level, then continuing to administer the treatment.

In an aspect, a disclosed method can comprise obtaining a biosample from the subject prior to treatment and detecting the expression level of one or more proteins in the pre-treatment biosample.

In an aspect, determining the level of one or more proteins in a biosample can comprise liquid chromatography with tandem mass spectrometry (LC-MS-MS), parallel reaction monitoring (PRM), or multiple reaction monitoring (MRM).

In an aspect, a disclosed reference expression level can comprise an expression level obtained from a biosample from a subject not having a dystonia. In an aspect, a disclosed reference expression level can comprise an aggregate expression level obtained from biosamples of subjects not having a dystonia. In an aspect, a reference biosample can comprise a biosample from a subject not having a dystonia or an aggregate of biosamples from subjects not having a dystonia.

In an aspect, obtaining a reference biosample can comprise obtaining a biosample from a subject not having a dystonia and determining the expression level of one or more proteins in the biosample. In an aspect, obtaining a reference biosample can comprise obtaining a biosample from subjects not having a dystonia and determining the expression level of one or more proteins in the biosamples.

In an aspect, the post-treatment expression level of the one or more proteins can represent an improvement over a pre-treatment expression level when the post-treatment expression level is more similar to a reference expression level than to the pre-treatment expression level.

In an aspect, a biosample can comprise tissues, cells, biopsies, blood, lymph, CFS, serum, plasma, urine, saliva, mucus, tears, or a combination thereof. In an aspect, a biosample can comprise extracellular vesicles or extracellular vesicles collected from cultured patient-derived cells. In an aspect, cultured patient-derived cells can comprise primary cells, immortalized cells, iPSC cells, or any combination thereof.

In an aspect, one or more disclosed proteins can be those proteins in Table 4.

In an aspect, one or more disclosed proteins can comprise isocitrate dehydrogenase [NADP] (mitochondrial), aldehyde dehydrogenase (mitochondrial), inosine triphosphate pyrophosphatase, eukaryotic translation EF1 epsilon-1, histone-binding protein RBBP4, integral membrane protein 2B, spliceosome RNA helicase Ddx39b, NEDD8-activating enzyme E1 catalytic subunit, exocyst complex component 4, importin subunit alpha-3, dynactin subunit 2, importin-11, protein transport protein Sec23A, glutamine-tRNA ligase, ferritin heavy chain, phospholipase A-2-activating protein, CCR4-NOT transcription complex subunit 1, single-stranded DNA-binding protein, mitochondrial, aspartate-tRNA ligase, cytoplasmic, heterogeneous nuclear ribonucleoprotein F, histone H3.1, serine hydroxymethyltransferase, mitochondrial, osteopontin, pre-mRNA-processing-splicing factor 8, ubiquitin thioesterase OTUB1, endothelial lipase, threonine-tRNA ligase, cytoplasmic, coatomer subunit beta′, heterogeneous nuclear ribonucleoprotein L, Regulator of nonsense transcripts 1, ATP-dependent RNA helicase A, prolyl endopeptidase, aldose reductase, protein transport protein Sec31A, EGF-containing fibulin-like ECM protein 1, S-methyl-5′-thioadenosine phosphorylase, probable ATP-dependent RNA helicase DDX5, 26S proteasome non-ATPase regulatory subunit 1, DNA replication licensing factor MCM2, prelamin-A/C, Pre-mRNA-splicing factor ATP-dependent RNA helicase DHX15, spectrin beta chain, non-erythrocytic 1, latent-transforming growth factor beta-binding protein 1, spectrin alpha chain, non-erythrocytic 1, collagen alpha-1(V) chain, ATP-dependent RNA helicase DDX3X, and any combination thereof.

In an aspect, one or more disclosed proteins can be isocitrate dehydrogenase [NADP](mitochondrial), aldehyde dehydrogenase (mitochondrial), inosine triphosphate pyrophosphatase, eukaryotic translation EF1 epsilon-1, histone-binding protein RBBP4, integral membrane protein 2B, spliceosome RNA helicase Ddx39b, NEDD8-activating enzyme E1 catalytic subunit, exocyst complex component 4, importin subunit alpha-3, dynactin subunit 2, importin-11, protein transport protein Sec23A, glutamine-tRNA ligase, ferritin heavy chain, phospholipase A-2-activating protein, CCR4-NOT transcription complex subunit 1, single-stranded DNA-binding protein, mitochondrial, aspartate-tRNA ligase, cytoplasmic, heterogeneous nuclear ribonucleoprotein F, histone H3.1, serine hydroxymethyltransferase, mitochondrial, osteopontin, pre-mRNA-processing-splicing factor 8, ubiquitin thioesterase OTUB1, and any combination thereof. In an aspect, one or more disclosed proteins can comprise 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, or 26 or more of the disclosed proteins.

In an aspect, one or more disclosed proteins can be associated with dysfunction in phosphor-eIF2α signaling. In an aspect, one or more disclosed proteins can be associated with dysfunction in the integrated stress response.

In an aspect, a dystonia can be focal dystonia, blepharospasm, cervical dystonia, oromandibular dystonia, task-specific or occupational dystonia, spasmodic dysphonia, generalized dystonia, segmental dystonia, DYT1-related dystonia, DYT6-related dystonia, DYT28-related dystonia, dopa-responsive dystonia, myoclonic dystonia, X-linked dystonia-Parkinsonism, rapid-onset dystonia-Parkinsonism, paroxysmal dystonia choreoathetosis, paroxysmal kinesigenic dystonia, paroxysmal nonkinesigenic dyskinesia, paroxysmal exertion-induced dyskinesia, primary dystonia, acquired dystonia, tardive dyskinesia, or tardive dystonia.

In an aspect of a disclosed method, treatment can comprise administering to the subject one or more agents that modulate the expression level of one or more differentially expressed proteins. In an aspect, modulating the expression level can comprise increasing the expression level, decreasing the expression level, or both. In an aspect, the expression level of one or more of the differentially expressed proteins can be increased or decreased.

In an aspect, a disclosed method can comprise treating a subject. In an aspect, treating a subject can comprise administering one or more agents that target eIF2α signaling, such as, for example, contributing to the phosphorylation or de-phosphorylation of eIF2α.

In an aspect, a disclosed agent can be ritonavir, nelfinavir, lopinavir, saquinavir, deshydroxy-lopinavir, cobicistat, deshydroxy-ritonavir, or any combination thereof. In an aspect, a disclosed agent can be ritonavir.

In an aspect, a disclosed method can comprise repeating one or more steps of the disclosed method. In an aspect, a disclosed method can comprise modifying an administering step.

In an aspect, disclosed differential expression can comprise at least a 2-fold change, at least at 5-fold change, at least a 7-fold change, at least a 10-fold change, or more than a 10-fold change between the two biosamples. In an aspect, disclosed differential expression can comprise at least a +1, +2, +3, −1, −2, or −3 Z-score. In an aspect, disclosed differential expression can comprise at least a Cohen's d effect size of at least 0.2, at least 0.5, at least 0.8, or greater than 0.8. In an aspect, disclosed differential expression can comprise at least a Cohen's d effect size of at least 1, at least 2, at least 3, or greater than 3.

In an aspect, a disclosed method can comprise generating a proteomic profile for one or both biosamples. Individual components of the proteomic profile include but are not limited to those members shown in Table 4. For the purposes of the present invention the proteomic profile comprises from least two to all 46 of the proteins listed in Table 4.

In an aspect, a disclosed method can comprise administering one or more additional therapeutic agents. One or more additional therapeutic agents can comprise any therapeutic agents disclosed herein.

2. miRNA Biomarkers

Disclosed herein is a method of treating a subject having a dystonia comprising obtaining a biosample from a subject after treatment; determining the expression level of one or more miRNAs in the post-treatment biosample, wherein if the post-treatment expression level represents an improvement over a pre-treatment expression level of the one or more miRNAs, or if the post-treatment expression level is within an acceptable range of a reference expression level, then continuing to administer the treatment.

In an aspect, a disclosed method can comprise obtaining a biosample from the subject prior to treatment and detecting the expression level of one or more miRNAs in the pre-treatment biosample. In an aspect, a disclosed method can comprise obtaining a reference biosample. In an aspect, obtaining a reference biosample can comprise obtaining a biosample from a subject not having a dystonia and determining the expression level of one or more miRNAs in the biosample. In an aspect, obtaining a reference biosample can comprise obtaining a biosample from subjects not having a dystonia and determining the expression level of one or more miRNAs in the biosamples.

In an aspect, a reference biosample can comprise a biosample from a subject not having a dystonia or an aggregate of biosamples from subjects not having a dystonia. In an aspect, a reference biosample can comprise a biosample from the subject prior to the onset of a dystonia.

In an aspect, determining the level of one or more miRNAs in a biosample can comprise using RNASeq or RT-qPCR. In an aspect, determining the level of one or more miRNAs can comprise a commercial assay (e.g., NanoStrin nCounter® assays), isothermal amplification-based assays, oligonucleotide-templated reactions, nanobead-based systems, and microfluidic-based assays for miRNA capture from biosamples and detection, and any combination thereof.

In an aspect, a biosample can comprise tissues, cells, biopsies, blood, lymph, CFS, serum, plasma, urine, saliva, mucus, tears, or a combination thereof. A biosample can comprise extracellular vesicles or extracellular vesicles collected from cultured patient-derived cells. In an aspect, cultured patient-derived cells can comprise primary cells, immortalized cells, iPSC cells, or any combination thereof.

In an aspect, one or more disclosed miRNAs can be those miRNAs in Table 7.

In an aspect, one or more disclosed miRNAs can comprise miR-135a-5p, miR-182-5p, miR-542-5p, miR-298-5p, miR-183-5p, miR-296-3p, miR-96-5p, miR-344d-3p, miR-5121, miR-140-3p, miR-344-3p, miR-187-3p, miR-130b-3p, miR-125b-1-3p, miR-34a-5p, miR-532-5p, miR-148a-3p, miR-3535, miR-362-5p, miR-192-5p, miR-34c-5p, miR-1291, miR-30b-5p, miR-362-3p, miR-671-5p, miR-31-5p, miR-22-3p, miR-199b-3p, miR-199a-3p, miR-30e-5p, miR-30c-5p, miR-30a-5p, miR-93-5p, miR-19b-3p, let-7i-5p, miR-103-3p, let-7f-5p, miR-26b-5p, and any combination thereof.

In an aspect, one or more disclosed miRNAs can be miR-135a-5p, miR-182-5p, miR-542-5p, miR-183-5p, miR-296-3p, miR-96-5p, miR-34a-5p, miR-22-3p, miR-199a-3p, and any combination thereof. In an aspect, one or more disclosed miRNAs can be miR-135a-5p, miR-183-5p, miR-296-3p, miR-96-5p, miR-34a-5p, miR-22-3p, miR-199a-3p, and any combination thereof.

In an aspect, one or more disclosed miRNAs can comprise 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, or 26 or more of the disclosed miRNAs.

In an aspect, one or more disclosed miRNAs can be associated with dysfunction in phosphor-eIF2α signaling. In an aspect, one or more disclosed miRNAs can be associated with dysfunction in the integrated stress response.

In an aspect, a dystonia can be focal dystonia, blepharospasm, cervical dystonia, oromandibular dystonia, task-specific or occupational dystonia, spasmodic dysphonia, generalized dystonia, segmental dystonia, DYT1-related dystonia, DYT6-related dystonia, DYT28-related dystonia, dopa-responsive dystonia, myoclonic dystonia, X-linked dystonia-Parkinsonism, rapid-onset dystonia-Parkinsonism, paroxysmal dystonia choreoathetosis, paroxysmal kinesigenic dystonia, paroxysmal nonkinesigenic dyskinesia, paroxysmal exertion-induced dyskinesia, primary dystonia, acquired dystonia, tardive dyskinesia, or tardive dystonia.

In an aspect, wherein treating a subject can comprise administering one or more agents that modulate the expression level of one or more differentially expressed miRNAs In an aspect, modulating the expression level can comprise increasing the expression level, decreasing the expression level, or both. In an aspect, the expression level of one or more of the differentially expressed miRNAs can be increased or decreased.

In an aspect, wherein treating a subject can comprise administering one or more agents that target eIF2α signaling, such as, for example, contributing to the phosphorylation or de-phosphorylation of eIF2α.

In an aspect, a disclosed agent can be ritonavir, nelfinavir, lopinavir, saquinavir, deshydroxy-lopinavir, cobicistat, deshydroxy-ritonavir, or any combination thereof. In an aspect, a disclosed agent can be ritonavir.

In an aspect, a disclosed method can comprise repeating one or more steps of the disclosed method. In an aspect, a disclosed method can comprise modifying an administering step.

In an aspect, disclosed differential expression can comprise at least a 2-fold change, at least at 5-fold change, at least a 7-fold change, at least a 10-fold change, or more than a 10-fold change between the two biosamples. In an aspect, disclosed differential expression can comprise at least a +1, +2, +3, −1, −2, or −3 Z-score. In an aspect, disclosed differential expression can comprise at least a Cohen's d effect size of at least 0.2, at least 0.5, at least 0.8, or greater than 0.8. In an aspect, disclosed differential expression can comprise at least a Cohen's d effect size of at least 1, at least 2, at least 3, or greater than 3.

In an aspect, a disclosed method can comprise administering one or more additional therapeutic agents. One or more additional therapeutic agents can comprise any therapeutic agents disclosed herein.

### G. Methods of Predicting Penetrance of a Dystonia

### 1. Protein Biomarkers

Disclosed herein is a method of predicting penetrance of a dystonia in a subject comprising obtaining a biosample from a subject; determining the expression level of one or more proteins in the biosample; identifying those proteins that are differentially expressed in the biosample when compared to a reference biosample; wherein the degree of differential expression predicts the likelihood of penetrance.

In an aspect, determining the level of one or more proteins in a biosample can comprise using liquid chromatography with tandem mass spectrometry (LC-MS-MS), parallel reaction monitoring (PRM), or multiple reaction monitoring (MRM).

In an aspect, a biosample can comprise tissues, cells, biopsies, blood, lymph, CFS, serum, plasma, urine, saliva, mucus, tears, or a combination thereof. A biosample can comprise extracellular vesicles or extracellular vesicles collected from cultured patient-derived cells. In an aspect, cultured patient-derived cells can comprise primary cells, immortalized cells, iPSC cells, or any combination thereof.

In an aspect, a disclosed method can comprise obtaining a reference biosample. In an aspect, obtaining a reference biosample can comprise obtaining a biosample from a subject not having a dystonia and determining the expression level of one or more proteins in the biosample. In an aspect, obtaining a reference biosample can comprise obtaining a biosample from subjects not having a dystonia and determining the expression level of one or more proteins in the biosamples.

In an aspect, a reference biosample can comprise a biosample from a subject not having a dystonia or an aggregate of biosamples from subjects not having a dystonia. In an aspect, a reference biosample can comprise a biosample from the subject prior to the onset of a dystonia.

In an aspect, disclosed differential expression can comprise at least a 2-fold change, at least at 5-fold change, at least a 7-fold change, at least a 10-fold change, or more than a 10-fold change between the two biosamples. In an aspect, disclosed differential expression can comprise at least a +1, +2, +3, −1, −2, or −3 Z-score. In an aspect, disclosed differential expression can comprise at least a Cohen's d effect size of at least 0.2, at least 0.5, at least 0.8, or greater than 0.8. In an aspect, disclosed differential expression can comprise at least a Cohen's d effect size of at least 1, at least 2, at least 3, or greater than 3.

In an aspect, when the differential expression of a protein comprises at least a 2-fold change, at least a 5-fold change, at least a 7-fold change, at least a 10-fold change, or more than a 10-fold change, the risk of penetrance increases. In an aspect, when the differential expression of a protein comprises at least +2 or −2 Z-score, the risk of penetrance increases. In an aspect, when the differential expression of a protein comprises at least +3 or −3 Z-score, the risk of penetrance increases.

In an aspect, one or more disclosed proteins can comprise isocitrate dehydrogenase [NADP] (mitochondrial), aldehyde dehydrogenase (mitochondrial), inosine triphosphate pyrophosphatase, eukaryotic translation EF1 epsilon-1, histone-binding protein RBBP4, integral membrane protein 2B, spliceosome RNA helicase Ddx39b, NEDD8-activating enzyme E1 catalytic subunit, exocyst complex component 4, importin subunit alpha-3, dynactin subunit 2, importin-11, protein transport protein Sec23A, glutamine-tRNA ligase, ferritin heavy chain, phospholipase A-2-activating protein, CCR4-NOT transcription complex subunit 1, single-stranded DNA-binding protein, mitochondrial, aspartate-tRNA ligase, cytoplasmic, heterogeneous nuclear ribonucleoprotein F, histone H3.1, serine hydroxymethyltransferase, mitochondrial, osteopontin, pre-mRNA-processing-splicing factor 8, ubiquitin thioesterase OTUB1, endothelial lipase, threonine-tRNA ligase, cytoplasmic, coatomer subunit beta′, heterogeneous nuclear ribonucleoprotein L, Regulator of nonsense transcripts 1, ATP-dependent RNA helicase A, prolyl endopeptidase, aldose reductase, protein transport protein Sec31A, EGF-containing fibulin-like ECM protein 1, S-methyl-5′-thioadenosine phosphorylase, probable ATP-dependent RNA helicase DDX5, 26S proteasome non-ATPase regulatory subunit 1, DNA replication licensing factor MCM2, prelamin-A/C, Pre-mRNA-splicing factor ATP-dependent RNA helicase DHX15, spectrin beta chain, non-erythrocytic 1, latent-transforming growth factor beta-binding protein 1, spectrin alpha chain, non-erythrocytic 1, collagen alpha-1(V) chain, ATP-dependent RNA helicase DDX3X, and any combination thereof.

In an aspect, one or more disclosed proteins can be isocitrate dehydrogenase [NADP](mitochondrial), aldehyde dehydrogenase (mitochondrial), inosine triphosphate pyrophosphatase, eukaryotic translation EF1 epsilon-1, histone-binding protein RBBP4, integral membrane protein 2B, spliceosome RNA helicase Ddx39b, NEDD8-activating enzyme E1 catalytic subunit, exocyst complex component 4, importin subunit alpha-3, dynactin subunit 2, importin-11, protein transport protein Sec23A, glutamine-tRNA ligase, ferritin heavy chain, phospholipase A-2-activating protein, CCR4-NOT transcription complex subunit 1, single-stranded DNA-binding protein, mitochondrial, aspartate-tRNA ligase, cytoplasmic, heterogeneous nuclear ribonucleoprotein F, histone H3.1, serine hydroxymethyltransferase, mitochondrial, osteopontin, pre-mRNA-processing-splicing factor 8, ubiquitin thioesterase OTUB1, and any combination thereof. In an aspect, one or more disclosed proteins can comprise 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, or 26 or more of the disclosed proteins.

In an aspect, one or more disclosed proteins can be associated with dysfunction in phosphor-eIF2α signaling. In an aspect, one or more disclosed proteins can be associated with dysfunction in the integrated stress response.

In an aspect, a dystonia can be focal dystonia, blepharospasm, cervical dystonia, oromandibular dystonia, task-specific or occupational dystonia, spasmodic dysphonia, generalized dystonia, segmental dystonia, DYT1-related dystonia, DYT6-related dystonia, DYT28-related dystonia, dopa-responsive dystonia, myoclonic dystonia, X-linked dystonia-Parkinsonism, rapid-onset dystonia-Parkinsonism, paroxysmal dystonia choreoathetosis, paroxysmal kinesigenic dystonia, paroxysmal nonkinesigenic dyskinesia, paroxysmal exertion-induced dyskinesia, primary dystonia, acquired dystonia, tardive dyskinesia, or tardive dystonia.

In an aspect, a disclosed method can comprise treating a subject having a dystonia.

In an aspect, treating a subject can comprise administering one or more agents that modulate the expression level of one or more differentially expressed proteins. In an aspect, modulating the expression level can comprise increasing the expression level, decreasing the expression level, or both. In an aspect, the expression level of one or more of the differentially expressed proteins can be increased or decreased.

In an aspect, a disclosed method can comprise treating a subject. In an aspect, treating a subject can comprise administering one or more agents that target eIF2α signaling, such as, for example, contributing to the phosphorylation or de-phosphorylation of eIF2α.

In an aspect, a disclosed agent can be ritonavir, nelfinavir, lopinavir, saquinavir, deshydroxy-lopinavir, cobicistat, deshydroxy-ritonavir, or any combination thereof. In an aspect, a disclosed agent can be ritonavir.

In an aspect, a disclosed method can comprise repeating one or more steps of the disclosed method. In an aspect, a disclosed method can comprise modifying an administering step.

In an aspect, disclosed differential expression can comprise at least a 2-fold change, at least at 5-fold change, at least a 7-fold change, at least a 10-fold change, or more than a 10-fold change between the two biosamples. In an aspect, disclosed differential expression can comprise at least a +1, +2, +3, −1, −2, or −3 Z-score. In an aspect, disclosed differential expression can comprise at least a Cohen's d effect size of at least 0.2, at least 0.5, at least 0.8, or greater than 0.8. In an aspect, disclosed differential expression can comprise at least a Cohen's d effect size of at least 1, at least 2, at least 3, or greater than 3.

In an aspect, a disclosed method can comprise generating a proteomic profile for one or both biosamples. Individual components of the proteomic profile include but are not limited to those members shown in Table 4. For the purposes of the present invention the proteomic profile comprises from least two to all 48 of the proteins listed in Table 4.

In an aspect, a disclosed method can comprise administering one or more additional therapeutic agents. One or more additional therapeutic agents can comprise any therapeutic agents disclosed herein.

2. miRNA Biomarkers

Disclosed herein is a method of predicting penetrance of a dystonia in a subject comprising obtaining a biosample from a subject; determining the expression level of one or more miRNAs in the biosample; identifying those miRNAs that are differentially expressed in the biosample when compared to a reference biosample; wherein the degree of differential expression predicts the likelihood of penetrance.

In an aspect, a disclosed method can comprise obtaining a biosample from the subject prior to treatment and detecting the expression level of one or more miRNAs in the pre-treatment biosample. In an aspect, a disclosed method can comprise obtaining a reference biosample. In an aspect, a reference biosample can comprise a biosample from a subject not having a dystonia or an aggregate of biosamples from subjects not having a dystonia. In an aspect, a reference biosample can comprise a biosample from the subject prior to the onset of a dystonia. In an aspect, obtaining a reference biosample can comprise obtaining a biosample from a subject not having a dystonia and determining the expression level of one or more miRNAs in the biosample. In an aspect, obtaining a reference biosample can comprise obtaining a biosample from subjects not having a dystonia and determining the expression level of one or more miRNAs in the biosamples.

In an aspect, determining the level of one or more miRNAs in a biosample can comprise using RNASeq or RT-qPCR. In an aspect, determining the level of one or more miRNAs can comprise a commercial assay (e.g., NanoStrin nCounter® assays), isothermal amplification-based assays, oligonucleotide-templated reactions, nanobead-based systems, and microfluidic-based assays for miRNA capture from biosamples and detection, and any combination thereof.

In an aspect, a biosample can comprise tissues, cells, biopsies, blood, lymph, CFS, serum, plasma, urine, saliva, mucus, tears, or a combination thereof. A biosample can comprise extracellular vesicles or extracellular vesicles collected from cultured patient-derived cells. In an aspect, cultured patient-derived cells can comprise primary cells, immortalized cells, iPSC cells, or any combination thereof.

In an aspect, one or more disclosed miRNAs can be those miRNAs in Table 7.

In an aspect, one or more disclosed miRNAs can comprise miR-135a-5p, miR-182-5p, miR-542-5p, miR-298-5p, miR-183-5p, miR-296-3p, miR-96-5p, miR-344d-3p, miR-5121, miR-140-3p, miR-344-3p, miR-187-3p, miR-130b-3p, miR-125b-1-3p, miR-34a-5p, miR-532-5p, miR-148a-3p, miR-3535, miR-362-5p, miR-192-5p, miR-34c-5p, miR-1291, miR-30b-5p, miR-362-3p, miR-671-5p, miR-31-5p, miR-22-3p, miR-199b-3p, miR-199a-3p, miR-30e-5p, miR-30c-5p, miR-30a-5p, miR-93-5p, miR-19b-3p, let-7i-5p, miR-103-3p, let-7f-5p, miR-26b-5p, and any combination thereof.

In an aspect, one or more disclosed miRNAs can be miR-135a-5p, miR-182-5p, miR-542-5p, miR-183-5p, miR-296-3p, miR-96-5p, miR-34a-5p, miR-22-3p, miR-199a-3p, and any combination thereof. In an aspect, one or more disclosed miRNAs can be miR-135a-5p, miR-183-5p, miR-296-3p, miR-96-5p, miR-34a-5p, miR-22-3p, miR-199a-3p, and any combination thereof.

In an aspect, one or more disclosed miRNAs can comprise 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, or 26 or more of the disclosed miRNAs.

In an aspect, one or more disclosed miRNAs can be associated with dysfunction in phosphor-eIF2α signaling. In an aspect, one or more disclosed miRNAs can be associated with dysfunction in the integrated stress response.

In an aspect, a dystonia can be focal dystonia, blepharospasm, cervical dystonia, oromandibular dystonia, task-specific or occupational dystonia, spasmodic dysphonia, generalized dystonia, segmental dystonia, DYT1-related dystonia, DYT6-related dystonia, DYT28-related dystonia, dopa-responsive dystonia, myoclonic dystonia, X-linked dystonia-Parkinsonism, rapid-onset dystonia-Parkinsonism, paroxysmal dystonia choreoathetosis, paroxysmal kinesigenic dystonia, paroxysmal nonkinesigenic dyskinesia, paroxysmal exertion-induced dyskinesia, primary dystonia, acquired dystonia, tardive dyskinesia, or tardive dystonia.

In an aspect, a disclosed method can comprise treating a subject having a dystonia.

In an aspect, treating a subject can comprise administering one or more agents that modulate the expression level of one or more differentially expressed miRNAs In an aspect, modulating the expression level can comprise increasing the expression level, decreasing the expression level, or both. In an aspect, the expression level of one or more of the differentially expressed miRNAs can be increased or decreased.

In an aspect, a disclosed method can comprise treating a subject. In an aspect, treating a subject can comprise administering one or more agents that target eIF2α signaling, such as, for example, contributing to the phosphorylation or de-phosphorylation of eIF2α.

In an aspect, a disclosed agent can be ritonavir, nelfinavir, lopinavir, saquinavir, deshydroxy-lopinavir, cobicistat, deshydroxy-ritonavir, or any combination thereof. In an aspect, a disclosed agent can be ritonavir.

In an aspect, a disclosed method can comprise repeating one or more steps of the disclosed method. In an aspect, a disclosed method can comprise modifying an administering step.

In an aspect, disclosed differential expression can comprise at least a 2-fold change, at least at 5-fold change, at least a 7-fold change, at least a 10-fold change, or more than a 10-fold change between the two biosamples. In an aspect, disclosed differential expression can comprise at least a +1, +2, +3, −1, −2, or −3 Z-score. In an aspect, disclosed differential expression can comprise at least a Cohen's d effect size of at least 0.2, at least 0.5, at least 0.8, or greater than 0.8. In an aspect, disclosed differential expression can comprise at least a Cohen's d effect size of at least 1, at least 2, at least 3, or greater than 3.

In an aspect, a disclosed method can comprise administering one or more additional therapeutic agents. One or more additional therapeutic agents can comprise any therapeutic agents disclosed herein.

In an aspect of a disclosed method, when the differential expression of one or more miRNAs comprises at least a 2-fold change, at least a 5-fold change, at least a 7-fold change, or at least a 10-fold change, the risk of penetrance can increase. In an aspect of a disclosed method, when the differential expression of one or more miRNAs comprises at least a +1, +2, +3, −1, −2, or −3 Z-score, the risk of penetrance can increase.

### H. Methods of Predicting Responsiveness to Treatment

### 1. Protein Biomarkers

Disclosed herein is a method of predicting responsiveness to a treatment, the method comprising obtaining a biosample from a subject having a dystonia; determining the expression level of one or more proteins in the biosample to create a proteomic profile; comparing the subject's proteomic profile to a proteomic profile of a treatment-responsive subject; and if the proteomic profiles are similar, then predicting that the subject having a dystonia will be responsive to the treatment, and if the proteomic profiles are dissimilar, then predicting that the subject having a dystonia will not be responsive to the treatment.

In an aspect, a disclosed proteomic profile of the subject having a dystonia can indicate dysfunction in phosphor-eIF2α signaling. In an aspect, disclosed proteomic profile the subject having a dystonia can indicate dysfunction in the integrated stress response.

In an aspect, determining the level of one or more proteins in a biosample can comprise using liquid chromatography with tandem mass spectrometry (LC-MS-MS), parallel reaction monitoring (PRM), or multiple reaction monitoring (MRM).

In an aspect, a disclosed proteomic profile of a treatment-responsive subject can comprise a proteomic profile generated from a treatment-responsive subject. In an aspect, a disclosed proteomic profile of a treatment-responsive subject can comprise an aggregate proteomic profile generated from one or more treatment-responsive subjects.

In an aspect, a biosample can comprise tissues, cells, biopsies, blood, lymph, CFS, serum, plasma, urine, saliva, mucus, tears, or a combination thereof. A biosample can comprise extracellular vesicles or extracellular vesicles collected from cultured patient-derived cells. In an aspect, cultured patient-derived cells can comprise primary cells, immortalized cells, iPSC cells, or any combination thereof.

In an aspect, one or more disclosed proteins can be those proteins in Table 4.

In an aspect, one or more disclosed proteins can comprise isocitrate dehydrogenase [NADP] (mitochondrial), aldehyde dehydrogenase (mitochondrial), inosine triphosphate pyrophosphatase, eukaryotic translation EF1 epsilon-1, histone-binding protein RBBP4, integral membrane protein 2B, spliceosome RNA helicase Ddx39b, NEDD8-activating enzyme E1 catalytic subunit, exocyst complex component 4, importin subunit alpha-3, dynactin subunit 2, importin-11, protein transport protein Sec23A, glutamine-tRNA ligase, ferritin heavy chain, phospholipase A-2-activating protein, CCR4-NOT transcription complex subunit 1, single-stranded DNA-binding protein, mitochondrial, aspartate-tRNA ligase, cytoplasmic, heterogeneous nuclear ribonucleoprotein F, histone H3.1, serine hydroxymethyltransferase, mitochondrial, osteopontin, pre-mRNA-processing-splicing factor 8, ubiquitin thioesterase OTUB1, endothelial lipase, threonine-tRNA ligase, cytoplasmic, coatomer subunit beta′, heterogeneous nuclear ribonucleoprotein L, Regulator of nonsense transcripts 1, ATP-dependent RNA helicase A, prolyl endopeptidase, aldose reductase, protein transport protein Sec31A, EGF-containing fibulin-like ECM protein 1, S-methyl-5′-thioadenosine phosphorylase, probable ATP-dependent RNA helicase DDX5, 26S proteasome non-ATPase regulatory subunit 1, DNA replication licensing factor MCM2, prelamin-A/C, Pre-mRNA-splicing factor ATP-dependent RNA helicase DHX15, spectrin beta chain, non-erythrocytic 1, latent-transforming growth factor beta-binding protein 1, spectrin alpha chain, non-erythrocytic 1, collagen alpha-1(V) chain, ATP-dependent RNA helicase DDX3X, and any combination thereof.

In an aspect, one or more disclosed proteins can be isocitrate dehydrogenase [NADP](mitochondrial), aldehyde dehydrogenase (mitochondrial), inosine triphosphate pyrophosphatase, eukaryotic translation EF1 epsilon-1, histone-binding protein RBBP4, integral membrane protein 2B, spliceosome RNA helicase Ddx39b, NEDD8-activating enzyme E1 catalytic subunit, exocyst complex component 4, importin subunit alpha-3, dynactin subunit 2, importin-11, protein transport protein Sec23A, glutamine-tRNA ligase, ferritin heavy chain, phospholipase A-2-activating protein, CCR4-NOT transcription complex subunit 1, single-stranded DNA-binding protein, mitochondrial, aspartate-tRNA ligase, cytoplasmic, heterogeneous nuclear ribonucleoprotein F, histone H3.1, serine hydroxymethyltransferase, mitochondrial, osteopontin, pre-mRNA-processing-splicing factor 8, ubiquitin thioesterase OTUB1, and any combination thereof. In an aspect, one or more disclosed proteins can comprise 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, or 26 or more of the disclosed proteins.

In an aspect, one or more disclosed proteins can be associated with dysfunction in phosphor-eIF2α signaling. In an aspect, one or more disclosed proteins can be associated with dysfunction in the integrated stress response.

In an aspect, a dystonia can be focal dystonia, blepharospasm, cervical dystonia, oromandibular dystonia, task-specific or occupational dystonia, spasmodic dysphonia, generalized dystonia, segmental dystonia, DYT1-related dystonia, DYT6-related dystonia, DYT28-related dystonia, dopa-responsive dystonia, myoclonic dystonia, X-linked dystonia-Parkinsonism, rapid-onset dystonia-Parkinsonism, paroxysmal dystonia choreoathetosis, paroxysmal kinesigenic dystonia, paroxysmal nonkinesigenic dyskinesia, paroxysmal exertion-induced dyskinesia, primary dystonia, acquired dystonia, tardive dyskinesia, or tardive dystonia.

In an aspect, a disclosed method can comprise treating a subject. In an aspect, a disclosed method can comprise treating a subject having a dystonia.

In an aspect, treating a subject can comprise administering one or more agents that modulate the expression level of one or more differentially expressed proteins. In an aspect, modulating the expression level can comprise increasing the expression level, decreasing the expression level, or both. In an aspect, the expression level of one or more of the differentially expressed proteins can be increased or decreased.

In an aspect, treating a subject can comprise administering one or more agents that target eIF2α signaling, such as, for example, contributing to the phosphorylation or de-phosphorylation of eIF2α.

In an aspect, a disclosed agent can be ritonavir, nelfinavir, lopinavir, saquinavir, deshydroxy-lopinavir, cobicistat, deshydroxy-ritonavir, or any combination thereof. In an aspect, a disclosed agent can be ritonavir.

In an aspect, a disclosed method can comprise repeating one or more steps of the disclosed method. In an aspect, a disclosed method can comprise modifying an administering step.

In an aspect, disclosed differential expression can comprise at least a 2-fold change, at least at 5-fold change, at least a 7-fold change, at least a 10-fold change, or more than a 10-fold change between the two biosamples. In an aspect, disclosed differential expression can comprise at least a +1, +2, +3, −1, −2, or −3 Z-score. In an aspect, disclosed differential expression can comprise at least a 2-fold change, at least at 5-fold change, at least a 7-fold change, at least a 10-fold change, or more than a 10-fold change between the two biosamples. In an aspect, disclosed differential expression can comprise at least a Cohen's d effect size of at least 0.2, at least 0.5, at least 0.8, or greater than 0.8. In an aspect, disclosed differential expression can comprise at least a Cohen's d effect size of at least 1, at least 2, at least 3, or greater than 3.

In an aspect, a disclosed method can comprise generating a proteomic profile for one or both biosamples. Individual components of the proteomic profile include but are not limited to those members shown in Table 4. For the purposes of the present invention the proteomic profile comprises from least two to all 46 of the proteins listed in Table 4.

In an aspect, a disclosed method can comprise administering one or more additional therapeutic agents. One or more additional therapeutic agents can comprise any therapeutic agents disclosed herein.

2. miRNA Biomarkers

Disclosed herein is a method of predicting responsiveness to a treatment comprising obtaining a biosample from a subject having a dystonia; determining the expression level of one or more miRNAs in the biosample to create a miRNA profile; comparing the subject's miRNA profile to a miRNA profile of a treatment-responsive subject; and if the profiles are similar, then predicting that the subject having a dystonia will be responsive to the treatment, and if the profiles are dissimilar, then predicting that the subject having a dystonia will not be responsive to the treatment.

In an aspect, a disclosed miRNA profile of a treatment-responsive subject can comprise a miRNA profile generated from a treatment-responsive subject. In an aspect, a disclosed miRNA profile of a treatment-responsive subject can comprise an aggregate miRNA profile generated from one or more treatment-responsive subjects.

In an aspect of a disclosed method, the miRNA profile of the subject having a dystonia can indicate dysfunction in phosphor-eIF2α signaling. In an aspect of a disclosed method, the miRNA profile of the subject having a dystonia can indicate dysfunction in the integrated stress response.

In an aspect, a disclosed method can comprise obtaining a biosample from the subject prior to treatment and detecting the expression level of one or more miRNAs in the pre-treatment biosample. In an aspect, a disclosed method can comprise obtaining a reference biosample. In an aspect, obtaining a reference biosample can comprise obtaining a biosample from a subject responsive to treatment and determining the expression level of one or more miRNAs in the biosample. In an aspect, obtaining a reference biosample can comprise obtaining a biosample from subjects responsive to treatment and determining the expression level of one or more miRNAs in the biosamples. In an aspect, obtaining a reference biosample can comprise obtaining a biosample from a subject not responsive to treatment and determining the expression level of one or more miRNAs in the biosample. In an aspect, obtaining a reference biosample can comprise obtaining a biosample from subjects not responsive to treatment and determining the expression level of one or more miRNAs in the biosamples.

In an aspect, determining the level of one or more miRNAs in a biosample can comprise using RNASeq or RT-qPCR. In an aspect, determining the level of one or more miRNAs can comprise a commercial assay (e.g., NanoStrin nCounter® assays), isothermal amplification-based assays, oligonucleotide-templated reactions, nanobead-based systems, and microfluidic-based assays for miRNA capture from biosamples and detection, and any combination thereof.

In an aspect, a biosample can comprise tissues, cells, biopsies, blood, lymph, CFS, serum, plasma, urine, saliva, mucus, tears, or a combination thereof. A biosample can comprise extracellular vesicles or extracellular vesicles collected from cultured patient-derived cells. In an aspect, cultured patient-derived cells can comprise primary cells, immortalized cells, iPSC cells, or any combination thereof.

In an aspect, one or more disclosed miRNAs can be those miRNAs in Table 7.

In an aspect, one or more disclosed miRNAs can comprise miR-135a-5p, miR-182-5p, miR-542-5p, miR-298-5p, miR-183-5p, miR-296-3p, miR-96-5p, miR-344d-3p, miR-5121, miR-140-3p, miR-344-3p, miR-187-3p, miR-130b-3p, miR-125b-1-3p, miR-34a-5p, miR-532-5p, miR-148a-3p, miR-3535, miR-362-5p, miR-192-5p, miR-34c-5p, miR-1291, miR-30b-5p, miR-362-3p, miR-671-5p, miR-31-5p, miR-22-3p, miR-199b-3p, miR-199a-3p, miR-30e-5p, miR-30c-5p, miR-30a-5p, miR-93-5p, miR-19b-3p, let-7i-5p, miR-103-3p, let-7f-5p, miR-26b-5p, and any combination thereof.

In an aspect, one or more disclosed miRNAs can be miR-135a-5p, miR-182-5p, miR-542-5p, miR-183-5p, miR-296-3p, miR-96-5p, miR-34a-5p, miR-22-3p, miR-199a-3p, and any combination thereof. In an aspect, one or more disclosed miRNAs can be miR-135a-5p, miR-183-5p, miR-296-3p, miR-96-5p, miR-34a-5p, miR-22-3p, miR-199a-3p, and any combination thereof.

In an aspect, one or more disclosed miRNAs can comprise 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, or 26 or more of the disclosed miRNAs.

In an aspect, one or more disclosed miRNAs can be associated with dysfunction in phosphor-eIF2α signaling. In an aspect, one or more disclosed miRNAs can be associated with dysfunction in the integrated stress response.

In an aspect, a dystonia can be focal dystonia, blepharospasm, cervical dystonia, oromandibular dystonia, task-specific or occupational dystonia, spasmodic dysphonia, generalized dystonia, segmental dystonia, DYT1-treated dystonia, DYT6-related dystonia, DYT28-related dystonia, dopa-responsive dystonia, myoclonic dystonia, X-linked dystonia-Parkinsonism, rapid-onset dystonia-Parkinsonism, paroxysmal dystonia choreoathetosis, paroxysmal kinesigenic dystonia, paroxysmal nonkinesigenic dyskinesia, paroxysmal exertion-induced dyskinesia, primary dystonia, acquired dystonia, tardive dyskinesia, or tardive dystonia.

In an aspect, a disclosed method can comprise treating a subject having a dystonia. In an aspect, wherein treating a subject can comprise administering one or more agents that modulate the expression level of one or more differentially expressed miRNAs. In an aspect, modulating the expression level can comprise increasing the expression level, decreasing the expression level, or both. In an aspect, the expression level of one or more of the differentially expressed miRNAs can be increased or decreased. In an aspect, wherein treating a subject can comprise administering one or more agents that target eIF2α signaling, such as, for example, contributing to the phosphorylation or de-phosphorylation of eIF2α.

In an aspect, a disclosed agent can be ritonavir, nelfinavir, lopinavir, saquinavir, deshydroxy-lopinavir, cobicistat, deshydroxy-ritonavir, or any combination thereof. In an aspect, a disclosed agent can be ritonavir.

In an aspect, a disclosed method can comprise repeating one or more steps of the disclosed method. In an aspect, a disclosed method can comprise modifying an administering step.

In an aspect, disclosed differential expression can comprise at least a 2-fold change, at least at 5-fold change, at least a 7-fold change, at least a 10-fold change, or more than a 10-fold change between the two biosamples. In an aspect, disclosed differential expression can comprise at least a +1, +2, +3, −1, −2, or −3 Z-score. In an aspect, disclosed differential expression can comprise at least a Cohen's d effect size of at least 0.2, at least 0.5, at least 0.8, or greater than 0.8. In an aspect, disclosed differential expression can comprise at least a Cohen's d effect size of at least 1, at least 2, at least 3, or greater than 3.

In an aspect, a disclosed method can comprise administering one or more additional therapeutic agents. One or more additional therapeutic agents can comprise any therapeutic agents disclosed herein.

### I. Kits

Disclosed herein is a kit comprising a disclosed pharmaceutical formulation with our without additional therapeutic agents to treat, prevent, inhibit, and/or ameliorate one or more symptoms or complications associated with a dystonia. Disclosed herein is a kit comprising the reagents necessary to perform one or more of the disclosed methods, such as, for example, PRM, MRM, or LC/MS/MS to detect one or more protein biomarkers of a dystonia. Disclosed herein is a kit comprising the reagents necessary to perform one or more of the disclosed methods, such as, for example, RT-qPCR, RNAseq, a commercial assay (e.g., NanoStrin nCounter® assays), isothermal amplification-based assays, oligonucleotide-templated reactions, nanobead-based systems, and microfluidic-based assays for miRNA capture from biosamples and detection, and any combination thereof.

In an aspect, a disclosed kit can comprise a protein biomarker panel. A disclosed biomarker panel can detect 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, or 26 or more of the disclosed proteins.

In an aspect, a disclosed kit can comprise a miRNA biomarker panel. A disclosed biomarker panel can detect 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, or 26 or more of the disclosed miRNAs.

“Agents” and “Therapeutic Agents” are known to the art and are described supra. In an aspect, the one or more agents can treat, prevent, inhibit, and/or ameliorate one or more comorbidities in a subject. In an aspect, one or more therapeutic agents can treat, inhibit, prevent, and/or ameliorate a dystonia symptom or a dystonia related complication.

In an aspect, a disclosed kit can comprise at least two components constituting the kit. Together, the components constitute a functional unit for a given purpose (such as, for example, treating a subject diagnosed with or suspected of having a dystonia). Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation. In an aspect, a kit for use in a disclosed method can comprise one or more containers holding a disclosed pharmaceutical formulation, a disclosed therapeutic agent, a disclosed reagent, or a combination thereof, and a label or package insert with instructions for use. In an aspect, suitable containers include, for example, bottles, vials, syringes, blister pack, etc. The containers can be formed from a variety of materials such as glass or plastic. The container can hold, for example, a disclosed pharmaceutical formulation and/or a disclosed therapeutic agent and can have a sterile access port (for example the container may be an intravenous solution bag or a vial having a stopper pierceable by a hypodermic injection needle). The label or package insert can indicate that a disclosed pharmaceutical formulation and/or a disclosed therapeutic agent can be used for treating, preventing, inhibiting, and/or ameliorating a dystonia or complications and/or symptoms associated with a dystonia. In an aspect, a disclosed kit can comprise additional components necessary for administration such as, for example, other buffers, diluents, filters, needles, and syringes.

In an aspect, a disclosed kit can comprise one or more therapeutic agents that target eIF2α signaling or target ISR dysregulation. In an aspect, one or more disclosed agents in a disclosed kit can modulate the expression level of one or more proteins in Table 4 and/or one or more miRNAs in Table 7.

### VII. EXAMPLES

EVs have been found to circulate through many different body fluids including blood and urine. The isolation of EVs may result in a large enrichment of low-abundant molecules that may have particular pathophysiological significance. EV cargo composition changes between cell types and physiological states as composition determines EV secretion and function. An important breakthrough was the discovery of nucleic acids in EVs such as mRNA and miRNA. RNA molecules present in EVs seem to follow selective incorporation as evidence points to their enrichment relative to the RNA profiles of the secreting cells. Interestingly, several studies have shown that EV-associated mRNAs and miRNAs can be functionally transferred to recipient cells. A physiological relevance for the presence of mRNAs and miRNAs in EVs including immunological and vascularization functions among others have already been reported.

Early identification of the factors driving dystonia can provide a treatment path to intervene before disease onset. EVs are suitable to identify biomarkers for dystonias and to monitor translational state integrity. Identifying biomarkers in DYT1 and other dystonias represents an opportunity to improve diagnosis, predict disease progression, and/or track the efficacy of treatment. The Examples set forth herein provide a panel of proteins and microRNAs as biomarkers for a dystonia (e.g., DYT1). The work described herein also classified these biomarkers as responsive to a putative therapeutic drug and as indicators of dysfunction in the “integrated stress response”, a pathway known to be involved in multiple forms of dystonia and other neurological diseases. Because DYT1 dystonia is part of a group of dystonias with ISR dysregulation, the biomarker panels described herein can be used to define subsets of biochemically similar dystonias when applied to individuals with sporadic and all other forms of dystonia. Besides diagnostic value, individuals with this biomarker array would then be candidates for particular therapeutic interventions. Details of candidate biomarker identification and selection are described below.

### Example 1

### Reproducible and High-Quality Exosome Purification

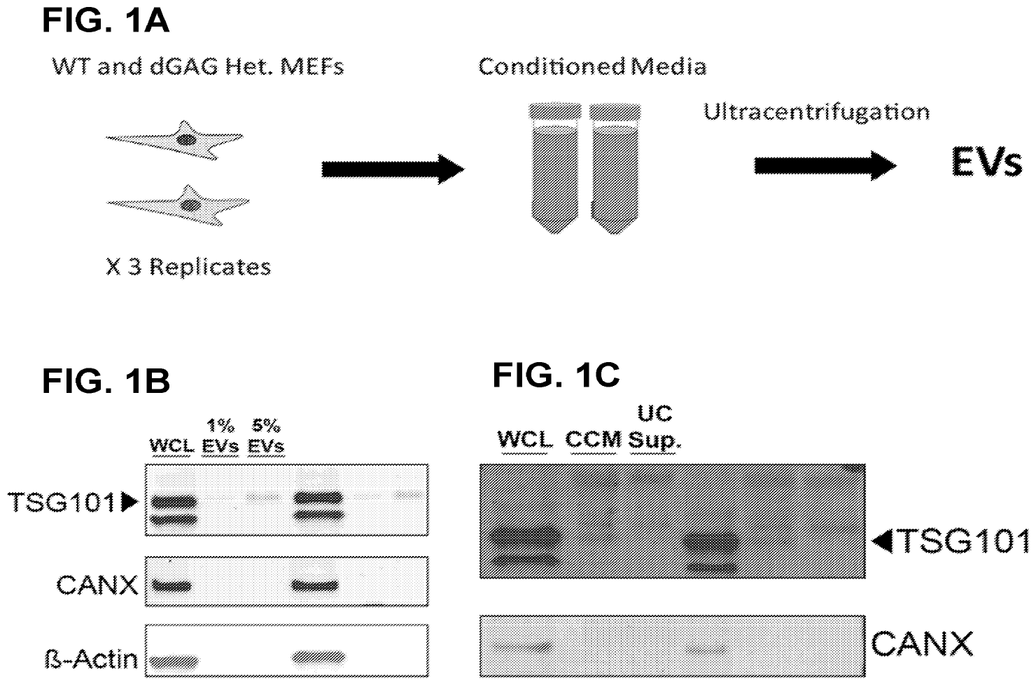

Methods for isolation and purification of exosomes and extracellular vesicles (EV) were extensively reviewed. Experiments were performed to arrive at a robust and reproducible protocol. The protocol used herein is that set forth in Théry et al, 2006, which is incorporated by reference for it teaching of this protocol. This protocol involves depleting serum-derived EVs from cell culture media by overnight ultracentrifugation and exchanging media for 1% FBS EV-depleted media. Media is then collected from cells following a 24-hour conditioning period and EVs are isolated by 18-hour ultracentrifugation at 100,000 g (FIG. 1A).

EVs were isolated from media of cultured murine embryonic fibroblasts (MEFs) prepared in-house according to protocols described by Xu 2005 from DYT1 knockin mice (Tor1a^{delGAG/+}) (Goodchild R E, et al. (2005) Neuron. 48:923-932) and littermate wild-type (WT) controls. The cultured MEFs from the DYT1 mice were exposed for 24 hours to Ritonavir (RTV) while the MEFs from WT mice were exposed to vehicle, ISRIB, or Salubrinal (SAL). The EVs isolated from these WT cells and DYT1 were characterized by untargeted proteomics and RNA-seq to identify differentially expressed proteins and RNAs as candidate biomarkers. Experimenter and Proteomics core staff were blinded to genotype of cell cultures throughout preparation and analysis phases. EV isolation was confirmed by Western immunoblot documenting presence of the exosomal marker (TSG101) and absence of the endoplasmic reticulum (ER) marker (CANX) (FIG. 1B (Cohort 1) and FIG. 1C (Cohort 2)).

### Example 2

### Summary of Proteomics and Metabolomics Shared Resource Methods

Following the bicinchoninic acid assay (BCA) analysis and Western immunoblot of a portion of samples for QC, 11.6 μg-129.2 μg of EV protein lysates in RIPA buffer were sent to the Duke Proteomics and Metabolomics Shared Resource core facility for LC/MS/MS quantitative proteomic analysis. Samples were first normalized to 20 μg and spiked with undigested casein at a total of 40 fmol/μg, 80 fmol/μg, or 160 fmol/μg, then reduced with 10 mM dithiolthreitol for 30 minute at 80° C. and alkylated with 20 mM iodoacetamide for 30 minutes at room temperature. Next, the samples were supplemented with a final concentration of 1.2% phosphoric acid and 741 μL of S-Trap (Protifi) binding buffer (90% MeOH/100 mM EAB). Proteins were trapped on the S-Trap, digested using 20 ng/μL sequencing grade trypsin (Promega) for 1 hour at 47° C., and eluted using 50 mM TEAB, followed by 0.2% FA, and lastly using 50% CAN/0.2% FA. All samples were then lyophilized to dryness and resuspended in 40 μL 1% TFA/2% acetonitrile containing 12.5 fmol/μL yeast alcohol dehydrogenase (ADH_YEAST). A QC Pool was created from 3 uL of each sample. All QC Pools were run periodically throughout the acquisition period.

Quantitative LC/MS/MS was performed on 2 μL of each sample, using a nanoAcquity UPLC system (Waters Corp) coupled to a Thermo Orbitrap Fusion Lumos high resolution accurate mass tandem mass spectrometer (Thermo) via a nanoelectrospray ionization source. Briefly, the sample was first trapped on a Symmetry C18 20 mm×180 μm trapping column (5 μL/min at 99.9/0.1 v/v water/acetonitrile), after which the analytical separation was performed using a 1.8 μm Acquity HSS T3 C18 75 μm×250 mm column (Waters Corp.) with a 90 minute linear gradient of 5% to 30% acetonitrile with 0.1% formic acid at a flow rate of 400 nanoliters/minute (nL/min) with a column temperature of 55° C. Data collection on the Fusion Lumos mass spectrometer was performed in a data-dependent acquisition (DDA) mode of acquisition with a r=120,000 (@ m/z 200) full MS scan from m/z 375-1500 with a target AGC value of 2e5 ions. MS/MS scans were acquired at Rapid scan rate (Ion Trap) with an AGC target of 5e3 ions and a max injection time of 100 ms. The total cycle time for MS and MS/MS scans was 2 seconds. A 20 second dynamic exclusion was employed to increase depth of coverage. The total analysis cycle time for each sample injection was approximately 2 hours.

Following 22 total UPLC-MS/MS analyses (excluding conditioning runs, but including 4 replicate QC data was imported into Proteome Discoverer 2.3 (Thermo Scientific Inc.), and analyses were aligned based on the accurate mass and retention time of detected ions (“features”) using Minora Feature Detector algorithm in Proteome Discoverer. Relative peptide abundance was calculated based on area-under-the-curve (AUC) of the selected ion chromatograms of the aligned features across all runs. The MS/MS data was searched against the SwissProt M. musculus database, SwissProt bovine database and an equal number of reversed sequence “decoys” for false discovery rate determination. Mascot Distiller and Mascot Server (v 2.5, Matrix Sciences) were utilized to produce fragment ion spectra and to perform the database searches. Database search parameters included fixed modification on Cys (carbamidomethyl) and variable modifications on Meth (oxidation) and Asn and Gln (deamidation). Peptide Validator and Protein FDR Validator nodes in Proteome Discoverer were used to annotate the data at a maximum 1% protein false discovery rate. Only peptides unique to M. musculus were exported for data analysis.

Raw protein abundances were normalized to total peptide signal detected in each sample (sample loading normalization) to account for differences in total protein. Missing values were imputed after sample loading normalization in the following manner. If less than half of the values were missing in a treatment group, values were imputed with an intensity derived from a normal distribution defined by measured values within the same intensity range (20 bins). If greater than half values are missing for a peptide in a group and a peptide intensity is >5e6, then it was concluded that peptide was misaligned and its measured intensity was set to 0. All remaining missing values were imputed with the lowest 5% of all detected values. MS/MS spectra were acquired for peptide sequencing by database searching. Following database searching and peptide scoring using Proteome Discoverer validation, the data was annotated at a 1% protein false discovery rate.

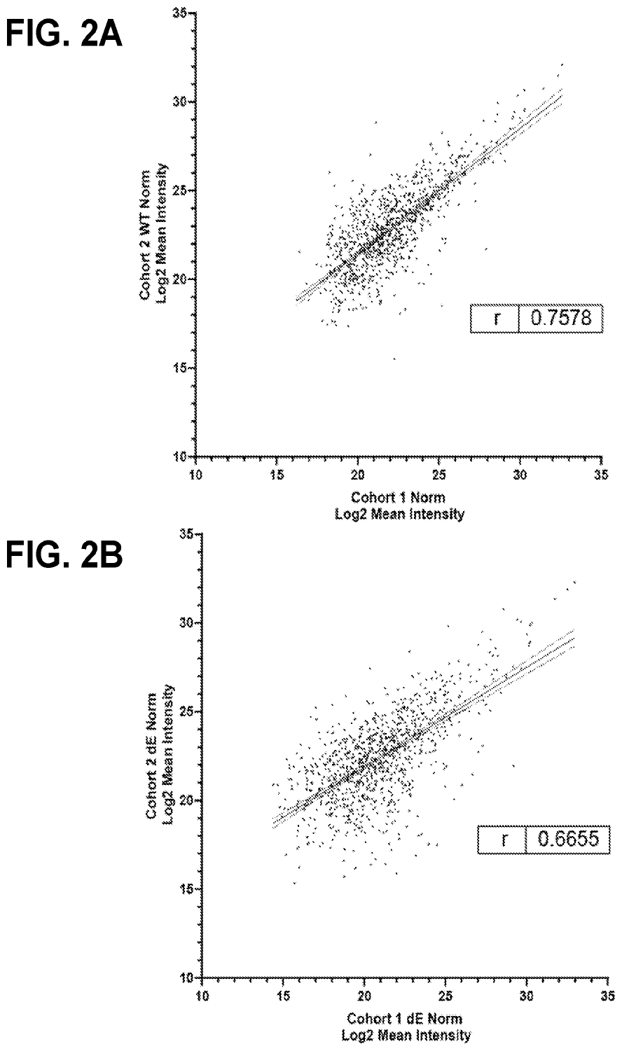

The data presented herein concerns the results from two separate proteomic sample sets. Cohort 1 established whether CV for technical replicates was within desired range. Cohort 1 included three (3) separate EV preps prepared from distinct tissue culture dishes of the same clonal cell line. One line for each genotype (WT and dGAG/+) was used to further pilot test for genotype differences. CV was within acceptable levels (27.2% and 33.4% for WT and dGAG/+, respectively) and potential for genotype differences was established in Cohort 1. Cohort 2 was expanded to include one biological replicate each of 3 distinct clonal cell line for each genotype. Qualitatively, >95% of proteins detected in Cohort 1 were present in Cohort 2. In addition, quantitatively, there was a significant correlation between results across cohorts. FIG. 2A shows results from Cohort 1 v. Cohort 2 for EVs isolated from WT cells while FIG. 2B shows results from Cohort 1 v. Cohort 2 for EVs isolated from DYT1 cells.

### Example 3

### Identification of Candidate Proteins

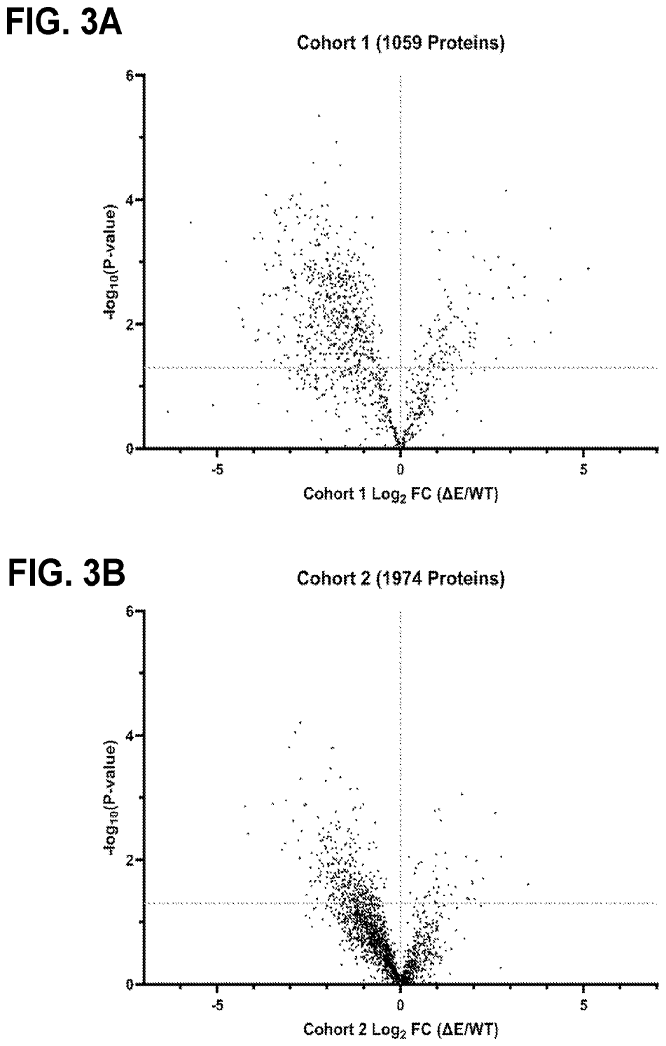

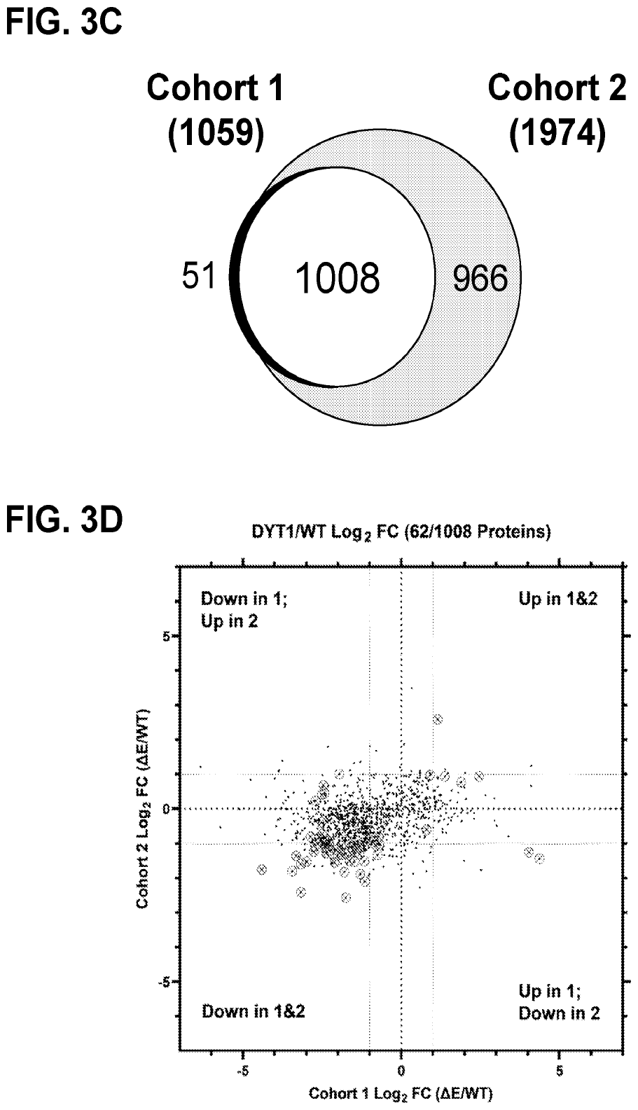

Using sample loading-normalized values for protein abundances, volcano plots of all EV proteins represented by more than 1 uniquely identifying peptide (UIP) and excluding bovine serum and human keratin-related protein contaminants are shown for each cohort. FIG. 3A shows a volcano plot showing fold change (DYT1(ΔE)/WT) and uncorrected p-value results for all proteins detected in the proteomic analysis of Cohort 1 while FIG. 3B shows the same analysis for Cohort 2. Candidate proteins were identified using 3 criteria: (i) p-value<0.05 in cohort 1, (ii) p-value<0.05 in cohort 2, and (iii) same directionality of fold change (FC) in both cohorts. FIG. 3C shows a Venn diagram demonstrating the overlap of detected proteins between Cohort 1 and Cohort 2. Under these criteria, 62 proteins were identified (+) with joint statistical significance between cohorts (compared to 2.52 hits/1008 proteins expected by chance) (FIG. 3D). Then, 54 of these 62 proteins showed concordant FC direction (FIG. 3D). These results indicate that a substantial fraction of the proteome showed quantitative proteomic differences in DYT1 and demonstrated that genotype-dependent proteomic differences are found in EV samples.

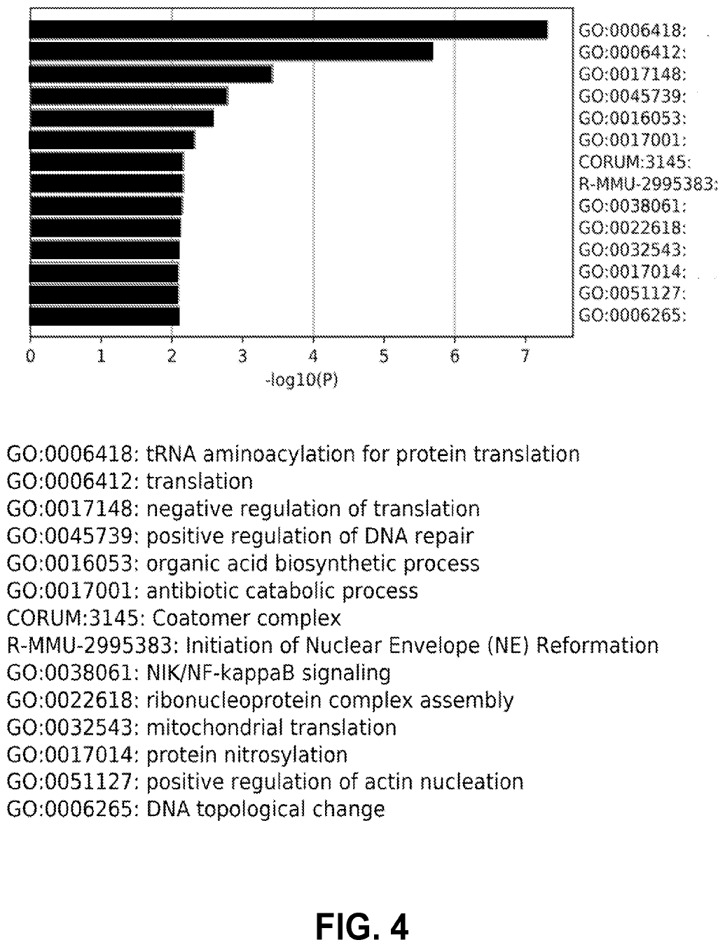

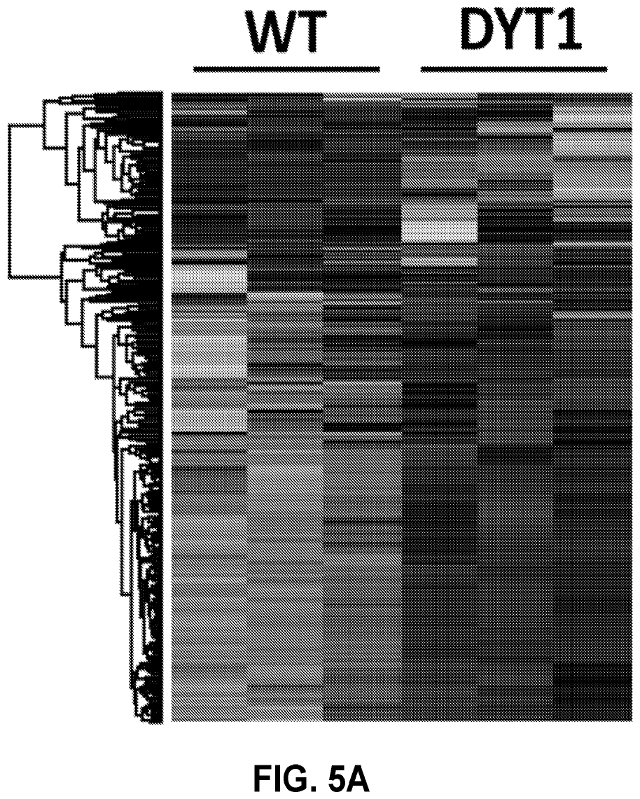

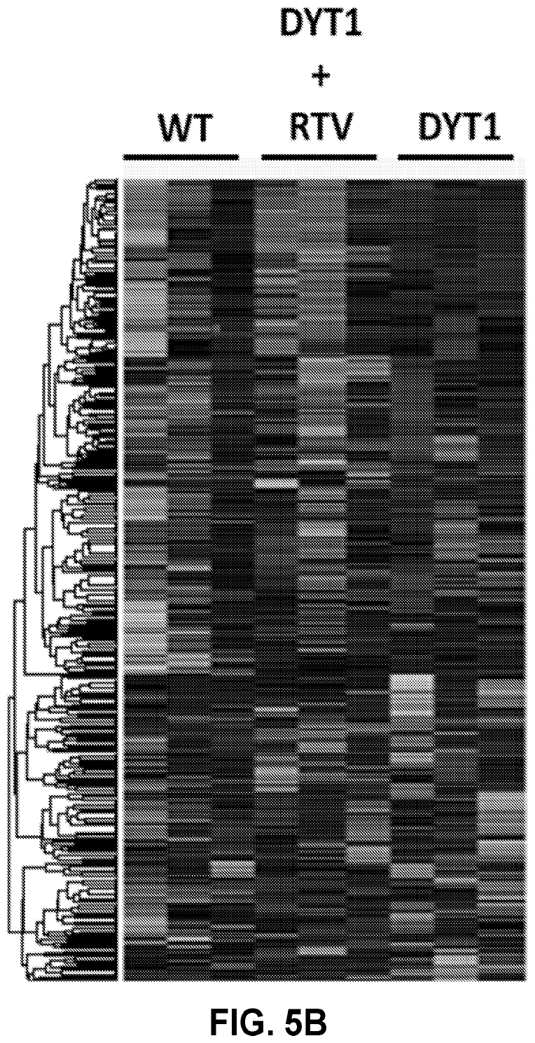

But, to determine whether eIF2alpha-dependent translational processes were altered in DYT1, broad proteomic differences were examined. Additional bioinformatic analyses for relationships among differentially expressed proteins were performed using the Metascape platform (Zhou Y, et al. (2019) Nat Commun. 10(1):1523). To generate an appropriately sized list for pathway enrichment, a subset of 202 proteins from the 1008 shared between cohorts (FIG. 3C) with a mean FC>2 and a Fisher's combined p-value<0.01 were identified. FIG. 4 shows the results of this analysis. Of note, several of the enriched pathways were associated with pathways previously associated with DYT1, such as nuclear envelope (Goodchild R E, et al. (2005) Neuron. 48:923-932), protein translation (Rittiner J E, et al. (2016) Neuron. 92:1238-1251) and NF-kappaB (Vaine C A, et al. (2017) Neurobiol Dis. 100:108-118). [0301] 1974 proteins were initially examined. A subset of proteins having a significant difference (p<0.05) in expression level between DYT1 cells and wild-type (WT) cells was identified. This subset was the “DYT1 Genotype Dependent Difference” subset and contained 363 proteins. See Table 1. FIG. 5A shows the hierarchical clustering of all proteins differentially expressed between WT cells and the DYT1 cells, wherein green represents up-regulated expression of proteins in DYT1 cells and red represents down-regulated expression of proteins in DYT1 cells as compared to WT cells.

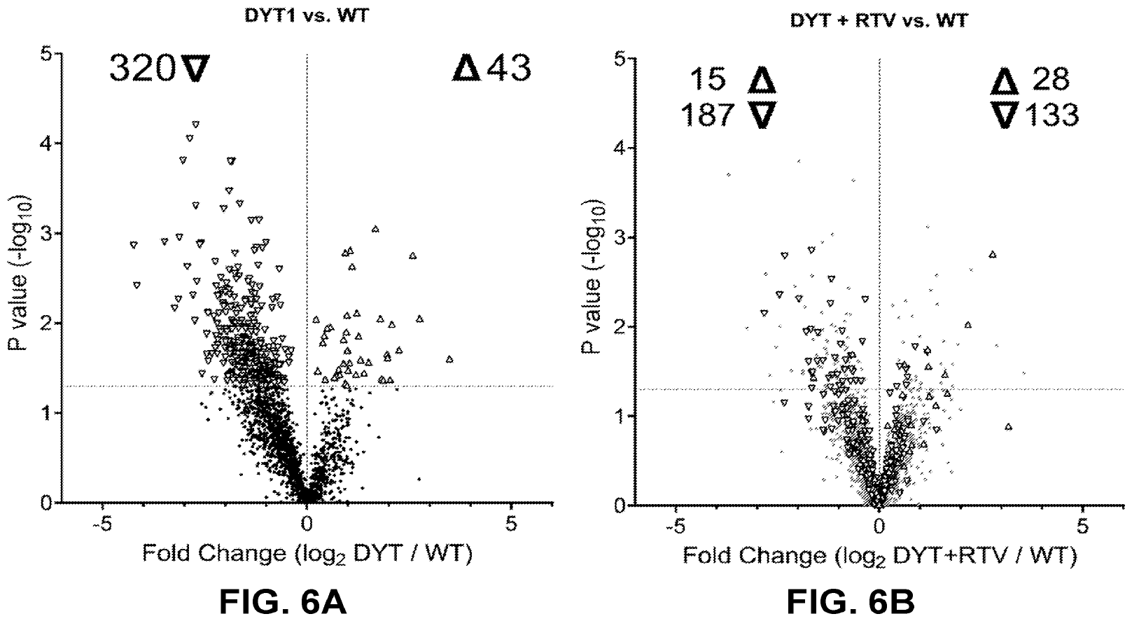

Beginning with the 363 proteins in the “DYT1 Genotype Dependent Differences” subset, the effect of ritonavir (RTV) in DYT1 cells and ISRIB in WT cells was examined. In DYT1 cells, RTV modulated expression in a therapeutic direction, meaning that the RTV direction was opposite that of the DYT1 genotype-dependent direction. FIG. 5B shows that the dysregulation of proteins in DYT1 cells was normalized by RTV. FIG. 6B shows the normalizing RTV effect on EV composition in DYT1 cells compared to WT cells (FIG. 6A) for the 363 proteins in this subset.

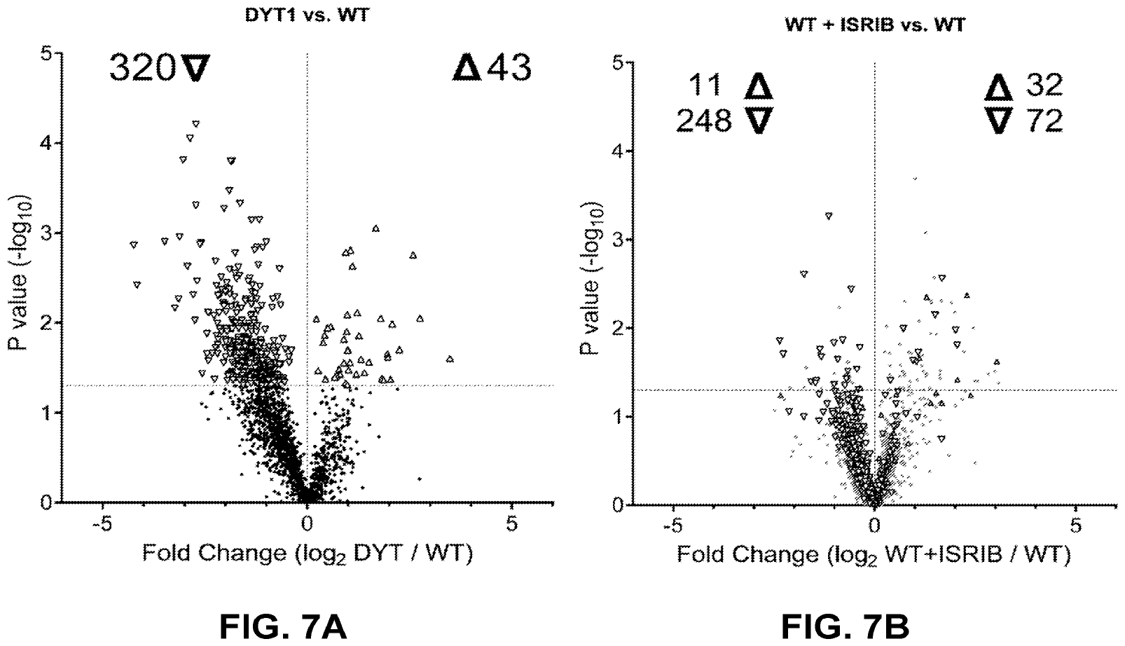

In WT cells, ISRIB modulated expression in a predicted pathological direction, meaning that the ISRIB direction was the same as that of the DYT1 genotype-dependent direction. This subset was the “RTV Therapeutic Efficacy with ISRIB Sensitivity” subset and contained 121 proteins. See Table 2. FIG. 7B shows that ISRIB biased distribution of the 363 proteins in WT cells towards a DYT1 disease model (FIG. 7A).

Beginning with the 363 proteins in the “DYT1 Genotype Dependent Differences” subset, the effect of RTV in DYT1 cells and ISRIB in WT cells was examined. Again, in DYT1 cells, RTV modulated expression in a therapeutic direction, meaning that the RTV direction was opposite that of the DYT1 genotype-dependent direction. In WT cells, ISRIB modulated expression in the predicted pathological direction (that is, in the opposite direction of DYT1 genotype-dependent direction). This subset was the “RTV Therapeutic Efficacy with ISRIB Insensitivity” subset and contained 33 proteins. See Table 3.

Having started with 1974 proteins, the top candidates were derived from a total of 121 proteins meeting QC, genotype, RTV, and ISRIB effect criteria as detailed above. The top 26 proteins were chosen based on summed Z score of Genotype and RTV effects. A second tier of 20 proteins were chosen based on the highest abundance proteins maintaining Z score>5. This subset was the “Protein Biomarker” subset and contained 46 proteins. See Table 4.

TABLE 1
The 363 Proteins of the “DYT1 Genotype Dependent Differences” Subset

| | | | [symbol] | ISRIH | SAL | | Abs Z | | | | | |
|---|---|---|---|---|---|---|---|---|---|---|---|---|
| | | | Direction | Direction | Direction | In | Score Sum | | | | | |
| | | | Opposite | Same as | Opposite | Human | (Geno | WT Mean | HetvWT | [symbol] vHet | ISRIBvWT | SALvHet |
| Accession | GN | Description | dE? | dE? | ISRIB? | Plasma | [symbol] | Abundance | Z Score | Z Score | Z Score | Z Score |
| [symbol] | [symbol] | Isocitrate dehydrogenase [NADP], | yes | yes | yes | yes | [symbol] | [symbol] | [symbol] | [symbol] | [symbol] | [symbol] |
| | | [symbol] | | | | | | | | | | |
| [symbol] | [symbol] | Aldehyde dehydrogenase, mitochondrial | yes | yes | yes | yes | [symbol] | [symbol] | [symbol] | [symbol] | [symbol] | [symbol] |
| [symbol] | [symbol] | Inosine triphosphate pyrophosphatase | yes | yes | yes | yes | [symbol] | [symbol] | [symbol] | [symbol] | [symbol] | [symbol] |
| [symbol] | [symbol] | [symbol] translation [symbol] 1 | yes | yes | yes | yes | [symbol] | [symbol] | [symbol] | [symbol] | [symbol] | [symbol] |
| | | epsilon-1 | | | | | | | | | | |
| [symbol] | [symbol] | Histone-binding protein RBBP4 | yes | yes | yes | yes | [symbol] | [symbol] | [symbol] | [symbol] | [symbol] | [symbol] |
| [symbol] | [symbol] | Integral membrane protein 2B | yes | yes | yes | yes | [symbol] | [symbol] | [symbol] | [symbol] | [symbol] | [symbol] |
| [symbol] | [symbol] | [symbol] RNA helicase [symbol] | yes | yes | yes | yes | [symbol] | [symbol] | [symbol] | [symbol] | [symbol] | [symbol] |
| [symbol] | [symbol] | [symbol] activating enzyme [symbol] catalytic | yes | yes | yes | yes | [symbol] | [symbol] | [symbol] | [symbol] | [symbol] | [symbol] |
| | | subunit | | | | | | | | | | |
| [symbol] | [symbol] | [symbol] complex component 4 | yes | yes | yes | yes | [symbol] | [symbol] | [symbol] | [symbol] | [symbol] | [symbol] |
| [symbol] | [symbol] | Importin subunit alpha-3 | yes | yes | NO | yes | [symbol] | [symbol] | [symbol] | [symbol] | [symbol] | [symbol] |
| [symbol] | [symbol] | Dynactin subunit 2 | yes | yes | yes | yes | [symbol] | [symbol] | [symbol] | [symbol] | [symbol] | [symbol] |
| [symbol] | [symbol] | Importin-11 | yes | yes | yes | yes | [symbol] | [symbol] | [symbol] | [symbol] | [symbol] | [symbol] |
| [symbol] | [symbol] | Protein transport protein [symbol] | yes | yes | NO | yes | [symbol] | [symbol] | [symbol] | [symbol] | [symbol] | [symbol] |
| [symbol] | [symbol] | Glutamine--tRNA ligase | yes | yes | yes | yes | [symbol] | [symbol] | [symbol] | [symbol] | [symbol] | [symbol] |
| [symbol] | [symbol] | Ferritin heavy chain | yes | yes | yes | yes | [symbol] | [symbol] | [symbol] | [symbol] | [symbol] | [symbol] |
| [symbol] | [symbol] | Phospholipase A-2-activating protein | yes | yes | yes | yes | [symbol] | [symbol] | [symbol] | [symbol] | [symbol] | [symbol] |
| [symbol] | [symbol] | CCR4- [symbol] transcription complex subunit 1 | yes | yes | yes | yes | [symbol] | [symbol] | [symbol] | [symbol] | [symbol] | [symbol] |
| [symbol] | [symbol] | Single-stranded DNA-binding protein, | yes | yes | yes | yes | [symbol] | [symbol] | [symbol] | [symbol] | [symbol] | [symbol] |
| | | [symbol] | | | | | | | | | | |
| [symbol] | [symbol] | Aspartate--tRNA ligase, cytoplasmic | yes | yes | yes | yes | [symbol] | [symbol] | [symbol] | [symbol] | [symbol] | [symbol] |
| [symbol] | [symbol] | Heterogeneous nuclear ribonucleoprotein 1 | yes | yes | yes | yes | [symbol] | [symbol] | [symbol] | [symbol] | [symbol] | [symbol] |
| [symbol] | [symbol] | Histone [symbol] | yes | yes | yes | yes | [symbol] | [symbol] | [symbol] | [symbol] | [symbol] | [symbol] |
| [symbol] | [symbol] | Serine hydroxymethyltransferase, | yes | yes | yes | yes | [symbol] | [symbol] | [symbol] | [symbol] | [symbol] | [symbol] |
| | | mitochondrial | | | | | | | | | | |
| [symbol] | [symbol] | [symbol] | yes | yes | yes | yes | [symbol] | [symbol] | [symbol] | [symbol] | [symbol] | [symbol] |
| [symbol] | [symbol] | Pre-mRNA-processing [symbol] | yes | yes | yes | yes | [symbol] | [symbol] | [symbol] | [symbol] | [symbol] | [symbol] |
| [symbol] | [symbol] | [symbol] | yes | yes | yes | yes | [symbol] | [symbol] | [symbol] | [symbol] | [symbol] | [symbol] |
| [symbol] | [symbol] | Endothelial lipase | yes | yes | NO | yes | [symbol] | [symbol] | [symbol] | [symbol] | [symbol] | [symbol] |
| [symbol] | [symbol] | Epoxide hydrolase 1 | yes | yes | NO | yes | [symbol] | [symbol] | [symbol] | [symbol] | [symbol] | [symbol] |
| [symbol] | [symbol] | [symbol] ubiquitin-protein ligase [symbol] | yes | yes | yes | yes | [symbol] | [symbol] | [symbol] | [symbol] | [symbol] | [symbol] |
| [symbol] | [symbol] | DNA- [symbol] | yes | yes | yes | yes | [symbol] | [symbol] | [symbol] | [symbol] | [symbol] | [symbol] |
| [symbol] | [symbol] | [symbol] transforming growth factor β-binding | yes | yes | yes | yes | [symbol] | [symbol] | [symbol] | [symbol] | [symbol] | [symbol] |
| | | protein 2 | | | | | | | | | | |
| [symbol] | [symbol] | GMP reductase 2 | yes | yes | NO | yes | [symbol] | [symbol] | [symbol] | [symbol] | [symbol] | [symbol] |
| [symbol] | [symbol] | translation initiation factor [symbol] subunit alpha | yes | yes | yes | yes | [symbol] | [symbol] | [symbol] | [symbol] | [symbol] | [symbol] |
| [symbol] | [symbol] | Ubiquitin carboxyl-terminal hydrolase [symbol] | yes | yes | yes | yes | [symbol] | [symbol] | [symbol] | [symbol] | [symbol] | [symbol] |
| [symbol] | [symbol] | [symbol] subunit epsilon | yes | yes | NO | yes | [symbol] | [symbol] | [symbol] | [symbol] | [symbol] | [symbol] |
| [symbol] | [symbol] | Hydroxymethylglutaryl-CoA synthese, [symbol] | yes | yes | yes | yes | [symbol] | [symbol] | [symbol] | [symbol] | [symbol] | [symbol] |
| [symbol] | [symbol] | Glutathione synthetase | yes | yes | yes | yes | [symbol] | [symbol] | [symbol] | [symbol] | [symbol] | [symbol] |
| [symbol] | [symbol] | [symbol] reductase | yes | yes | yes | yes | [symbol] | [symbol] | [symbol] | [symbol] | [symbol] | [symbol] |
| [symbol] | [symbol] | Serine threonine-protein kinase [symbol] | yes | yes | yes | yes | [symbol] | [symbol] | [symbol] | [symbol] | [symbol] | [symbol] |
| [symbol] | [symbol] | Mannose-1-phosphate guanyltransferase beta | yes | yes | yes | yes | [symbol] | [symbol] | [symbol] | [symbol] | [symbol] | [symbol] |
| [symbol] | [symbol] | Threonine--tRNA ligase, cytoplasmic | yes | yes | yes | yes | [symbol] | [symbol] | [symbol] | [symbol] | [symbol] | [symbol] |
| [symbol] | [symbol] | Rab3 GTPase-activating protein non- | yes | yes | NO | yes | [symbol] | [symbol] | [symbol] | [symbol] | [symbol] | [symbol] |
| | | catalytic subunit | | | | | | | | | | |
| [symbol] | [symbol] | [symbol] subunit beta′ | yes | yes | NO | yes | [symbol] | [symbol] | [symbol] | [symbol] | [symbol] | [symbol] |
| [symbol] | [symbol] | AP-1 complex subunit beta-1 | yes | yes | yes | yes | [symbol] | [symbol] | [symbol] | [symbol] | [symbol] | [symbol] |
| [symbol] | [symbol] | Dipeptidyl peptidase 3 | yes | yes | yes | yes | [symbol] | [symbol] | [symbol] | [symbol] | [symbol] | [symbol] |
| [symbol] | [symbol] | Receptor-type TYR-protein phosphatase [symbol] | yes | yes | yes | yes | [symbol] | [symbol] | [symbol] | [symbol] | [symbol] | [symbol] |
| [symbol] | [symbol] | Heterogeneous nuclear ribonucleoprotein [symbol] | yes | yes | yes | yes | [symbol] | [symbol] | [symbol] | [symbol] | [symbol] | [symbol] |
| [symbol] | [symbol] | Protein PBDC 1 | yes | yes | NO | yes | [symbol] | [symbol] | [symbol] | [symbol] | [symbol] | [symbol] |
| [symbol] | [symbol] | Regulator of nonsense transcripts 1 | yes | yes | NO | yes | [symbol] | [symbol] | [symbol] | [symbol] | [symbol] | [symbol] |
| [symbol] | [symbol] | [symbol] -dependent RNA helicase A | yes | yes | yes | yes | [symbol] | [symbol] | [symbol] | [symbol] | [symbol] | [symbol] |
| [symbol] | [symbol] | Eukaryotic translation mutation factor 4 | yes | yes | yes | yes | [symbol] | [symbol] | [symbol] | [symbol] | [symbol] | [symbol] |
| | | gamma 2 | | | | | | | | | | |
| [symbol] | [symbol] | Integrin beta-5 | yes | yes | yes | yes | [symbol] | [symbol] | [symbol] | [symbol] | [symbol] | [symbol] |
| [symbol] | [symbol] | [symbol] type protein ATPase subunit [symbol] | yes | yes | NO | yes | [symbol] | [symbol] | [symbol] | [symbol] | [symbol] | [symbol] |
| [symbol] | [symbol] | Prolyl endopeptidase | yes | yes | yes | yes | [symbol] | [symbol] | [symbol] | [symbol] | [symbol] | [symbol] |
| [symbol] | [symbol] | [symbol] -1-phosphate isomerase | yes | yes | yes | yes | [symbol] | [symbol] | [symbol] | [symbol] | [symbol] | [symbol] |
| [symbol] | [symbol] | Aldose reductase | yes | yes | yes | yes | [symbol] | [symbol] | [symbol] | [symbol] | [symbol] | [symbol] |
| [symbol] | [symbol] | Protein transport protein Sec31A | yes | yes | yes | yes | [symbol] | [symbol] | [symbol] | [symbol] | [symbol] | [symbol] |
| [symbol] | [symbol] | [symbol] -containing [symbol] -like [symbol] protein 1 | yes | yes | yes | yes | [symbol] | [symbol] | [symbol] | [symbol] | [symbol] | [symbol] |
| [symbol] | [symbol] | [symbol] multifunctional enzyme type 2 | yes | yes | NO | yes | [symbol] | [symbol] | [symbol] | [symbol] | [symbol] | [symbol] |
| [symbol] | [symbol] | S-methyl-5′-thioadenosine phosphorylase | yes | yes | yes | yes | [symbol] | [symbol] | [symbol] | [symbol] | [symbol] | [symbol] |
| [symbol] | [symbol] | Sec1 [symbol] domain-containing protein 1 | yes | yes | NO | yes | [symbol] | [symbol] | [symbol] | [symbol] | [symbol] | [symbol] |
| [symbol] | [symbol] | Probable ATP-dependent RNA helicase [symbol] | yes | yes | yes | yes | [symbol] | [symbol] | [symbol] | [symbol] | [symbol] | [symbol] |
| [symbol] | [symbol] | Alpha- [symbol] | yes | yes | yes | yes | [symbol] | [symbol] | [symbol] | [symbol] | [symbol] | [symbol] |
| [symbol] | [symbol] | [symbol] homology domain-containing | yes | yes | yes | yes | [symbol] | [symbol] | [symbol] | [symbol] | [symbol] | [symbol] |
| | | family O member 2 | | | | | | | | | | |
| [symbol] | [symbol] | Citrate synthase, mitochondrial | yes | yes | yes | yes | [symbol] | [symbol] | [symbol] | [symbol] | [symbol] | [symbol] |
| [symbol] | [symbol] | [symbol] | yes | yes | yes | yes | [symbol] | [symbol] | [symbol] | [symbol] | [symbol] | [symbol] |
| [symbol] | [symbol] | 26S proteosome non-ATPase regulatory | yes | yes | yes | yes | [symbol] | [symbol] | [symbol] | [symbol] | [symbol] | [symbol] |
| | | subunit 1 | | | | | | | | | | |
| [symbol] | [symbol] | Alpha [symbol] | yes | yes | yes | yes | [symbol] | [symbol] | [symbol] | [symbol] | [symbol] | [symbol] |
| [symbol] | [symbol] | Macrophage-capping protein | yes | yes | yes | yes | [symbol] | [symbol] | [symbol] | [symbol] | [symbol] | [symbol] |
| [symbol] | [symbol] | DNA replication licensing factor MCM2 | yes | yes | NO | yes | [symbol] | [symbol] | [symbol] | [symbol] | [symbol] | [symbol] |
| [symbol] | [symbol] | [symbol] domain-containing receptor 2 | yes | yes | NO | yes | [symbol] | [symbol] | [symbol] | [symbol] | [symbol] | [symbol] |
| [symbol] | [symbol] | Signal recognition particle subunit SRP68 | yes | yes | yes | yes | [symbol] | [symbol] | [symbol] | [symbol] | [symbol] | [symbol] |
| [symbol] | [symbol] | Protein disulfide-isomerase A6 | yes | yes | yes | yes | [symbol] | [symbol] | [symbol] | [symbol] | [symbol] | [symbol] |
| [symbol] | [symbol] | Cytoplasmic [symbol] hydratase | yes | yes | yes | yes | [symbol] | [symbol] | [symbol] | [symbol] | [symbol] | [symbol] |
| [symbol] | [symbol] | [symbol] | yes | yes | yes | yes | [symbol] | [symbol] | [symbol] | [symbol] | [symbol] | [symbol] |
| [symbol] | [symbol] | [symbol] antigen | yes | yes | yes | yes | [symbol] | [symbol] | [symbol] | [symbol] | [symbol] | [symbol] |
| [symbol] | [symbol] | [symbol] decarboxylase | yes | yes | yes | yes | [symbol] | [symbol] | [symbol] | [symbol] | [symbol] | [symbol] |
| [symbol] | [symbol] | [symbol] type-1 domain- [symbol] | yes | yes | yes | yes | [symbol] | [symbol] | [symbol] | [symbol] | [symbol] | [symbol] |
| [symbol] | [symbol] | [symbol] -dependent [symbol] | yes | yes | yes | yes | [symbol] | [symbol] | [symbol] | [symbol] | [symbol] | [symbol] |
| [symbol] | [symbol] | Pre-mRNA-splicing factor ATP-dependent | yes | yes | yes | yes | [symbol] | [symbol] | [symbol] | [symbol] | [symbol] | [symbol] |
| | | RNA helicase [symbol] | | | | | | | | | | |
| [symbol] | [symbol] | AP-3 complex subunit delta-1 | yes | yes | NO | yes | [symbol] | [symbol] | [symbol] | [symbol] | [symbol] | [symbol] |
| [symbol] | [symbol] | Collagen alpha- [symbol] chain | yes | yes | yes | yes | [symbol] | [symbol] | [symbol] | [symbol] | [symbol] | [symbol] |
| [symbol] | [symbol] | Coatomer subunit gamma-2 | yes | yes | yes | yes | [symbol] | [symbol] | [symbol] | [symbol] | [symbol] | [symbol] |
| [symbol] | [symbol] | Phosphatidylinositol-binding [symbol] | yes | yes | yes | yes | [symbol] | [symbol] | [symbol] | [symbol] | [symbol] | [symbol] |
| | | assembly protein | | | | | | | | | | |
| [symbol] | [symbol] | GTP-binding protein SAR1a | yes | yes | yes | yes | [symbol] | [symbol] | [symbol] | [symbol] | [symbol] | [symbol] |
| [symbol] | [symbol] | Multiple epidermal growth factor-like | yes | yes | yes | yes | [symbol] | [symbol] | [symbol] | [symbol] | [symbol] | [symbol] |
| | | domain protein 10 | | | | | | | | | | |
| [symbol] | [symbol] | Tryptophan--tRNA ligase, cytoplasmic | yes | yes | yes | yes | [symbol] | [symbol] | [symbol] | [symbol] | [symbol] | [symbol] |
| [symbol] | [symbol] | [symbol] protein sorting-associated protein 26B | yes | yes | NO | yes | [symbol] | [symbol] | [symbol] | [symbol] | [symbol] | [symbol] |
| [symbol] | [symbol] | Spectrin beta chain, non- [symbol] | yes | yes | yes | yes | [symbol] | [symbol] | [symbol] | [symbol] | [symbol] | [symbol] |
| [symbol] | [symbol] | [symbol] -5 | yes | yes | yes | yes | [symbol] | [symbol] | [symbol] | [symbol] | [symbol] | [symbol] |
| [symbol] | [symbol] | Nectin-2 | yes | yes | NO | yes | [symbol] | [symbol] | [symbol] | [symbol] | [symbol] | [symbol] |
| [symbol] | [symbol] | Latent-transforming growth factor β-binding | yes | yes | yes | yes | [symbol] | [symbol] | [symbol] | [symbol] | [symbol] | [symbol] |
| | | protein 1 | | | | | | | | | | |
| [symbol] | [symbol] | [symbol] alpha chain, non- [symbol] | yes | yes | yes | yes | [symbol] | [symbol] | [symbol] | [symbol] | [symbol] | [symbol] |
| [symbol] | [symbol] | [symbol] -like protein 1 | yes | yes | NO | yes | [symbol] | [symbol] | [symbol] | [symbol] | [symbol] | [symbol] |
| [symbol] | [symbol] | Glycerol-3-phosphate phosphatase | yes | yes | yes | yes | [symbol] | [symbol] | [symbol] | [symbol] | [symbol] | [symbol] |
| [symbol] | [symbol] | Coagulation factor [symbol] chain | yes | yes | yes | yes | [symbol] | [symbol] | [symbol] | [symbol] | [symbol] | [symbol] |
| [symbol] | [symbol] | [symbol] | yes | yes | yes | yes | [symbol] | [symbol] | [symbol] | [symbol] | [symbol] | [symbol] |
| [symbol] | [symbol] | [symbol] hydrogen exchanger 1 | yes | yes | yes | yes | [symbol] | [symbol] | [symbol] | [symbol] | [symbol] | [symbol] |
| [symbol] | [symbol] | Cullin-3 | yes | yes | yes | yes | [symbol] | [symbol] | [symbol] | [symbol] | [symbol] | [symbol] |
| [symbol] | [symbol] | Collagen alpha- [symbol] chain | yes | yes | NO | yes | [symbol] | [symbol] | [symbol] | [symbol] | [symbol] | [symbol] |
| [symbol] | [symbol] | Ectonucleotide pyrophosphatase | yes | yes | NO | yes | [symbol] | [symbol] | [symbol] | [symbol] | [symbol] | [symbol] |
| | | [symbol] member 2 | | | | | | | | | | |
| [symbol] | [symbol] | Thioredoxin-like protein 1 | yes | yes | yes | yes | [symbol] | [symbol] | [symbol] | [symbol] | [symbol] | [symbol] |
| [symbol] | [symbol] | Fibroblast growth factor receptor 1 | yes | yes | yes | yes | [symbol] | [symbol] | [symbol] | [symbol] | [symbol] | [symbol] |
| [symbol] | [symbol] | Collagen alpha [symbol] chain | yes | yes | NO | yes | [symbol] | [symbol] | [symbol] | [symbol] | [symbol] | [symbol] |
| [symbol] | [symbol] | Eukaryotic translation [symbol] factor 4B | yes | yes | NO | yes | [symbol] | [symbol] | [symbol] | [symbol] | [symbol] | [symbol] |
| [symbol] | [symbol] | [symbol] DNA-binding protein [symbol] | yes | yes | yes | yes | [symbol] | [symbol] | [symbol] | [symbol] | [symbol] | [symbol] |
| [symbol] | [symbol] | [symbol] -related protein Rab- [symbol] | yes | yes | NO | yes | [symbol] | [symbol] | [symbol] | [symbol] | [symbol] | [symbol] |
| [symbol] | [symbol] | [symbol] -dependent RNA helicase DDX3X | yes | yes | yes | yes | [symbol] | [symbol] | [symbol] | [symbol] | [symbol] | [symbol] |
| [symbol] | [symbol] | Interleukin enhancer-binding factor 2 | yes | yes | yes | yes | [symbol] | [symbol] | [symbol] | [symbol] | [symbol] | [symbol] |
| [symbol] | [symbol] | Purine nucleoside phosphorylase | yes | yes | yes | yes | [symbol] | [symbol] | [symbol] | [symbol] | [symbol] | [symbol] |
| [symbol] | [symbol] | 26S proteasome non-ATPase regulatory | yes | yes | NO | yes | [symbol] | [symbol] | [symbol] | [symbol] | [symbol] | [symbol] |
| | | subunit 4 | | | | | | | | | | |
| [symbol] | [symbol] | Neurogenic locus notch homolog protein 3 | yes | yes | yes | yes | [symbol] | [symbol] | [symbol] | [symbol] | [symbol] | [symbol] |
| [symbol] | [symbol] | Syntaxin-binding protein 2 | yes | yes | yes | yes | [symbol] | [symbol] | [symbol] | [symbol] | [symbol] | [symbol] |
| [symbol] | [symbol] | Fumarate hydratase, mitochondrial | yes | yes | yes | yes | [symbol] | [symbol] | [symbol] | [symbol] | [symbol] | [symbol] |
| [symbol] | [symbol] | NADH-cytochrome b5 reductase 3 | yes | yes | NO | yes | [symbol] | [symbol] | [symbol] | [symbol] | [symbol] | [symbol] |
| [symbol] | [symbol] | Delta- [symbol] acid dehydratase | yes | yes | yes | yes | [symbol] | [symbol] | [symbol] | [symbol] | [symbol] | [symbol] |
| [symbol] | [symbol] | Ubiquitin-like modifier-activating enzyme [symbol] | yes | yes | NO | yes | [symbol] | [symbol] | [symbol] | [symbol] | [symbol] | [symbol] |
| [symbol] | [symbol] | Acyl-CoA-binding protein | yes | yes | NO | yes | [symbol] | [symbol] | [symbol] | [symbol] | [symbol] | [symbol] |
| [symbol] | [symbol] | Protein disulfide-isomer A3 | yes | yes | yes | yes | [symbol] | [symbol] | [symbol] | [symbol] | [symbol] | [symbol] |
| [symbol] | [symbol] | [symbol] and metalloproteinase with | yes | yes | yes | yes | [symbol] | [symbol] | [symbol] | [symbol] | [symbol] | [symbol] |
| | | thrombospondin motifs 5 | | | | | | | | | | |
| [symbol] | [symbol] | Hepatoma-derived growth factor | yes | yes | yes | yes | [symbol] | [symbol] | [symbol] | [symbol] | [symbol] | [symbol] |
| [symbol] | [symbol] | Focal adhesion kinase 1 | yes | yes | NO | yes | [symbol] | [symbol] | [symbol] | [symbol] | [symbol] | [symbol] |
| [symbol] | [symbol] | [symbol] -like 2 | yes | yes | NO | yes | [symbol] | [symbol] | [symbol] | [symbol] | [symbol] | [symbol] |
| [symbol] | [symbol] | [symbol] assembly protein 1-like 4 | yes | NO | yes | yes | [symbol] | [symbol] | [symbol] | [symbol] | [symbol] | [symbol] |
| [symbol] | [symbol] | [symbol] and adenovirus receptor | yes | NO | yes | yes | [symbol] | [symbol] | [symbol] | [symbol] | [symbol] | [symbol] |
| | | homolog | | | | | | | | | | |
| [symbol] | [symbol] | RNA-binding protein [symbol] | yes | NO | NO | yes | [symbol] | [symbol] | [symbol] | [symbol] | [symbol] | [symbol] |
| [symbol] | [symbol] | Inositol monophosphate 1 | yes | NO | NO | yes | [symbol] | [symbol] | [symbol] | [symbol] | [symbol] | [symbol] |
| [symbol] | [symbol] | Calponin-3 | yes | NO | NO | yes | [symbol] | [symbol] | [symbol] | [symbol] | [symbol] | [symbol] |
| [symbol] | [symbol] | General vesicular transport factor p115 | yes | NO | yes | yes | [symbol] | [symbol] | [symbol] | [symbol] | [symbol] | [symbol] |
| [symbol] | [symbol] | [symbol] -1 | yes | NO | yes | yes | [symbol] | [symbol] | [symbol] | [symbol] | [symbol] | [symbol] |
| [symbol] | [symbol] | Nuclear receptor-binding protein | yes | NO | yes | yes | [symbol] | [symbol] | [symbol] | [symbol] | [symbol] | [symbol] |
| [symbol] | [symbol] | [symbol] -associated membrane protein- | yes | NO | NO | yes | [symbol] | [symbol] | [symbol] | [symbol] | [symbol] | [symbol] |
| | | associated protein A | | | | | | | | | | |
| [symbol] | [symbol] | Ubiquitin-conjugating enzyme [symbol] | yes | NO | NO | yes | [symbol] | [symbol] | [symbol] | [symbol] | [symbol] | [symbol] |
| [symbol] | [symbol] | Heterogeneous nuclear ribonucleoprotein H | yes | NO | NO | yes | [symbol] | [symbol] | [symbol] | [symbol] | [symbol] | [symbol] |
| [symbol] | [symbol] | Protein [symbol] | yes | NO | yes | yes | [symbol] | [symbol] | [symbol] | [symbol] | [symbol] | [symbol] |
| [symbol] | [symbol] | Glutathione S-transferase omega-1 | yes | NO | NO | yes | [symbol] | [symbol] | [symbol] | [symbol] | [symbol] | [symbol] |
| [symbol] | [symbol] | [symbol] | yes | NO | NO | yes | [symbol] | [symbol] | [symbol] | [symbol] | [symbol] | [symbol] |
| [symbol] | [symbol] | [symbol] myosin- [symbol] | yes | NO | yes | yes | [symbol] | [symbol] | [symbol] | [symbol] | [symbol] | [symbol] |
| [symbol] | [symbol] | 60S ribosomal protein [symbol] | yes | NO | yes | yes | [symbol] | [symbol] | [symbol] | [symbol] | [symbol] | [symbol] |
| [symbol] | [symbol] | Phospholipase D3 | yes | NO | NO | yes | [symbol] | [symbol] | [symbol] | [symbol] | [symbol] | [symbol] |
| [symbol] | [symbol] | Complement C1q [symbol] -related protein 3 | yes | NO | NO | yes | [symbol] | [symbol] | [symbol] | [symbol] | [symbol] | [symbol] |
| [symbol] | [symbol] | Ras-related protein Rab-6A | yes | NO | NO | yes | [symbol] | [symbol] | [symbol] | [symbol] | [symbol] | [symbol] |
| [symbol] | [symbol] | Transformer-2 protein homolog beta | yes | NO | NO | yes | [symbol] | [symbol] | [symbol] | [symbol] | [symbol] | [symbol] |
| [symbol] | [symbol] | Trascriptional activator protein [symbol] -alpha | yes | NO | yes | yes | [symbol] | [symbol] | [symbol] | [symbol] | [symbol] | [symbol] |
| [symbol] | [symbol] | Protein disulfide-isomerase A4 | yes | NO | NO | yes | [symbol] | [symbol] | [symbol] | [symbol] | [symbol] | [symbol] |
| [symbol] | [symbol] | Nerine arginine-rich splicing factor [symbol] | yes | NO | yes | yes | [symbol] | [symbol] | [symbol] | [symbol] | [symbol] | [symbol] |
| [symbol] | [symbol] | Lupus [symbol] protein homolog | yes | NO | NO | yes | [symbol] | [symbol] | [symbol] | [symbol] | [symbol] | [symbol] |
| [symbol] | [symbol] | Importin-7 | yes | NO | yes | yes | [symbol] | [symbol] | [symbol] | [symbol] | [symbol] | [symbol] |
| [symbol] | [symbol] | Alpha-crystallin B chain | yes | NO | NO | yes | [symbol] | [symbol] | [symbol] | [symbol] | [symbol] | [symbol] |
| [symbol] | [symbol] | Integrin beta- 3 | yes | NO | NO | yes | [symbol] | [symbol] | [symbol] | [symbol] | [symbol] | [symbol] |
| [symbol] | [symbol] | Insulin-like growth factor 1 receptor | yes | NO | NO | yes | [symbol] | [symbol] | [symbol] | [symbol] | [symbol] | [symbol] |
| [symbol] | [symbol] | Extended synaptotagmin-1 | yes | NO | yes | yes | [symbol] | [symbol] | [symbol] | [symbol] | [symbol] | [symbol] |
| [symbol] | [symbol] | Proenkephalin-A | yes | NO | NO | yes | [symbol] | [symbol] | [symbol] | [symbol] | [symbol] | [symbol] |
| [symbol] | [symbol] | [symbol] membrane protein 2 | yes | NO | NO | yes | [symbol] | [symbol] | [symbol] | [symbol] | [symbol] | [symbol] |
| [symbol] | [symbol] | Choline transporter-like protein 1 | yes | NO | NO | yes | [symbol] | [symbol] | [symbol] | [symbol] | [symbol] | [symbol] |
| [symbol] | [symbol] | [symbol] von Willebrand factor type A, [symbol] | yes | NO | NO | yes | [symbol] | [symbol] | [symbol] | [symbol] | [symbol] | [symbol] |
| | | and pentraxin domain-containing protein 1 | | | | | | | | | | |
| [symbol] | [symbol] | Interleukin enhancer-binding factor 3 | NO | yes | NO | yes | [symbol] | [symbol] | [symbol] | [symbol] | [symbol] | [symbol] |
| [symbol] | [symbol] | Apolipoprotein M | NO | yes | yes | yes | [symbol] | [symbol] | [symbol] | [symbol] | [symbol] | [symbol] |
| [symbol] | [symbol] | [symbol] 2 | NO | yes | NO | yes | [symbol] | [symbol] | [symbol] | [symbol] | [symbol] | [symbol] |
| [symbol] | [symbol] | [symbol] | NO | yes | NO | yes | [symbol] | [symbol] | [symbol] | [symbol] | [symbol] | [symbol] |
| [symbol] | [symbol] | [symbol] -1 | NO | yes | NO | yes | [symbol] | [symbol] | [symbol] | [symbol] | [symbol] | [symbol] |
| [symbol] | [symbol] | WW domain-binding protein 2 | NO | yes | NO | yes | [symbol] | [symbol] | [symbol] | [symbol] | [symbol] | [symbol] |
| [symbol] | [symbol] | Leucyl-cystinyl aminopeptidase | NO | yes | yes | yes | [symbol] | [symbol] | [symbol] | [symbol] | [symbol] | [symbol] |
| [symbol] | [symbol] | Pigment epithelium-derived factor | NO | NO | yes | yes | [symbol] | [symbol] | [symbol] | [symbol] | [symbol] | [symbol] |
| [symbol] | [symbol] | Alpha- [symbol] | NO | NO | NO | yes | [symbol] | [symbol] | [symbol] | [symbol] | [symbol] | [symbol] |
| [symbol] | [symbol] | Calnexin | NO | NO | yes | yes | [symbol] | [symbol] | [symbol] | [symbol] | [symbol] | [symbol] |
| [symbol] | [symbol] | Cullin-5 | yes | yes | yes | NO | [symbol] | [symbol] | [symbol] | [symbol] | [symbol] | [symbol] |
| [symbol] | [symbol] | 26S proteasome non-ATPase regulatory | yes | yes | yes | NO | [symbol] | [symbol] | [symbol] | [symbol] | [symbol] | [symbol] |
| | | subunit 8 | | | | | | | | | | |
| [symbol] | [symbol] | DNA-directed RNA polymerase II subunit | yes | yes | yes | NO | [symbol] | [symbol] | [symbol] | [symbol] | [symbol] | [symbol] |
| | | RPB3 | | | | | | | | | | |
| [symbol] | [symbol] | Nuclear cap-binding protein subunit 1 | yes | yes | yes | NO | [symbol] | [symbol] | [symbol] | [symbol] | [symbol] | [symbol] |
| [symbol] | [symbol] | N-alpha-acetyltransferase 10 | yes | yes | NO | NO | [symbol] | [symbol] | [symbol] | [symbol] | [symbol] | [symbol] |
| [symbol] | [symbol] | Glucose-6-phosphate 1-dehydrogenase [symbol] | yes | yes | NO | NO | [symbol] | [symbol] | [symbol] | [symbol] | [symbol] | [symbol] |
| [symbol] | [symbol] | DNA-directed RNA polymerases I, II, and III | yes | yes | yes | NO | [symbol] | [symbol] | [symbol] | [symbol] | [symbol] | [symbol] |
| | | subunit RPABC3 | | | | | | | | | | |
| [symbol] | [symbol] | Immunity-related GTPase family M protein 1 | yes | yes | NO | NO | [symbol] | [symbol] | [symbol] | [symbol] | [symbol] | [symbol] |
| [symbol] | [symbol] | Serine threonine-protein phosphatase 2A 56 | yes | yes | NO | NO | [symbol] | [symbol] | [symbol] | [symbol] | [symbol] | [symbol] |
| | | kDa regulatory subunit gamma isoform | | | | | | | | | | |
| [symbol] | [symbol] | DNA [symbol] licensing factor MCM4 | yes | yes | NO | NO | [symbol] | [symbol] | [symbol] | [symbol] | [symbol] | [symbol] |
| [symbol] | [symbol] | Cullin-2 | yes | yes | yes | NO | [symbol] | [symbol] | [symbol] | [symbol] | [symbol] | [symbol] |
| [symbol] | [symbol] | Cell division cycle protein 27 homolog | yes | yes | NO | NO | [symbol] | [symbol] | [symbol] | [symbol] | [symbol] | [symbol] |
| [symbol] | [symbol] | Microtubule-associated protein [symbol] | yes | yes | yes | NO | [symbol] | [symbol] | [symbol] | [symbol] | [symbol] | [symbol] |
| [symbol] | [symbol] | Developmentally-regulated GTP-binding | yes | yes | NO | NO | [symbol] | [symbol] | [symbol] | [symbol] | [symbol] | [symbol] |
| | | protein 2 | | | | | | | | | | |
| [symbol] | [symbol] | [symbol] -related [symbol] -associated actin- | yes | yes | NO | NO | [symbol] | [symbol] | [symbol] | [symbol] | [symbol] | [symbol] |
| | | dependent regulator [symbol] chromatin subfamily | | | | | | | | | | |
| | | A member 5 | | | | | | | | | | |
| [symbol] | [symbol] | [symbol] ubiquitin-protein ligase [symbol] | yes | yes | NO | NO | [symbol] | [symbol] | [symbol] | [symbol] | [symbol] | [symbol] |
| [symbol] | [symbol] | Dynactin subunit 4 | yes | yes | NO | NO | [symbol] | [symbol] | [symbol] | [symbol] | [symbol] | [symbol] |
| [symbol] | [symbol] | Double-strand-break repair protein rad21 | yes | yes | yes | NO | [symbol] | [symbol] | [symbol] | [symbol] | [symbol] | [symbol] |
| | | homolog | | | | | | | | | | |
| [symbol] | [symbol] | NudC domain-containing protein 1 | yes | yes | NO | NO | [symbol] | [symbol] | [symbol] | [symbol] | [symbol] | [symbol] |
| [symbol] | [symbol] | Mediator [symbol] RNA polymerase II transcription | yes | yes | yes | NO | [symbol] | [symbol] | [symbol] | [symbol] | [symbol] | [symbol] |
| | | subunit 23 | | | | | | | | | | |
| [symbol] | [symbol] | Metastasis-associated protein [symbol] | yes | yes | yes | NO | [symbol] | [symbol] | [symbol] | [symbol] | [symbol] | [symbol] |
| [symbol] | [symbol] | [symbol] -promoting complex subunit 1 | yes | yes | NO | NO | [symbol] | [symbol] | [symbol] | [symbol] | [symbol] | [symbol] |
| [symbol] | [symbol] | Sorting nexin-1 | yes | yes | yes | NO | [symbol] | [symbol] | [symbol] | [symbol] | [symbol] | [symbol] |
| [symbol] | [symbol] | [symbol] repeat-containing protein 5A | yes | yes | yes | NO | [symbol] | [symbol] | [symbol] | [symbol] | [symbol] | [symbol] |
| [symbol] | [symbol] | Probable ubiquitin carboxyl-terminal | yes | yes | NO | NO | [symbol] | [symbol] | [symbol] | [symbol] | [symbol] | [symbol] |
| | | hydrolase [symbol] | | | | | | | | | | |
| [symbol] | [symbol] | Acyl-protein thioesterase 2 | yes | yes | yes | NO | [symbol] | [symbol] | [symbol] | [symbol] | [symbol] | [symbol] |
| [symbol] | [symbol] | Cell [symbol] cycle protein [symbol] homolog | yes | yes | yes | NO | [symbol] | [symbol] | [symbol] | [symbol] | [symbol] | [symbol] |
| [symbol] | [symbol] | Protein RMD5 homolog A | yes | yes | NO | NO | [symbol] | [symbol] | [symbol] | [symbol] | [symbol] | [symbol] |
| [symbol] | [symbol] | Cullin-7 | yes | yes | yes | NO | [symbol] | [symbol] | [symbol] | [symbol] | [symbol] | [symbol] |
| [symbol] | [symbol] | [symbol] -1 | yes | yes | yes | NO | [symbol] | [symbol] | [symbol] | [symbol] | [symbol] | [symbol] |
| [symbol] | [symbol] | Cytosolic purine [symbol] -nucleotidase | yes | yes | NO | NO | [symbol] | [symbol] | [symbol] | [symbol] | [symbol] | [symbol] |
| [symbol] | [symbol] | ATP-dependent RNA helicase [symbol] | yes | yes | yes | NO | [symbol] | [symbol] | [symbol] | [symbol] | [symbol] | [symbol] |
| [symbol] | [symbol] | Ran-binding protein 10 | yes | yes | yes | NO | [symbol] | [symbol] | [symbol] | [symbol] | [symbol] | [symbol] |
| [symbol] | [symbol] | 116 kDa [symbol] small nuclear ribonucleoprotein | yes | yes | yes | NO | [symbol] | [symbol] | [symbol] | [symbol] | [symbol] | [symbol] |
| | | component | | | | | | | | | | |
| [symbol] | [symbol] | Elongin-B | yes | yes | yes | NO | [symbol] | [symbol] | [symbol] | [symbol] | [symbol] | [symbol] |
| [symbol] | [symbol] | [symbol] kinase | yes | yes | yes | NO | [symbol] | [symbol] | [symbol] | [symbol] | [symbol] | [symbol] |
| [symbol] | [symbol] | DNA-directed RNA polymerases I, II, and III | yes | yes | yes | NO | [symbol] | [symbol] | [symbol] | [symbol] | [symbol] | [symbol] |
| | | subunit [symbol] | | | | | | | | | | |
| [symbol] | [symbol] | Histone-arginine methyltransterase CARM1 | yes | yes | yes | NO | [symbol] | [symbol] | [symbol] | [symbol] | [symbol] | [symbol] |
| [symbol] | [symbol] | AP-3 complex subunit [symbol] | yes | yes | yes | NO | [symbol] | [symbol] | [symbol] | [symbol] | [symbol] | [symbol] |
| [symbol] | [symbol] | Golgi reassembly-stacking protein 2 | yes | yes | yes | NO | [symbol] | [symbol] | [symbol] | [symbol] | [symbol] | [symbol] |
| [symbol] | [symbol] | [symbol] complex subunit 2 | yes | yes | NO | NO | [symbol] | [symbol] | [symbol] | [symbol] | [symbol] | [symbol] |
| [symbol] | [symbol] | Anaphase-promoting complex subunit 2 | yes | yes | yes | NO | [symbol] | [symbol] | [symbol] | [symbol] | [symbol] | [symbol] |
| [symbol] | [symbol] | Gamma-tubulin complex component 2 | yes | yes | yes | NO | [symbol] | [symbol] | [symbol] | [symbol] | [symbol] | [symbol] |
| [symbol] | [symbol] | Protein O-GlcNAcase | yes | yes | yes | NO | [symbol] | [symbol] | [symbol] | [symbol] | [symbol] | [symbol] |
| [symbol] | [symbol] | Nuclear [symbol] complex protein [symbol] | yes | yes | yes | NO | [symbol] | [symbol] | [symbol] | [symbol] | [symbol] | [symbol] |
| [symbol] | [symbol] | DNA-directed RNA polymerase II subunit | yes | yes | yes | NO | [symbol] | [symbol] | [symbol] | [symbol] | [symbol] | [symbol] |
| | | RPB2 | | | | | | | | | | |
| [symbol] | [symbol] | Lysine-specific histone demethylase [symbol] | yes | yes | NO | NO | [symbol] | [symbol] | [symbol] | [symbol] | [symbol] | [symbol] |
| [symbol] | [symbol] | DNA replication licensing factor [symbol] | yes | yes | yes | NO | [symbol] | [symbol] | [symbol] | [symbol] | [symbol] | [symbol] |
| [symbol] | [symbol] | DNA ligase 1 | yes | yes | yes | NO | [symbol] | [symbol] | [symbol] | [symbol] | [symbol] | [symbol] |
| [symbol] | [symbol] | Splicing factor 3B subunit 4 | yes | yes | NO | NO | [symbol] | [symbol] | [symbol] | [symbol] | [symbol] | [symbol] |
| [symbol] | [symbol] | Coiled-coil domain-containing protein 22 | yes | yes | yes | NO | [symbol] | [symbol] | [symbol] | [symbol] | [symbol] | [symbol] |
| [symbol] | [symbol] | Protein [symbol] 14 homolog | yes | yes | yes | NO | [symbol] | [symbol] | [symbol] | [symbol] | [symbol] | [symbol] |
| [symbol] | [symbol] | Phosphoribosyl pyrophosphate synthase- | yes | yes | yes | NO | [symbol] | [symbol] | [symbol] | [symbol] | [symbol] | [symbol] |
| | | associated protein 1 | | | | | | | | | | |
| [symbol] | [symbol] | WD repeat-containing protein [symbol] | yes | yes | yes | NO | [symbol] | [symbol] | [symbol] | [symbol] | [symbol] | [symbol] |
| [symbol] | [symbol] | Metastasis-associated protein [symbol] | yes | yes | yes | NO | [symbol] | [symbol] | [symbol] | [symbol] | [symbol] | [symbol] |
| [symbol] | [symbol] | E3 ubiquitin-protein ligase [symbol] | yes | yes | NO | NO | [symbol] | [symbol] | [symbol] | [symbol] | [symbol] | [symbol] |
| [symbol] | [symbol] | Ribosome production factor 2 homolog | yes | yes | yes | NO | [symbol] | [symbol] | [symbol] | [symbol] | [symbol] | [symbol] |
| [symbol] | [symbol] | DNA [symbol] | yes | yes | yes | NO | [symbol] | [symbol] | [symbol] | [symbol] | [symbol] | [symbol] |
| [symbol] | [symbol] | 60S [symbol] subunit biogenesis protein | yes | yes | yes | NO | [symbol] | [symbol] | [symbol] | [symbol] | [symbol] | [symbol] |
| | | [symbol] homolog | | | | | | | | | | |
| [symbol] | [symbol] | DNA-directed RNA polymerase II subunit | yes | yes | yes | NO | [symbol] | [symbol] | [symbol] | [symbol] | [symbol] | [symbol] |
| | | RPB1 | | | | | | | | | | |
| [symbol] | [symbol] | DNA replication licensing factor MCM5 | yes | yes | NO | NO | [symbol] | [symbol] | [symbol] | [symbol] | [symbol] | [symbol] |
| [symbol] | [symbol] | DNA-directed RNA polymerase [symbol] subunit | yes | yes | NO | NO | [symbol] | [symbol] | [symbol] | [symbol] | [symbol] | [symbol] |
| | | RPA2 | | | | | | | | | | |
| [symbol] | [symbol] | Dual specificity mitogen-activated protein | yes | yes | NO | NO | [symbol] | [symbol] | [symbol] | [symbol] | [symbol] | [symbol] |
| | | kinase kinase 4 | | | | | | | | | | |
| [symbol] | [symbol] | Structural maintenance of [symbol] | yes | yes | NO | NO | [symbol] | [symbol] | [symbol] | [symbol] | [symbol] | [symbol] |
| | | protein 2 | | | | | | | | | | |
| [symbol] | [symbol] | Anaphase-promoting complex subunit 5 | yes | yes | yes | NO | [symbol] | [symbol] | [symbol] | [symbol] | [symbol] | [symbol] |
| [symbol] | [symbol] | Glycosylated lysosomal membrane protein | yes | yes | yes | NO | [symbol] | [symbol] | [symbol] | [symbol] | [symbol] | [symbol] |
| [symbol] | [symbol] | p21-activated protein kinase-interacting | yes | yes | yes | NO | [symbol] | [symbol] | [symbol] | [symbol] | [symbol] | [symbol] |
| | | protein 1 | | | | | | | | | | |
| [symbol] | [symbol] | Ribosome biogenesis protein BOP1 | yes | yes | yes | NO | [symbol] | [symbol] | [symbol] | [symbol] | [symbol] | [symbol] |
| [symbol] | [symbol] | Anaphase-promoting complex subunit 4 | yes | yes | NO | NO | [symbol] | [symbol] | [symbol] | [symbol] | [symbol] | [symbol] |
| [symbol] | [symbol] | ATP- binding cassette sub-family [symbol] member 1 | yes | yes | yes | NO | [symbol] | [symbol] | [symbol] | [symbol] | [symbol] | [symbol] |
| [symbol] | [symbol] | Acetyl-CoA carboxylase 1 | yes | yes | NO | NO | [symbol] | [symbol] | [symbol] | [symbol] | [symbol] | [symbol] |
| [symbol] | [symbol] | [symbol] -conjucating enxyme [symbol] | yes | yes | yes | NO | [symbol] | [symbol] | [symbol] | [symbol] | [symbol] | [symbol] |
| [symbol] | [symbol] | [symbol] small nucleolar RNA-associated protein | yes | yes | NO | NO | [symbol] | [symbol] | [symbol] | [symbol] | [symbol] | [symbol] |
| | | 15 homolog | | | | | | | | | | |
| [symbol] | [symbol] | [symbol] small nuclear ribonucleoprotein 40 kDa | yes | yes | yes | NO | [symbol] | [symbol] | [symbol] | [symbol] | [symbol] | [symbol] |
| | | protein | | | | | | | | | | |
| [symbol] | [symbol] | Exosome complex exonuclease RRP44 | yes | yes | yes | NO | [symbol] | [symbol] | [symbol] | [symbol] | [symbol] | [symbol] |
| [symbol] | [symbol] | Anaphase-promoting complex subunit 7 | yes | yes | NO | NO | [symbol] | [symbol] | [symbol] | [symbol] | [symbol] | [symbol] |
| [symbol] | [symbol] | Methyl-CpG-binding domain protein [symbol] | yes | yes | NO | NO | [symbol] | [symbol] | [symbol] | [symbol] | [symbol] | [symbol] |
| [symbol] | [symbol] | Glycylpeptide N-tetradecanoyltransferase 1 | yes | yes | yes | NO | [symbol] | [symbol] | [symbol] | [symbol] | [symbol] | [symbol] |
| [symbol] | [symbol] | Serine threonine-protein phosphatase 6 | yes | yes | yes | NO | [symbol] | [symbol] | [symbol] | [symbol] | [symbol] | [symbol] |
| | | catalytic subinit | | | | | | | | | | |
| [symbol] | [symbol] | transportin-3 | yes | yes | yes | NO | [symbol] | [symbol] | [symbol] | [symbol] | [symbol] | [symbol] |
| [symbol] | [symbol] | Protein transport protein [symbol] | yes | yes | NO | NO | [symbol] | [symbol] | [symbol] | [symbol] | [symbol] | [symbol] |
| [symbol] | [symbol] | WASH complex subunit 5 | yes | yes | NO | NO | [symbol] | [symbol] | [symbol] | [symbol] | [symbol] | [symbol] |
| [symbol] | [symbol] | Protein arginine N-methyltransferase 3 | yes | yes | yes | NO | [symbol] | [symbol] | [symbol] | [symbol] | [symbol] | [symbol] |
| [symbol] | [symbol] | Serine threonine-protein phosphatase 2A 55 | yes | yes | yes | NO | [symbol] | [symbol] | [symbol] | [symbol] | [symbol] | [symbol] |
| | | kDa regulatory subunit B alpha isoform | | | | | | | | | | |
| [symbol] | [symbol] | DNA-directed RNA polymerases I and III | yes | yes | yes | NO | [symbol] | [symbol] | [symbol] | [symbol] | [symbol] | [symbol] |
| | | subunit RPAC1 | | | | | | | | | | |
| [symbol] | [symbol] | DNA replication licensing factor MCM6 | yes | yes | yes | NO | [symbol] | [symbol] | [symbol] | [symbol] | [symbol] | [symbol] |
| [symbol] | [symbol] | WD repeat-containing protein 26 | yes | yes | NO | NO | [symbol] | [symbol] | [symbol] | [symbol] | [symbol] | [symbol] |
| [symbol] | [symbol] | Acetoacetyl-CoA [symbol] | yes | yes | yes | NO | [symbol] | [symbol] | [symbol] | [symbol] | [symbol] | [symbol] |
| [symbol] | [symbol] | WASH complex subunit 4 | yes | yes | NO | NO | [symbol] | [symbol] | [symbol] | [symbol] | [symbol] | [symbol] |
| [symbol] | [symbol] | [symbol] interacting protein | yes | yes | yes | NO | [symbol] | [symbol] | [symbol] | [symbol] | [symbol] | [symbol] |
| [symbol] | [symbol] | Small nuclear ribonucleoprotein-associated | yes | yes | yes | NO | [symbol] | [symbol] | [symbol] | [symbol] | [symbol] | [symbol] |
| | | protein B | | | | | | | | | | |
| [symbol] | [symbol] | WD repeat-containing protein [symbol] | yes | yes | yes | NO | [symbol] | [symbol] | [symbol] | [symbol] | [symbol] | [symbol] |
| [symbol] | [symbol] | Scavenger receptor class B member 1 | yes | yes | yes | NO | [symbol] | [symbol] | [symbol] | [symbol] | [symbol] | [symbol] |
| [symbol] | [symbol] | Ankyrin repeat domain-containing protein | yes | yes | NO | NO | [symbol] | [symbol] | [symbol] | [symbol] | [symbol] | [symbol] |
| | | 13A | | | | | | | | | | |
| [symbol] | [symbol] | Proteasome adapter and [symbol] protein [symbol] | yes | yes | NO | NO | [symbol] | [symbol] | [symbol] | [symbol] | [symbol] | [symbol] |
| [symbol] | [symbol] | Glycylpeptide N-tetradexanoyltransferase 2 | yes | yes | NO | NO | [symbol] | [symbol] | [symbol] | [symbol] | [symbol] | [symbol] |
| [symbol] | [symbol] | Rho-related GTP-binding protein [symbol] | yes | yes | yes | NO | [symbol] | [symbol] | [symbol] | [symbol] | [symbol] | [symbol] |
| [symbol] | [symbol] | ATP-dependent RNA helicase DHX36 | yes | yes | NO | NO | [symbol] | [symbol] | [symbol] | [symbol] | [symbol] | [symbol] |
| [symbol] | [symbol] | Ubiquitin conjugation factor [symbol] | yes | yes | yes | NO | [symbol] | [symbol] | [symbol] | [symbol] | [symbol] | [symbol] |
| [symbol] | [symbol] | Protein [symbol] homolog 1 | yes | yes | yes | NO | [symbol] | [symbol] | [symbol] | [symbol] | [symbol] | [symbol] |
| [symbol] | [symbol] | Proteasome activator complex subunit 4 | yes | yes | NO | NO | [symbol] | [symbol] | [symbol] | [symbol] | [symbol] | [symbol] |
| [symbol] | [symbol] | Methylosome protein 50 | yes | yes | NO | NO | [symbol] | [symbol] | [symbol] | [symbol] | [symbol] | [symbol] |
| [symbol] | [symbol] | transcriptional repressor [symbol] -beta | yes | yes | NO | NO | [symbol] | [symbol] | [symbol] | [symbol] | [symbol] | [symbol] |
| [symbol] | [symbol] | [symbol] small [symbol] RNA-associated protein 4 | yes | yes | NO | NO | [symbol] | [symbol] | [symbol] | [symbol] | [symbol] | [symbol] |
| | | homolog | | | | | | | | | | |
| [symbol] | [symbol] | Protein [symbol] -4 | yes | yes | yes | NO | [symbol] | [symbol] | [symbol] | [symbol] | [symbol] | [symbol] |
| [symbol] | [symbol] | Importin-4 | yes | yes | NO | NO | [symbol] | [symbol] | [symbol] | [symbol] | [symbol] | [symbol] |
| [symbol] | [symbol] | [symbol] -binding protein 1 homolog | yes | yes | yes | NO | [symbol] | [symbol] | [symbol] | [symbol] | [symbol] | [symbol] |
| [symbol] | [symbol] | [symbol] complex subunit 1 | yes | yes | NO | NO | [symbol] | [symbol] | [symbol] | [symbol] | [symbol] | [symbol] |
| [symbol] | [symbol] | Protein [symbol] homolog | yes | yes | yes | NO | [symbol] | [symbol] | [symbol] | [symbol] | [symbol] | [symbol] |
| [symbol] | [symbol] | [symbol] peptide receptor [symbol] | yes | yes | yes | NO | [symbol] | [symbol] | [symbol] | [symbol] | [symbol] | [symbol] |
| [symbol] | [symbol] | Brain-specific [symbol] inhibitor 1- | yes | yes | yes | NO | [symbol] | [symbol] | [symbol] | [symbol] | [symbol] | [symbol] |
| | | associated protein 2 | | | | | | | | | | |
| [symbol] | [symbol] | Ras-related GTP-binding protein [symbol] | yes | yes | yes | NO | [symbol] | [symbol] | [symbol] | [symbol] | [symbol] | [symbol] |
| [symbol] | [symbol] | Rho-related GTP-binding protein [symbol] | yes | yes | NO | NO | [symbol] | [symbol] | [symbol] | [symbol] | [symbol] | [symbol] |
| [symbol] | [symbol] | [symbol] | yes | yes | NO | NO | [symbol] | [symbol] | [symbol] | [symbol] | [symbol] | [symbol] |
| [symbol] | [symbol] | [symbol] | yes | yes | yes | NO | [symbol] | [symbol] | [symbol] | [symbol] | [symbol] | [symbol] |
| [symbol] | [symbol] | RNA transcription, translation and transport | yes | yes | yes | NO | [symbol] | [symbol] | [symbol] | [symbol] | [symbol] | [symbol] |
| | | factor protein | | | | | | | | | | |
| [symbol] | [symbol] | Ubiquitin carboxyl-terminal hydrolase | yes | yes | yes | NO | [symbol] | [symbol] | [symbol] | [symbol] | [symbol] | [symbol] |
| | | isozyme [symbol] | | | | | | | | | | |
| [symbol] | [symbol] | tRNA [symbol] -methyltransferase | yes | yes | yes | NO | [symbol] | [symbol] | [symbol] | [symbol] | [symbol] | [symbol] |
| | | non-catalytic subunit [symbol] | | | | | | | | | | |
| [symbol] | [symbol] | Probable ATP-dependent RNA helicase | yes | yes | yes | NO | [symbol] | [symbol] | [symbol] | [symbol] | [symbol] | [symbol] |
| | | [symbol] | | | | | | | | | | |
| [symbol] | [symbol] | Small nuclear ribonucleoprotein G | yes | yes | yes | NO | [symbol] | [symbol] | [symbol] | [symbol] | [symbol] | [symbol] |
| [symbol] | [symbol] | Protein [symbol] | yes | yes | NO | NO | [symbol] | [symbol] | [symbol] | [symbol] | [symbol] | [symbol] |
| [symbol] | [symbol] | Lethal [symbol] larvae protein homolog 2 | yes | yes | NO | NO | [symbol] | [symbol] | [symbol] | [symbol] | [symbol] | [symbol] |
| [symbol] | [symbol] | [symbol] domain [symbol] member [symbol] | yes | yes | yes | NO | [symbol] | [symbol] | [symbol] | [symbol] | [symbol] | [symbol] |
| [symbol] | [symbol] | Cleavage and [symbol] specifically | yes | yes | NO | NO | [symbol] | [symbol] | [symbol] | [symbol] | [symbol] | [symbol] |
| | | factor subunit [symbol] | | | | | | | | | | |
| [symbol] | [symbol] | Mitogen-activated protein kinase kinase | yes | yes | yes | NO | [symbol] | [symbol] | [symbol] | [symbol] | [symbol] | [symbol] |
| | | kinase kinase 4 | | | | | | | | | | |
| [symbol] | [symbol] | Nucleolar transcription factor 1 | yes | yes | yes | NO | [symbol] | [symbol] | [symbol] | [symbol] | [symbol] | [symbol] |
| [symbol] | [symbol] | [symbol] | yes | yes | yes | NO | [symbol] | [symbol] | [symbol] | [symbol] | [symbol] | [symbol] |
| [symbol] | [symbol] | [symbol] myosin [symbol] | yes | yes | NO | NO | [symbol] | [symbol] | [symbol] | [symbol] | [symbol] | [symbol] |
| [symbol] | [symbol] | [symbol] light chain [symbol] | yes | yes | yes | NO | [symbol] | [symbol] | [symbol] | [symbol] | [symbol] | [symbol] |
| [symbol] | [symbol] | Type 2 [symbol] | yes | yes | NO | NO | [symbol] | [symbol] | [symbol] | [symbol] | [symbol] | [symbol] |
| | | 4-phosphatase | | | | | | | | | | |
| [symbol] | [symbol] | Syntaxin-12 | yes | yes | NO | NO | [symbol] | [symbol] | [symbol] | [symbol] | [symbol] | [symbol] |
| [symbol] | [symbol] | Pre-mRNA-processing factor [symbol] | yes | yes | yes | NO | [symbol] | [symbol] | [symbol] | [symbol] | [symbol] | [symbol] |
| [symbol] | [symbol] | Netrin-4 | yes | yes | yes | NO | [symbol] | [symbol] | [symbol] | [symbol] | [symbol] | [symbol] |
| [symbol] | [symbol] | Ubiquitin carboxyl-terminal hydrolase 24 | yes | yes | NO | NO | [symbol] | [symbol] | [symbol] | [symbol] | [symbol] | [symbol] |
| [symbol] | [symbol] | [symbol] -3b | yes | yes | NO | NO | [symbol] | [symbol] | [symbol] | [symbol] | [symbol] | [symbol] |
| [symbol] | [symbol] | [symbol] protein | yes | yes | NO | NO | [symbol] | [symbol] | [symbol] | [symbol] | [symbol] | [symbol] |
| [symbol] | [symbol] | Phosphatidylinositol 5-phosphate 4-kinase | yes | yes | yes | NO | [symbol] | [symbol] | [symbol] | [symbol] | [symbol] | [symbol] |
| | | type-2 gamma | | | | | | | | | | |
| [symbol] | [symbol] | Ras-related protein [symbol] | yes | yes | yes | NO | [symbol] | [symbol] | [symbol] | [symbol] | [symbol] | [symbol] |
| [symbol] | [symbol] | [symbol] complex subunit [symbol] | yes | yes | NO | NO | [symbol] | [symbol] | [symbol] | [symbol] | [symbol] | [symbol] |
| [symbol] | [symbol] | Ribonucleoside-diphosphate reductase | yes | yes | yes | NO | [symbol] | [symbol] | [symbol] | [symbol] | [symbol] | [symbol] |
| | | subunit [symbol] | | | | | | | | | | |
| [symbol] | [symbol] | Multidrug resistance-associated protein 1 | yes | yes | yes | NO | [symbol] | [symbol] | [symbol] | [symbol] | [symbol] | [symbol] |
| [symbol] | [symbol] | [symbol] exchange transporter 5 | yes | yes | NO | NO | [symbol] | [symbol] | [symbol] | [symbol] | [symbol] | [symbol] |
| [symbol] | [symbol] | [symbol] -like protein 2B | yes | yes | NO | NO | [symbol] | [symbol] | [symbol] | [symbol] | [symbol] | [symbol] |
| [symbol] | [symbol] | Probable ATP-dependent RNA helicase DDX58 | yes | NO | NO | NO | [symbol] | [symbol] | [symbol] | [symbol] | [symbol] | [symbol] |
| [symbol] | [symbol] | Nucleolar protein 56 | yes | NO | NO | NO | [symbol] | [symbol] | [symbol] | [symbol] | [symbol] | [symbol] |
| [symbol] | [symbol] | ATP-dependent RNA helicase DDX42 | yes | NO | NO | NO | [symbol] | [symbol] | [symbol] | [symbol] | [symbol] | [symbol] |
| [symbol] | [symbol] | PDZ domain-containing protein [symbol] | yes | NO | NO | NO | [symbol] | [symbol] | [symbol] | [symbol] | [symbol] | [symbol] |
| [symbol] | [symbol] | BAG family molecular chaperone regulator 2 | yes | NO | NO | NO | [symbol] | [symbol] | [symbol] | [symbol] | [symbol] | [symbol] |
| [symbol] | [symbol] | ADP- [symbol] factor-like protein 1 | yes | NO | NO | NO | [symbol] | [symbol] | [symbol] | [symbol] | [symbol] | [symbol] |
| [symbol] | [symbol] | Structural maintenance of chromosomes | yes | NO | NO | NO | [symbol] | [symbol] | [symbol] | [symbol] | [symbol] | [symbol] |
| | | protein 1A | | | | | | | | | | |
| [symbol] | [symbol] | Nucleolar protein [symbol] | yes | NO | yes | NO | [symbol] | [symbol] | [symbol] | [symbol] | [symbol] | [symbol] |
| [symbol] | [symbol] | RNA polymerase-associated protein [symbol] | yes | NO | NO | NO | [symbol] | [symbol] | [symbol] | [symbol] | [symbol] | [symbol] |
| | | homolog | | | | | | | | | | |
| [symbol] | [symbol] | Translation mutation factor [symbol] -2B subunit | yes | NO | yes | NO | [symbol] | [symbol] | [symbol] | [symbol] | [symbol] | [symbol] |
| | | beta | | | | | | | | | | |
| [symbol] | [symbol] | [symbol] glutamic acid [symbol] and leucine-rich | yes | NO | NO | NO | [symbol] | [symbol] | [symbol] | [symbol] | [symbol] | [symbol] |
| | | protein 1 | | | | | | | | | | |
| [symbol] | [symbol] | Mediator of RNA polymerase II transcription | yes | NO | NO | NO | [symbol] | [symbol] | [symbol] | [symbol] | [symbol] | [symbol] |
| | | subunit 20 | | | | | | | | | | |
| [symbol] | [symbol] | [symbol] domain-containing protein 2 | yes | NO | yes | NO | [symbol] | [symbol] | [symbol] | [symbol] | [symbol] | [symbol] |
| [symbol] | [symbol] | RNA polymerase II-associated factor 1 | yes | NO | NO | NO | [symbol] | [symbol] | [symbol] | [symbol] | [symbol] | [symbol] |
| | | homolog | | | | | | | | | | |
| [symbol] | [symbol] | Pre-mRNA-processing factor 40 homolog A | yes | NO | NO | NO | [symbol] | [symbol] | [symbol] | [symbol] | [symbol] | [symbol] |
| [symbol] | [symbol] | Charged [symbol] protein [symbol] | yes | NO | NO | NO | [symbol] | [symbol] | [symbol] | [symbol] | [symbol] | [symbol] |
| [symbol] | [symbol] | Squamous cell carcinoma antigen recognized | yes | NO | yes | NO | [symbol] | [symbol] | [symbol] | [symbol] | [symbol] | [symbol] |
| | | [symbol] | | | | | | | | | | |
| [symbol] | [symbol] | [symbol] -containing protein 2 | yes | NO | NO | NO | [symbol] | [symbol] | [symbol] | [symbol] | [symbol] | [symbol] |
| [symbol] | [symbol] | Beta-2-syntrophin | yes | NO | yes | NO | [symbol] | [symbol] | [symbol] | [symbol] | [symbol] | [symbol] |
| [symbol] | [symbol] | Oxysterol-binding protein-related protein [symbol] | yes | NO | NO | NO | [symbol] | [symbol] | [symbol] | [symbol] | [symbol] | [symbol] |
| [symbol] | [symbol] | Periodic tryptophan protein 1 homolog | yes | NO | yes | NO | [symbol] | [symbol] | [symbol] | [symbol] | [symbol] | [symbol] |
| [symbol] | [symbol] | Heterogeneous nuclear ribonucleoprotein 1- | yes | NO | NO | NO | [symbol] | [symbol] | [symbol] | [symbol] | [symbol] | [symbol] |
| | | like | | | | | | | | | | |
| [symbol] | [symbol] | [symbol] -2 | yes | NO | NO | NO | [symbol] | [symbol] | [symbol] | [symbol] | [symbol] | [symbol] |
| [symbol] | [symbol] | Glutamate-rich WD repeat-containing protein | yes | NO | NO | NO | [symbol] | [symbol] | [symbol] | [symbol] | [symbol] | [symbol] |
| | | 1 | | | | | | | | | | |
| [symbol] | [symbol] | Serine arginine repetitive matrix protein 2 | yes | NO | yes | NO | [symbol] | [symbol] | [symbol] | [symbol] | [symbol] | [symbol] |
| [symbol] | [symbol] | Splicing factor 45 | yes | NO | yes | NO | [symbol] | [symbol] | [symbol] | [symbol] | [symbol] | [symbol] |
| [symbol] | [symbol] | DDB1- and [symbol] associated factor [symbol] | yes | NO | yes | NO | [symbol] | [symbol] | [symbol] | [symbol] | [symbol] | [symbol] |
| [symbol] | [symbol] | [symbol] -like repeat and [symbol] 1-like domain- | yes | NO | NO | NO | [symbol] | [symbol] | [symbol] | [symbol] | [symbol] | [symbol] |
| | | containing protein 3 | | | | | | | | | | |
| [symbol] | [symbol] | Metalloproteinase inhibitor 3 | yes | NO | NO | NO | [symbol] | [symbol] | [symbol] | [symbol] | [symbol] | [symbol] |
| [symbol] | [symbol] | [symbol] repeat-containing protein 3 | yes | NO | NO | NO | [symbol] | [symbol] | [symbol] | [symbol] | [symbol] | [symbol] |
| [symbol] | [symbol] | [symbol] -containing protein 4 | yes | NO | NO | NO | [symbol] | [symbol] | [symbol] | [symbol] | [symbol] | [symbol] |
| [symbol] | [symbol] | Endoplasmic [symbol] protein [symbol] | yes | NO | NO | NO | [symbol] | [symbol] | [symbol] | [symbol] | [symbol] | [symbol] |
| [symbol] | [symbol] | Protein kinase [symbol] and casein kinase II | yes | NO | NO | NO | [symbol] | [symbol] | [symbol] | [symbol] | [symbol] | [symbol] |
| | | substrate protein [symbol] | | | | | | | | | | |
| [symbol] | [symbol] | N-alpha-acetyltransferase [symbol] | yes | NO | NO | NO | [symbol] | [symbol] | [symbol] | [symbol] | [symbol] | [symbol] |
| [symbol] | [symbol] | [symbol] acid receptor 6 | yes | NO | NO | NO | [symbol] | [symbol] | [symbol] | [symbol] | [symbol] | [symbol] |
| [symbol] | [symbol] | Protein [symbol] | yes | NO | NO | NO | [symbol] | [symbol] | [symbol] | [symbol] | [symbol] | [symbol] |
| [symbol] | [symbol] | Kinetochore-associated protein 1 | yes | NO | yes | NO | [symbol] | [symbol] | [symbol] | [symbol] | [symbol] | [symbol] |
| [symbol] | [symbol] | Cyclin-dependent kinase [symbol] | yes | NO | NO | NO | [symbol] | [symbol] | [symbol] | [symbol] | [symbol] | [symbol] |
| [symbol] | [symbol] | Small ubiquitin-related modifier [symbol] | yes | NO | yes | NO | [symbol] | [symbol] | [symbol] | [symbol] | [symbol] | [symbol] |
| [symbol] | [symbol] | [symbol] -associated [symbol] -like protein [symbol] | yes | NO | NO | NO | [symbol] | [symbol] | [symbol] | [symbol] | [symbol] | [symbol] |
| [symbol] | [symbol] | High mobility group protein [symbol] | yes | NO | NO | NO | [symbol] | [symbol] | [symbol] | [symbol] | [symbol] | [symbol] |
| [symbol] | [symbol] | Cleavage stimulation factor subunit 1 | yes | NO | yes | NO | [symbol] | [symbol] | [symbol] | [symbol] | [symbol] | [symbol] |
| [symbol] | [symbol] | [symbol] | yes | NO | yes | NO | [symbol] | [symbol] | [symbol] | [symbol] | [symbol] | [symbol] |
| [symbol] | [symbol] | Protein strawberry [symbol] homolog 1 | yes | NO | yes | NO | [symbol] | [symbol] | [symbol] | [symbol] | [symbol] | [symbol] |
| [symbol] | [symbol] | Protein [symbol] homolog B | yes | NO | NO | NO | [symbol] | [symbol] | [symbol] | [symbol] | [symbol] | [symbol] |
| [symbol] | [symbol] | [symbol] develpoment protein 1 | yes | NO | NO | NO | [symbol] | [symbol] | [symbol] | [symbol] | [symbol] | [symbol] |
| [symbol] | [symbol] | Protein transport protein [symbol] subunit beta | yes | NO | NO | NO | [symbol] | [symbol] | [symbol] | [symbol] | [symbol] | [symbol] |
| [symbol] | [symbol] | Carboxy-terminal domain RNA polymerase | NO | yes | yes | NO | [symbol] | [symbol] | [symbol] | [symbol] | [symbol] | [symbol] |
| | | II polypeptide A small phosphatase 1 | | | | | | | | | | |
| [symbol] | [symbol] | Protocadherin-19 | NO | yes | NO | NO | [symbol] | [symbol] | [symbol] | [symbol] | [symbol] | [symbol] |
| [symbol] | [symbol] | [symbol] | NO | yes | NO | NO | [symbol] | [symbol] | [symbol] | [symbol] | [symbol] | [symbol] |
| [symbol] | [symbol] | [symbol] myosin- [symbol] | NO | yes | NO | NO | [symbol] | [symbol] | [symbol] | [symbol] | [symbol] | [symbol] |
| [symbol] | [symbol] | Phospholipid phosphatase 3 | NO | yes | yes | NO | [symbol] | [symbol] | [symbol] | [symbol] | [symbol] | [symbol] |
| [symbol] | [symbol] | [symbol] domain-binding protein 4 | NO | yes | NO | NO | [symbol] | [symbol] | [symbol] | [symbol] | [symbol] | [symbol] |
| [symbol] | [symbol] | DnaJ homolog subfamily C member [symbol] | NO | yes | NO | NO | [symbol] | [symbol] | [symbol] | [symbol] | [symbol] | [symbol] |
| [symbol] | [symbol] | Developmentally-regulated [symbol] -binding | NO | yes | yes | NO | [symbol] | [symbol] | [symbol] | [symbol] | [symbol] | [symbol] |
| | | protein 1 | | | | | | | | | | |
| [symbol] | [symbol] | Macrophage [symbol] attacher | NO | yes | NO | NO | [symbol] | [symbol] | [symbol] | [symbol] | [symbol] | [symbol] |

[symbol]

indicates data missing or illegible when filed

TABLE 2
The 121 Proteins of the “RTV Therapeutic Efficacy with ISRIB Sensitivity” Subset

| | | | [symbol] | [symbol] | [symbol] | | Abs Z | | | | | |
|---|---|---|---|---|---|---|---|---|---|---|---|---|
| | | | Direction | Direction | Direction | | Score Sum | | | | | |
| | | | Opposite | Same as | Opposite | In Human | (Geno + | WT Mean | HetvWT | [symbol] vHet | [symbol] vWT | [symbol] vHet |
| Accession | GN | Description | [symbol] | [symbol] | [symbol] | Plasma | RTV) | Abundance | Z Score | Z Score | Z Score | Z Score |
| [symbol] | [symbol] | [symbol] dehydrogenase [NADP], [symbol] | yes | yes | yes | yes | [symbol] | [symbol] | [symbol] | [symbol] | [symbol] | [symbol] |
| [symbol] | [symbol] | Aldehyde dehydrogenase, [symbol] | yes | yes | yes | yes | [symbol] | [symbol] | [symbol] | [symbol] | [symbol] | [symbol] |
| [symbol] | [symbol] | [symbol] | yes | yes | yes | yes | [symbol] | [symbol] | [symbol] | [symbol] | [symbol] | [symbol] |
| [symbol] | [symbol] | [symbol] epsilon-1 | yes | yes | yes | yes | [symbol] | [symbol] | [symbol] | [symbol] | [symbol] | [symbol] |
| [symbol] | [symbol] | [symbol] protein RBBP4 | yes | yes | yes | yes | [symbol] | [symbol] | [symbol] | [symbol] | [symbol] | [symbol] |
| [symbol] | [symbol] | Integral membrane protein 2B | yes | yes | yes | yes | [symbol] | [symbol] | [symbol] | [symbol] | [symbol] | [symbol] |
| [symbol] | [symbol] | [symbol] RNA helicase [symbol] | yes | yes | yes | yes | [symbol] | [symbol] | [symbol] | [symbol] | [symbol] | [symbol] |
| [symbol] | [symbol] | [symbol] activating enzyme [symbol] | yes | yes | yes | yes | [symbol] | [symbol] | [symbol] | [symbol] | [symbol] | [symbol] |
| | | catalytic subunit | | | | | | | | | | |
| [symbol] | [symbol] | [symbol] component 4 | yes | yes | yes | yes | [symbol] | [symbol] | [symbol] | [symbol] | [symbol] | [symbol] |
| [symbol] | [symbol] | [symbol] subunit alpha [symbol] | yes | yes | NO | yes | [symbol] | [symbol] | [symbol] | [symbol] | [symbol] | [symbol] |
| [symbol] | [symbol] | [symbol] subunit 2 | yes | yes | yes | yes | [symbol] | [symbol] | [symbol] | [symbol] | [symbol] | [symbol] |
| [symbol] | [symbol] | [symbol] | yes | yes | yes | yes | [symbol] | [symbol] | [symbol] | [symbol] | [symbol] | [symbol] |
| [symbol] | [symbol] | [symbol] transport [symbol] | yes | yes | NO | yes | [symbol] | [symbol] | [symbol] | [symbol] | [symbol] | [symbol] |
| [symbol] | [symbol] | Glutamine--tRNA ligase | yes | yes | yes | yes | [symbol] | [symbol] | [symbol] | [symbol] | [symbol] | [symbol] |
| [symbol] | [symbol] | [symbol] heavy chain | yes | yes | yes | yes | [symbol] | [symbol] | [symbol] | [symbol] | [symbol] | [symbol] |
| [symbol] | [symbol] | [symbol] protein | yes | yes | yes | yes | [symbol] | [symbol] | [symbol] | [symbol] | [symbol] | [symbol] |
| [symbol] | [symbol] | [symbol] complex subunit 1 | yes | yes | yes | yes | [symbol] | [symbol] | [symbol] | [symbol] | [symbol] | [symbol] |
| [symbol] | [symbol] | [symbol] binding protein [symbol] | yes | yes | yes | yes | [symbol] | [symbol] | [symbol] | [symbol] | [symbol] | [symbol] |
| [symbol] | [symbol] | [symbol] | yes | yes | yes | yes | [symbol] | [symbol] | [symbol] | [symbol] | [symbol] | [symbol] |
| [symbol] | [symbol] | [symbol] | yes | yes | yes | yes | [symbol] | [symbol] | [symbol] | [symbol] | [symbol] | [symbol] |
| [symbol] | [symbol] | Histone [symbol] | yes | yes | yes | yes | [symbol] | [symbol] | [symbol] | [symbol] | [symbol] | [symbol] |
| [symbol] | [symbol] | [symbol] | yes | yes | yes | yes | [symbol] | [symbol] | [symbol] | [symbol] | [symbol] | [symbol] |
| [symbol] | [symbol] | [symbol] | yes | yes | yes | yes | [symbol] | [symbol] | [symbol] | [symbol] | [symbol] | [symbol] |
| [symbol] | [symbol] | [symbol] factor [symbol] | yes | yes | yes | yes | [symbol] | [symbol] | [symbol] | [symbol] | [symbol] | [symbol] |
| [symbol] | [symbol] | [symbol] | yes | yes | yes | yes | [symbol] | [symbol] | [symbol] | [symbol] | [symbol] | [symbol] |
| [symbol] | [symbol] | [symbol] lipase | yes | yes | NO | yes | [symbol] | [symbol] | [symbol] | [symbol] | [symbol] | [symbol] |
| [symbol] | [symbol] | [symbol] hydrolase 1 | yes | yes | NO | yes | [symbol] | [symbol] | [symbol] | [symbol] | [symbol] | [symbol] |
| [symbol] | [symbol] | [symbol] -protein lipase [symbol] | yes | yes | yes | yes | [symbol] | [symbol] | [symbol] | [symbol] | [symbol] | [symbol] |
| [symbol] | [symbol] | [symbol] | yes | yes | yes | yes | [symbol] | [symbol] | [symbol] | [symbol] | [symbol] | [symbol] |
| [symbol] | [symbol] | [symbol] growth factor beta- | yes | yes | yes | yes | [symbol] | [symbol] | [symbol] | [symbol] | [symbol] | [symbol] |
| | | binding protein 2 | | | | | | | | | | |
| [symbol] | [symbol] | [symbol] reductase 2 | yes | yes | NO | yes | [symbol] | [symbol] | [symbol] | [symbol] | [symbol] | [symbol] |
| [symbol] | [symbol] | [symbol] factor [symbol] subunit alpha | yes | yes | yes | yes | [symbol] | [symbol] | [symbol] | [symbol] | [symbol] | [symbol] |
| [symbol] | [symbol] | [symbol] -terminal hydrolase [symbol] | yes | yes | yes | yes | [symbol] | [symbol] | [symbol] | [symbol] | [symbol] | [symbol] |
| [symbol] | [symbol] | [symbol] subunit epsilon | yes | yes | NO | yes | [symbol] | [symbol] | [symbol] | [symbol] | [symbol] | [symbol] |
| [symbol] | [symbol] | [symbol] -CoA synthase [symbol] | yes | yes | yes | yes | [symbol] | [symbol] | [symbol] | [symbol] | [symbol] | [symbol] |
| [symbol] | [symbol] | [symbol] | yes | yes | yes | yes | [symbol] | [symbol] | [symbol] | [symbol] | [symbol] | [symbol] |
| [symbol] | [symbol] | [symbol] reductase | yes | yes | yes | yes | [symbol] | [symbol] | [symbol] | [symbol] | [symbol] | [symbol] |
| [symbol] | [symbol] | [symbol] kinase [symbol] | yes | yes | yes | yes | [symbol] | [symbol] | [symbol] | [symbol] | [symbol] | [symbol] |
| [symbol] | [symbol] | [symbol] beta | yes | yes | yes | yes | [symbol] | [symbol] | [symbol] | [symbol] | [symbol] | [symbol] |
| [symbol] | [symbol] | Threonine tRNA ligase [symbol] | yes | yes | yes | yes | [symbol] | [symbol] | [symbol] | [symbol] | [symbol] | [symbol] |
| [symbol] | [symbol] | [symbol] GTPase-activating protein non [symbol] subunit | yes | yes | NO | yes | [symbol] | [symbol] | [symbol] | [symbol] | [symbol] | [symbol] |
| [symbol] | [symbol] | [symbol] subunit beta | yes | yes | NO | yes | [symbol] | [symbol] | [symbol] | [symbol] | [symbol] | [symbol] |
| [symbol] | [symbol] | [symbol] subunit beta [symbol] | yes | yes | yes | yes | [symbol] | [symbol] | [symbol] | [symbol] | [symbol] | [symbol] |
| [symbol] | [symbol] | [symbol] peptidase [symbol] | yes | yes | yes | yes | [symbol] | [symbol] | [symbol] | [symbol] | [symbol] | [symbol] |
| [symbol] | [symbol] | Receptor-type [symbol] protein [symbol] | yes | yes | yes | yes | [symbol] | [symbol] | [symbol] | [symbol] | [symbol] | [symbol] |
| [symbol] | [symbol] | Heterogenous [symbol] | yes | yes | yes | yes | [symbol] | [symbol] | [symbol] | [symbol] | [symbol] | [symbol] |
| [symbol] | [symbol] | Protein [symbol] | yes | yes | NO | yes | [symbol] | [symbol] | [symbol] | [symbol] | [symbol] | [symbol] |
| [symbol] | [symbol] | Regulator [symbol] | yes | yes | NO | yes | [symbol] | [symbol] | [symbol] | [symbol] | [symbol] | [symbol] |
| [symbol] | [symbol] | [symbol] dependent RNA helicase [symbol] | yes | yes | yes | yes | [symbol] | [symbol] | [symbol] | [symbol] | [symbol] | [symbol] |
| [symbol] | [symbol] | [symbol] translation [symbol] factor 4 gamma 2 | yes | yes | yes | yes | [symbol] | [symbol] | [symbol] | [symbol] | [symbol] | [symbol] |
| [symbol] | [symbol] | Integrin beta- [symbol] | yes | yes | yes | yes | [symbol] | [symbol] | [symbol] | [symbol] | [symbol] | [symbol] |
| [symbol] | [symbol] | [symbol] ATPase subunit II | yes | yes | NO | yes | [symbol] | [symbol] | [symbol] | [symbol] | [symbol] | [symbol] |
| [symbol] | [symbol] | Prolyl endopeptidase | yes | yes | yes | yes | [symbol] | [symbol] | [symbol] | [symbol] | [symbol] | [symbol] |
| [symbol] | [symbol] | [symbol] | yes | yes | yes | yes | [symbol] | [symbol] | [symbol] | [symbol] | [symbol] | [symbol] |
| [symbol] | [symbol] | Aldose reductase | yes | yes | yes | yes | [symbol] | [symbol] | [symbol] | [symbol] | [symbol] | [symbol] |
| [symbol] | [symbol] | Protein transport protein [symbol] | yes | yes | yes | yes | [symbol] | [symbol] | [symbol] | [symbol] | [symbol] | [symbol] |
| [symbol] | [symbol] | [symbol] -like [symbol] protein 1 | yes | yes | yes | yes | [symbol] | [symbol] | [symbol] | [symbol] | [symbol] | [symbol] |
| [symbol] | [symbol] | [symbol] enzyme type 2 | yes | yes | NO | yes | [symbol] | [symbol] | [symbol] | [symbol] | [symbol] | [symbol] |
| [symbol] | [symbol] | [symbol] phosphatase | yes | yes | yes | yes | [symbol] | [symbol] | [symbol] | [symbol] | [symbol] | [symbol] |
| [symbol] | [symbol] | [symbol] domain-containing protein [symbol] | yes | yes | NO | yes | [symbol] | [symbol] | [symbol] | [symbol] | [symbol] | [symbol] |
| [symbol] | [symbol] | Probable ATP-dependent RNA helicase DDX5 | yes | yes | yes | yes | [symbol] | [symbol] | [symbol] | [symbol] | [symbol] | [symbol] |
| [symbol] | [symbol] | Alpha [symbol] | yes | yes | yes | yes | [symbol] | [symbol] | [symbol] | [symbol] | [symbol] | [symbol] |
| [symbol] | [symbol] | [symbol] homology domain-containing family O member 2 | yes | yes | yes | yes | [symbol] | [symbol] | [symbol] | [symbol] | [symbol] | [symbol] |
| [symbol] | [symbol] | Citrate synthase [symbol] | yes | yes | yes | yes | [symbol] | [symbol] | [symbol] | [symbol] | [symbol] | [symbol] |
| [symbol] | [symbol] | [symbol] | yes | yes | yes | yes | [symbol] | [symbol] | [symbol] | [symbol] | [symbol] | [symbol] |
| [symbol] | [symbol] | 26S [symbol] regulatory subunit [symbol] | yes | yes | yes | yes | [symbol] | [symbol] | [symbol] | [symbol] | [symbol] | [symbol] |
| [symbol] | [symbol] | Alpha [symbol] | yes | yes | yes | yes | [symbol] | [symbol] | [symbol] | [symbol] | [symbol] | [symbol] |
| [symbol] | [symbol] | Macrophage [symbol] protein | yes | yes | yes | yes | [symbol] | [symbol] | [symbol] | [symbol] | [symbol] | [symbol] |
| [symbol] | [symbol] | DNA [symbol] | yes | yes | NO | yes | [symbol] | [symbol] | [symbol] | [symbol] | [symbol] | [symbol] |
| [symbol] | [symbol] | [symbol] domain-containing receptor 2 | yes | yes | NO | yes | [symbol] | [symbol] | [symbol] | [symbol] | [symbol] | [symbol] |
| [symbol] | [symbol] | Signal [symbol] particle subunit [symbol] | yes | yes | yes | yes | [symbol] | [symbol] | [symbol] | [symbol] | [symbol] | [symbol] |
| [symbol] | [symbol] | [symbol] | yes | yes | yes | yes | [symbol] | [symbol] | [symbol] | [symbol] | [symbol] | [symbol] |
| [symbol] | [symbol] | [symbol] | yes | yes | yes | yes | [symbol] | [symbol] | [symbol] | [symbol] | [symbol] | [symbol] |
| [symbol] | [symbol] | [symbol] | yes | yes | yes | yes | [symbol] | [symbol] | [symbol] | [symbol] | [symbol] | [symbol] |
| [symbol] | [symbol] | [symbol] antigen | yes | yes | yes | yes | [symbol] | [symbol] | [symbol] | [symbol] | [symbol] | [symbol] |
| [symbol] | [symbol] | [symbol] | yes | yes | yes | yes | [symbol] | [symbol] | [symbol] | [symbol] | [symbol] | [symbol] |
| [symbol] | [symbol] | [symbol] domain-containing protein [symbol] | yes | yes | yes | yes | [symbol] | [symbol] | [symbol] | [symbol] | [symbol] | [symbol] |
| [symbol] | [symbol] | Pre- [symbol] factor ATP-dependent | yes | yes | yes | yes | [symbol] | [symbol] | [symbol] | [symbol] | [symbol] | [symbol] |
| | | RNA helicase DHX15 | | | | | | | | | | |
| [symbol] | [symbol] | AP- [symbol] delta [symbol] | yes | yes | NO | yes | [symbol] | [symbol] | [symbol] | [symbol] | [symbol] | [symbol] |
| [symbol] | [symbol] | Collagen alpha [symbol] | yes | yes | yes | yes | [symbol] | [symbol] | [symbol] | [symbol] | [symbol] | [symbol] |
| [symbol] | [symbol] | [symbol] subunit gamma 2 | yes | yes | yes | yes | [symbol] | [symbol] | [symbol] | [symbol] | [symbol] | [symbol] |
| [symbol] | [symbol] | [symbol] protein | yes | yes | yes | yes | [symbol] | [symbol] | [symbol] | [symbol] | [symbol] | [symbol] |
| [symbol] | [symbol] | GTP-binding protein [symbol] | yes | yes | yes | yes | [symbol] | [symbol] | [symbol] | [symbol] | [symbol] | [symbol] |
| [symbol] | [symbol] | Multiple [symbol] -like domain protein [symbol] | yes | yes | yes | yes | [symbol] | [symbol] | [symbol] | [symbol] | [symbol] | [symbol] |
| [symbol] | [symbol] | [symbol] --tRNA ligase [symbol] | yes | yes | yes | yes | [symbol] | [symbol] | [symbol] | [symbol] | [symbol] | [symbol] |
| [symbol] | [symbol] | [symbol] protein [symbol] protein 26B | yes | yes | NO | yes | [symbol] | [symbol] | [symbol] | [symbol] | [symbol] | [symbol] |
| [symbol] | [symbol] | [symbol] | yes | yes | yes | yes | [symbol] | [symbol] | [symbol] | [symbol] | [symbol] | [symbol] |
| [symbol] | [symbol] | [symbol] | yes | yes | yes | yes | [symbol] | [symbol] | [symbol] | [symbol] | [symbol] | [symbol] |
| [symbol] | [symbol] | Nectin-2 | yes | yes | NO | yes | [symbol] | [symbol] | [symbol] | [symbol] | [symbol] | [symbol] |
| [symbol] | [symbol] | [symbol] growth factor beta-binding protein 1 | yes | yes | yes | yes | [symbol] | [symbol] | [symbol] | [symbol] | [symbol] | [symbol] |
| [symbol] | [symbol] | [symbol] | yes | yes | yes | yes | [symbol] | [symbol] | [symbol] | [symbol] | [symbol] | [symbol] |
| [symbol] | [symbol] | [symbol] like protein 1 | yes | yes | NO | yes | [symbol] | [symbol] | [symbol] | [symbol] | [symbol] | [symbol] |
| [symbol] | [symbol] | Glycerol- [symbol] | yes | yes | yes | yes | [symbol] | [symbol] | [symbol] | [symbol] | [symbol] | [symbol] |
| [symbol] | [symbol] | [symbol] factor [symbol] | yes | yes | yes | yes | [symbol] | [symbol] | [symbol] | [symbol] | [symbol] | [symbol] |
| [symbol] | [symbol] | [symbol] | yes | yes | yes | yes | [symbol] | [symbol] | [symbol] | [symbol] | [symbol] | [symbol] |
| [symbol] | [symbol] | [symbol] hydrogen [symbol] | yes | yes | yes | yes | [symbol] | [symbol] | [symbol] | [symbol] | [symbol] | [symbol] |
| [symbol] | [symbol] | [symbol] | yes | yes | yes | yes | [symbol] | [symbol] | [symbol] | [symbol] | [symbol] | [symbol] |
| [symbol] | [symbol] | Collagen alpha- [symbol] | yes | yes | NO | yes | [symbol] | [symbol] | [symbol] | [symbol] | [symbol] | [symbol] |
| [symbol] | [symbol] | [symbol] member 2 | yes | yes | NO | yes | [symbol] | [symbol] | [symbol] | [symbol] | [symbol] | [symbol] |
| [symbol] | [symbol] | [symbol] -like protein [symbol] | yes | yes | yes | yes | [symbol] | [symbol] | [symbol] | [symbol] | [symbol] | [symbol] |
| [symbol] | [symbol] | Fibroblast growth factor receptor 1 | yes | yes | yes | yes | [symbol] | [symbol] | [symbol] | [symbol] | [symbol] | [symbol] |
| [symbol] | [symbol] | Collagen alpha- [symbol] | yes | yes | NO | yes | [symbol] | [symbol] | [symbol] | [symbol] | [symbol] | [symbol] |
| [symbol] | [symbol] | [symbol] translation [symbol] factor 4B | yes | yes | NO | yes | [symbol] | [symbol] | [symbol] | [symbol] | [symbol] | [symbol] |
| [symbol] | [symbol] | [symbol] DNA-binding protein [symbol] | yes | yes | yes | yes | [symbol] | [symbol] | [symbol] | [symbol] | [symbol] | [symbol] |
| [symbol] | [symbol] | Ras-related protein [symbol] | yes | yes | NO | yes | [symbol] | [symbol] | [symbol] | [symbol] | [symbol] | [symbol] |
| [symbol] | [symbol] | ATP-dependent RNA helicase [symbol] | yes | yes | yes | yes | [symbol] | [symbol] | [symbol] | [symbol] | [symbol] | [symbol] |
| [symbol] | [symbol] | Interleukin enhancer-binding factor 2 | yes | yes | yes | yes | [symbol] | [symbol] | [symbol] | [symbol] | [symbol] | [symbol] |
| [symbol] | [symbol] | Purine [symbol] | yes | yes | yes | yes | [symbol] | [symbol] | [symbol] | [symbol] | [symbol] | [symbol] |
| [symbol] | [symbol] | 26S [symbol] regulatory subunit 4 | yes | yes | NO | yes | [symbol] | [symbol] | [symbol] | [symbol] | [symbol] | [symbol] |
| [symbol] | [symbol] | [symbol] homolog protein 3 | yes | yes | yes | yes | [symbol] | [symbol] | [symbol] | [symbol] | [symbol] | [symbol] |
| [symbol] | [symbol] | [symbol] -binding protein 2 | yes | yes | yes | yes | [symbol] | [symbol] | [symbol] | [symbol] | [symbol] | [symbol] |
| [symbol] | [symbol] | [symbol] mitochondrial | yes | yes | yes | yes | [symbol] | [symbol] | [symbol] | [symbol] | [symbol] | [symbol] |
| [symbol] | [symbol] | [symbol] 3 | yes | yes | NO | yes | [symbol] | [symbol] | [symbol] | [symbol] | [symbol] | [symbol] |
| [symbol] | [symbol] | Delta- [symbol] | yes | yes | yes | yes | [symbol] | [symbol] | [symbol] | [symbol] | [symbol] | [symbol] |
| [symbol] | [symbol] | Ubiquitin like [symbol] | yes | yes | NO | yes | [symbol] | [symbol] | [symbol] | [symbol] | [symbol] | [symbol] |
| [symbol] | [symbol] | [symbol] -binding protein | yes | yes | NO | yes | [symbol] | [symbol] | [symbol] | [symbol] | [symbol] | [symbol] |
| [symbol] | [symbol] | Protein [symbol] A3 | yes | yes | yes | yes | [symbol] | [symbol] | [symbol] | [symbol] | [symbol] | [symbol] |
| [symbol] | [symbol] | A [symbol] and metalloproteinase with | yes | yes | yes | yes | [symbol] | [symbol] | [symbol] | [symbol] | [symbol] | [symbol] |
| | | [symbol] motifs 5 | | | | | | | | | | |
| [symbol] | [symbol] | [symbol] growth factor | yes | yes | yes | yes | [symbol] | [symbol] | [symbol] | [symbol] | [symbol] | [symbol] |
| [symbol] | [symbol] | [symbol] adhesion kinase 1 | yes | yes | NO | yes | [symbol] | [symbol] | [symbol] | [symbol] | [symbol] | [symbol] |
| [symbol] | [symbol] | [symbol] like 2 | yes | yes | NO | yes | [symbol] | [symbol] | [symbol] | [symbol] | [symbol] | [symbol] |

[symbol]

indicates data missing or illegible when filed

TABLE 3
The 33 Proteins of the “RTV Therapeutic Efficacy with ISRIB Insensitivity” Subset

| | | | RTV | ISRIB | Sal | | Abs Z | | | | | |
|---|---|---|---|---|---|---|---|---|---|---|---|---|
| | | | Direction | Direction | Direction | | Score Sum | | | | | |
| | | | Opposite | Same as | Opposite | In Human | (Geno + | WT Mean | HetvWT | RTVvHet | ISRIBvWT | SALvHet |
| Accession | GN | Description | dE? | dE? | ISRIB? | Plasma | RTV) | Abundance | Z Score | Z Score | Z Score | Z Score |
| Q78ZA7 | [symbol] | Nucelosome assembly protein 1-like 4 | yes | NO | yes | yes | 50.77 | 2.29E+06 | −45.04 | 5.74 | 2.43 | −2.26 |
| P97792 | Cxadr | [symbol] and adenovirus receptor homolog | yes | NO | yes | yes | 16.73 | 4.16E+06 | −12.85 | 3.88 | 1.72 | −0.13 |
| P56959 | Fus | RNA-binding protein FUS | yes | NO | NO | yes | 14.88 | 1.18E+06 | −2.94 | 11.94 | 0.85 | 2.28 |
| Q55023 | Impa1 | Inositol monophosphatase 1 | yes | NO | NO | yes | 14.51 | 7.37E+05 | −3.62 | 10.89 | 1.73 | 3.95 |
| Q9DAW9 | Cnn3 | Calponin-3 | yes | NO | NO | yes | 13.28 | 1.75E+06 | −8.50 | 4.78 | 2.76 | 1.96 |
| Q9Z1Z0 | [symbol] | General vesicular transport factor p115 | yes | NO | yes | yes | 12.14 | 1.66E+06 | −3.02 | 9.12 | 2.44 | −0.59 |
| D3YXG0 | [symbol] | [symbol] | yes | NO | yes | yes | 11.51 | 9.50E+06 | 7.93 | −3.58 | −2.25 | 0.86 |
| Q99J45 | Nrbp1 | Nuclear receptor-binding protein | yes | NO | yes | yes | 9.54 | 5.42E+05 | −3.20 | 6.35 | 0.18 | −2.12 |
| Q9WV55 | Vapa | Vesicle-associated membrane protein- | yes | NO | NO | yes | 8.93 | 7.68E+05 | −7.02 | 1.91 | 3.18 | 0.62 |
| | | associated protein A | | | | | | | | | | |
| P61089 | [symbol] | Ubiquitin-conjugating enzyme [symbol] | yes | NO | NO | yes | 8.62 | 1.06E+06 | −2.60 | 6.02 | 0.41 | 8.07 |
| Q35737 | Hnrnph1 | Heterogenous nuclear ribonucleoprotein II | yes | NO | NO | yes | 8.31 | 1.18E+06 | −4.08 | 4.23 | 2.47 | 0.44 |
| P50543 | S100a11 | Protein S100-A11 | yes | NO | yes | yes | 8.18 | 5.73E+06 | −3.69 | 4.49 | 0.33 | −0.12 |
| Q09131 | Gsto1 | Glutathione S-transferase omega-1 | yes | NO | NO | yes | 8.09 | 6.66E+06 | −4.63 | 3.45 | 0.04 | 2.11 |
| P12025 | Mdk | Midkine | yes | NO | NO | yes | 7.88 | 6.99E+05 | −4.24 | 3.64 | 1.33 | 0.59 |
| [symbol] | Myo18a | Unconventional myosin-XVIIIa | yes | NO | yes | yes | 7.78 | 3.49E+06 | −2.87 | 4.91 | 2.27 | −1.53 |
| P67984 | Rpl22 | 60S ribosomal protein L22 | yes | NO | yes | yes | 7.66 | 7.30E+06 | −6.07 | 1.59 | 2.38 | −0.27 |
| Q35405 | Pld3 | Phospholipase D3 | yes | NO | NO | yes | 7.47 | 5.59E+05 | −6.86 | 0.61 | 1.18 | 0.57 |
| Q9FS30 | [symbol] | Complement C1q tumor necrosis factor- | yes | NO | NO | yes | 6.95 | 1.65E+07 | 3.71 | −3.24 | −0.18 | −1.10 |
| | | related protein 3 | | | | | | | | | | |
| P35279 | Rab6a | Ras-related protein Rab-6A | yes | NO | NO | yes | 6.80 | 1.61E+06 | −3.66 | 3.14 | 0.44 | 1.92 |
| P62996 | Tra2b | Transformer-2 protein homolog beta | yes | NO | NO | yes | 6.63 | 1.51E+06 | −3.35 | 3.28 | 0.54 | 3.10 |
| P42669 | Pura | Transcriptional activator protein Pur-alpha | yes | NO | yes | yes | 6.04 | 2.78E+06 | −3.68 | 2.36 | 0.52 | −1.14 |
| P08003 | Pdia4 | Protein disulfide-isomerase A4 | yes | NO | NO | yes | 5.92 | 4.54E+06 | −3.67 | 2.25 | 1.37 | 1.08 |
| Q8BL97 | [symbol] | Serine arginine-rich splicing factor 7 | yes | NO | yes | yes | 5.72 | 1.94E+07 | −2.81 | 2.91 | 0.13 | −0.89 |
| P32067 | Ssb | Lupus La protein homolog | yes | NO | NO | yes | 5.45 | 4.28E+06 | −2.66 | 2.79 | 0.41 | 0.09 |
| Q9EPL8 | Ipo7 | Importin-7 | yes | NO | yes | yes | 5.25 | 2.54E+07 | −3.01 | 2.25 | 0.30 | −0.95 |
| P23927 | Cryab | Alpha-crystallin B chain | yes | NO | NO | yes | 5.18 | 4.11E+05 | −4.08 | 1.11 | 0.39 | 3.46 |
| Q54890 | Itgb3 | Integrin beta-3 | yes | NO | NO | yes | 4.87 | 1.79E+06 | −2.91 | 1.96 | 0.14 | 2.90 |
| Q60751 | [symbol] | Insulin-like growth factor 1 receptor | yes | NO | NO | yes | 4.86 | 3.32E+06 | −4.23 | 0.63 | 0.75 | 0.24 |
| Q3U7R1 | Esyt1 | Extended synaptotagmin-1 | yes | NO | yes | yes | 4.61 | 1.52E+06 | −3.36 | 1.25 | 0.18 | −0.64 |
| P22005 | Penk | Proenkephalin-A | yes | NO | NO | yes | 4.31 | 1.35E+06 | 2.02 | −2.29 | −0.11 | −2.09 |
| Q35114 | Scarb2 | Lysosome membrane protein 2 | yes | NO | NO | yes | 3.91 | 5.51E+05 | −3.66 | 0.24 | 0.09 | 1.13 |
| Q6X893 | [symbol] | Choline transporter-like protein 1 | yes | NO | NO | yes | 3.67 | 3.01E+06 | −2.83 | 0.84 | 0.80 | 1.03 |
| A2A0A0 | Svep1 | Sushi. von Willebrand factor type A, EGF | yes | NO | NO | yes | 2.64 | 2.02E+06 | 2.25 | −0.40 | −0.50 | −1.18 |
| | | and pentraxin domain-containing protein 1 | | | | | | | | | | |

[symbol]

indicates data missing or illegible when filed

TABLE 4
The 46 Proteins of the “Protein Biomarker” Subset

| | | | RTV | ISRIB | Sal | | Abs Z | | | | | |
|---|---|---|---|---|---|---|---|---|---|---|---|---|
| | | | Direction | Direction | Direction | In | Score Sum | | | | | |
| | | | Opposite | Same as | Opposite | Human | (Geno + | WT Mean | HetvWT | RTVvHet | ISRIBvWT | SALvHet |
| Accession | GN | Description | dE? | dE? | ISRIB? | Plasma | RTV) | Abundance | Z Score | Z Score | Z Score | Z Score |
| P54071 | Idh2 | Isocitrate dehydrogenase [NADP] [symbol] | yes | yes | yes | yes | 26.73 | 1.52E+06 | −3.12 | 23.61 | −2.01 | 0.46 |
| | | mitochondrial | | | | | | | | | | |
| P47738 | Aldh2 | Aldehyde dehydrogenase, mitochondrial | yes | yes | yes | yes | 25.29 | 2.43E+05 | −7.08 | 18.21 | −7.25 | 0.22 |
| Q9D892 | Itpa | Inosine triphosphate pyrophosphatase | yes | yes | yes | yes | 23.68 | 6.45E+05 | −10.88 | 12.80 | −1.25 | 4.47 |
| Q9D1M4 | [symbol] | Eukaryotic translation EF1 epsilon-1 | yes | yes | yes | yes | 20.67 | 3.08E+06 | −3.77 | 16.90 | −3.65 | 7.47 |
| Q60972 | Rbbp4 | Histone-binding protein RBBP4 | yes | yes | yes | yes | 18.10 | 3.99E+06 | −4.22 | 13.88 | −1.73 | 0.61 |
| Q89051 | [symbol] | Integral membrane protein 2B | yes | yes | yes | yes | 17.16 | 1.16E+06 | −5.63 | 11.53 | −2.13 | 1.23 |
| Q9Z1N5 | Ddx39b | Spliceosome RNA helicase Ddx39b | yes | yes | yes | yes | 16.28 | 4.68E+05 | −2.49 | 13.79 | −1.12 | 8.14 |
| Q8C878 | [symbol] | NEDD8-activating enzyme E1 catalytic | yes | yes | yes | yes | 14.97 | 9.06E+05 | −4.95 | 10.01 | −1.68 | 1.17 |
| | | subunit | | | | | | | | | | |
| Q35382 | Exoc4 | Exocyst complex component 4 | yes | yes | yes | yes | 14.59 | 6.18E+05 | −6.89 | 7.70 | −1.51 | 0.87 |
| Q35343 | Kpna4 | Importin subunit alpha-3 | yes | yes | NO | yes | 14.28 | 2.73E+06 | −11.90 | 2.38 | −3.18 | −3.56 |
| Q99KJ8 | Dctn2 | Dynactin subunit 2 | yes | yes | yes | yes | 13.94 | 2.16E+06 | −8.43 | 5.51 | −0.53 | 1.58 |
| Q8K2V6 | Ipo11 | Importin-11 | yes | yes | yes | yes | 13.53 | 2.07E+06 | −3.78 | 9.75 | −1.97 | 0.76 |
| Q01405 | Sec23a | Protein transport protein Sec23A | yes | yes | NO | yes | 13.20 | 1.78E+07 | −5.06 | 8.14 | −0.95 | −0.14 |
| Q8BML9 | Qars | Glutamine--tRNA ligase | yes | yes | yes | yes | 13.17 | 9.15E+06 | −5.96 | 7.21 | −2.14 | 1.62 |
| P09528 | [symbol] | Ferritin heavy chain | yes | yes | yes | yes | 12.58 | 2.27E+06 | −12.27 | 0.31 | −3.46 | 0.34 |
| P27612 | [symbol] | Phospholipase A-2-activating protein | yes | yes | yes | yes | 12.45 | 2.10E+06 | −3.77 | 8.69 | −2.39 | 3.71 |
| Q6ZQ08 | Cnot1 | CCR4-NOT transcription complex subunit 1 | yes | yes | yes | yes | 12.30 | 4.31E+06 | −6.76 | 5.54 | −2.56 | 0.71 |
| Q9CYR0 | Ssbp1 | Single-stranded DNA-binding protein, | yes | yes | yes | yes | 12.21 | 5.61E+05 | −8.91 | 3.30 | −1.49 | 0.33 |
| | | mitochondrial | | | | | | | | | | |
| Q922B2 | Dars | Aspartate--tRNA ligase, cytoplasmic | yes | yes | yes | yes | 12.17 | 1.17E+07 | −2.85 | 9.32 | −1.13 | 1.39 |
| Q9Z2X1 | Hnrnpf | Heterogeneous nuclear ribonucleoprotein F | yes | yes | yes | yes | 12.02 | 9.49E+05 | −3.98 | 8.04 | −0.18 | 3.85 |
| P68433 | Hist1h3a | Histone H3.1 | yes | yes | yes | yes | 11.98 | 6.56E+08 | −3.88 | 8.09 | −0.97 | 4.04 |
| Q9CZN7 | Shmt2 | Serine hydroxymethyltransferase, | yes | yes | yes | yes | 11.93 | 4.69E+06 | −2.95 | 8.98 | −1.44 | 6.36 |
| | | mitochondrial | | | | | | | | | | |
| P10923 | Spp1 | Osteopontin | yes | yes | yes | yes | 11.35 | 6.30E+06 | 4.42 | −6.93 | 2.67 | −4.91 |
| Q99PV0 | Prpf8 | Pre-mRNA-processing-splicing factor 8 | yes | yes | yes | yes | 11.34 | 4.64E+07 | −3.39 | 7.95 | −0.01 | 2.09 |
| Q7TQB | Otub1 | Ubiquitin thioesterase OTUB1 | yes | yes | yes | yes | 11.30 | 4.71E+06 | −5.45 | 5.84 | −2.26 | 4.33 |
| Q9WVG5 | Lipg | Endothelial lipase | yes | yes | NO | yes | 11.27 | 1.58E+06 | −8.39 | 2.88 | −2.47 | −0.75 |
| Q9D0R2 | Tars | Threonine--tRNA ligase, cytoplasmic | yes | yes | yes | yes | 9.50 | 1.92E+07 | −3.21 | 6.28 | −1.16 | 1.95 |
| Q55029 | Copb2 | Coatomer subunit beta′ | yes | yes | NO | yes | 9.40 | 3.42E+07 | −3.26 | 6.13 | −1.11 | −0.12 |
| Q8R081 | [symbol] | Heterogeneous nuclear ribonucleoprotein 1 [symbol] | yes | yes | yes | yes | 9.12 | 6.14E+06 | −2.95 | 6.17 | −0.32 | 1.08 |
| [symbol] | [symbol] | Regulator of nonsense transcripts 1 | yes | yes | NO | yes | 9.02 | 6.08E+06 | −4.78 | 4.24 | −1.84 | −0.64 |
| Q70133 | Dhx9 | ATP-dependent RNA helicase A | yes | yes | yes | yes | 9.00 | 6.51E+06 | −2.77 | 6.23 | −1.27 | 0.08 |
| [symbol] | Prep | Prolyl endopeptidase | yes | yes | yes | yes | 8.56 | 9.03E+06 | −3.15 | 5.40 | −1.11 | 0.60 |
| P45376 | Akr1b1 | Aldose reductase | yes | yes | yes | yes | 8.44 | 6.34E+06 | −3.12 | 5.32 | −0.22 | 3.31 |
| [symbol] | [symbol] | Protein transport protein Sec31A | yes | yes | yes | yes | 8.41 | 5.92E+06 | −2.91 | 5.50 | −1.34 | 0.61 |
| Q8BPB5 | [symbol] | EGF-containing fibulin-like ECM protein 1 | yes | yes | yes | yes | 8.34 | 7.05E+06 | 3.74 | −4.60 | 0.07 | −3.65 |
| Q9CQ65 | Mtap | S-methyl-5′-thioadenosine phosphorylase | yes | yes | yes | yes | 8.12 | 6.29E+06 | −4.62 | 3.50 | −0.85 | 1.73 |
| Q61656 | Ddx5 | Probable ATP-dependent RNA helicase | yes | yes | yes | yes | 8.05 | 8.31E+06 | −3.57 | 4.48 | −0.92 | 2.12 |
| | | DDX5 | | | | | | | | | | |
| Q3TXS7 | Psmd1 | 26S proteasome non-ATPase regulatory | yes | yes | yes | yes | 7.84 | 1.94E+07 | −3.50 | 4.33 | −0.65 | 1.22 |
| | | subunit 1 | | | | | | | | | | |
| P97310 | Mcm2 | DNA replication licensing factor MCM2 | yes | yes | NO | yes | 7.66 | 1.28E+07 | −3.70 | 3.96 | −2.10 | −0.30 |
| P48678 | [symbol] | Prelamin- [symbol] | yes | yes | yes | yes | 7.44 | 1.46E+07 | −3.47 | 3.97 | −0.19 | 2.54 |
| Q35286 | Dhx15 | Pre-mRNA-splicing factor ATP-dependent | yes | yes | yes | yes | 7.27 | 2.27E+07 | −2.69 | 4.59 | −1.19 | 1.73 |
| | | RNA helicase DHX15 | | | | | | | | | | |
| Q62261 | [symbol] | Spectrin beta chain, non-crythrocytic 1 | yes | yes | yes | yes | 6.23 | 3.52E+07 | −2.95 | 3.27 | −0.82 | 0.78 |
| Q8CG19 | Ltbp1 | Latent-transforming growth factor beta- | yes | yes | yes | yes | 6.03 | 3.35E+07 | 2.96 | −3.08 | 2.17 | −1.21 |
| | | binding protein 1 | | | | | | | | | | |
| P16546 | Sptan1 | Spectrin alpha chain, non-erythrocytic 1 | yes | yes | yes | yes | 6.02 | 2.31E+07 | −2.81 | 3.22 | −0.40 | 0.14 |
| Q88207 | Col5a1 | Collagen alpha- [symbol] chain | yes | yes | NO | yes | 5.55 | 2.07E+07 | 4.66 | −0.89 | 0.06 | 0.43 |
| Q62167 | Ddx3x | ATP-dependent RNA helicase DDX3X | yes | yes | yes | yes | 5.09 | 2.01E+07 | −2.85 | 2.24 | −1.08 | 0.67 |

[symbol]

indicates data missing or illegible when filed

### Example 4

### Summary of RNA Extraction and Sequencing Methods

In addition to proteomic analysis, total RNA-seq and microRNA-seq was performed on RNA isolated in parallel from the EV samples of Cohort 2. As with the proteomic analysis, one biological replicate was prepared from 3 distinct biological clonal cell lines for each genotype. Here, EVs were isolated from conditioned cell culture media by 18 hour ultracentrifugation at 100,000 g. RNA was extracted using the Qiagen miRNeasy Micro Kit (Cat. No. 217084) according to the manufacturer's protocol. Two RNA fractions were extracted from each sample, one enriched in coding and non-coding RNA species>200 nt (“Total RNA” fraction) and one enriched for small RNAs<200 nt (“miRNA” fraction).

RNA samples were then submitted to the Duke Sequencing and Genomic Technologies shared resource for further quality control and sequencing. Samples were assessed for concentration and integrity by Qubit and Fragment Analyzer. The miRNA fractions were 4.16 ng/μL-47.57 ng/μL and total RNA fractions were 3.8 ng/μL-43.6 ng/μL. The small RNA library was prepared using the QIAseq miRNA Library Kit (Cat No./ID: 331505) and sequenced on the Illumina NextSeq 500 High-Output platform with 75 bp single-reads. The total RNA library was prepared using the Illumina TruSeq Stranded Total RNA-seq Kit with Ribozero Gold ribosomal RNA reduction and sequenced on the Illumina NovaSeq 6000 S1 platform with 100 bp paired-end reads. Sequencer lane pooling and read depth are attached in the Excel data summary (See “RNA QC” Tab).

Data processing and analysis were performed in collaboration with the Duke Genomic Analysis and Bioinformatics shared resource. RNA-seq data was processed using the TrimGalore toolkit (http://www.bioinformatics.babraham.ac.uk/projects/trim_galore), which employs Cutadapt(Martin M. (2011) EMBnet.journal. 17(1):10-12) to trim low quality bases and Illumina sequencing adapters from the 3′ end of the reads. Only reads that were 20 nt or longer after trimming were kept for further analysis. Reads were mapped to the mouse genome and transcriptome (Kersey P J, et al. (2012) Nucleic Acids Res. 40(Database issue):D91-97) using the STAR RNA-seq alignment tool (Dobin A, et al. (2013). Bioinformatics. 29(1):15-21). If the reads mapped to a single genomic location, then they were kept for subsequent analysis. Gene counts were compiled using the HTSeq tool (http://www-huber.embl.de/users/anders/HTSeq/). Only genes that had at least 10 reads in any given library were used in subsequent analysis. Normalization and differential expression was carried out using the DESeq26 Bioconductor (Huber W, et al. (2015) Nat Methods. 12(2):115-121) package with the R statistical programming environment (www.r-project.org). The false discovery rate was calculated to control for multiple hypothesis testing and p-values were adjusted to an FDR of 5%.

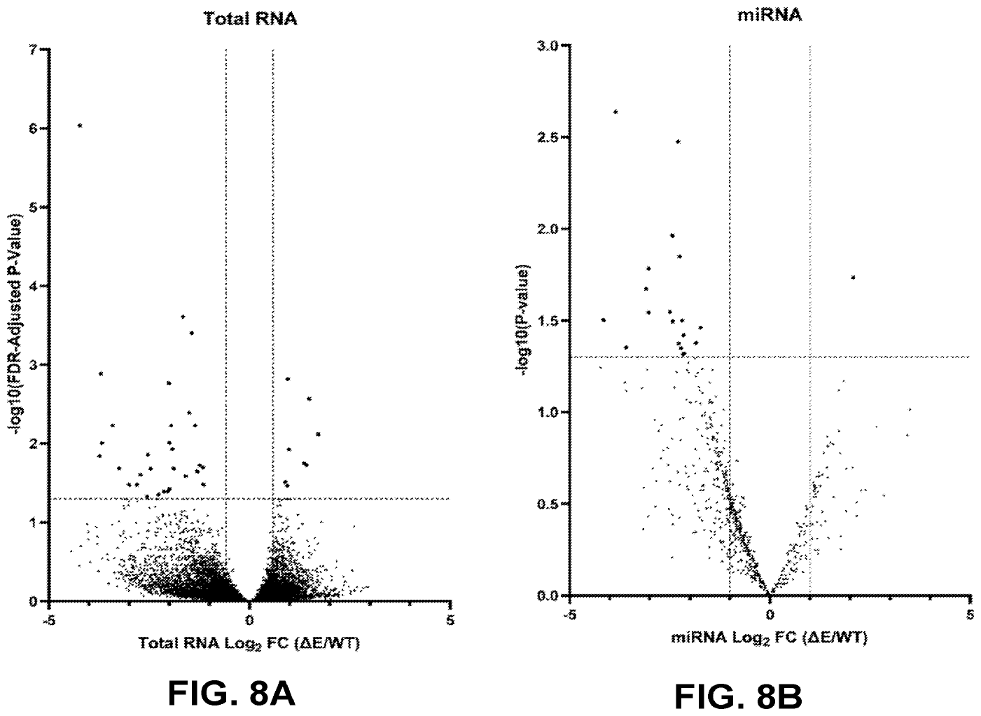

The DYT1 genotype effects on RNA species in EVs was examined. Of 14,729 coding and non-coding large RNAs with signal present in all samples, 39 genes were significantly differentially expressed in DYT1 compared to WT (>1.5 FC, <0.05 FDR-adjusted p-value). Twenty miRNAs were significantly different (>+−2 FC, <0.05 p-value). FIG. 8A shows the differential expression of EV-derived total RNAseq in DYT1 cells compared to WT cells while FIG. 8B shows the differential expressions of EV-derived miRNA-seq in DYT1 cells compared to WT cells. In summary, RNA species may also offer candidate biomarkers for DYT1 genotype difference in EV biospecimens. The discovery set identified here serves as a reference for future replications.

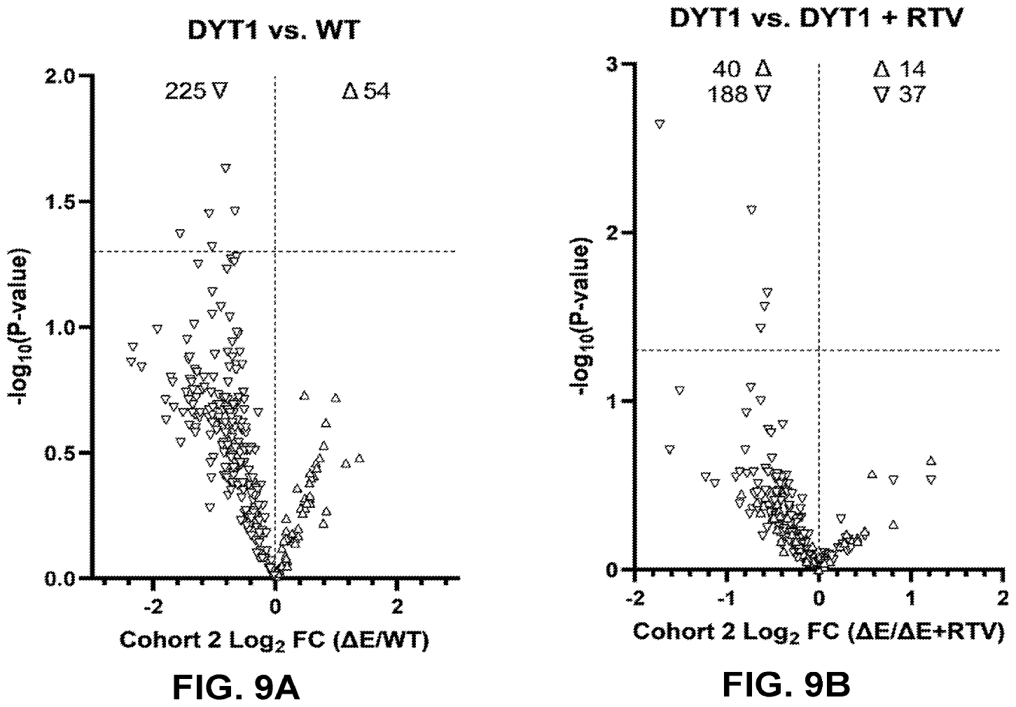

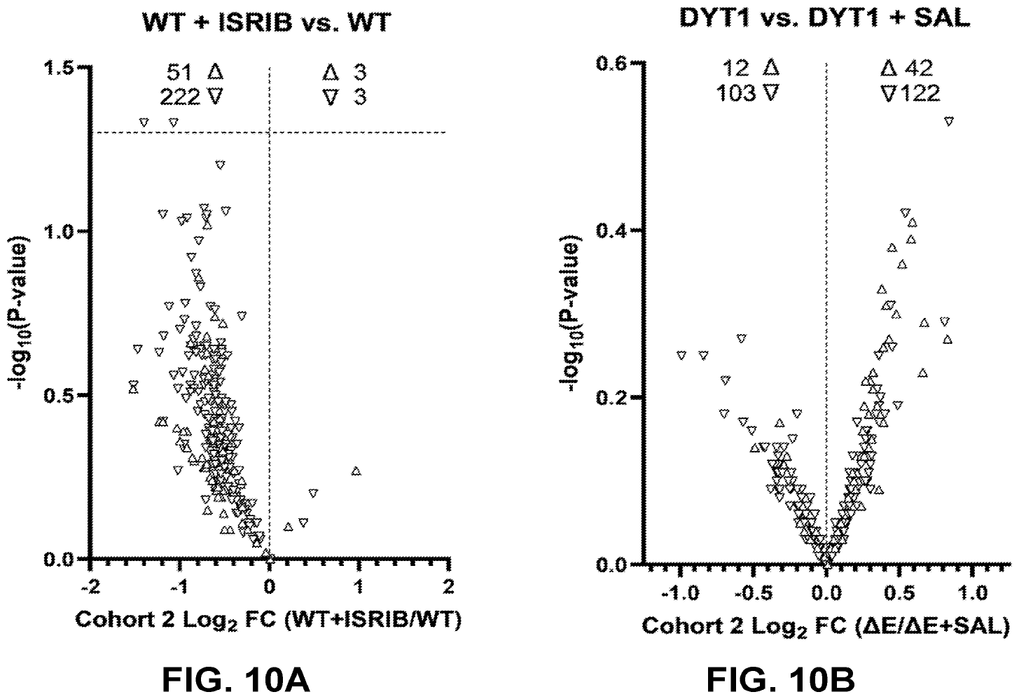

694 miRNAs were initially examined with respect to QC, genotype, RTV effects, and ISRIB effects. This subset was the “Full miRNA” subset. See Table 5. FIG. 9A and FIG. 9B show the RTV effect on EV composition in DYT1 cells compared to WT cells for 279 miRNAs, respectively. Similarly, FIG. 10A shows the effect on 279 miRNAs in WT cells treated with ISRIB compared to non-treated WT cells while FIG. 10B shows the effect on the same 279 miRNAs in DYT1 cells treated with SAL compared to non-treated DYT1 cells.

Beginning with the 694 miRNAs in the “Full miRNA” subset, more than 60 molecules were detected in either WT cells or DYT1 cells (WT or Het UMI>60). In DYT1 cells, RTV modulated expression in a therapeutic direction; that is, in a direction opposite of that of DYT1 genotype-dependent direction. The candidate miRNAs were ranked by the sum of genotype-dependent absolute Z-scores and RTV-induced absolute Z-scores (Abs Z Score Sum (Geno+RTV)). This subset was the “miRNA-Ritonavir Therapeutic Efficacy” subset and contained 201 miRNAs. See Table 6.

Beginning with the 201 miRNAs in the “miRNA-Ritonavir Therapeutic Efficacy” subset, the top 25 miRNAs were identified using a summed Z score of genotype and RTV effects. Then, 13 additional miRNAs were identified using the highest abundance (total UMI) maintaining a Z score>2. This was the “Candidate miRNA” subset and contained 37 miRNAs. See Table 7.

TABLE 5
The 694 miRNAs of the “Full miRNA” Subset

| | RTV | ISRIB | Sal | | Abs Z | | | | | | |
|---|---|---|---|---|---|---|---|---|---|---|---|
| | Direction | Direction | Direction | | Score Sum | | | | | | |
| Mature | Opposite | Same as | Opposite | WT or Het | (Geno + | WT Mean | Het Mean | HetvWT | RTVvHet | ISRIBvWT | SALvHet |
| miRNA | dE? | dE? | ISRIB? | UMI >60 | RTV) | UMIs | UMIs | Z Score | Z Score | Z Score | Z Score |
| mmu-miR-135a-5p | yes | yes | yes | yes | 10.43 | 1.47E+03 | 2.86E+02 | −2.37 | 8.06 | −1.21 | 0.71 |
| mmu-miR-182-5p | yes | yes | yes | yes | 7.27 | 1.19E+04 | 4.87E+03 | −1.94 | 5.33 | −0.64 | 0.01 |
| mmu-miR-542-5p | yes | yes | yes | yes | 7.20 | 8.10E+01 | 4.63E+01 | −5.20 | 2.00 | −0.40 | 0.39 |
| mmu-miR-298-5p | yes | yes | yes | yes | 6.72 | 2.53E+03 | 1.54E+03 | −3.12 | 3.59 | −0.18 | 0.16 |
| mmu-miR-183-5p | yes | yes | NO | yes | 5.55 | 6.18E+03 | 2.33E+03 | −2.14 | 3.42 | −0.62 | −0.06 |
| mmu-miR-296-3p | yes | yes | yes | yes | 5.10 | 6.46E+02 | 4.32E+02 | −2.02 | 3.08 | −0.34 | 0.05 |
| mmu-miR-96-5p | yes | yes | yes | yes | 5.01 | 3.00E+03 | 1.09E+03 | −1.91 | 3.10 | −0.64 | 0.13 |
| mmu-miR-344d-3p | yes | yes | NO | yes | 4.51 | 3.52E+02 | 7.00E+01 | −2.53 | 1.98 | −1.60 | −1.00 |
| mmu-miR-5121 | yes | yes | yes | yes | 4.47 | 1.23E+02 | 5.37E+01 | −1.82 | 2.65 | −0.20 | 0.22 |
| mmu-miR-140-3p | yes | yes | yes | yes | 4.39 | 1.73E+03 | 1.20E+03 | −1.85 | 2.54 | −1.40 | 0.50 |
| mmu-miR-344-3p | yes | yes | yes | yes | 4.24 | 2.61E+02 | 8.87E+01 | −2.95 | 1.30 | −1.22 | 0.44 |
| mmu-miR-187-3p | yes | yes | yes | yes | 3.96 | 2.84E+02 | 1.64E+02 | −2.83 | 1.13 | −1.00 | 0.30 |
| mmu-miR- [symbol] | yes | yes | yes | yes | 3.92 | 6.47E+02 | 2.37E+02 | −2.42 | 1.50 | −1.94 | 0.32 |
| mmu-miR- [symbol] | yes | yes | NO | yes | 3.85 | 4.16E+02 | 2.68E+02 | −2.83 | 1.02 | −2.45 | −0.41 |
| mmu-miR-34a-5p | yes | yes | NO | yes | 3.80 | 2.52E+04 | 1.50E+04 | −2.42 | 1.38 | −1.51 | −0.03 |
| mmu-miR-532-5p | yes | yes | NO | yes | 3.77 | 6.22E+03 | 2.95E+03 | −3.24 | 0.52 | −1.84 | −0.06 |
| mmu-miR-148a-3p | yes | yes | NO | yes | 3.69 | 4.29E+03 | 2.76E+03 | −1.84 | 1.86 | −2.39 | −0.02 |
| mmu-miR-3535 | yes | yes | NO | yes | 3.67 | 7.16E+02 | 4.16E+02 | −2.24 | 1.43 | −0.98 | −0.66 |
| mmu-miR-362-5p | yes | yes | NO | yes | 3.62 | 5.71E+02 | 2.38E+02 | −2.79 | 0.83 | −1.98 | −0.10 |
| mmu-miR-192-5p | yes | yes | yes | yes | 3.38 | 8.32E+02 | 4.07E+02 | −2.85 | 0.54 | −1.40 | 0.09 |
| mmu-miR- [symbol] | yes | yes | yes | yes | 3.36 | 9.36E+03 | 5.77E+03 | −2.03 | 1.33 | −1.49 | 0.07 |
| mmu-miR-1291 | yes | yes | NO | yes | 3.34 | 2.77E+02 | 9.43E+01 | −1.43 | 1.91 | −0.30 | −0.06 |
| mmu-miR- [symbol] | yes | yes | yes | yes | 3.25 | 2.06E+03 | 1.04E+03 | 2.02 | 1.23 | 0.80 | 0.15 |
| mmu-miR-362-3p | yes | yes | yes | yes | 3.23 | 7.78E+02 | 1.70E+02 | −2.18 | 1.05 | −1.63 | 0.47 |
| mmu-miR-671-5p | yes | yes | yes | yes | 3.19 | 1.52E+03 | 1.05E+03 | −1.43 | 1.76 | −1.04 | 0.26 |
| mmu-miR-224-5p | yes | yes | NO | yes | 3.17 | 3.13E+03 | 1.97E+03 | −2.90 | 0.28 | −1.90 | −0.27 |
| mmu-miR-346-5p | yes | yes | yes | yes | 3.13 | 1.47E+03 | 5.94E+02 | −1.58 | 1.55 | −1.05 | 0.38 |
| mmu-miR- [symbol] | yes | yes | yes | yes | 3.11 | 1.50E+02 | 4.57E+01 | −2.06 | 1.05 | −1.40 | 0.01 |
| mmu-miR-219a-5p | yes | yes | yes | yes | 3.07 | 2.45E+02 | 9.13E+01 | −1.99 | 1.08 | −1.03 | 0.34 |
| mmu-miR-31-5p | yes | yes | NO | yes | 3.05 | 3.03E+04 | 1.68E+04 | −1.64 | 1.41 | −0.48 | −0.14 |
| mmu-miR-340-5p | yes | yes | yes | yes | 3.02 | 1.22E+03 | 4.90E+02 | −1.93 | 1.09 | −1.41 | 0.31 |
| mmu-miR-33-5p | yes | yes | yes | yes | 3.01 | 2.07E+02 | 8.40E+01 | −1.49 | 1.52 | −0.60 | 0.26 |
| mmu-miR- [symbol] | yes | yes | yes | yes | 3.00 | 3.27E+03 | 1.27E+03 | −1.83 | 1.17 | −0.73 | 0.40 |
| mmu-miR-500-3p | yes | yes | yes | yes | 2.97 | 5.20E+02 | 2.33E+02 | −1.88 | 1.09 | −1.14 | 0.10 |
| mmu-miR-194-5p | yes | yes | yes | yes | 2.90 | 1.44E+03 | 7.05E+02 | −2.44 | 0.46 | −1.11 | 0.18 |
| mmu-miR- [symbol] | yes | yes | yes | yes | 2.89 | 1.08E+04 | 4.36E+03 | −1.45 | 1.15 | −0.63 | 0.35 |
| mmu-miR-700-3p | yes | yes | yes | yes | 2.84 | 2.18E+02 | 1.18E+02 | −2.35 | 0.49 | −1.35 | 0.16 |
| mmu-miR- [symbol] | yes | yes | NO | yes | 2.83 | 5.07E+03 | 3.32E+03 | −2.25 | 0.58 | −1.34 | −0.07 |
| mmu-let-7i-3p | yes | yes | yes | yes | 2.75 | 2.23E+02 | 1.17E+02 | −1.60 | 1.15 | −0.31 | 0.35 |
| mmu-miR-188-5p | yes | yes | NO | yes | 2.73 | 3.25E+02 | 1.29E+02 | −2.40 | 0.34 | −1.71 | −0.05 |
| mmu-miR- [symbol] | yes | yes | yes | yes | 2.73 | 1.12E+03 | 2.94E+02 | −2.22 | 0.52 | −1.26 | 0.64 |
| mmu-miR-99a-5p | yes | yes | NO | yes | 2.71 | 2.06E+03 | 9.94E+02 | −1.99 | 0.73 | −1.70 | −0.17 |
| mmu-miR-652-3p | yes | yes | yes | yes | 2.71 | 7.18E+02 | 2.96E+02 | −1.78 | 0.93 | −0.83 | 0.34 |
| mmu-miR-22-3p | yes | yes | yes | yes | 2.70 | 3.04E+04 | 1.33E+04 | −1.44 | 1.26 | −1.17 | 0.17 |
| mmu-miR-335-5p | yes | yes | NO | yes | 2.69 | 1.54E+03 | 4.44E+02 | −1.67 | 1.02 | −0.65 | −0.35 |
| mmu-miR-214-5p | yes | yes | yes | yes | 2.68 | 1.95E+02 | 1.00E+02 | −1.58 | 1.09 | −0.50 | 0.04 |
| mmu-miR- [symbol] | yes | yes | yes | yes | 2.65 | 2.47E+02 | 7.80E+01 | −1.77 | 0.88 | −0.73 | 0.26 |
| mmu-miR- [symbol] | yes | yes | NO | yes | 2.61 | 1.15E+05 | 6.65E+04 | −1.44 | 1.17 | −0.84 | −0.10 |
| mmu-miR-31-3p | yes | yes | NO | yes | 2.61 | 4.70E+02 | 1.84E+02 | −1.78 | 0.83 | −0.86 | −0.03 |
| mmu-miR-32-5p | yes | yes | yes | yes | 2.61 | 8.74E+02 | 2.72E+02 | −1.85 | 0.75 | −1.14 | 0.26 |
| mmu-miR-199a-3p | yes | yes | NO | yes | 2.60 | 2.30E+05 | 1.27E+05 | −1.53 | 1.07 | −0.84 | −0.10 |
| mmu-miR- [symbol] | yes | yes | yes | yes | 2.59 | 1.36E+04 | 8.25E+03 | −1.22 | 1.38 | −0.70 | 0.42 |
| mmu-miR- [symbol] | yes | yes | yes | yes | 2.58 | 1.93E+04 | 1.02E+04 | −1.51 | 1.07 | −0.84 | 0.07 |
| mmu-miR-378a-3p | yes | yes | NO | yes | 2.57 | 1.75E+03 | 1.21E+03 | −1.32 | 1.25 | −1.49 | −0.12 |
| mmu-miR-146a-5p | yes | yes | yes | yes | 2.56 | 5.66E+02 | 2.76E+02 | −2.46 | 0.10 | −0.93 | 0.02 |
| mmu-miR-708-5p | yes | yes | yes | yes | 2.51 | 2.83E+02 | 1.59E+02 | −1.23 | 1.28 | −3.87 | 0.58 |
| mmu-miR-542-3p | yes | yes | yes | yes | 2.51 | 5.04E+02 | 1.98E+02 | −1.70 | 0.81 | −0.46 | 0.33 |
| mmu-miR-30a-5p | yes | yes | NO | yes | 2.50 | 3.72E+04 | 2.44E+04 | −1.53 | 0.97 | −1.43 | −0.09 |
| mmu-miR-320-3p | yes | yes | NO | yes | 2.47 | 3.27E+03 | 2.69E+03 | −1.72 | 0.76 | −1.53 | −0.21 |
| mmu-miR-3102-3p | yes | yes | yes | yes | 2.47 | 9.93E+01 | 6.30E+01 | −1.52 | 0.95 | −1.55 | 0.35 |
| mmu-miR-1981-5p | yes | yes | yes | yes | 2.47 | 2.38E+02 | 1.77E+02 | −1.13 | 1.34 | −1.22 | 0.21 |
| mmu-miR-93-5p | yes | yes | NO | yes | 2.43 | 4.94E+04 | 3.19E+04 | −2.13 | 0.30 | −1.12 | −0.01 |
| mmu-miR-339-5p | yes | yes | yes | yes | 2.43 | 1.76E+03 | 1.24E+03 | −1.56 | 0.87 | −0.87 | 0.06 |
| mmu-miR-674-5p | yes | yes | NO | yes | 2.43 | 1.11E+03 | 7.61E+02 | −1.38 | 1.05 | −0.75 | −0.32 |
| mmu-miR-181d-5p | yes | yes | NO | yes | 2.42 | 2.60E+02 | 1.10E+02 | −1.64 | 0.78 | −1.08 | −0.01 |
| mmu-miR-30a-3p | yes | yes | NO | yes | 2.38 | 8.07E+02 | 4.72E+02 | −1.68 | 0.70 | −1.18 | −0.09 |
| mmu-miR-101a-3p | yes | yes | yes | yes | 2.38 | 1.43E+03 | 5.58E+02 | −1.51 | 0.87 | −0.66 | 0.48 |
| mmu-miR- [symbol] | yes | yes | yes | yes | 2.38 | 4.66E+04 | 1.74E+04 | −1.70 | 0.68 | −0.73 | 0.17 |
| mmu-miR-155-5p | yes | yes | NO | yes | 2.36 | 8.50E+03 | 5.23E+03 | −1.91 | 0.45 | −1.26 | −0.20 |
| mmu-let-7i-5p | yes | yes | NO | yes | 2.35 | 6.62E+04 | 4.53E+04 | −1.38 | 0.97 | −0.95 | −0.06 |
| mmu-miR-107-3p | yes | yes | yes | yes | 2.32 | 2.46E+03 | 1.43E+03 | −1.57 | 0.74 | −1.02 | 0.03 |
| mmu-miR- [symbol] | yes | yes | yes | yes | 2.30 | 2.88E+03 | 1.43E+03 | −1.26 | 1.04 | −0.56 | 0.29 |
| mmu-miR-598-3p | yes | yes | yes | yes | 2.30 | 3.55E+02 | 1.83E+02 | −1.51 | 0.78 | −0.78 | 0.06 |
| mmu-miR-103-3p | yes | yes | NO | yes | 2.29 | 4.19E+04 | 2.69E+04 | −1.81 | 0.48 | −0.84 | −0.10 |
| mmu-miR-199a-5p | yes | yes | NO | yes | 2.27 | 5.69E+03 | 3.14E+03 | −1.52 | 0.75 | −0.50 | −0.13 |
| mmu-miR-138-5p | yes | yes | NO | yes | 2.24 | 1.16E+03 | 4.38E+02 | −2.09 | 0.15 | −1.66 | −0.14 |
| mmu-miR-503-5p | yes | yes | NO | yes | 2.23 | 1.16E+03 | 7.28E+02 | −1.59 | 0.64 | −0.47 | −0.28 |
| mmu-let-7f-5p | yes | yes | NO | yes | 2.22 | 1.53E+05 | 9.57E+04 | −1.51 | 0.71 | −1.16 | −0.04 |
| mmu-miR-126a-3p | yes | yes | yes | yes | 2.21 | 6.90E+02 | 5.35E+02 | −0.94 | 1.27 | −0.20 | 0.13 |
| mmu-miR- [symbol] | yes | yes | yes | yes | 2.19 | 1.73E+03 | 4.97E+02 | −1.85 | 0.34 | −1.67 | 0.68 |
| mmu-miR-18a-5p | yes | yes | yes | yes | 2.19 | 1.68E+03 | 7.16E+02 | −1.59 | 0.60 | −0.54 | 0.21 |
| mmu-miR-378a-5p | yes | yes | yes | yes | 2.18 | 1.11E+02 | 9.47E+01 | −0.89 | 1.28 | −0.81 | 0.24 |
| mmu-miR- [symbol] | yes | yes | NO | yes | 2.15 | 8.72E+03 | 4.87E+03 | −1.58 | 0.57 | −1.10 | 0.00 |
| mmu-miR-19a-3p | yes | yes | yes | yes | 2.12 | 5.21E+03 | 1.83E+03 | −1.62 | 0.49 | −0.65 | 0.15 |
| mmu-miR- [symbol] | yes | yes | yes | yes | 2.11 | 2.18E+02 | 1.39E+02 | −1.09 | 1.02 | −1.11 | 0.04 |
| mmu-miR-301a-5p | yes | yes | yes | yes | 2.11 | 6.10E+01 | 3.23E+01 | −1.54 | 0.57 | −0.59 | 0.14 |
| mmu-miR-675-3p | yes | yes | yes | yes | 2.10 | 1.27E+02 | 6.30E+01 | −1.86 | 0.25 | −2.24 | 0.34 |
| mmu-miR-27a-3p | yes | yes | yes | yes | 2.10 | 1.30E+04 | 6.09E+03 | −1.44 | 0.66 | −0.88 | 0.21 |
| mmu-miR- [symbol] | yes | yes | yes | yes | 2.08 | 2.96E+04 | 1.64E+04 | −1.35 | 0.73 | −1.11 | 0.05 |
| mmu-miR-3068-5p | yes | yes | NO | yes | 2.06 | 2.02E+02 | 1.15E+02 | −1.30 | 0.76 | −0.88 | −0.02 |
| mmu-miR-190a-5p | yes | yes | yes | yes | 2.06 | 2.41E+03 | 9.02E+02 | −1.43 | 0.62 | −0.38 | 0.04 |
| mmu-miR-872-5p | yes | yes | yes | yes | 2.05 | 3.32E+03 | 1.71E+03 | −1.37 | 0.68 | −1.26 | 0.19 |
| mmu-miR- [symbol] | yes | NO | yes | yes | 2.05 | 2.86E+02 | 1.40E+02 | −1.51 | 0.54 | 0.31 | 0.04 |
| mmu-miR- [symbol] | yes | yes | NO | yes | 2.04 | 2.00E+03 | 1.37E+03 | −1.89 | 0.15 | −1.54 | −0.23 |
| mmu-miR- [symbol] | yes | yes | yes | yes | 2.02 | 3.16E+02 | 1.40E+02 | −1.73 | 0.28 | −0.97 | 0.15 |
| mmu-miR-98-5p | yes | yes | NO | yes | 2.00 | 2.72E+03 | 1.51E+03 | −1.58 | 0.42 | −0.75 | −0.22 |
| mmu-let-7g-5p | yes | yes | NO | yes | 1.98 | 2.85E+04 | 1.65E+04 | −1.48 | 0.50 | −1.37 | −0.16 |
| mmu-miR-322-5p | yes | yes | NO | yes | 1.97 | 5.19E+03 | 3.18E+03 | −1.22 | 0.75 | −0.40 | −0.07 |
| mmu-miR-152-3p | yes | yes | NO | yes | 1.94 | 1.07E+04 | 7.69E+03 | −1.18 | 0.76 | −1.28 | −0.02 |
| mmu-miR-350-3p | yes | yes | yes | yes | 1.94 | 2.48E+03 | 1.17E+03 | −1.53 | 0.41 | −0.49 | 0.08 |
| mmu-miR-181a-5p | yes | yes | NO | yes | 1.93 | 7.53E+03 | 4.37E+03 | −1.62 | 0.31 | −1.45 | −0.23 |
| mmu-miR-299a-3p | yes | yes | yes | yes | 1.92 | 7.63E+01 | 5.33E+01 | −0.61 | 1.31 | −1.31 | 0.39 |
| mmu-miR-151-5p | yes | yes | yes | yes | 1.90 | 1.94E+02 | 1.28E+02 | −1.58 | 0.32 | −0.91 | 0.12 |
| mmu-miR-25-3p | yes | yes | NO | yes | 1.89 | 3.40E+04 | 1.61E+04 | −1.18 | 0.71 | −1.12 | −0.04 |
| mmu-miR-451a | yes | NO | NO | yes | 1.89 | 1.90E+02 | 2.16E+02 | 0.52 | −1.37 | −1.27 | 0.38 |
| mmu-miR- [symbol] | yes | yes | NO | yes | 1.88 | 1.23E+02 | 6.07E+01 | −1.57 | 0.31 | −0.68 | −0.29 |
| mmu-miR- [symbol] | yes | yes | NO | yes | 1.88 | 3.80E+03 | 2.15E+03 | −1.34 | 0.54 | −1.35 | −0.32 |
| mmu-miR-30d-5p | yes | yes | NO | yes | 1.86 | 1.36E+04 | 1.08E+04 | −1.16 | 0.70 | −1.38 | −0.11 |
| mmu-miR- [symbol] | yes | yes | NO | yes | 1.85 | 4.03E+02 | 2.45E+02 | −1.36 | 0.49 | −0.94 | −0.06 |
| mmu-miR-483-5p | yes | yes | NO | yes | 1.83 | 2.15E+02 | 1.27E+02 | −1.82 | 0.01 | −2.93 | −1.22 |
| mmu-miR-149-5p | yes | yes | yes | yes | 1.82 | 1.10E+03 | 7.87E+02 | −1.49 | 0.33 | −1.27 | 0.03 |
| mmu-miR-503-3p | yes | yes | NO | yes | 1.82 | 6.40E+01 | 5.00E+01 | −0.83 | 0.99 | −0.76 | −0.70 |
| mmu-miR-136-3p | yes | yes | yes | yes | 1.81 | 7.97E+01 | 6.73E+01 | −0.29 | 1.52 | −0.88 | 0.08 |
| mmu-miR-361-5p | yes | yes | NO | yes | 1.79 | 4.46E+03 | 2.84E+03 | −1.08 | 0.71 | −0.70 | −0.15 |
| mmu-miR-582-3p | yes | yes | NO | yes | 1.78 | 1.11E+02 | 6.90E+01 | −1.58 | 0.20 | −1.08 | −0.45 |
| mmu-let-7a-1-3p | yes | yes | yes | yes | 1.76 | 3.85E+02 | 2.23E+02 | −1.14 | 0.62 | −0.93 | 0.28 |
| mmu-let-7-2-3p | yes | yes | yes | yes | 1.76 | 3.85E+02 | 2.23E+02 | −1.14 | 0.62 | −0.93 | 0.28 |
| mmu-miR-186-5p | yes | yes | yes | yes | 1.76 | 1.01E+04 | 5.70E+03 | −1.42 | 0.33 | −0.99 | 0.00 |
| mmu-miR-324-3p | yes | yes | yes | yes | 1.75 | 7.93E+02 | 5.32E+02 | −0.92 | 0.82 | −0.50 | 0.16 |
| mmu-miR-223-3p | yes | NO | yes | yes | 1.72 | 1.16E+02 | 1.93E+02 | 1.14 | −0.59 | −1.00 | −0.01 |
| mmu-miR- [symbol] | yes | yes | yes | yes | 1.72 | 2.05E+04 | 1.13E+04 | −1.19 | 0.53 | −0.45 | 0.38 |
| mmu-miR-16-5p | yes | yes | NO | yes | 1.71 | 5.91E+05 | 3.54E+05 | −1.50 | 0.21 | −1.22 | −0.01 |
| mmu-miR-1843a-5p | yes | yes | yes | yes | 1.70 | 7.67E+01 | 4.67E+01 | −1.42 | 0.28 | −1.23 | 0.07 |
| mmu-miR- [symbol] | yes | yes | NO | yes | 1.70 | 3.88E+02 | 2.89E+02 | −0.91 | 0.78 | −1.37 | −0.37 |
| mmu-miR-185-5p | yes | yes | yes | yes | 1.69 | 3.04E+03 | 2.19E+03 | −1.16 | 0.53 | −0.81 | 0.15 |
| mmu-miR-124-3p | yes | NO | yes | yes | 1.67 | 6.80E+01 | 7.00E+01 | 0.07 | −1.59 | −1.71 | −0.72 |
| mmu-miR-191-5p | yes | yes | yes | yes | 1.62 | 2.08E+04 | 1.42E+04 | −1.34 | 0.28 | −1.22 | 0.04 |
| mmu-miR-23a-3p | yes | yes | yes | yes | 1.62 | 4.74E+04 | 3.15E+04 | −1.16 | 0.46 | −1.43 | 0.05 |
| mmu-miR-17-3p | yes | yes | yes | yes | 1.59 | 6.98E+02 | 3.97E+02 | −1.57 | 0.02 | −1.43 | 0.10 |
| mmu-miR-206-3p | yes | yes | NO | yes | 1.58 | 5.64E+03 | 3.47E+03 | −1.38 | 0.20 | −1.56 | −0.27 |
| mmu-miR-15a-5p | yes | yes | yes | yes | 1.58 | 5.73E+03 | 2.74E+03 | −1.36 | 0.22 | −0.57 | 0.17 |
| mmu-miR-450a-5p | yes | NO | yes | yes | 1.53 | 1.39E+02 | 6.67E+01 | −1.15 | 0.39 | 0.01 | 0.09 |
| mmu-miR-20a-5p | yes | yes | NO | yes | 1.51 | 2.72E+04 | 1.33E+04 | −1.42 | 0.09 | −0.84 | −0.01 |
| mmu-miR-322-3p | yes | yes | NO | yes | 1.47 | 2.71E+02 | 1.77E+02 | −1.20 | 0.28 | −0.98 | −0.26 |
| mmu-miR-29a-3p | yes | yes | yes | yes | 1.44 | 1.12E+05 | 6.90E+04 | −1.15 | 0.29 | −0.75 | 0.17 |
| mmu-miR- [symbol] | yes | yes | yes | yes | 1.42 | 1.39E+03 | 8.58E+02 | −0.92 | 0.51 | −0.80 | 0.16 |
| mmu-miR- [symbol] | yes | yes | NO | yes | 1.42 | 2.77E+02 | 2.24E+02 | −0.55 | 0.87 | −0.72 | −0.27 |
| mmu-miR-222-3p | yes | yes | NO | yes | 1.42 | 3.72E+03 | 2.63E+03 | −1.25 | 0.16 | −1.61 | −0.53 |
| mmu-miR-128-3p | yes | yes | NO | yes | 1.42 | 4.26E+03 | 2.81E+03 | −1.24 | 0.18 | −1.04 | −0.03 |
| mmu-miR-21a-5p | yes | yes | NO | yes | 1.41 | 3.21E+05 | 1.79E+05 | −1.24 | 0.17 | −1.01 | −0.03 |
| mmu-miR- [symbol] | yes | yes | yes | yes | 1.39 | 6.87E+01 | 4.03E+01 | −0.81 | 0.58 | −0.50 | 0.29 |
| mmu-miR-421-3p | yes | yes | NO | yes | 1.38 | 1.16E+03 | 6.99E+02 | −1.13 | 0.25 | −0.89 | −0.01 |
| mmu-miR-335-3p | yes | yes | NO | yes | 1.35 | 8.20E+01 | 3.97E+01 | −1.04 | 0.31 | −0.23 | −0.41 |
| mmu-miR-1839-5p | yes | yes | NO | yes | 1.34 | 1.25E+03 | 7.66E+02 | −1.33 | 0.01 | −2.22 | −0.04 |
| mmu-miR-26a-5p | yes | yes | NO | yes | 1.33 | 5.71E+04 | 4.03E+04 | −0.91 | 0.42 | −1.13 | −0.13 |
| mmu-miR-130a-3p | yes | yes | NO | yes | 1.32 | 1.21E+04 | 7.63E+03 | −1.02 | 0.30 | −0.49 | −0.03 |
| mmu-miR-122-5p | yes | NO | yes | yes | 1.32 | 8.81E+02 | 1.31E+03 | 0.83 | −0.50 | −0.85 | −0.07 |
| mmu-miR-7a-1-3p | yes | yes | NO | yes | 1.32 | 1.35E+02 | 8.60E+01 | −1.20 | 0.12 | −1.50 | −0.29 |
| mmu-miR-143-3p | yes | yes | yes | yes | 1.32 | 1.44E+05 | 8.98E+04 | −0.99 | 0.33 | −0.93 | 0.03 |
| mmu-miR-288-3p | yes | yes | NO | yes | 1.31 | 3.20E+03 | 2.18E+03 | −1.05 | 0.27 | −0.89 | −0.11 |
| mmu-miR-210-3p | yes | yes | yes | yes | 1.31 | 1.02E+03 | 6.37E+02 | −1.02 | 0.29 | −0.28 | 0.03 |
| mmu-miR-425-3p | yes | yes | NO | yes | 1.30 | 6.33E+01 | 4.27E+01 | −1.09 | 0.21 | −0.92 | −0.03 |
| mmu-miR-17-5p | yes | yes | NO | yes | 1.28 | 1.10E+03 | 6.44E+02 | −1.23 | 0.04 | −0.50 | −0.15 |
| mmu-let-7j | yes | yes | yes | yes | 1.27 | 3.54E+03 | 7.90E+03 | 1.12 | −0.15 | 0.67 | −0.38 |
| mmu-let-7d-5p | yes | yes | NO | yes | 1.25 | 2.08E+04 | 1.56E+04 | −0.95 | 0.30 | −0.87 | −0.32 |
| mmu-miR-872-3p | yes | yes | yes | yes | 1.22 | 8.19E+02 | 4.70E+02 | −1.01 | 0.21 | −0.61 | 0.10 |
| mmu-miR- [symbol] | yes | yes | yes | yes | 1.21 | 3.56E+04 | 2.19E+04 | −0.92 | 0.38 | −1.38 | 0.13 |
| mmu-miR-326-3p | yes | yes | yes | yes | 1.20 | 8.27E+01 | 6.67E+01 | −0.64 | 0.55 | −0.17 | 0.07 |
| mmu-miR-126a-5p | yes | yes | yes | yes | 1.18 | 4.16E+02 | 3.31E+02 | −0.65 | 0.54 | −0.55 | 0.27 |
| mmu-let- [symbol] | yes | yes | NO | yes | 1.18 | 2.34E+04 | 1.87E+04 | −0.87 | 0.31 | −0.47 | −0.38 |
| mmu-miR-376a-3p | yes | yes | NO | yes | 1.16 | 1.26E+02 | 1.08E+02 | −0.27 | 0.89 | −0.90 | −0.32 |
| mmu-miR-143-5p | yes | yes | NO | yes | 1.15 | 1.76E+03 | 1.20E+03 | −0.77 | 0.38 | −0.79 | −0.11 |
| mmu-miR-24-3p | yes | yes | yes | yes | 1.14 | 9.37E+04 | 7.44E+04 | −0.71 | 0.43 | −1.12 | 0.02 |
| mmu-let-7a-5p | yes | yes | NO | yes | 1.06 | 1.29E+05 | 1.04E+05 | −0.88 | 0.19 | −0.83 | −0.49 |
| mmu-miR-214-3p | yes | yes | NO | yes | 1.06 | 6.83E+03 | 3.94E+03 | −1.05 | 0.01 | −0.84 | −0.43 |
| mmu-miR-324-5p | yes | yes | NO | yes | 1.05 | 1.20E+03 | 9.42E+02 | −0.80 | 0.25 | −0.84 | −0.07 |
| mmu-miR-93-3p | yes | yes | NO | yes | 1.02 | 2.32E+02 | 1.80E+02 | −0.95 | 0.07 | −1.45 | −0.35 |
| mmu-miR-676-3p | yes | yes | NO | yes | 1.02 | 6.90E+02 | 4.52E+02 | −1.00 | 0.02 | −1.97 | −0.24 |
| mmu-miR-29a-5p | yes | yes | yes | yes | 0.99 | 1.80E+02 | 1.10E+02 | −0.84 | 0.15 | −0.78 | 0.16 |
| mmu-miR-455-3p | yes | yes | yes | yes | 0.97 | 8.27E+01 | 9.37E+01 | 0.42 | −0.55 | 0.28 | −0.35 |
| mmu-miR-296-5p | yes | yes | yes | yes | 0.96 | 2.71E+02 | 2.27E+02 | −0.60 | 0.37 | −0.79 | 0.16 |
| mmu-miR-129-2-3p | yes | NO | NO | yes | 0.93 | 2.76E+02 | 3.16E+02 | 0.21 | −0.72 | −1.46 | 0.11 |
| mmu-miR-491-5p | yes | yes | NO | yes | 0.92 | 8.17E+01 | 7.10E+01 | −0.73 | 0.18 | −2.61 | −0.27 |
| mmu-miR-145a-5p | yes | yes | NO | yes | 0.89 | 1.46E+04 | 1.11E+04 | −0.63 | 0.26 | −0.64 | −0.03 |
| mmu-miR-142a-3p | yes | NO | NO | yes | 0.81 | 2.17E+02 | 2.39E+02 | 0.43 | −0.38 | −2.35 | 0.46 |
| mmu-miR-1198-5p | yes | yes | NO | yes | 0.80 | 6.12E+02 | 5.43E+02 | −0.61 | 0.19 | −1.13 | −0.20 |
| mmu-miR- [symbol] | yes | yes | NO | yes | 0.80 | 1.72E+02 | 1.43E+02 | −0.74 | 0.06 | −0.99 | −0.49 |
| mmu-miR- [symbol] | yes | yes | NO | yes | 0.78 | 9.48E+03 | 7.21E+03 | −0.67 | 0.12 | −1.42 | −0.09 |
| mmu-miR-136-5p | yes | yes | NO | yes | 0.77 | 7.37E+01 | 6.17E+01 | −0.24 | 0.54 | −0.72 | −0.49 |
| mmu-miR-140-5p | yes | NO | NO | yes | 0.69 | 3.18E+02 | 3.56E+02 | 0.20 | −0.49 | −1.03 | 0.14 |
| mmu-miR-669o-3p | yes | yes | yes | yes | 0.67 | 1.42E+03 | 9.69E+02 | −0.59 | 0.08 | −0.53 | 0.26 |
| mmu-miR-669a-3p | yes | yes | yes | yes | 0.67 | 1.57E+04 | 1.07E+04 | −0.59 | 0.08 | −0.53 | 0.26 |
| mmu-miR-486a-5p | yes | NO | yes | yes | 0.66 | 2.03E+02 | 2.30E+02 | 0.23 | −0.43 | −1.07 | −0.29 |
| mmu-miR-449a-5p | yes | yes | yes | yes | 0.66 | 1.79E+02 | 1.34E+02 | −0.56 | 0.10 | −1.22 | 0.26 |
| mmu-miR-218-5p | yes | yes | NO | yes | 0.66 | 6.32E+04 | 4.67E+04 | −0.50 | 0.16 | −1.01 | −0.03 |
| mmu-miR-1298-5p | yes | yes | yes | yes | 0.65 | 7.30E+01 | 6.53E+01 | −0.23 | 0.42 | −1.22 | 0.20 |
| mmu-miR-92a-3p | yes | yes | NO | yes | 0.57 | 1.77E+04 | 1.48E+04 | −0.47 | 0.10 | −1.41 | −0.13 |
| mmu-miR-193a-5p | yes | yes | yes | yes | 0.55 | 2.66E+02 | 2.41E+02 | −0.32 | 0.23 | −1.47 | 0.00 |
| mmu-miR-345-3p | yes | NO | yes | yes | 0.52 | 1.75E+02 | 1.70E+02 | −0.04 | 0.48 | 0.53 | 0.24 |
| mmu-miR-674-3p | yes | yes | NO | yes | 0.52 | 5.63E+02 | 4.71E+02 | −0.38 | 0.13 | −0.66 | −0.03 |
| mmu-miR-497a-5p | yes | yes | yes | yes | 0.52 | 1.38E+02 | 1.32E+02 | −0.08 | 0.44 | −0.56 | 0.10 |
| mmu-miR-150-5p | yes | NO | NO | yes | 0.51 | 5.93E+01 | 7.70E+01 | 0.46 | −0.05 | −0.80 | 0.39 |
| mmu-miR-423-5p | yes | yes | yes | yes | 0.51 | 2.20E+03 | 2.10E+03 | −0.20 | 0.31 | −0.68 | 0.04 |
| mmu-let- [symbol] | yes | yes | NO | yes | 0.49 | 3.83E+04 | 3.24E+04 | −0.31 | 0.18 | −0.90 | −0.05 |
| mmu-miR- [symbol] | yes | yes | NO | yes | 0.48 | 7.69E+02 | 6.16E+02 | −0.41 | 0.07 | −0.87 | −0.06 |
| mmu-miR-669d-5p | yes | yes | yes | yes | 0.47 | 6.67E+01 | 5.40E+01 | −0.28 | 0.19 | −0.17 | 0.10 |
| mmu-miR-369-3p | yes | yes | NO | yes | 0.26 | 2.00E+02 | 1.92E+02 | −0.10 | 0.17 | −1.35 | −0.14 |
| mmu-miR-484 | yes | NO | yes | yes | 0.21 | 1.98E+03 | 2.04E+03 | 0.09 | −0.12 | −1.31 | −0.15 |
| mmu-miR- [symbol] | yes | yes | yes | yes | 0.18 | 2.48E+02 | 2.35E+02 | −0.11 | 0.07 | −0.74 | 0.25 |
| mmu-let- [symbol] | yes | yes | NO | yes | 0.16 | 1.08E+05 | 1.02E+05 | −0.13 | 0.03 | −1.24 | −0.21 |
| mmu-miR-129-5p | yes | NO | NO | yes | 0.03 | 1.78E+03 | 1.81E+03 | 0.03 | 0.00 | −1.61 | 0.09 |
| mmu-miR-501-3p | NO | yes | NO | yes | 3.97 | 1.78E+03 | 1.12E+03 | −3.67 | −0.30 | −2.55 | −0.56 |
| mmu-miR-200a-3p | NO | yes | NO | yes | 2.39 | 1.02E+02 | 7.20E+01 | −1.07 | −1.32 | −0.84 | −0.28 |
| mmu-miR- [symbol] | NO | yes | NO | yes | 2.25 | 6.37E+01 | 4.43E+01 | −0.85 | −1.40 | −1.21 | −0.50 |
| mmu-miR-1a-3p | NO | yes | NO | yes | 2.18 | 1.09E+02 | 4.80E+01 | −1.66 | −0.52 | −1.64 | −0.07 |
| mmu-miR-299a-5p | NO | NO | yes | yes | 1.92 | 1.58E+02 | 3.15E+02 | 1.58 | 0.34 | −0.70 | −0.47 |
| mmu-miR-540-3p | NO | NO | yes | yes | 1.78 | 1.56E+02 | 2.18E+02 | 0.80 | 0.98 | −0.71 | −0.58 |
| mmu-miR-154-5p | NO | NO | yes | yes | 1.75 | 2.06E+02 | 2.79E+02 | 0.73 | 1.02 | −0.56 | −0.56 |
| mmu-miR-411-5p | NO | NO | yes | yes | 1.74 | 3.62E+02 | 4.84E+02 | 0.73 | 1.00 | −0.77 | −0.61 |
| mmu-miR-361-3p | NO | yes | NO | yes | 1.73 | 7.13E+01 | 5.10E+01 | −1.52 | −0.22 | −0.32 | −0.36 |
| mmu-miR-425-5p | NO | yes | NO | yes | 1.70 | 3.44E+03 | 2.41E+03 | −1.47 | −0.29 | −1.39 | −0.01 |
| mmu-miR-328-3p | NO | NO | NO | yes | 1.70 | 1.58E+02 | 2.21E+02 | 1.61 | 0.09 | −0.07 | 0.18 |
| mmu-miR-434-3p | NO | NO | yes | yes | 1.67 | 3.58E+02 | 6.35E+02 | 1.40 | 0.28 | −0.59 | −0.94 |
| mmu-miR-337-5p | NO | NO | yes | yes | 1.66 | 1.02E+02 | 1.42E+02 | 0.71 | 0.95 | −0.59 | −0.36 |
| mmu-miR-409-3p | NO | NO | yes | yes | 1.66 | 1.87E+02 | 2.97E+02 | 1.11 | 0.56 | −0.58 | −0.57 |
| mmu-miR-9-5p | NO | NO | yes | yes | 1.64 | 2.61E+03 | 6.79E+03 | 1.17 | 0.46 | −0.38 | −0.10 |
| mmu-miR-134-5p | NO | NO | yes | yes | 1.63 | 3.27E+02 | 4.80E+02 | 0.77 | 0.86 | −0.16 | −0.07 |
| mmu-miR-379-5p | NO | NO | yes | yes | 1.63 | 7.53E+02 | 1.07E+03 | 0.81 | 0.82 | −0.54 | −0.56 |
| mmu-miR-1191a | NO | yes | NO | yes | 1.57 | 1.86E+02 | 1.20E+02 | −1.16 | −0.41 | −0.94 | −0.12 |
| mmu-miR-127-3p | NO | NO | yes | yes | 1.55 | 8.24E+02 | 1.43E+03 | 1.22 | 0.33 | −0.50 | −0.49 |
| mmu-miR-673-5p | NO | NO | yes | yes | 1.53 | 5.30E+01 | 7.23E+01 | 0.66 | 0.87 | −1.02 | −0.77 |
| mmu-miR-329-5p | NO | NO | yes | yes | 1.52 | 9.13E+01 | 1.22E+02 | 0.72 | 0.80 | −0.64 | −0.57 |
| mmu-miR-541-5p | NO | NO | yes | yes | 1.48 | 1.28E+03 | 2.04E+03 | 1.04 | 0.44 | −0.60 | −0.75 |
| mmu-miR-540-5p | NO | NO | yes | yes | 1.45 | 5.23E+01 | 6.40E+01 | 0.41 | 1.04 | −0.76 | −0.12 |
| mmu-miR-369-5p | NO | NO | yes | yes | 1.43 | 1.99E+02 | 3.09E+02 | 0.99 | 0.45 | −0.54 | −0.66 |
| mmu-miR-664-3p | NO | yes | NO | yes | 1.43 | 6.63E+01 | 4.43E+01 | −1.28 | −0.15 | −2.17 | −0.13 |
| mmu-miR-672-5p | NO | yes | NO | yes | 1.42 | 2.68E+02 | 1.52E+02 | −1.06 | −0.36 | −0.81 | −0.32 |
| mmu-miR-342-3p | NO | yes | NO | yes | 1.40 | 6.10E+03 | 4.41E+03 | −1.21 | −0.19 | −1.25 | −0.28 |
| mmu-miR-455-5p | NO | yes | NO | yes | 1.40 | 3.27E+02 | 1.79E+02 | −1.36 | −0.03 | −0.74 | −0.17 |
| mmu-miR-494-3p | NO | NO | yes | yes | 1.39 | 5.77E+02 | 8.70E+02 | 0.94 | 0.44 | −0.95 | −0.31 |
| mmu-miR-532-3p | NO | yes | NO | yes | 1.35 | 2.41E+02 | 1.81E+02 | −1.17 | −0.18 | −0.70 | −0.42 |
| mmu-miR-434-5p | NO | NO | yes | yes | 1.35 | 2.22E+02 | 3.27E+02 | 0.94 | 0.41 | −0.72 | −0.92 |
| mmu-miR-1983 | NO | yes | NO | yes | 1.33 | 3.02E+03 | 1.69E+03 | −1.15 | −0.18 | −0.88 | −0.11 |
| mmu-miR-574-3p | NO | NO | yes | yes | 1.33 | 3.00E+02 | 3.84E+02 | 0.87 | 0.46 | −0.60 | −0.23 |
| mmu-miR-382-5p | NO | NO | yes | yes | 1.33 | 6.54E+02 | 9.74E+02 | 0.74 | 0.59 | −0.29 | −0.69 |
| mmu-miR- [symbol] | NO | yes | NO | yes | 1.23 | 1.65E+02 | 9.23E+01 | −1.02 | −0.21 | −0.39 | −0.18 |
| mmu-miR-300-3p | NO | NO | NO | yes | 1.22 | 4.99E+02 | 5.09E+02 | 0.04 | 1.19 | −0.88 | 0.12 |
| mmu-miR-409-5p | NO | NO | yes | yes | 1.21 | 1.00E+02 | 1.25E+02 | 0.44 | 0.78 | −0.51 | −0.50 |
| mmu-miR-9-3p | NO | NO | NO | yes | 1.13 | 2.74E+02 | 2.86E+02 | 0.12 | 1.01 | −1.88 | 0.35 |
| mmu-miR-423-3p | NO | yes | NO | yes | 1.11 | 1.20E+03 | 9.79E+02 | −0.85 | −0.26 | −1.34 | −0.12 |
| mmu-miR-341-3p | NO | NO | yes | yes | 1.11 | 4.07E+01 | 6.03E+01 | 1.01 | 0.10 | −0.25 | −0.89 |
| mmu-miR-381-3p | NO | NO | yes | yes | 1.10 | 2.79E+02 | 3.14E+02 | 0.27 | 0.83 | −0.97 | −0.51 |
| mmu-miR-431-5p | NO | NO | yes | yes | 1.08 | 4.18E+02 | 6.20E+02 | 0.94 | 0.14 | −0.56 | −0.81 |
| mmu-miR- [symbol] | NO | yes | NO | yes | 1.05 | 1.14E+03 | 5.45E+02 | −0.73 | −0.32 | −0.48 | −0.71 |
| mmu-miR-132-3p | NO | yes | yes | yes | 1.03 | 1.82E+03 | 1.29E+03 | −0.68 | −0.35 | −2.78 | 0.10 |
| mmu-miR- [symbol] | NO | yes | yes | yes | 1.01 | 3.67E+04 | 2.29E+04 | −0.98 | −0.03 | −0.85 | 0.08 |
| mmu-miR-28a-5p | NO | yes | NO | yes | 0.99 | 4.68E+02 | 3.42E+02 | −0.62 | −0.37 | −0.88 | −0.29 |
| mmu-miR- [symbol] | NO | yes | NO | yes | 0.98 | 8.10E+03 | 6.13E+03 | −0.82 | −0.15 | −0.71 | −0.16 |
| mmu-miR- [symbol] | NO | yes | NO | yes | 0.97 | 4.91E+02 | 3.41E+02 | −0.92 | −0.05 | −0.69 | −0.09 |
| mmu-miR-221-3p | NO | yes | NO | yes | 0.94 | 3.42E+04 | 2.71E+04 | −0.72 | −0.22 | −2.30 | −0.46 |
| mmu-miR-21a-3p | NO | yes | NO | yes | 0.91 | 1.13E+02 | 9.50E+01 | −0.39 | −0.52 | −0.48 | −0.34 |
| mmu-miR-196a-5p | NO | NO | yes | yes | 0.89 | 3.55E+03 | 6.15E+03 | 0.57 | 0.32 | −0.25 | −0.25 |
| mmu-miR-744-5p | NO | yes | NO | yes | 0.89 | 2.75E+03 | 2.29E+03 | −0.85 | −0.04 | −0.93 | −0.28 |
| mmu-miR-10a-5p | NO | NO | yes | yes | 0.89 | 7.90E+02 | 1.41E+03 | 0.67 | 0.21 | −0.41 | −0.69 |
| mmu-miR- [symbol] | NO | yes | NO | yes | 0.88 | 2.03E+02 | 1.90E+02 | −0.13 | −0.75 | −1.07 | −0.80 |
| mmu-miR-323-3p | NO | NO | yes | yes | 0.86 | 5.80E+01 | 8.30E+01 | 0.75 | 0.11 | −0.90 | −0.96 |
| mmu-miR- [symbol] | NO | NO | yes | yes | 0.85 | 2.43E+02 | 2.47E+02 | 0.03 | 0.83 | −0.79 | −0.10 |
| mmu-miR-212-3p | NO | yes | NO | yes | 0.85 | 1.45E+02 | 1.16E+02 | −0.41 | −0.44 | −0.87 | −0.02 |
| mmu-miR-365-3p | NO | yes | NO | yes | 0.81 | 1.56E+04 | 1.22E+04 | −0.65 | −0.16 | −0.90 | −0.06 |
| mmu-let-7d-3p | NO | NO | yes | yes | 0.76 | 9.91E+02 | 1.12E+03 | 0.62 | 0.14 | −0.73 | −0.31 |
| mmu-miR- [symbol] | NO | NO | NO | yes | 0.75 | 1.39E+02 | 1.46E+02 | 0.10 | 0.66 | −1.01 | 0.02 |
| mmu-miR- [symbol] | NO | yes | NO | yes | 0.75 | 2.63E+04 | 2.27E+04 | −0.50 | −0.25 | −0.93 | −0.25 |
| mmu-miR-467a-5p | NO | yes | NO | yes | 0.74 | 4.23E+02 | 3.03E+02 | −0.67 | −0.06 | −0.41 | −0.08 |
| mmu-miR-701-5p | NO | yes | NO | yes | 0.72 | 9.83E+01 | 8.87E+01 | −0.48 | −0.24 | −1.09 | −0.29 |
| mmu-miR-22-5p | NO | yes | NO | yes | 0.69 | 1.70E+02 | 1.57E+02 | −0.20 | −0.49 | −0.69 | −0.36 |
| mmu-miR-1193-3p | NO | NO | yes | yes | 0.66 | 7.07E+01 | 9.20E+01 | 0.53 | 0.13 | −1.26 | −0.41 |
| mmu-miR-100-5p | NO | yes | NO | yes | 0.63 | 3.90E+03 | 3.37E+03 | −0.27 | −0.36 | −2.22 | −0.10 |
| mmu-miR-466d-3p | NO | yes | yes | yes | 0.63 | 1.81E+02 | 1.31E+02 | −0.55 | −0.08 | −0.91 | 0.23 |
| mmu-miR-151-3p | NO | NO | yes | yes | 0.59 | 1.25E+03 | 1.34E+03 | 0.34 | 0.25 | −0.64 | −0.29 |
| mmu-miR-669f-3p | NO | yes | NO | yes | 0.53 | 7.80E+01 | 5.93E+01 | −0.44 | −0.09 | −0.71 | −0.01 |
| mmu-miR- [symbol] | NO | NO | NO | yes | 0.51 | 9.75E+02 | 1.23E+03 | 0.41 | 0.10 | −1.94 | 0.05 |
| mmu-miR-125a-5p | NO | NO | yes | yes | 0.49 | 2.81E+04 | 3.42E+04 | 0.48 | 0.01 | −0.46 | −0.25 |
| mmu-miR-351-5p | NO | yes | yes | yes | 0.41 | 1.30E+03 | 1.31E+03 | 0.03 | 0.38 | 0.01 | −0.45 |
| mmu-miR-195a-5p | NO | NO | NO | yes | 0.34 | 2.22E+02 | 2.39E+02 | 0.15 | 0.19 | −1.62 | 0.06 |
| mmu-miR- [symbol] | NO | yes | yes | yes | 0.27 | 2.09E+02 | 1.80E+02 | −0.24 | −0.03 | −0.94 | 0.09 |
| mmu-miR- [symbol] | NO | yes | NO | yes | 0.21 | 6.86E+05 | 6.62E+05 | −0.10 | −0.10 | −1.03 | −0.20 |
| mmu-miR- [symbol] | NO | NO | yes | yes | 0.20 | 2.54E+02 | 2.63E+02 | 0.06 | 0.14 | −0.45 | −0.21 |
| mmu-miR-615-3p | NO | NO | yes | yes | 0.14 | 6.77E+01 | 7.77E+01 | 0.14 | 0.00 | −0.26 | −0.60 |
| mmu-miR-331-3p | NO | yes | NO | yes | 0.02 | 1.21E+02 | 1.20E+02 | −0.02 | 0.00 | −0.32 | −0.12 |
| mmu-miR-182-3p | [symbol] | yes | [symbol] | NO | [symbol] | 1.67E+00 | 0.00E+00 | −5.00 | [symbol] | −2.83 | [symbol] |
| mmu-miR-7211-3p | [symbol] | yes | [symbol] | NO | [symbol] | 1.67E+00 | 0.00E+00 | −5.00 | [symbol] | −5.00 | [symbol] |
| mmu-miR-7675-3p | [symbol] | yes | [symbol] | NO | [symbol] | 1.33E+00 | 0.00E+00 | −1.51 | [symbol] | −1.06 | [symbol] |
| mmu-miR-706-4-5p | [symbol] | [symbol] | [symbol] | NO | [symbol] | 1.00E+00 | 0.00E+00 | [symbol] | [symbol] | [symbol] | [symbol] |
| mmu-miR-363-3p | [symbol] | NO | yes | NO | [symbol] | 3.33E+01 | 0.00E+00 | −1.00 | [symbol] | 2.12 | 1.00 |
| mmu-miR-7081-3p | [symbol] | [symbol] | [symbol] | NO | [symbol] | 0.00E+00 | 2.00E+00 | [symbol] | 0.00 | 1.00 | −4.00 |
| mmu-miR-410-5p | [symbol] | [symbol] | [symbol] | NO | [symbol] | 0.00E+00 | 0.00E+00 | [symbol] | 4.00 | 1.00 | 1.00 |
| mmu-miR-1247-5p | [symbol] | [symbol] | [symbol] | NO | [symbol] | 0.00E+00 | 0.00E+00 | [symbol] | [symbol] | 1.00 | 1.00 |
| mmu-miR-5099 | [symbol] | [symbol] | [symbol] | NO | [symbol] | 0.00E+00 | 0.00E+00 | [symbol] | 1.00 | [symbol] | 1.00 |
| mmu-miR-7035-3p | [symbol] | [symbol] | [symbol] | NO | [symbol] | 0.00E+00 | 0.00E+00 | [symbol] | [symbol] | [symbol] | 5.00 |
| mmu-miR- [symbol] | yes | NO | yes | NO | 14.66 | 7.00E+00 | 1.17E+01 | 14.00 | −0.66 | 0.00 | −0.76 |
| mmu-miR-3098-3p | yes | yes | [symbol] | NO | 8.02 | 6.67E+01 | 4.00E+00 | 5.00 | −3.02 | 1.51 | [symbol] |
| mmu-miR-7648-3p | yes | yes | yes | NO | 8.00 | 0.00E+00 | 1.33E+00 | 4.00 | −4.00 | 1.00 | −1.41 |
| mmu-miR-205-5p | yes | NO | yes | NO | 7.50 | 2.67E+01 | 4.43E+01 | 2.43 | −5.07 | −1.14 | −3.03 |
| mmu-miR-6996-5p | yes | yes | [symbol] | NO | 6.00 | 1.33E+00 | 0.00E+00 | −4.00 | 2.00 | −4.00 | [symbol] |
| mmu-miR-677-5p | yes | yes | yes | NO | 5.95 | 1.67E+00 | 3.00E+00 | 0.76 | −5.20 | −1.00 | −1.00 |
| mmu-miR-3065-5p | yes | yes | NO | NO | 5.75 | 5.00E+00 | 2.67E+00 | −2.21 | 3.54 | −1.75 | −0.22 |
| mmu-miR-7051-5p | yes | yes | yes | NO | 5.73 | 1.33E+00 | 0.00E+00 | −4.00 | 1.73 | −1.41 | 1.00 |
| mmu-miR-5114 | yes | yes | yes | NO | 5.67 | 1.37E+01 | 2.67E+00 | −0.94 | 4.73 | −0.42 | 1.20 |
| mmu-miR-1251-5p | yes | yes | NO | NO | 5.54 | 1.03E+01 | 1.33E+00 | −2.00 | 3.54 | −0.95 | 0.00 |
| mmu-miR-183-3p | yes | yes | yes | NO | 5.50 | 3.00E+00 | 6.67E+01 | −3.50 | 2.00 | −2.65 | 0.89 |
| mmu-miR-7658-5p | yes | NO | yes | NO | 5.50 | 2.33E+00 | 0.00E+00 | −3.50 | 2.00 | 1.34 | 1.00 |
| mmu-miR-5709-5p | yes | yes | yes | NO | 5.45 | 3.67E+00 | 6.67E+01 | −1.88 | 3.58 | −0.71 | 0.28 |
| mmu-miR-1955-3p | yes | yes | yes | NO | 5.37 | 1.30E+01 | 5.67E+00 | −5.05 | 0.32 | −4.11 | 0.72 |
| mmu-miR-154-3p | yes | yes | yes | NO | 5.33 | 1.33E+00 | 2.67E+00 | 2.83 | −2.50 | 0.55 | −0.55 |
| mmu-miR-7213-5p | yes | yes | yes | NO | 5.30 | 4.67E+00 | 6.67E+01 | −4.24 | 1.06 | −0.62 | 0.90 |
| mmu-miR-505-5p | yes | yes | yes | NO | 5.23 | 1.17E+01 | 5.67E+00 | −4.02 | 1.20 | −0.67 | 1.02 |
| mmu-miR-15a-3p | yes | yes | NO | NO | 5.17 | 3.40E+01 | 7.67E+00 | −2.69 | 2.48 | −1.60 | −0.06 |
| mmu-miR-3058-5p | yes | yes | yes | NO | 5.16 | 1.40E+01 | 7.00E+00 | −3.83 | 1.32 | −2.14 | 0.17 |
| mmu-miR-297a-3p | yes | yes | yes | NO | 5.11 | 3.40E+01 | 7.33E+00 | −1.36 | 3.75 | −0.80 | 0.85 |
| mmu-miR-12189-3p | yes | yes | yes | NO | 5.00 | 3.00E+00 | 3.33E+01 | −4.00 | 1.00 | −0.87 | 1.34 |
| mmu-miR-152-5p | yes | yes | yes | NO | 4.71 | 3.17E+01 | 1.60E+01 | −2.32 | 2.38 | −0.82 | 0.27 |
| mmu-miR-7219-3p | yes | [symbol] | yes | NO | 4.69 | 0.00E+00 | 2.00E+00 | 3.46 | −1.22 | [symbol] | −2.00 |
| mmu-miR-3097-3p | yes | yes | yes | NO | 4.57 | 3.00E+00 | 1.00E+00 | −1.55 | 3.02 | −0.50 | 0.77 |
| mmu-miR-450a-2-3p | yes | yes | yes | NO | 4.54 | 2.70E+01 | 7.00E+00 | −2.05 | 2.50 | −0.66 | 0.29 |
| mmu-miR- [symbol] | yes | NO | yes | NO | 4.50 | 2.33E+00 | 0.00E+00 | −3.50 | 1.00 | 0.76 | 1.00 |
| mmu-miR-107-5p | yes | yes | yes | NO | 4.24 | 2.33E+00 | 3.33E+01 | −2.12 | 2.12 | −2.12 | 1.00 |
| mmu-miR-7069-3p | yes | yes | yes | NO | 4.23 | 1.00E+00 | 0.00E+00 | −1.73 | 2.50 | −1.73 | 2.00 |
| mmu-miR- [symbol] | yes | yes | yes | NO | 4.21 | 2.37E+01 | 9.67E+00 | −2.28 | 1.93 | −1.57 | 0.74 |
| mmu-miR-3081-3p | yes | yes | yes | NO | 4.15 | 1.50E+01 | 4.67E+00 | −3.42 | 0.73 | −1.62 | 0.41 |
| mmu-miR-141-3p | yes | NO | yes | NO | 4.13 | 6.33E+00 | 2.00E+00 | −2.98 | 1.15 | 0.16 | 0.85 |
| mmu-miR-6939-3p | yes | yes | NO | NO | 4.12 | 1.33E+00 | 3.33E+01 | −2.12 | 2.00 | −2.12 | −1.00 |
| mmu-miR-7061-5p | yes | [symbol] | NO | NO | 4.12 | 1.00E+00 | 3.33E+01 | −2.00 | 2.12 | [symbol] | −1.00 |
| mmu-miR-1927 | yes | NO | yes | NO | 4.07 | 2.00E+00 | 3.33E+01 | −2.50 | 1.57 | 0.00 | 1.26 |
| mmu-miR-6900-3p | yes | yes | yes | NO | 3.91 | 2.00E+00 | 3.33E+01 | −2.50 | 1.41 | −1.51 | 1.06 |
| mmu-miR-873a-5p | yes | yes | NO | NO | 3.84 | 3.00E+00 | 1.00E+00 | −3.46 | 0.38 | −2.45 | −1.00 |
| mmu-miR-700-5p | yes | yes | yes | NO | 3.80 | 1.57E+01 | 9.00E+00 | −1.75 | 2.05 | −1.20 | 0.44 |
| mmu-miR- [symbol] | yes | yes | yes | NO | 3.72 | 1.70E+01 | 6.67E+00 | −2.89 | 0.83 | −2.88 | 0.38 |
| mmu-miR-670-3p | yes | yes | yes | NO | 3.73 | 2.67E+00 | 1.00E+00 | −2.50 | 1.22 | −0.55 | 0.50 |
| mmu-miR-3572-3p | yes | yes | yes | NO | 3.65 | 2.33E+00 | 0.00E+00 | −2.65 | 1.00 | −0.23 | 2.00 |
| mmu-miR-7652-3p | yes | yes | yes | NO | 3.62 | 3.67E+00 | 5.67E+00 | 1.60 | −2.01 | 0.28 | −0.74 |
| mmu-miR-147-3p | yes | yes | yes | NO | 3.59 | 4.70E+01 | 1.67E+01 | −1.64 | 1.95 | −0.76 | 1.05 |
| mmu-miR- [symbol] | yes | yes | yes | NO | 3.56 | 5.07E+01 | 9.33E+00 | −2.54 | 1.02 | −1.44 | 0.62 |
| mmu-miR-3099-3p | yes | yes | NO | NO | 3.54 | 5.60E+01 | 1.07E+01 | −2.87 | 0.67 | −0.40 | −1.20 |
| mmu-miR-704 | yes | NO | yes | NO | 3.54 | 6.67E+01 | 1.33E+00 | 1.41 | −2.12 | −0.71 | −2.12 |
| mmu-miR- [symbol] | yes | yes | yes | NO | 3.52 | 3.67E+00 | 0.00E+00 | −2.52 | 1.00 | −1.57 | 1.00 |
| mmu-miR-708-3p | yes | yes | yes | NO | 3.52 | 4.67E+00 | 1.33E+00 | −1.96 | 1.55 | −1.18 | 0.78 |
| mmu-miR-6935-5p | yes | NO | yes | NO | 3.49 | 3.00E+00 | 1.67E+00 | −2.00 | 1.49 | 0.32 | 0.56 |
| mmu-miR-421-5p | yes | yes | yes | NO | 3.46 | 5.67E+00 | 1.67E+00 | −1.70 | 1.77 | −1.27 | 0.71 |
| mmu-miR-495-5p | yes | NO | yes | NO | 3.46 | 3 33E+01 | 1.67E+00 | 2.83 | −0.63 | 0.00 | −0.63 |
| mmu-let-7f-2-3p | yes | yes | yes | NO | 3.42 | 1.43E+01 | 3.00E+00 | −1.98 | 1.44 | −0.74 | 0.81 |
| mmu-miR-3101-5p | yes | yes | yes | NO | 3.33 | 1.10E+01 | 2.67E+00 | −2.67 | 0.67 | −1.39 | 0.69 |
| mmu-miR-6539 | yes | NO | yes | NO | 3.26 | 4.00E+00 | 1.33E+00 | −1.26 | 2.00 | 0.28 | 1.21 |
| mmu-miR-9769-3p | yes | yes | yes | NO | 3.21 | 2.00E+00 | 3.33E+01 | −2.50 | 0.71 | −2.00 | 0.71 |
| mmu-miR- [symbol] | yes | yes | yes | NO | 3.20 | 5.67E+00 | 3.00E+00 | −1.71 | 1.49 | −0.85 | 0.32 |
| mmu-miR-7031-5p | yes | yes | yes | NO | 3.18 | 1.67E+00 | 6.67E+01 | −1.06 | 2.12 | −0.90 | 0.50 |
| mmu-miR-7654-5p | yes | yes | yes | NO | 3.17 | 3.77E+01 | 2.47E+01 | −1.98 | 1.19 | −0.17 | 0.07 |
| mmu-miR-3084-3p | yes | yes | NO | NO | 3.15 | 1.27E+01 | 5.33E+00 | −2.67 | 0.49 | −4.02 | 0.00 |
| mmu-miR- [symbol] | yes | NO | yes | NO | 3.15 | 5.00E+00 | 2.00E+00 | −1.30 | 1.85 | 0.32 | 0.76 |
| mmu-miR-483-3p | yes | NO | yes | NO | 3.13 | 2.00E+00 | 3.33E+01 | −2.50 | 0.63 | 0.29 | 1.05 |
| mmu-miR-7668-3p | yes | yes | NO | NO | 3.12 | 1.00E+00 | 3.33E+01 | −1.00 | 2.12 | −1.73 | 0.00 |
| mmu-miR-1941-3p | yes | yes | yes | NO | 3.11 | 2.67E+00 | 3.33E+01 | −2.47 | 0.63 | −1.07 | 0.73 |
| mmu-miR-19a-5p | yes | yes | yes | NO | 3.08 | 1.00E+01 | 1.33E+00 | −1.66 | 1.41 | −0.69 | 0.66 |
| mmu-miR- [symbol] | yes | NO | NO | NO | 3.07 | 3.33E+00 | 5.00E+00 | 1.58 | −1.49 | −1.77 | 0.00 |
| mmu-miR-30d-3p | yes | yes | yes | NO | 3.02 | 1.83E+01 | 7.33E+00 | −2.12 | 0.90 | −1.07 | 0.58 |
| mmu-miR-6915-5p | yes | NO | yes | NO | 3.00 | 1.00E+00 | 1.67E+00 | 2.00 | −1.00 | 0.00 | −2.83 |
| mmu-miR- [symbol] | yes | NO | yes | NO | 3.00 | 2.33E+00 | 3.00E+00 | 0.50 | −2.50 | −0.67 | −0.87 |
| mmu-miR-1931 | yes | NO | NO | NO | 3.00 | 3.33E+01 | 1.00E+00 | 2.00 | −1.00 | 0.00 | 0.00 |
| mmu-miR-1938 | yes | yes | NO | NO | 2.98 | 4.67E+00 | 1.33E+00 | −2.24 | 0.74 | −2.35 | 0.00 |
| mmu-miR-3079-5p | yes | yes | yes | NO | 2.95 | 1.03E+01 | 5.67E+00 | −1.98 | 0.97 | −0.08 | 0.18 |
| mmu-miR-339-3p | yes | yes | yes | NO | 2.93 | 1.10E+01 | 5.00E+00 | −2.45 | 0.48 | −2.46 | 0.89 |
| mmu-miR-7008-5p | yes | yes | NO | NO | 2.85 | 2.00E+00 | 6.67E+01 | −1.51 | 1.34 | −2.00 | −0.45 |
| mmu-miR-3091-5p | yes | yes | NO | NO | 2.85 | 3.67E+00 | 1.00E+00 | −2.00 | 0.85 | −0.40 | −1.00 |
| mmu-miR-7655-5p | yes | NO | yes | NO | 2.83 | 6.67E+01 | 1.67E+00 | 2.12 | −0.71 | −0.71 | −5.00 |
| mmu-miR-181d-3p | yes | yes | NO | NO | 2.83 | 2.67E+00 | 6.67E+01 | −2.12 | 0.71 | −0.46 | 0.00 |
| mmu-miR-24-2-5p | yes | yes | yes | NO | 2.80 | 4.67E+00 | 1.00E+00 | −2.08 | 0.72 | −0.63 | 0.65 |
| mmu-miR-7666-3p | yes | yes | yes | NO | 2.76 | 3.00E+00 | 6.67E+01 | −1.49 | 1.26 | −1.07 | 1.41 |
| mmu-miR-3154 | yes | yes | yes | NO | 2.76 | 1.67E+00 | 3.00E+00 | 2.00 | −0.76 | 0.60 | −0.55 |
| mmu-miR- [symbol] | yes | NO | yes | NO | 2.73 | 3.33E+01 | 0.00E+00 | −1.00 | 1.73 | 0.63 | 2.50 |
| mmu-miR-7669-5p | yes | yes | yes | NO | 2.70 | 1.77E+01 | 7.00E+00 | −1.55 | 1.15 | −1.43 | 0.32 |
| mmu-miR-3057-5p | yes | NO | yes | NO | 2.65 | 3.57E+01 | 5.00E+01 | 1.03 | −1.62 | −0.84 | −0.42 |
| mmu-miR-8106 | yes | yes | NO | NO | 2.62 | 1.67E+00 | 6.67E+01 | −2.12 | 0.50 | −0.35 | 0.00 |
| mmu-miR-6538 | yes | yes | NO | NO | 2.62 | 1.67E+00 | 6.67E+01 | −2.12 | 0.50 | −2.12 | 0.00 |
| mmu-miR-5622-5p | yes | yes | yes | NO | 2.58 | 6.33E+00 | 2.33E+00 | −2.35 | 0.22 | −3.40 | 0.16 |
| mmu-miR-582-5p | yes | yes | NO | NO | 2.53 | 4.67E+00 | 2.33E+00 | −1.87 | 0.66 | −0.68 | −0.39 |
| mmu-miR-670-5p | yes | yes | yes | NO | 2.52 | 7.00E+00 | 1.33E+00 | −2.29 | 0.22 | −2.44 | 0.81 |
| mmu-miR-5113 | yes | NO | yes | NO | 2.50 | 1.67E+00 | 1.00E+00 | −0.50 | 2.00 | 0.25 | 0.55 |
| mmu-let-7a-2-3p | yes | NO | NO | NO | 2.50 | 1.00E+10 | 6.67E+01 | −0.38 | 2.12 | 0.00 | −0.45 |
| mmu-miR-452-3p | yes | yes | NO | NO | 2.49 | 4.27E+01 | 2.17E+01 | −1.74 | 0.75 | −1.71 | −0.24 |
| mmu-miR-188-3p | yes | yes | yes | NO | 2.48 | 1.77E+01 | 4.00E+00 | −1.98 | 0.50 | −0.51 | 0.43 |
| mmu-miR-6911-3p | yes | yes | yes | NO | 2.47 | 2.00E+00 | 1.00E+00 | −1.22 | 1.25 | −0.50 | 0.63 |
| mmu-miR-1943-3p | yes | yes | yes | NO | 2.45 | 6.67E+01 | 3.33E+01 | −0.45 | 2.00 | −1.00 | 2.12 |
| mmu-miR-98-3p | yes | yes | yes | NO | 2.43 | 5.37E+01 | 2.33E+01 | −2.00 | 0.44 | −0.82 | 0.22 |
| mmu-miR-5622-3p | yes | yes | yes | NO | 2.40 | 2.33E+00 | 3.33E+01 | −1.06 | 1.34 | −0.35 | 1.34 |
| mmu-miR-5623-3p | yes | NO | yes | NO | 2.40 | 6.67E+01 | 1.67E+00 | 1.06 | −1.34 | −0.45 | −2.50 |
| mmu-miR-300-5p | yes | NO | yes | NO | 2.40 | 3.33E+00 | 2.00E+00 | −0.76 | 1.64 | 0.20 | 0.40 |
| mmu-miR-1929-5p | yes | yes | yes | NO | 2.39 | 4.33E+00 | 1.33E+00 | −2.18 | 0.21 | −2.18 | 0.33 |
| mmu-miR-450a-1-3p | yes | yes | yes | NO | 2.36 | 9.67E+00 | 3.33E+00 | −1.68 | 0.68 | −1.04 | 0.12 |
| mmu-miR-770-3p | yes | NO | yes | NO | 2.35 | 5.00E+00 | 1.07E+01 | 1.51 | −0.84 | −0.27 | −2.34 |
| mmu-miR-16-1-3p | yes | yes | yes | NO | 2.35 | 3.97E+01 | 1.37E+01 | −1.94 | 0.41 | −1.26 | 0.29 |
| mmu-miR-204-5p | yes | yes | yes | NO | 2.34 | 3.33E+01 | 1.67E+00 | 1.79 | −0.55 | 1.00 | −0.55 |
| mmu-miR-410-3p | yes | NO | yes | NO | 2.34 | 4.00E+00 | 6.67E+00 | 2.00 | −0.34 | −1.10 | −1.37 |
| mmu-miR-100-3p | yes | NO | yes | NO | 2.32 | 2.00E+00 | 3.00E+00 | 0.61 | −1.71 | −0.25 | −0.69 |
| mmu-miR-679-3p | yes | NO | yes | NO | 2.28 | 2.00E+00 | 4.00E+00 | 1.41 | −0.87 | −1.26 | −1.49 |
| mmu-miR- [symbol] | yes | yes | yes | NO | 2.27 | 1.70E+01 | 3.67E+00 | −1.36 | 0.91 | −0.80 | 0.85 |
| mmu-miR- [symbol] | yes | yes | yes | NO | 2.27 | 1.70E+01 | 3.67E+00 | −1.36 | 0.91 | −1.03 | 0.85 |
| mmu-miR-6967-5p | yes | NO | yes | NO | 2.27 | 6.67E+00 | 9.33E+00 | 1.21 | −1.06 | −0.59 | −1.46 |
| mmu-miR-1934-3p | yes | yes | yes | NO | 2.25 | 8.67E+00 | 7.00E+00 | −0.55 | 1.70 | −0.50 | 0.37 |
| mmu-miR- [symbol] | yes | yes | yes | NO | 2.25 | 2.97E+01 | 1.80E+01 | −1.93 | 0.31 | −1.50 | 0.32 |
| mmu-miR-192-3p | yes | yes | yes | NO | 2.22 | 9.67E+00 | 4.00E+00 | −1.43 | 0.79 | −1.38 | 1.51 |
| mmu-miR- [symbol] | yes | yes | yes | NO | 2.21 | 6.00E+00 | 1.33E+00 | −1.47 | 0.74 | −1.70 | 1.10 |
| mmu-miR-6540-3p | yes | yes | yes | NO | 2.19 | 1.50E+01 | 5.00E+00 | −1.50 | 0.70 | −0.66 | 0.66 |
| mmu-let- [symbol] | yes | NO | NO | NO | 2.16 | 2.00E+00 | 1.33E+00 | −0.55 | 1.60 | 0.00 | −0.89 |
| mmu-miR-3069-3p | yes | yes | yes | NO | 2.11 | 2.33E+00 | 6.67E+01 | −1.51 | 0.60 | −0.45 | 1.00 |
| mmu-miR-877-3p | yes | yes | yes | NO | 2.11 | 3.30E+01 | 2.23E+01 | −0.81 | 1.30 | −0.93 | 0.73 |
| mmu-miR-27a-5p | yes | NO | yes | NO | 2.10 | 1.40E+01 | 2.07E+01 | 1.08 | −1.02 | −1.62 | −1.48 |
| mmu-miR-190a-3p | yes | yes | NO | NO | 2.09 | 1.83E+01 | 5.00E+00 | −1.37 | 0.72 | −1.01 | −0.60 |
| mmu-miR-219a-1-3p | yes | yes | NO | NO | 2.03 | 9.33E+00 | 5.33E+00 | −1.39 | 0.63 | −0.60 | −0.19 |
| mmu-miR-5123 | yes | yes | yes | NO | 2.02 | 2.40E+01 | 1.47E+01 | −1.06 | 0.96 | −1.13 | 0.04 |
| mmu-miR-3075-5p | yes | yes | NO | NO | 2.01 | 1.17E+01 | 7.33E+00 | −1.51 | 0.50 | −2.01 | −0.29 |
| mmu-miR-34a-3p | yes | yes | [symbol] | NO | 2.00 | 2.00E+00 | 1.00E+00 | −1.00 | 1.00 | −0.87 | [symbol] |
| mmu-miR-222-5p | yes | NO | yes | NO | 2.00 | 1.03E+01 | 1.57E+01 | 0.86 | −1.14 | −2.94 | −1.43 |
| mmu-miR-1306-3p | yes | yes | yes | NO | 2.00 | 5.00E+00 | 1.40E+01 | 0.97 | −1.02 | 0.78 | −0.92 |
| mmu-miR-669a-3-3p | yes | NO | yes | NO | 1.95 | 4.67E+00 | 2.67E+00 | −0.85 | 1.10 | 0.09 | 1.43 |
| mmu-miR-12194-3p | yes | yes | yes | NO | 1.93 | 1.00E+00 | 2.67E+00 | 1.58 | −0.35 | 0.76 | −2.12 |
| mmu-miR-6944-3p | yes | yes | yes | NO | 1.93 | 4.37E+01 | 1.70E+01 | −1.12 | 0.81 | −0.88 | 0.21 |
| mmu-miR-292a-5p | yes | yes | NO | NO | 1.92 | 2.67E+00 | 6.67E+01 | −1.60 | 0.32 | −0.89 | −0.71 |
| mmu-miR-1953 | yes | yes | NO | NO | 1.91 | 2.93E+01 | 2.07E+01 | −1.32 | 0.59 | −0.30 | −0.40 |
| mmu-miR-7063-5p | yes | yes | yes | NO | 1.90 | 2.67E+00 | 1.00E+00 | −1.58 | 0.32 | −0.50 | 0.6l |
| mmu-miR-5627-3p | yes | yes | yes | NO | 1.89 | 1.00E+00 | 1.33E+00 | 1.00 | −0.89 | 0.76 | −2.12 |
| mmu-miR-193a-3p | yes | yes | yes | NO | 1.87 | 3.83E+01 | 1.33E+01 | −1.55 | 0.31 | −1.43 | 0.38 |
| mmu-miR-501-5p | yes | yes | yes | NO | 1.85 | 5.40E+01 | 2.63E+01 | −1.19 | 0.66 | −0.78 | 0.31 |
| mmu-miR- [symbol] | yes | yes | NO | NO | 1.80 | 4.23E+01 | 2.10E+01 | −1.22 | 0.58 | −0.07 | −0.12 |
| mmu-miR-377-3p | yes | yes | yes | NO | 1.80 | 3.67E+01 | 3.20E+01 | 0.29 | 1.51 | 0.82 | 1.31 |
| mmu-miR-125a-3p | yes | NO | NO | NO | 1.77 | 1.37E+01 | 9.00E+00 | −0.62 | 1.15 | 0.21 | 0.00 |
| mmu-miR-5616-5p | yes | yes | yes | NO | 1.76 | 2.00E+00 | 6.67E+01 | −1.26 | 0.50 | −0.28 | 0.32 |
| mmu-miR-1929-3p | yes | NO | yes | NO | 1.76 | 1.67E+00 | 2.00E+00 | 0.25 | −1.51 | −0.80 | −0.76 |
| mmu-miR-3062-5p | yes | yes | NO | NO | 1.74 | 1.90E+01 | 8.67E+00 | −1.36 | 0.38 | −0.51 | −0.08 |
| mmu-miR-669m-3p | yes | yes | yes | NO | 1.74 | 4.67E+00 | 1.33E+00 | −0.85 | 0.89 | −0.14 | 1.00 |
| mmu-miR-7068-3p | yes | yes | NO | NO | 1.71 | 3.00E+00 | 1.33E+00 | −1.25 | 0.46 | −1.26 | −1.06 |
| mmu-miR- [symbol] | yes | yes | yes | NO | 1.71 | 4.00E+00 | 2.33E+00 | −0.69 | 1.01 | −0.69 | 0.91 |
| mmu-miR-485-3p | yes | NO | Yes | NO | 1.70 | 5.67E+00 | 8.67E+00 | 0.49 | −1.21 | −1.10 | −0.44 |
| mmu-miR-3058-3p | yes | NO | NO | NO | 1.70 | 1.33E+00 | 3.00E+00 | 1.39 | −0.32 | −2.00 | 0.00 |
| mmu-miR-203-3p | yes | yes | yes | NO | 1.67 | 4.00E+01 | 1.97E+01 | −1.34 | 0.33 | −1.32 | 0.37 |
| mmu-miR-675-5p | yes | yes | yes | NO | 1.65 | 6.33E+00 | 2.67E+00 | −1.40 | 0.25 | −0.76 | 0.42 |
| mmu-miR-3105-3p | yes | NO | yes | NO | 1.64 | 1.67E+00 | 2.33E+00 | 0.53 | −1.11 | 0.00 | −1.00 |
| mmu-miR-1199-5p | yes | yes | NO | NO | 1.63 | 1.47E+01 | 5.67E+00 | −1.39 | 0.25 | −0.55 | −0.91 |
| mmu-miR-486a-3p | yes | yes | yes | NO | 1.61 | 2.33E+00 | 1.00E+00 | −0.85 | 0.76 | −0.39 | 0.38 |
| mmu-miR-7682-3p | yes | yes | yes | NO | 1.60 | 1.20E+01 | 4.67E+00 | −1.48 | 0.12 | −0.88 | 0.09 |
| mmu-miR-185-3p | yes | NO | yes | NO | 1.59 | 7.00E+00 | 6.67E+00 | −0.11 | 1.49 | 0.00 | 0.11 |
| mmu-miR-744-3p | yes | yes | yes | NO | 1.56 | 6.00E+00 | 4.33E+00 | −0.50 | 1.06 | −0.08 | 0.63 |
| mmu-miR-148a-5p | yes | yes | yes | NO | 1.56 | 6.00E+00 | 4.00E+00 | −0.77 | 0.78 | −0.42 | 0.29 |
| mmu-miR-16-2-3p | yes | yes | NO | NO | 1.53 | 3.23E+01 | 2.03E+01 | −0.90 | 0.63 | −0.30 | −0.19 |
| mmu-miR-505-3p | yes | yes | NO | NO | 1.52 | 5.97E+01 | 3.67E+01 | −1.40 | 0.13 | −1.50 | −0.56 |
| mmu-miR-3102-3p.2-38 | yes | NO | NO | NO | 1.51 | 2.67E+00 | 1.67E+00 | −0.80 | 0.71 | 0.21 | −2.83 |
| mmu-miR-186-3p | yes | yes | NO | NO | 1.50 | 3.77E+01 | 2.30E+01 | −1.34 | 0.17 | −0.91 | −0.13 |
| mmu-miR-6986-5p | yes | yes | NO | NO | 1.50 | 1.67E+00 | 1.00E+00 | −1.00 | 0.50 | −1.00 | 0.00 |
| mmu-miR-1969 | yes | yes | NO | NO | 1.49 | 4.13E+01 | 2.33E+01 | −1.41 | 0.09 | −1.65 | −0.26 |
| mmu-miR- [symbol] | yes | yes | NO | NO | 1.47 | 9.67E+00 | 6.00E+00 | −1.17 | 0.30 | −0.69 | −0.17 |
| mmu-miR-330-5p | yes | yes | NO | NO | 1.46 | 5.13E+01 | 3.40E+01 | −1.37 | 0.08 | −0.22 | −0.38 |
| mmu-miR-1298-3p | yes | NO | yes | NO | 1.45 | 1.00E+00 | 2.00E+00 | 0.65 | −0.80 | 0.00 | −0.33 |
| mmu-miR-6997-5p | yes | yes | NO | NO | 1.44 | 3.67E+00 | 1.67E+00 | −1.06 | 0.38 | −1.86 | −0.76 |
| mmu-miR- [symbol] | yes | yes | yes | NO | 1.44 | 2.00E+01 | 1.03E+01 | −1.11 | 0.33 | −1.08 | 0.04 |
| mmu-miR-7226-3p | yes | NO | yes | NO | 1.43 | 2.33E+00 | 3.33E+00 | 0.80 | −0.63 | −1.06 | −1.51 |
| mmu-miR-1982-3p | yes | NO | yes | NO | 1.42 | 2.67E+00 | 5.00E+00 | 0.92 | −0.50 | −1.34 | −0.76 |
| mmu-miR-1981-3p | yes | NO | NO | NO | 1.40 | 5.67E+00 | 3.67E+00 | −0.90 | 0.50 | 0.11 | −0.55 |
| mmu-miR-7008-3p | yes | yes | yes | NO | 1.38 | 1.33E+00 | 2.33E+00 | 1.06 | −0.32 | 0.35 | −1.26 |
| mmu-miR-181a-2-3p | yes | yes | yes | NO | 1.38 | 4.43E+01 | 2.93E+01 | −1.00 | 0.38 | −0.07 | 0.02 |
| mmu-miR-145a-3p | yes | yes | yes | NO | 1.35 | 2.60E+01 | 1.50E+01 | −0.83 | 0.52 | −0.83 | 0.34 |
| mmu-miR-669p-3p | yes | yes | yes | NO | 1.34 | 2.80E+01 | 1.40E+01 | −0.96 | 0.39 | −1.09 | 0.39 |
| mmu-miR-1964-3p | yes | yes | yes | NO | 1.34 | 3.47E+01 | 2.40E+01 | −1.14 | 0.20 | −0.84 | 0.24 |
| mmu-miR- [symbol] | yes | yes | NO | NO | 1.33 | 5.33E+00 | 3.33E+00 | −0.57 | 0.76 | −0.19 | −0.63 |
| mmu-miR-351-3p | yes | NO | NO | NO | 1.32 | 1.77E+01 | 1.60E+01 | −0.55 | 0.77 | 0.04 | −0.46 |
| mmu-miR-127-5p | yes | yes | NO | NO | 1.30 | 2.17E+01 | 1.87E+01 | −0.31 | 1.00 | −0.99 | 0.00 |
| mmu-miR-448-3p | yes | yes | NO | NO | 1.30 | 2.30E+01 | 1.27E+01 | −1.24 | 0.06 | −1.52 | −0.13 |
| mmu-miR-26a-2-3p | yes | yes | NO | NO | 1.29 | 1.13E+01 | 7.67E+00 | −0.61 | 0.68 | −1.15 | −0.69 |
| mmu-miR-770-5p | yes | NO | yes | NO | 1.29 | 3.00E+00 | 3.67E+00 | 0.28 | −1.01 | 0.00 | −0.16 |
| mmu-miR- [symbol] | yes | yes | NO | NO | 1.29 | 3.37E+01 | 1.97E+01 | −1.19 | 0.10 | −0.48 | −0.07 |
| mmu-miR-187-5p | yes | NO | NO | NO | 1.28 | 2.33E+00 | 2.67E+00 | 0.22 | −1.06 | 0.00 | 0.34 |
| mmu-miR-1249-3p | yes | yes | NO | NO | 1.28 | 5.13E+01 | 3.77E+01 | −0.99 | 0.29 | −0.87 | 0.00 |
| mmu-miR-6945-3p | yes | yes | NO | NO | 1.28 | 5.33E+00 | 4.00E+00 | −0.69 | 0.59 | −0.85 | −0.29 |
| mmu-miR-676-5p | yes | yes | NO | NO | 1.26 | 5.27E+01 | 2.97E+01 | −1.12 | 0.14 | −2.51 | −0.02 |
| mmu-miR- [symbol] | yes | yes | yes | NO | 1.26 | 8.33E+00 | 7.00E+00 | −0.48 | 0.78 | −1.24 | 0.26 |
| mmu-miR-493-5p | yes | yes | NO | NO | 1.25 | 1.73E+01 | 1.60E+01 | −0.14 | 1.11 | −0.35 | −0.83 |
| mmu-miR-3105-5p | yes | yes | NO | NO | 1.24 | 9.67E+00 | 4.33E+00 | −0.99 | 0.25 | −0.76 | 0.00 |
| mmu-miR-466f | yes | yes | yes | NO | 1.24 | 2.00E+01 | 1.13E+01 | −0.71 | 0.53 | −0.10 | 0.49 |
| mmu-miR- [symbol] | yes | yes | yes | NO | 1.24 | 8.33E+00 | 5.67E+00 | −0.60 | 0.64 | −0.95 | 0.63 |
| mmu-miR-3080-5p | yes | yes | NO | NO | 1.23 | 2.33E+00 | 1.33E+00 | −0.80 | 0.43 | −1.00 | 0.00 |
| mmu-miR-6940-3p | yes | yes | NO | NO | 1.21 | 1.67E+00 | 2.67E+00 | 0.51 | −0.71 | 0.53 | 0.00 |
| mmu-miR-12202-3p | yes | NO | yes | NO | 1.21 | 6.00E+00 | 3.67E+00 | −0.88 | 0.34 | 0.00 | 0.19 |
| mmu-miR-99a-3p | yes | yes | yes | NO | 1.21 | 4.33E+00 | 1.67E+00 | −0.99 | 0.21 | −0.94 | 0.17 |
| mmu-miR-760-3p | yes | NO | NO | NO | 1.20 | 7.33E+00 | 9.67E+00 | 0.57 | −0.63 | −0.71 | 0.39 |
| mmu-miR-1188-5p | yes | NO | yes | NO | 1.19 | 9.00E+00 | 1.40E+01 | 0.92 | −0.27 | −0.71 | −0.06 |
| mmu-miR-92a-1-5p | yes | NO | yes | NO | 1.16 | 1.40E+01 | 2.10E+01 | 0.53 | −0.63 | −1.49 | −0.63 |
| mmu-miR-1197-3p | yes | yes | yes | NO | 1.15 | 2.00E+00 | 1.67E+00 | −0.25 | 0.90 | −0.20 | 0.38 |
| mmu-miR-3112-3p | yes | yes | NO | NO | 1.13 | 2.67E+00 | 2.00E+00 | −0.50 | 0.63 | −1.25 | −0.25 |
| mmu-miR- [symbol] | yes | yes | yes | NO | 1.13 | 3.33E+00 | 3.00E+00 | −0.28 | 0.85 | −1.34 | 0.43 |
| mmu-miR-8114 | yes | yes | yes | NO | 1.12 | 4.70E+01 | 3.33E+01 | −1.00 | 0.12 | −0.39 | 0.47 |
| mmu-miR-666-3p | yes | yes | NO | NO | 1.10 | 2.53E+01 | 2.23E+01 | −0.18 | 0.92 | −0.82 | −1.12 |
| mmu-miR- [symbol] | yes | yes | yes | NO | 1.09 | 1.00E+01 | 7.67E+00 | −0.40 | 0.69 | −1.42 | 0.19 |
| mmu-miR-26a-1-3p | yes | yes | NO | NO | 1.09 | 5.00E+00 | 4.00E+00 | −0.30 | 0.79 | −0.98 | −0.21 |
| mmu-miR-25-5p | yes | NO | yes | NO | 1.08 | 1.70E+01 | 1.90E+01 | 0.28 | −0.80 | −1.13 | −0.20 |
| mmu-miR-5p | yes | yes | NO | NO | 1.07 | 1.43E+01 | 1.00E+01 | −0.96 | 0.11 | −1.39 | −0.68 |
| mmu-miR-221-5p | yes | NO | yes | NO | 1.07 | 2.50E+01 | 3.10E+01 | 0.33 | −0.74 | −1.61 | −1.19 |
| mmu-miR-130a-5p | yes | yes | yes | NO | 1.04 | 6.67E+00 | 4.00E+00 | −0.88 | 0.16 | −0.40 | 0.53 |
| mmu-miR-3076-5p | yes | yes | yes | NO | 1.00 | 1.33E+00 | 1.00E+00 | −0.50 | 0.50 | −0.50 | 1.22 |
| mmu-miR-1187 | yes | NO | yes | NO | 0.99 | 1.33E+00 | 1.67E+00 | 0.27 | −0.73 | −4.00 | −0.73 |
| mmu-miR- [symbol] | yes | NO | NO | NO | 0.98 | 2.23E+01 | 1.80E+01 | −0.56 | 0.42 | 0.13 | −0.31 |
| mmu-miR-1934-5p | yes | NO | NO | NO | 0.96 | 1.93E+01 | 2.33E+01 | 0.51 | −0.45 | −0.59 | 0.44 |
| mmu-miR-383-5p | yes | NO | yes | NO | 0.96 | 3.00E+00 | 9.00E+00 | 0.70 | −0.26 | −0.15 | −0.37 |
| mmu-miR-1843a-3p | yes | yes | yes | NO | 0.95 | 1.33E+00 | 2.00E+00 | 0.63 | −0.32 | 0.32 | −1.22 |
| mmu-miR-8103 | yes | yes | yes | NO | 0.95 | 1.27E+01 | 8.33E+00 | −0.87 | 0.08 | −0.59 | 0.15 |
| mmu-miR-3064-5p | yes | yes | yes | NO | 0.95 | 6.00E+00 | 7.00E+00 | 0.71 | −0.24 | 0.40 | −3.27 |
| mmu-miR-338-3p | yes | yes | yes | NO | 0.91 | 4.53E+01 | 3.03E+01 | −0.76 | 0.15 | −1.37 | 0.32 |
| mmu-miR-138-2-3p | yes | yes | NO | NO | 0.90 | 5.00E+00 | 4.33E+00 | −0.63 | 0.27 | −1.58 | 0.00 |
| mmu-miR- [symbol] | yes | yes | yes | NO | 0.90 | 1.70E+01 | 1.50E+01 | −0.20 | 0.69 | −0.09 | 0.34 |
| mmu-miR-661-5p | yes | yes | yes | NO | 0.89 | 1.67E+01 | 2.07E+01 | 0.52 | 0.37 | 0.27 | 2.82 |
| mmu-miR-23a-5p | yes | NO | yes | NO | 0.83 | 5.00E+00 | 5.33E+00 | 0.13 | −0.71 | −0.63 | −0.54 |
| mmu-miR- [symbol] | yes | yes | NO | NO | 0.81 | 8.67E+00 | 5.67E+00 | −0.48 | 0.33 | −0.73 | −0.11 |
| mmu-miR- [symbol] | yes | NO | NO | NO | 0.80 | 2.00E+00 | 1.67E+00 | −0.21 | 0.59 | 0.50 | −0.73 |
| mmu-miR-6958-3p | yes | yes | NO | NO | 0.78 | 4.33E+00 | 4.00E+00 | −0.32 | 0.46 | −0.35 | −0.18 |
| mmu-miR-7018-3p | yes | yes | yes | NO | 0.76 | 7.00E+00 | 7.67E+00 | 0.38 | −0.38 | 0.15 | −1.58 |
| mmu-miR-466k | yes | yes | yes | NO | 0.73 | 1.70E+01 | 1.47E+01 | −0.28 | 0.45 | −0.18 | 0.17 |
| mmu-miR-6960-5p | yes | NO | yes | NO | 0.69 | 5.67E+00 | 6.00E+00 | 0.21 | −0.48 | −1.96 | −0.69 |
| mmu-miR- [symbol] | yes | yes | yes | NO | 0.69 | 1.77E+01 | 1.83E+01 | 0.10 | −0.59 | 0.07 | −0.20 |
| mmu-miR-3061-5p | yes | yes | yes | NO | 0.67 | 7.67E+00 | 7.33E+00 | −0.11 | 0.56 | −2.20 | 0.09 |
| mmu-miR-412-5p | yes | NO | yes | NO | 0.66 | 1.37E+01 | 1.40E+01 | 0.04 | −0.63 | −0.40 | −0.68 |
| mmu-miR- [symbol] | yes | NO | yes | NO | 0.66 | 1.80E+01 | 2.10E+01 | 0.28 | −0.38 | −0.12 | −0.38 |
| mmu-miR-452-5p | yes | yes | NO | NO | 0.66 | 1.17E+01 | 9.00E+00 | −0.54 | 0.12 | −0.13 | −0.72 |
| mmu-miR-1930-3p | yes | NO | NO | NO | 0.63 | 2.33E+00 | 2.00E+00 | −0.32 | 0.32 | 1.58 | −0.32 |
| mmu-let- [symbol] | yes | NO | yes | NO | 0.63 | 4.43E+01 | 4.77E+01 | 0.50 | −0.13 | −0.05 | −0.26 |
| mmu-miR-139-5p | yes | NO | yes | NO | 0.57 | 8.67E+00 | 9.33E+00 | 0.14 | −0.43 | −0.71 | −0.28 |
| [symbol] | yes | NO | yes | NO | 0.57 | 5.67E+00 | 7.00E+00 | 0.21 | 0.36 | 0.06 | 0.96 |
| mmu-miR-183-3p | yes | yes | yes | NO | 0.55 | 3.33E+00 | 3.00E+00 | −0.09 | 0.46 | −0.85 | 0.20 |
| mmu-miR-1968-5p | yes | NO | yes | NO | 0.54 | 7.33E+00 | 8.33E+00 | 0.30 | −0.24 | −0.17 | −0.97 |
| mmu-miR- [symbol] | yes | yes | NO | NO | 0.54 | 1.70E+01 | 1.17E+01 | −0.43 | 0.11 | −0.74 | −0.02 |
| mmu-miR-669o-5p | yes | yes | yes | NO | 0.53 | 3.33E+00 | 3.00E+00 | −0.13 | 0.40 | −0.45 | 0.32 |
| mmu-miR-671-3p | yes | NO | Yes | NO | 0.48 | 1.03E+01 | 1.33E+01 | 0.44 | −0.04 | −0.51 | −0.14 |
| mmu-miR-467d-5p | yes | yes | NO | NO | 0.45 | 4.90E+01 | 4.53E+01 | −0.18 | 0.28 | −1.47 | −0.29 |
| mmu-miR-132-5p | yes | yes | yes | NO | 0.39 | 1.67E+00 | 2.33E+00 | 0.25 | −0.14 | 0.20 | −0.13 |
| mmu-miR-466n-5p | yes | NO | NO | NO | 0.38 | 2.67E+00 | 2.33E+00 | −0.14 | 0.24 | 0.45 | 0.00 |
| mmu-miR-466m-3p | yes | NO | NO | NO | 0.32 | 4.17E+01 | 4.23E+01 | 0.02 | −0.30 | −0.84 | 0.01 |
| mmu-miR-137-3p | yes | yes | NO | NO | 0.32 | 1.03E+01 | 1.23E+01 | 0.21 | −0.11 | 1.07 | 0.08 |
| mmu-miR-142a-5p | yes | yes | yes | NO | 0.28 | 3.07E+01 | 2.90E+01 | −0.18 | 0.11 | −1.49 | 0.54 |
| mmu-miR- [symbol] | yes | yes | NO | NO | 0.26 | 7.67E+00 | 7.33E+00 | −0.10 | 0.16 | −0.18 | −0.06 |
| mmu-miR-1264-3p | yes | NO | yes | NO | 0.14 | 5.30E+01 | 5.40E+01 | 0.04 | −0.10 | −1.80 | −0.23 |
| mmu-miR-1950 | NO | yes | yes | NO | 4.84 | 4.00E+00 | 1.67E+00 | −3.50 | −1.34 | −1.07 | 0.67 |
| mmu-miR-3095-3p | NO | yes | NO | NO | 4.35 | 1.90E+01 | 1.60E+01 | −0.41 | −3.94 | −0.86 | −2.04 |
| mmu-miR-1947-5p | NO | yes | NO | NO | 4.00 | 1.50E+01 | 8.33E+00 | −2.44 | −1.56 | −2.01 | −0.24 |
| mmu-miR-3066-5p | NO | yes | yes | NO | 3.73 | 2.77E+01 | 1.30E+01 | −3.65 | −0.07 | −2.79 | 0.44 |
| mmu-miR-7212-5p | NO | yes | NO | NO | 3.60 | 4.00E+00 | 1.00E+00 | −2.60 | −1.00 | −2.60 | −0.50 |
| mmu-miR- [symbol] | NO | yes | NO | NO | 3.50 | 4.00E+00 | 6.67E+01 | −2.50 | −1.00 | −3.46 | −1.00 |
| mmu-miR- [symbol] | NO | yes | NO | NO | 3.50 | 4.00E+00 | 6.67E+01 | −2.50 | −1.00 | −3.46 | −1.00 |
| mmu-miR- [symbol] | NO | yes | NO | NO | 3.50 | 2.00E+00 | 3.33E+01 | −2.50 | −1.00 | −2.50 | 0.00 |
| mmu-miR-465a-3p | NO | yes | NO | NO | 3.50 | 2.00E+00 | 3.33E+01 | −2.50 | −1.00 | −3.46 | −1.00 |
| mmu-miR- [symbol] | NO | yes | yes | NO | 3.24 | 7.33E+00 | 6.67E+01 | −2.24 | −1.00 | −1.46 | 0.45 |
| mmu-miR-384-5p | NO | yes | yes | NO | 3.12 | 1.33E+00 | 3.33E+01 | −2.12 | −1.00 | −2.12 | 0.45 |
| mmu-miR-7661-3p | NO | yes | yes | NO | 3.11 | 4.00E+00 | 1.33E+00 | −2.22 | −0.89 | −1.22 | 0.67 |
| mmu-miR-6916-5p | NO | yes | NO | NO | 3.11 | 2.33E+00 | 1.00E+00 | −1.11 | −2.00 | −1.94 | −2.00 |
| mmu-miR-7688-5p | NO | yes | NO | NO | 2.92 | 3.13E+01 | 2.03E+01 | −1.94 | −0.93 | −2.03 | −0.14 |
| mmu-miR-106a-5p | NO | yes | yes | NO | 2.89 | 5.33E+00 | 1.67E+00 | −2.46 | −0.43 | −2.83 | 0.14 |
| mmu-miR- [symbol] | NO | yes | NO | NO | 2.78 | 4.77E+01 | 2.13E+01 | −2.22 | −0.56 | −1.43 | −0.14 |
| mmu-miR-3061-3p | NO | yes | yes | NO | 2.69 | 8.67E+00 | 3.33E+00 | −1.09 | −1.60 | −1.11 | 0.42 |
| mmu-miR-666-5p | NO | yes | yes | NO | 2.61 | 6.00E+00 | 1.60E+01 | 1.85 | 0.76 | 0.74 | −0.50 |
| mmu-miR-1336-3p | NO | yes | NO | NO | 2.58 | 6.67E+00 | 3.00E+00 | −2.20 | −0.38 | −2.92 | −0.50 |
| mmu-miR-376a-5p | NO | NO | yes | NO | 2.56 | 3.00E+00 | 4.67E+00 | 1.07 | 1.49 | 0.00 | −0.43 |
| mmu-miR-7655-3p | NO | yes | yes | NO | 2.44 | 2.00E+00 | 1.67E+00 | −0.32 | −2.12 | −0.50 | 0.21 |
| mmu-miR-7009-5p | NO | NO | NO | NO | 2.39 | 3.33E+01 | 1.33E+00 | 2.12 | 0.27 | 0.00 | 0.35 |
| mmu-miR-6913-5p | NO | yes | NO | NO | 2.38 | 1.33E+00 | 1.00E+00 | −0.38 | −2.00 | −0.71 | −0.50 |
| mmu-miR-879-5p | NO | NO | yes | NO | 2.34 | 1.33E+00 | 3.33E+01 | −1.34 | −1.00 | 0.00 | 1.06 |
| mmu-miR-380-3p | NO | NO | yes | NO | 2.34 | 1.10E+01 | 1.73E+01 | 1.73 | 0.61 | −0.23 | −0.31 |
| mmu-miR-144-3p | NO | yes | yes | NO | 2.33 | 1.97E+01 | 9.67E+00 | −1.93 | −0.40 | −0.69 | 1.17 |
| mmu-miR-295-3p | NO | yes | NO | NO | 2.28 | 4.00E+00 | 6.67E+01 | −1.28 | −1.00 | −0.77 | 0.00 |
| mmu-miR-3072-3p | NO | NO | yes | NO | 7.75 | 2.33E+00 | 4.00E+00 | 1.25 | 1.00 | −0.71 | −1.00 |
| mmu-miR- [symbol] | NO | yes | NO | NO | 2.23 | 0.00E+00 | 1.00E+00 | 1.73 | 0.50 | 1.00 | 0.00 |
| mmu-miR-6910-3p | NO | yes | yes | NO | 2.20 | 3.00E+00 | 6.67E+01 | −1.75 | −0.45 | −1.55 | 0.73 |
| mmu-miR-6937-3p | NO | yes | NO | NO | 2.12 | 1.33E+00 | 3.33E+01 | −2.12 | 0.00 | −2.12 | 0.00 |
| mmu-miR-6984-3p | NO | yes | NO | NO | 2.11 | 0.00E+00 | 1.00E+00 | 1.73 | 0.38 | 2.50 | 0.00 |
| mmu-mik-215-5p | NO | yes | yes | NO | 2.07 | 2 07E+01 | 7.33E+00 | −1.84 | −0.23 | −1.64 | 0.19 |
| mmu-miR- [symbol] | NO | yes | yes | NO | 2.06 | 4 00E+01 | 2.40E+01 | −1.95 | −0.11 | −5.60 | 0.02 |
| mmu-miR-3068-3p | NO | yes | NO | NO | 2.03 | 5.33E+00 | 2.67E+00 | −1.21 | −0.82 | −0.93 | 0.00 |
| mmu-miR-7060-5p | NO | yes | yes | NO | 2.00 | 1.67E+00 | 1.00E+00 | −1.00 | −1.00 | −1.34 | 0.50 |
| mmu-miR-1912-3p | NO | yes | yes | NO | 2.00 | 2.00E+00 | 6.67E+01 | −2.00 | 0.00 | −0.46 | 0.85 |
| mmu-miR-7065-3p | NO | yes | yes | NO | 2.00 | 1.00E+00 | 3.33E+01 | −2.00 | 0.00 | −2.00 | −1.00 |
| mmu-miR-1945 | NO | yes | yes | NO | 2.00 | 1.57E+01 | 1.07E+01 | −1.82 | −0.18 | −0.24 | 0.45 |
| mmu-miR-184-3p | NO | yes | NO | NO | 1.99 | 3.67E+00 | 2.33E+00 | −0.65 | −1.34 | −0.53 | −1.12 |
| mmu-miR- [symbol] | NO | NO | yes | NO | 1.97 | 2.50E+01 | 2.67E+01 | 0.11 | 1.86 | −0.65 | −0.22 |
| mmu-miR-543-5p | NO | yes | NO | NO | 1.91 | 2.00E+00 | 4.00E+00 | 0.87 | 1.04 | 0.35 | 0.19 |
| mmu-miR-342-5p | NO | yes | NO | NO | 1.89 | 3.00E+00 | 2.33E+00 | −1.00 | −0.89 | −1.00 | −0.89 |
| mmu-miR-32-3p | NO | yes | NO | NO | 1.87 | 3.67E+00 | 2.67E+00 | 0.53 | 1.31 | 0.35 | 0.13 |
| mmu-miR-1933-3p | NO | yes | NO | NO | 1.86 | 8.33E+00 | 5.33E+00 | −0.99 | −0.88 | −1.66 | −0.22 |
| mmu-miR-7025-3p | NO | NO | yes | NO | 1.86 | 1.33E+00 | 2.33E+00 | 1.06 | 0.80 | −2.12 | −2.12 |
| mmu-miR-7075-3p | NO | yes | yes | NO | 1.86 | 4.33E+00 | 1.67E+00 | −1.41 | −0.45 | −1.86 | 0.21 |
| mmu-miR-202-5p | NO | yes | NO | NO | 1.85 | 3.00E+00 | 6.67E+01 | −1.40 | −0.45 | −0.85 | −0.45 |
| mmu-miR-5134-5p | NO | yes | NO | NO | 1.80 | 2.67E+00 | 1.67E+00 | −0.80 | −1.00 | −0.20 | 0.00 |
| mmu-miR-7068-5p | NO | NO | NO | NO | 1.78 | 8.33E+00 | 8.00E+00 | −0.10 | −1.68 | 0.00 | −1.16 |
| mmu-miR-3089-3p | NO | yes | NO | NO | 1.77 | 1.23E+01 | 8.33E+00 | −1.05 | −0.72 | −2.03 | −0.45 |
| mmu-miR-466p-5p | NO | yes | yes | NO | 1.76 | 1.00E+01 | 5.00E+00 | −1.26 | −0.49 | −0.44 | 0.55 |
| mmu-miR-1970 | yes | NO | NO | NO | 1.73 | 1.00E+00 | 1.00E+00 | 0.00 | 1.73 | 0.00 | −0.50 |
| mmu-miR-874-3p | NO | NO | NO | NO | 1.71 | 9.00E+00 | 1.50E+01 | 1.16 | 0.55 | −0.34 | 0.07 |
| mmu-miR-375-3p | NO | yes | NO | NO | 1.71 | 9.00E+00 | 4.67E+00 | −0.92 | −0.79 | −1.29 | −0.36 |
| mmu-miR-7070-3p | NO | yes | NO | NO | 1.71 | 1.00E+00 | 6.67E+01 | −1.00 | −0.71 | −1.00 | −0.71 |
| mmu-miR-3474 | NO | yes | NO | NO | 1.70 | 3.33E+01 | 1.33E+00 | 1.34 | 0.35 | 0.71 | 0.00 |
| mmu-miR-3108-5p | NO | yes | NO | NO | 1.68 | 4.10E+01 | 3.07E+01 | −1.07 | −0.62 | −0.87 | −0.09 |
| mmu-miR-270-3p | NO | NO | yes | NO | 1.64 | 4.40E+01 | 5.83E+01 | 0.64 | 1.00 | −0.45 | −0.79 |
| mmu-miR-429-3p | NO | yes | yes | NO | 1.63 | 5.80E+01 | 3.63E+01 | −0.97 | −0.65 | −0.72 | 0.10 |
| mmu-miR-18a-3p | NO | NO | yes | NO | 1.62 | 5.20E+01 | 4.40E+01 | −0.36 | −1.27 | 0.04 | 0.53 |
| mmu-miR-1958 | NO | yes | NO | yes | 1.60 | 2.67E+00 | 6.67E+01 | −1.60 | 0.00 | −1.39 | −0.71 |
| mmu-miR-6418-3p | NO | yes | yes | NO | 1.60 | 2.00E+00 | 1.00E+00 | −1.22 | −0.38 | −2.50 | 0.63 |
| mmu-miR-672-3p | NO | yes | NO | NO | 1.57 | 5.67E+00 | 3.00E+00 | −0.87 | −0.71 | −0.90 | −0.69 |
| mmu-miR-1941-5p | NO | yes | NO | NO | 1.53 | 1.03E+01 | 8.00E+00 | −0.92 | −0.61 | −1.86 | −0.76 |
| mmu-miR-8112 | NO | yes | yes | NO | 1.51 | 3.67E+00 | 1.33E+00 | −1.24 | −0.28 | −0.71 | 1.06 |
| mmu-miR-6948-3p | NO | yes | NO | NO | 1.51 | 2.00E+00 | 6.67E+01 | −1.51 | 0.00 | −1.51 | 0.00 |
| mmu-miR-3079-3p | NO | NO | NO | NO | 1.51 | 1.33E+00 | 1.33E+00 | 0.00 | −1.51 | 2.50 | −1.06 |
| mmu-miR-7235-3p | NO | yes | yes | NO | 1.50 | 1.00E+00 | 6.67E+01 | −0.50 | −1.00 | −2.00 | 0.89 |
| mmu-miR-380-5p | NO | NO | yes | NO | 1.49 | 2.03E+01 | 2.97E+01 | 0.86 | 0.63 | −0.63 | −1.29 |
| mmu-miR-667-3p | NO | NO | NO | NO | 1.47 | 1.30E+01 | 2.40E+01 | 1.03 | 0.45 | −0.56 | 0.03 |
| mmu-miR-210-5p | NO | yes | NO | NO | 1.45 | 5.33E+00 | 4.67E+00 | −0.34 | −1.11 | −1.11 | −0.14 |
| mmu-miR-3063-3p | NO | yes | yes | NO | 1.43 | 9.33E+00 | 8.00E+00 | −0.51 | −0.92 | −0.90 | 0.25 |
| mmu-miR- [symbol] | NO | yes | yes | NO | 1.42 | 1.70E+01 | 1.23E+01 | −0.91 | −0.50 | −1.12 | 0.74 |
| mmu-miR- [symbol] | NO | yes | NO | NO | 1.39 | 1.50E+01 | 1.10E+01 | −0.77 | −0.51 | −0.97 | −0.85 |
| mmu-miR- [symbol] | NO | yes | NO | NO | 1.37 | 5.00E+00 | 2.33E+00 | −1.37 | 0.00 | −1.04 | −0.20 |
| mmu-miR- [symbol] | NO | NO | NO | NO | 1.37 | 7.33E+00 | 6.67E+00 | −0.13 | −1.24 | 2.00 | −0.90 |
| mmu-miR-485-5p | NO | yes | NO | NO | 1.37 | 1.03E+01 | 1.73E+01 | 0.64 | 0.73 | 0.04 | 0.17 |
| mmu-miR-1948-3p | NO | yes | NO | NO | 1.36 | 1.67E+00 | 1.33E+00 | −0.30 | −1.06 | −0.76 | −0.32 |
| mmu-miR-1948-5p | NO | NO | yes | NO | 1.34 | 6.67E+01 | 1.67E+00 | 1.34 | 0.00 | −2.00 | −1.06 |
| mmu-miR-103-2-5p | NO | yes | NO | NO | 1.34 | 3.33E+01 | 6.67E+01 | 0.45 | 0.89 | 0.45 | 0.00 |
| mmu-miR-3074-5p | NO | yes | yes | NO | 1.34 | 3.90E+01 | 2.60E+01 | −1.07 | −0.27 | −0.81 | 0.21 |
| mmu-miR-433-3p | NO | NO | yes | NO | 1.33 | 1.93E+01 | 3.20E+01 | 1.18 | 0.15 | −0.94 | −0.30 |
| mmu-miR-665-3p | NO | NO | yes | NO | 1.29 | 4.70E+01 | 5.97E+01 | 0.58 | 0.71 | −0.80 | −0.80 |
| mmu-miR-297a-5p | NO | NO | yes | NO | 1.27 | 2.00E+00 | 1.00E+00 | −0.77 | −0.50 | 0.52 | 0.32 |
| mmu-miR-6540-5p | NO | yes | NO | NO | 1.27 | 2.40E+01 | 1.43E+01 | −0.86 | −0.41 | −0.93 | −0.29 |
| mmu-miR-5103 | NO | yes | NO | NO | 1.26 | 2.00E+00 | 6.67E+01 | −1.26 | 0.00 | −1.58 | −0.71 |
| mmu-miR-3066-3p | NO | yes | NO | NO | 1.25 | 9.67E+00 | 6.67E+00 | −1.01 | −0.24 | −1.51 | −0.31 |
| mmu-miR-7659-5p | NO | NO | NO | NO | 1.25 | 2.33E+00 | 1.67E+00 | −0.45 | −0.80 | 0.00 | 0.00 |
| mmu-miR-3094-5p | NO | NO | NO | NO | 1.23 | 1.00E+00 | 1.33E+00 | 0.23 | 1.00 | −1.00 | 0.21 |
| mmu-miR- [symbol] | NO | yes | yes | NO | 1.21 | 5.00E+00 | 4.00E+00 | −0.34 | −0.88 | −0.21 | 0.37 |
| mmu-miR-337-3p | NO | NO | yes | NO | 1.17 | 4.90E+01 | 5.73E+01 | 0.39 | 0.78 | −0.89 | −0.37 |
| mmu-miR-5624-3p | NO | yes | NO | NO | 1.17 | 2.33E+00 | 1.33E+00 | −0.67 | −0.50 | −1.12 | −0.89 |
| mmu-miR-496a-3p | NO | NO | yes | NO | 1.12 | 6.00E+00 | 8.67E+00 | 0.69 | 0.44 | −0.57 | −0.79 |
| mmu-miR-499-5p | NO | yes | NO | NO | 1.10 | 1.50E+01 | 1.23E+01 | −0.49 | −0.61 | −2.47 | −0.15 |
| mmu-miR-491-3p | NO | yes | NO | NO | 1.10 | 3.00E+00 | 2.00E+00 | −0.46 | −0.63 | −0.21 | −1.73 |
| mmu-miR-329-3p | NO | NO | yes | NO | 1.09 | 2.27E+01 | 3.70E+01 | 1.04 | 0.05 | −0.45 | −0.76 |
| mmu-miR-7689-3p | NO | yes | NO | NO | 1.08 | 2.23E+01 | 1.50E+01 | −0.79 | −0.28 | −0.75 | −0.20 |
| mmu-miR- [symbol] | NO | NO | yes | NO | 1.06 | 3.43E+01 | 5.10E+01 | 0.83 | 0.23 | −0.57 | −1.12 |
| mmu-miR-3112-5p | NO | yes | NO | NO | 1.06 | 2.33E+00 | 1.33E+00 | −1.06 | 0.00 | −3.54 | −1.06 |
| mmu-miR-877-5p | NO | yes | yes | NO | 1.05 | 3.70E+01 | 2.50E+01 | −0.83 | −0.21 | −0.88 | 0.04 |
| mmu-miR- [symbol] | NO | yes | yes | NO | 1.03 | 2.10E+01 | 1.60E+01 | −0.46 | −0.58 | −0.51 | 0.30 |
| mmu-miR-3110-5p | NO | yes | NO | NO | 1.02 | 3.30E+01 | 2.37E+01 | −0.97 | −0.06 | −0.88 | −0.16 |
| mmu-miR-194-2-3p | NO | NO | yes | NO | 1.01 | 2.00E+00 | 2.67E+00 | 0.76 | 0.25 | 0.00 | −1.41 |
| mmu-miR-6988-3p | NO | NO | NO | NO | 1.00 | 1.67E+00 | 1.00E+00 | −1.00 | 0.00 | 0.66 | −1.00 |
| mmu-miR-702-5p | NO | NO | NO | NO | 1.00 | 3.33E+01 | 3.33E+01 | 0.00 | −1.00 | 2.00 | 0.00 |
| mmu-miR-7024-5p | NO | NO | NO | NO | 1.00 | 1.33E+00 | 1.33E+00 | 0.00 | −1.00 | −1.06 | −0.89 |
| mmu-miR-191-3p | NO | NO | NO | NO | 0.94 | 1.33E+00 | 1.33E+00 | 0.00 | 0.94 | 0.00 | 0.18 |
| mmu-miR-345-5p | NO | NO | NO | NO | 0.92 | 5.73E+01 | 5.00E+01 | −0.46 | −0.46 | 0.01 | −0.33 |
| mmu-miR-3077-3p | NO | yes | yes | NO | 0.90 | 1.67E+00 | 6.67E+01 | −0.90 | 0.00 | −0.63 | 0.89 |
| mmu-miR-144-5p | NO | yes | NO | NO | 0.90 | 7.67E+00 | 6.33E+00 | −0.38 | −0.52 | −0.94 | −0.09 |
| mmu-miR-7667-3p | NO | NO | NO | NO | 0.89 | 6.67E+01 | 6.67E+01 | 0.00 | 0.89 | −0.71 | 0.00 |
| mmu-miR-20a-3p | NO | yes | NO | NO | 0.89 | 1.87E+01 | 1.33E+01 | −0.72 | −0.16 | −2.58 | −0.27 |
| mmu-miR-218-1-3p | NO | yes | yes | NO | 0.88 | 2.47E+01 | 1.73E+01 | −0.84 | −0.04 | −0.41 | 0.05 |
| mmu-miR-466a-5p | NO | yes | yes | NO | 0.87 | 1.17E+01 | 8.00E+00 | −0.72 | −0.15 | −0.54 | 0.43 |
| mmu-miR-350-5p | NO | yes | NO | NO | 0.84 | 1.70E+01 | 1.13E+01 | −0.63 | −0.21 | −1.09 | −0.08 |
| mmu-miR-3069-5p | NO | yes | yes | NO | 0.83 | 1.00E+00 | 6.67E+01 | −0.38 | −0.45 | −0.50 | 0.38 |
| mmu-miR-543-3p | NO | NO | yes | NO | 0.78 | 1.87E+01 | 2.40E+01 | 0.66 | 0.12 | −0.20 | −0.24 |
| mmu-miR-6952-3p | NO | NO | NO | NO | 0.78 | 1.67E+00 | 2.33E+00 | 0.42 | 0.36 | 0.00 | 0.20 |
| mmu-miR- [symbol] | NO | yes | NO | NO | 0.77 | 1.17E+01 | 1.10E+01 | −0.17 | −0.60 | −0.62 | −0.12 |
| mmu-miR-7033-5p | NO | yes | NO | NO | 0.73 | 2.67E+00 | 2.33E+00 | −0.15 | −0.59 | −0.33 | −1.34 |
| mmu-miR-702-3p | NO | yes | NO | NO | 0.68 | 2.50E+01 | 2.23E+01 | −0.51 | −0.17 | −0.84 | −0.8I |
| mmu-miR-3060-3p | NO | yes | NO | NO | 0.68 | 1.53E+01 | 1.30E+01 | −0.36 | −0.32 | −0.66 | −0.20 |
| mmu-miR-133a-3p | NO | yes | NO | NO | 0.66 | 2.20E+01 | 1.87E+01 | −0.25 | −0.40 | −0.20 | −0.74 |
| mmu-miR-6975-3p | NO | NO | yes | NO | 0.65 | 2.00E+00 | 3.00E+00 | 0.65 | 0.00 | −0.17 | −0.92 |
| mmu-miR-5128 | NO | NO | yes | NO | 0.63 | 4.33E+00 | 5.33E+00 | 0.40 | 0.23 | −1.01 | −0.76 |
| mmu-miR-5626-5p | NO | yes | yes | NO | 0.63 | 1.67E+00 | 1.00E+00 | −0.63 | 0.00 | −1.06 | 0.32 |
| mmu-miR-365-2-5p | NO | NO | yes | NO | 0.63 | 1.00E+00 | 1.67E+00 | 0.63 | 0.00 | −0.38 | −1.06 |
| mmu-miR-1960 | NO | yes | NO | NO | 0.61 | 4.33E+00 | 3.67E+00 | −0.27 | −0.34 | −0.80 | −0.51 |
| mmu-miR- [symbol] | NO | yes | yes | NO | 0.57 | 8.67E+00 | 1.00E+01 | 0.25 | 0.33 | 0.44 | −0.14 |
| mmu-miR- [symbol] | NO | yes | NO | NO | 0.57 | 3.00E+00 | 2.33E+00 | −0.36 | −0.21 | −1.15 | −0.76 |
| mmu-miR- [symbol] | NO | yes | yes | NO | 0.56 | 1.17E+01 | 1.13E+01 | −0.06 | −0.50 | −0.24 | 0.04 |
| mmu-miR- [symbol] | NO | NO | NO | NO | 0.56 | 2.33E+00 | 2.33E+00 | 0.00 | 0.56 | 0.29 | −0.20 |
| mmu-miR- [symbol] | NO | yes | NO | NO | 0.55 | 1.67E+00 | 1.00E+00 | −0.55 | 0.00 | −1.00 | 0.00 |
| mmu-miR-6913-3p | NO | NO | NO | NO | 0.55 | 1.00E+00 | 1.00E+00 | 0.00 | 0.55 | −1.00 | −2.00 |
| mmu-miR-6967-3p | NO | NO | yes | NO | 0.52 | 2.00E+01 | 1.47E+01 | −0.46 | −0.06 | 0.35 | 0.29 |
| mmu-miR-679-5p | NO | yes | yes | NO | 0.50 | 6.67E+01 | 1.00E+00 | 0.50 | 0.00 | 0.32 | −1.73 |
| mmu-miR-212-5p | NO | yes | yes | NO | 0.49 | 1.80E+01 | 1.77E+01 | −0.03 | −0.46 | −1.18 | 0.08 |
| mmu-miR-467a-3p | NO | yes | yes | NO | 0.49 | 6.67E+00 | 1.33E+01 | 0.49 | 0.00 | 0.50 | −0.73 |
| mmu-miR-495-3p | NO | NO | NO | NO | 0.48 | 8.67E+00 | 9.00E+00 | 0.08 | 0.40 | −0.82 | 0.13 |
| mmu-miR-3098-5p | NO | yes | NO | NO | 0.46 | 5.33E+00 | 5.00E+00 | −0.25 | −0.21 | −0.28 | −0.55 |
| mmu-miR-669f-5p | NO | yes | NO | NO | 0.44 | 1.00E+00 | 1.33E+00 | 0.20 | 0.24 | 1.49 | 0.38 |
| mmu-miR-668-3p | NO | NO | NO | NO | 0.42 | 3.00E+00 | 3.33E+00 | 0.10 | 0.31 | −1.49 | 0.34 |
| mmu-miR- [symbol] | NO | yes | yes | NO | 0.40 | 5.00E+00 | 4.67E+00 | −0.12 | −0.28 | −0.17 | 0.34 |
| mmu-miR-1930-5p | NO | yes | NO | NO | 0.38 | 4.00E+00 | 3.33E+00 | −0.38 | 0.00 | 0.71 | −1.60 |
| mmu-miR-3072-5p | NO | NO | NO | NO | 0.38 | 1.00E+00 | 1.00E+00 | 0.00 | −0.38 | 0.00 | 0.00 |
| mmu-miR-6936-3p | NO | NO | yes | NO | 0.38 | 1.00E+00 | 1.33E+00 | 0.38 | 0.00 | −0.38 | −0.71 |
| mmu-miR- [symbol] | NO | NO | NO | NO | 0.36 | 6.33E+00 | 6.33E+00 | 0.00 | 0.36 | −0.56 | −0.75 |
| mmu-miR-467d-3p | NO | yes | NO | NO | 0.32 | 3.20E+01 | 2.83E+01 | −0.21 | −0.11 | −0.71 | −0.01 |
| mmu-miR- [symbol] | NO | NO | NO | NO | 0.25 | 2.33E+00 | 2.00E+00 | −0.25 | 0.00 | 0.00 | 0.25 |
| mmu-miR-1943-5p | NO | NO | NO | NO | 0.23 | 5.33E+00 | 5.33E+00 | 0.00 | 0.23 | −1.07 | −0.08 |
| mmu-miR-7224-3p | NO | yes | yes | NO | 0.22 | 1.93E+01 | 1.87E+01 | −0.06 | −0.17 | −1.07 | 0.30 |
| mmu-miR-147-5p | NO | yes | NO | NO | 0.20 | 1.03E+01 | 9.67E+00 | −0.13 | −0.07 | −0.31 | −0.16 |
| mmu-miR-181a-1-3p | NO | NO | yes | NO | 0.18 | 2.47E+01 | 2.53E+01 | 0.10 | 0.08 | −0.20 | −0.12 |
| mmu-miR-3088-3p | NO | NO | NO | NO | 0.18 | 3.33E+00 | 3.33E+00 | 0.00 | 0.18 | −0.45 | −1.34 |
| [symbol] | NO | NO | NO | NO | 0.10 | 3.67E+00 | 3.67E+00 | 0.00 | 0.10 | 1.39 | 0.00 |
| mmu-miR-3076-3p | NO | NO | NO | NO | 0.00 | 1.67E+00 | 1.67E+00 | 0.00 | 0.00 | 0.25 | 0.00 |
| mmu-miR-331-5p | NO | NO | NO | NO | 0.00 | 1.33E+00 | 1.33E+00 | 0.00 | 0.00 | 0.56 | 0.66 |
| mmu-miR-1955-5p | NO | NO | NO | NO | 0.00 | 6.67E+01 | 6.67E+01 | 0.00 | 0.00 | 1.21 | 1.06 |

[symbol]

indicates data missing or illegible when filed

TABLE 6
The 201 miRNAs in the “miRNA - Ritonavir Therapeutic Efficacy” Subset

| | RTV | ISRIB | Sal | | Abs Z | | | | | | |
|---|---|---|---|---|---|---|---|---|---|---|---|
| | Direc- | Direc- | Direc- | | Score | | | | | | |
| | tion | tion | tion | WT or | Sum | WT | Het | | | | |
| Mature | Opposite | Same as | Opposite | Het | (Geno + | Mean | Mean | HetvWT | RTVvHet | ISRIBvWT | SALvHet |
| miRNA | dE? | dE? | ISRIB? | [symbol] >60 | RTV) | [symbol] | [symbol] | Z Score | Z Score | Z Score | Z Score |
| mmu-miR-135a-5p | yes | yes | yes | yes | 10.43 | 1.47E+03 | 2.86E+02 | −2.37 | 8.06 | −1.21 | 0.71 |
| mmu-miR-182-5p | yes | yes | yes | yes | 7.27 | 1.19E+04 | 4.87E+03 | −1.94 | 5.33 | −0.64 | 0.01 |
| mmu-miR-542-5p | yes | yes | yes | yes | 7.20 | 8.10E+01 | 4.63E+01 | −5.20 | 2.00 | −0.40 | 0.39 |
| mmu-miR-298-5p | yes | yes | yes | yes | 6.72 | 2.53E+03 | 1.54E+03 | −3.12 | 3.59 | −0.18 | 0.16 |
| mmu-miR-183-5p | yes | yes | NO | yes | 5.55 | 6.18E+03 | 2.33E+03 | −2.14 | 3.42 | −0.62 | −0.06 |
| mmu-miR-296-3p | yes | yes | yes | yes | 5.10 | 6.46E+02 | 4.32E+02 | −2.02 | 3.08 | −0.34 | 0.05 |
| mmu-miR-96-5p | yes | yes | yes | yes | 5.01 | 3.00E+03 | 1.09E+03 | −1.91 | 3.10 | −0.64 | 0.13 |
| mmu-miR-344d-3p | yes | yes | NO | yes | 4.51 | 3.52E+02 | 7.00E+01 | −2.53 | 1.98 | −1.60 | −1.00 |
| mmu-miR-5121 | yes | yes | yes | yes | 4.47 | 1.23E+02 | 5.37E+01 | −1.82 | 2.65 | −0.20 | 0.22 |
| mmu-miR-140-3p | yes | yes | yes | yes | 4.39 | 1.73E+03 | 1.20E+03 | −1.85 | 2.54 | −1.40 | 0.50 |
| mmu-miR-344-3p | yes | yes | yes | yes | 4.24 | 2.61E+02 | 8.87E+01 | −2.95 | 1.30 | −1.22 | 0.44 |
| mmu-miR-187-3p | yes | yes | yes | yes | 3.96 | 2.84E+02 | 1.64E+02 | −2.83 | 1.13 | −1.00 | 0.30 |
| mmu-miR-130b-3p | yes | yes | yes | yes | 3.92 | 6.47E+02 | 2.37E+02 | −2.42 | 1.50 | −1.94 | 0.32 |
| mmu-miR-125b-1-3p | yes | yes | NO | yes | 3.85 | 4.16E+02 | 2.68E+02 | −2.83 | 1.02 | −2.45 | −0.41 |
| mmu-miR-34a-5p | yes | yes | NO | yes | 3.80 | 2.52E+04 | 1.50E+04 | −2.42 | 1.38 | −1.51 | −0.03 |
| mmu-miR-532-5p | yes | yes | NO | yes | 3.77 | 6.22E+03 | 2.95E+03 | −3.24 | 0.52 | −1.84 | −0.06 |
| mmu-miR-148a-3p | yes | yes | NO | yes | 3.69 | 4.29E+03 | 2.76E+03 | −1.84 | 1.86 | −2.39 | −0.02 |
| mmu-miR-3535 | yes | yes | NO | yes | 3.67 | 7.16E+02 | 4.16E+02 | −2.24 | 1.43 | −0.98 | −0.66 |
| mmu-miR-362-5p | yes | yes | NO | yes | 3.62 | 5.71E+02 | 2.38E+02 | −2.79 | 0.83 | −1.98 | −0.10 |
| mmu-miR-192-5p | yes | yes | yes | yes | 3.38 | 8.32E+02 | 4.07E+02 | −2.85 | 0.54 | −1.40 | 0.09 |
| mmu-miR-34c-5p | yes | yes | yes | yes | 3.36 | 9.36E+03 | 5.77E+03 | −2.03 | 1.33 | −1.49 | 0.07 |
| mmu-miR-1291 | yes | yes | NO | yes | 3.34 | 2.77E+02 | 9.43E+01 | −1.43 | 1.91 | −0.30 | −0.06 |
| mmu-miR-30b-5p | yes | yes | yes | yes | 3.25 | 2.06E+03 | 1.04E+03 | −2.02 | 1.23 | −0.80 | 0.15 |
| mmu-miR-362-3p | yes | yes | yes | yes | 3.23 | 7.78E+02 | 1.70E+02 | −2.18 | 1.05 | −1.63 | 0.47 |
| mmu-miR-671-5p | yes | yes | yes | yes | 3.19 | 1.52E+03 | 1.05E+03 | −1.43 | 1.76 | −1.04 | 0.26 |
| mmu-miR-224-5p | yes | yes | NO | yes | 3.17 | 3.13E+03 | 1.97E+03 | −2.90 | 0.28 | −1.90 | −0.27 |
| mmu-miR-34b-5p | yes | yes | yes | yes | 3.13 | 1.47E+03 | 5.94E+02 | −1.58 | 1.55 | −1.05 | 0.38 |
| mmu-miR-301b-3p | yes | yes | yes | yes | 3.11 | 1.50E+02 | 4.57E+01 | −2.06 | 1.05 | −1.40 | 0.01 |
| mmu-miR-219a-5p | yes | yes | yes | yes | 3.07 | 2.45E+02 | 9.13E+01 | −1.99 | 1.08 | −1.03 | 0.34 |
| mmu-miR-31-5p | yes | yes | NO | yes | 3.05 | 3.03E+04 | 1.68E+04 | −1.64 | 1.41 | −0.48 | −0.14 |
| mmu-miR-340-5p | yes | yes | yes | yes | 3.02 | 1.22E+03 | 4.90E+02 | −1.93 | 1.09 | −1.41 | 0.31 |
| mmu-miR-33-5p | yes | yes | yes | yes | 3.01 | 2.07E+02 | 8.40E+01 | −1.49 | 1.52 | −0.60 | 0.26 |
| mmu-miR-29c-3p | yes | yes | yes | yes | 3.00 | 3.27E+03 | 1.27E+03 | −1.83 | 1.17 | −0.73 | 0.40 |
| mmu-miR-500-3p | yes | yes | yes | yes | 2.97 | 5.20E+02 | 2.33E+02 | −1.88 | 1.09 | −1.14 | 0.10 |
| mmu-miR-194-5p | yes | yes | yes | yes | 2.90 | 1.44E+03 | 7.05E+02 | −2.44 | 0.46 | −1.11 | 0.18 |
| mmu-miR-301a-3p | yes | yes | yes | yes | 2.89 | 1.08E+04 | 4.36E+03 | −1.45 | 1.45 | −0.63 | 0.35 |
| mmu-miR-700-3p | yes | yes | yes | yes | 2.84 | 2.18E+02 | 1.18E+02 | −2.35 | 0.49 | −1.35 | 0.16 |
| mmu-miR-148b-3p | yes | yes | NO | yes | 2.83 | 5.07E+03 | 3.32E+03 | −2.25 | 0.58 | −1.34 | −0.07 |
| mmu-let-7i-3p | yes | yes | yes | yes | 2.75 | 2.23E+02 | 1.17E+02 | −1.60 | 1.15 | −0.31 | 0.35 |
| mmu-miR-188-5p | yes | yes | NO | yes | 2.73 | 3.25E+02 | 1.29E+02 | −2.40 | 0.34 | −1.71 | −0.05 |
| mmu-miR-101b-3p | yes | yes | yes | yes | 2.73 | 1.12E+03 | 2.94E+02 | −2.22 | 0.52 | −1.26 | 0.64 |
| mmu-miR-99a-5p | yes | yes | NO | yes | 2.71 | 2.06E+03 | 9.94E+02 | −1.99 | 0.73 | −1.70 | −0.17 |
| mmu-miR-652-3p | yes | yes | yes | yes | 2.71 | 7.18E+02 | 2.96E+02 | −1.78 | 0.93 | −0.83 | 0.34 |
| mmu-miR-22-3p | yes | yes | yes | yes | 2.70 | 2.04E+04 | 1.33E+04 | −1.44 | 1.26 | −1.17 | 0.17 |
| mmu-miR-335-Sp | yes | yes | NO | yes | 2.69 | 1.54E+03 | 4.44E+02 | −1.67 | 1.02 | −0.65 | −0.35 |
| mmu-miR-214-5p | yes | yes | yes | yes | 2.68 | 1.95E+02 | 1.00E+02 | −1.58 | 1.09 | −0.50 | 0.04 |
| mmu-miR-181c-5p | yes | yes | yes | yes | 2.65 | 2.47E+02 | 7.80E+01 | −1.77 | 0.88 | −0.73 | 0.26 |
| mmu-miR-199b-3p | yes | yes | NO | yes | 2.61 | 1.15E+05 | 6.65E+04 | −1.44 | 1.17 | −0.84 | −0.10 |
| mmu-miR-31-3p | yes | yes | NO | yes | 2.61 | 4.70E+02 | 1.84E+02 | −1.78 | 0.83 | −0.86 | −0.03 |
| mmu-miR-32-5p | yes | yes | yes | yes | 2.61 | 8.74E+02 | 2.72E+02 | −1.85 | 0.75 | −1.14 | 0.26 |
| mmu-miR-199a-3p | yes | yes | NO | yes | 2.60 | 2.30E+05 | 1.27E+05 | −1.53 | 1.07 | −0.84 | −0.10 |
| mmu-miR-30c-5p | yes | yes | yes | yes | 2.59 | 1.36E+04 | 8.25E+03 | −1.22 | 1.38 | −0.70 | 0.42 |
| mmu-miR-30c-5p | yes | yes | yes | yes | 2.58 | 1.93E+04 | 1.02E+04 | −1.51 | 1.07 | −0.84 | 0.07 |
| mmu-miR-378a-3p | yes | yes | NO | yes | 2.57 | 1.75E+03 | 1.21E+03 | −1.32 | 1.25 | −1.49 | −0.12 |
| mmu-miR-146a-5p | yes | yes | yes | yes | 2.56 | 5.66E+02 | 2.76E+02 | −2.46 | 0.10 | −0.93 | 0.02 |
| mmu-miR-708-5p | yes | yes | yes | yes | 2.51 | 2.83E+02 | 1.59E+02 | −1.23 | 1.28 | −3.87 | 0.58 |
| mmu-miR-542-3p | yes | yes | yes | yes | 2.51 | 5.04E+02 | 1.98E+02 | −1.70 | 0.81 | −0.46 | 0.33 |
| mmu-miR-30a-5p | yes | yes | NO | yes | 2.50 | 3.72E+04 | 2.44E+04 | −1.53 | 0.97 | −1.43 | −0.09 |
| mmu-miR-320-3p | yes | yes | NO | yes | 2.47 | 3.27E+03 | 2.69E+03 | −1.72 | 0.76 | −1.53 | −0.21 |
| mmu-miR-3102-3p | yes | yes | yes | yes | 2.47 | 9.93E+01 | 6.30E+01 | −1.52 | 0.95 | −1.55 | 0.35 |
| mmu-miR-1981-5p | yes | yes | yes | yes | 2.47 | 2.38E+02 | 1.77E+02 | −1.13 | 1.34 | −1.22 | 0.21 |
| mmu-miR-93-5p | yes | yes | NO | yes | 2.43 | 4.94E+04 | 3.19E+04 | −2.13 | 0.30 | −1.12 | −0.01 |
| mmu-miR-339-5p | yes | yes | yes | yes | 2.43 | 1.76E+03 | 1.24E+03 | −1.56 | 0.87 | −0.87 | 0.06 |
| mmu-miR-674-5p | yes | yes | NO | yes | 2.43 | 1.11E+03 | 7.61E+02 | −1.38 | 1.05 | −0.75 | −0.32 |
| mmu-miR-181d-5p | yes | yes | NO | yes | 2.42 | 2.60E+02 | 1.10E+02 | −1.64 | 0.78 | −1.08 | −0.01 |
| mmu-miR-30a-3p | yes | yes | NO | yes | 2.38 | 8.07E+02 | 4.72E+02 | −1.68 | 0.70 | −1.18 | −0.09 |
| mmu-miR-101a-3p | yes | yes | yes | yes | 2.38 | 1.43E+03 | 5.58E+02 | −1.51 | 0.87 | −0.66 | 0.48 |
| mmu-miR-19b-3p | yes | yes | yes | yes | 2.38 | 4.66E+04 | 1.74E+04 | −1.70 | 0.68 | −0.73 | 0.17 |
| mmu-miR-155-5p | yes | yes | NO | yes | 2.36 | 8.50E+03 | 5.23E+03 | −1.91 | 0.45 | −1.26 | −0.20 |
| mmu-let-7i-5p | yes | yes | NO | yes | 2.35 | 6.62E+04 | 4.53E+04 | −1.38 | 0.97 | −0.95 | −0.06 |
| mmu-miR-107-3p | yes | yes | yes | yes | 2.32 | 2.46E+03 | 1.43E+03 | −1.57 | 0.74 | −1.02 | 0.03 |
| mmu-miR-199b-5p | yes | yes | yes | yes | 2.30 | 2.88E+03 | 1.43E+03 | −1.26 | 1.04 | −0.56 | 0.29 |
| mmu-miR-598-3p | yes | yes | yes | yes | 2.30 | 3.55E+02 | 1.83E+02 | −1.51 | 0.78 | −0.78 | 0.06 |
| mmu-miR-103-3p | yes | yes | NO | yes | 2.29 | 4.19E+04 | 2.69E+04 | −1.81 | 0.48 | −0.84 | −0.10 |
| mmu-miR-199a-5p | yes | yes | NO | yes | 2.27 | 5.69E+03 | 3.14E+03 | −1.52 | 0.75 | −0.50 | −0.13 |
| mmu-miR-138-5p | yes | yes | NO | yes | 2.24 | 1.16E+03 | 4.38E+02 | −2.09 | 0.15 | −1.66 | −0.14 |
| mmu-miR-503-5p | yes | yes | NO | yes | 2.23 | 1.16E+03 | 7.28E+02 | −1.59 | 0.64 | −0.47 | −0.28 |
| mmu-let-71-5p | yes | yes | NO | yes | 2.22 | 1.53E+05 | 9.57E+04 | −1.51 | 0.71 | −1.16 | −0.04 |
| mmu-miR-126a-3p | yes | yes | yes | yes | 2.21 | 6.90E+02 | 5.35E+02 | −0.94 | 1.27 | −0.20 | 0.13 |
| mmu-miR-156-3p | yes | yes | yes | yes | 2.19 | 1.73E+03 | 4.97E+02 | −1.85 | 0.34 | −1.67 | 0.68 |
| mmu-miR-18a-5p | yes | yes | yes | yes | 2.19 | 1.68E+03 | 7.16E+02 | −1.59 | 0.60 | −0.54 | 0.21 |
| mmu-miR-378a-5p | yes | yes | yes | yes | 2.18 | 1.11E+02 | 9.47E+01 | −0.89 | 1.28 | −0.81 | 0.24 |
| mmu-miR-196b-5p | yes | yes | NO | yes | 2.15 | 8.72E+03 | 4.87E+03 | −1.58 | 0.57 | −1.10 | 0.00 |
| mmu-miR-19a-3p | yes | yes | yes | yes | 2.12 | 5.21E+03 | 1.83E+03 | −1.62 | 0.49 | −0.65 | 0.15 |
| mmu-miR-378c | yes | yes | yes | yes | 2.11 | 2.18E+02 | 1.39E+02 | −1.09 | 1.02 | −1.11 | 0.04 |
| mmu-miR-301a-5p | yes | yes | yes | yes | 2.11 | 6.10E+01 | 3.23E+01 | −1.54 | 0.57 | −0.59 | 0.14 |
| mmu-miR-675-3p | yes | yes | yes | yes | 2.10 | 1.27E+02 | 6.30E+01 | −1.86 | 0.25 | −2.24 | 0.34 |
| mmu-miR-27a-3p | yes | yes | yes | yes | 2.10 | 1.30E+04 | 6.09E+03 | −1.44 | 0.66 | −0.88 | 0.21 |
| mmu-miR-26b-5p | yes | yes | yes | yes | 2.08 | 2.96E+04 | 1.64E+04 | −1.35 | 0.73 | −1.11 | 0.05 |
| mmu-miR-3068-5p | yes | yes | NO | yes | 2.06 | 2.02E+02 | 1.15E+02 | −1.30 | 0.76 | −0.88 | −0.02 |
| mmu-miR-190a-5p | yes | yes | yes | yes | 2.06 | 2.41E+03 | 9.02E+02 | −1.43 | 0.62 | −0.38 | 0.04 |
| mmu-miR-872-5p | yes | yes | yes | yes | 2.05 | 3.32E+03 | 1.71E+03 | −1.37 | 0.68 | −1.26 | 0.19 |
| mmu-miR-106b-5p | yes | NO | yes | yes | 2.05 | 2.86E+02 | 1.40E+02 | −1.51 | 0.54 | 0.31 | 0.04 |
| mmu-miR-106b-3p | yes | yes | NO | yes | 2.04 | 2.00E+03 | 1.37E+03 | −1.89 | 0.15 | −1.54 | −0.23 |
| mmu-miR-190b-5p | yes | yes | yes | yes | 2.02 | 3.16E+02 | 1.40E+02 | −1.73 | 0.28 | −0.97 | 0.15 |
| mmu-miR-98-5p | yes | yes | NO | yes | 2.00 | 2.72E+03 | 1.51E+03 | −1.58 | 0.42 | −0.75 | −0.22 |
| mmu-let-7g-5p | yes | yes | NO | yes | 1.98 | 2.85E+04 | 1.65E+04 | −1.48 | 0.50 | −1.37 | −0.16 |
| mmu-miR-322-5p | yes | yes | NO | yes | 1.97 | 5.19E+03 | 3.18E+03 | −1.22 | 0.75 | −0.40 | −0.07 |
| mmu-miR-152-3p | yes | yes | NO | yes | 1.94 | 1.07E+04 | 7.69E+03 | −1.18 | 0.76 | −1.28 | −0.02 |
| mmu-miR-350-3p | yes | yes | yes | yes | 1.94 | 2.48E+03 | 1.17E+03 | −1.53 | 0.41 | −0.49 | 0.08 |
| mmu-miR-181a-5p | yes | yes | NO | yes | 1.93 | 7.53E+03 | 4.37E+03 | −1.62 | 0.31 | −1.45 | −0.23 |
| mmu-miR-299a-3p | yes | yes | yes | yes | 1.92 | 7.63E+01 | 5.33E+01 | −0.61 | 1.31 | −1.31 | 0.39 |
| mmu-miR-151-5p | yes | yes | yes | yes | 1.90 | 1.94E+02 | 1.28E+02 | −1.58 | 0.32 | −0.91 | 0.12 |
| mmu-miR-25-3p | yes | yes | NO | yes | 1.89 | 3.40E+04 | 2.61E+04 | −1.18 | 0.71 | −1.12 | −0.04 |
| mmu-miR-451a | yes | NO | NO | yes | 1.89 | 1.90E+02 | 2.16E+02 | 0.52 | −1.37 | −1.27 | 0.38 |
| mmu-miR-450b-3p | yes | yes | NO | yes | 1.88 | 1.23E+02 | 6.07E+01 | −1.57 | 0.31 | −0.68 | −0.29 |
| mmu-miR-181b-5p | yes | yes | NO | yes | 1.88 | 3.80E+03 | 2.15E+03 | −1.34 | 0.54 | −1.35 | −0.32 |
| mmu-miR-30d-5p | yes | yes | NO | yes | 1.86 | 1.36E+04 | 1.08E+04 | −1.16 | 0.70 | −1.38 | −0.11 |
| mmu-miR-30e-3p | yes | yes | NO | yes | 1.85 | 4.03E+02 | 2.45E+02 | −1.36 | 0.49 | −0.94 | −0.06 |
| mmu-miR-483-5p | yes | yes | NO | yes | 1.83 | 2.15E+02 | 1.27E+02 | −1.82 | 0.01 | −2.93 | −1.22 |
| mmu-miR-149-5p | yes | yes | yes | yes | 1.82 | 1.10E+03 | 7.87E+02 | −1.49 | 0.33 | −1.27 | 0.03 |
| mmu-miR-503-3p | yes | yes | NO | yes | 1.82 | 6.40E+01 | 5.00E+01 | −0.83 | 0.99 | −0.76 | −0.70 |
| mmu-miR-136-3p | yes | yes | yes | yes | 1.81 | 7.97E+01 | 6.73E+01 | −0.29 | 1.52 | −0.88 | 0.08 |
| mmu-miR-361-5p | yes | yes | NO | yes | 1.79 | 4.46E+03 | 2.84E+03 | −1.08 | 0.71 | −0.70 | −0.15 |
| mmu-miR-582-3p | yes | yes | NO | yes | 1.78 | 1.11E+02 | 6.90E+01 | −1.58 | 0.20 | −1.08 | −0.45 |
| mmu-let-7a-1-3p | yes | yes | yes | yes | 1.76 | 3.85E+02 | 2.23E+02 | −1.14 | 0.62 | −0.93 | 0.28 |
| mmu-let-7c-2-3p | yes | yes | yes | yes | 1.76 | 3.85E+02 | 2.23E+02 | −1.14 | 0.62 | −0.93 | 0.28 |
| mmu-miR-186-5p | yes | yes | yes | yes | 1.76 | 1.01E+04 | 5.70E+03 | −1.42 | 0.33 | −0.99 | 0.00 |
| mmu-miR-324-3p | yes | yes | yes | yes | 1.75 | 7.93E+02 | 5.32E+02 | −0.92 | 0.82 | −0.50 | 0.16 |
| mmu-miR-223-3p | yes | NO | yes | yes | 1.72 | 1.16E+02 | 1.93E+02 | 1.14 | −0.59 | −1.00 | −0.01 |
| mmu-miR-29b-3p | yes | yes | yes | yes | 1.72 | 2.05E+04 | 1.13E+04 | −1.19 | 0.53 | −0.45 | 0.38 |
| mmu-miR-16-5p | yes | yes | NO | yes | 1.71 | 5.91E+05 | 3.54E+05 | −1.50 | 0.21 | −1.22 | −0.01 |
| mmu-miR-1843a-5p | yes | yes | yes | yes | 1.70 | 7.67E+01 | 4.67E+01 | −1.42 | 0.28 | −1.23 | 0.07 |
| mmu-miR-34c-3p | yes | yes | NO | yes | 1.70 | 3.88E+02 | 2.89E+02 | −0.91 | 0.78 | −1.27 | −0.37 |
| mmu-miR-185-5p | yes | yes | yes | yes | 1.69 | 3.04E+03 | 2.19E+03 | −1.16 | 0.53 | −0.81 | 0.15 |
| mmu-miR-124-3p | yes | NO | yes | yes | 1.67 | 6.80E+01 | 7.00E+01 | 0.07 | −1.59 | −1.71 | −0.72 |
| mmu-miR-191-5p | yes | yes | yes | yes | 1.62 | 2.08E+04 | 1.42E+04 | −1.34 | 0.28 | −1.22 | 0.04 |
| mmu-miR-23a-3p | yes | yes | yes | yes | 1.62 | 4.74E+04 | 3.15E+04 | −1.16 | 0.46 | −1.43 | 0.05 |
| mmu-miR-17-3p | yes | yes | yes | yes | 1.59 | 6.98E+02 | 3.97E+02 | −1.57 | 0.02 | −1.43 | 0.10 |
| mmu-miR-206-3p | yes | yes | NO | yes | 1.58 | 5.64E+03 | 3.47E+03 | −1.38 | 0.20 | −1.56 | −0.27 |
| mmu-miR-15a-5p | yes | yes | yes | yes | 1.58 | 5.73E+03 | 2.74E+03 | −1.36 | 0.22 | −0.57 | 0.17 |
| mmu-miR-450a-5p | yes | NO | yes | yes | 1.53 | 1.39E+02 | 6.67E+01 | −1.15 | 0.39 | 0.01 | 0.09 |
| mmu-miR-20a-5p | yes | yes | NO | yes | 1.51 | 2.72E+04 | 1.33E+04 | −1.42 | 0.09 | −0.84 | −0.01 |
| mmu-miR-322-3p | yes | yes | NO | yes | 1.47 | 2.71E+02 | 1.77E+02 | −1.20 | 0.28 | −0.98 | −0.26 |
| mmu-miR-29a-3p | yes | yes | yes | yes | 1.44 | 1.12E+05 | 6.90E+04 | −1.15 | 0.29 | −0.75 | 0.17 |
| mmu-miR-374b-5p | yes | yes | yes | yes | 1.42 | 1.39E+03 | 8.58E+02 | −0.92 | 0.51 | −0.80 | 0.16 |
| mmu-miR-34b-3p | yes | yes | NO | yes | 1.42 | 2.77E+02 | 2.24E+02 | −0.55 | 0.87 | −0.72 | −0.27 |
| mmu-miR-222-3p | yes | yes | NO | yes | 1.42 | 3.72E+03 | 2.63E+03 | −1.25 | 0.16 | −1.61 | −0.53 |
| mmu-miR-128-3p | yes | yes | NO | yes | 1.42 | 4.26E+03 | 2.81E+03 | −1.24 | 0.18 | −1.04 | −0.03 |
| mmu-miR-21a-5p | yes | yes | NO | yes | 1.41 | 3.21E+05 | 1.79E+05 | −1.24 | 0.17 | −1.01 | −0.03 |
| mmu-miR-669b-5p | yes | yes | yes | yes | 1.39 | 6.87E+01 | 4.03E+01 | −0.81 | 0.58 | −0.50 | 0.29 |
| mmu-miR-421-3p | yes | yes | NO | yes | 1.38 | 1.16E+03 | 6.99E+02 | −1.13 | 0.25 | −0.89 | −0.01 |
| mmu-miR-335-3p | yes | yes | NO | yes | 1.35 | 8.20E+01 | 3.97E+01 | −1.04 | 0.31 | −0.23 | −0.41 |
| mmu-miR-1839-5p | yes | yes | NO | yes | 1.34 | 1.25E+03 | 7.66E+02 | −1.33 | 0.01 | −2.22 | −0.04 |
| mmu-miR-26a-5p | yes | yes | NO | yes | 1.33 | 5.71E+04 | 4.03E+04 | −0.91 | 0.42 | −1.13 | −0.13 |
| mmu-miR-130a-3p | yes | yes | NO | yes | 1.32 | 1.21E+04 | 7.63E+03 | −1.02 | 0.30 | −0.49 | −0.03 |
| mmu-miR-122-5p | yes | NO | yes | yes | 1.32 | 8.81E+02 | 1.31E+03 | 0.83 | −0.50 | −0.85 | −0.07 |
| mmu-miR-7a-1-3p | yes | yes | NO | yes | 1.32 | 1.35E+02 | 8.60E+01 | −1.20 | 0.12 | −1.50 | −0.29 |
| mmu-miR-143-3p | yes | yes | yes | yes | 1.32 | 1.44E+05 | 8.98E+04 | −0.99 | 0.33 | −0.93 | 0.03 |
| mmu-miR-28a-3p | yes | yes | NO | yes | 1.31 | 3.20E+03 | 2.18E+03 | −1.05 | 0.27 | −0.89 | −0.11 |
| mmu-miR-210-3p | yes | yes | yes | yes | 1.31 | 1.02E+03 | 6.37E+02 | −1.02 | 0.29 | −0.28 | 0.03 |
| mmu-miR-425-3p | yes | yes | NO | yes | 1.30 | 6.33E+01 | 4.27E+01 | −1.09 | 0.21 | −0.92 | −0.03 |
| mmu-miR-17-5p | yes | yes | NO | yes | 1.28 | 1.10E+03 | 6.44E+02 | −1.23 | 0.04 | −0.50 | −0.15 |
| mmu-let-7j | yes | yes | yes | yes | 1.27 | 3.54E+03 | 7.90E+03 | 1.12 | −0.15 | 0.67 | −0.38 |
| mmu-let-7d-5p | yes | yes | NO | yes | 1.25 | 2.08E+04 | 1.56E+04 | −0.95 | 0.30 | −0.87 | −0.32 |
| mmu-miR-872-3p | yes | yes | yes | yes | 1.22 | 8.19E+02 | 4.70E+02 | −1.01 | 0.21 | −0.61 | 0.10 |
| mmu-miR-27b-3p | yes | yes | yes | yes | 1.21 | 3.56E+04 | 2.19E+04 | −0.92 | 0.28 | −1.38 | 0.13 |
| mmu-miR-326-3p | yes | yes | yes | yes | 1.20 | 8.27E+01 | 6.67E+01 | −0.64 | 0.55 | −0.17 | 0.07 |
| mmu-miR-126a-5p | yes | yes | yes | yes | 1.18 | 4.16E+02 | 3.31E+02 | −0.65 | 0.54 | −0.55 | 0.27 |
| mmu-let-7e-5p | yes | yes | NO | yes | 1.18 | 2.34E+04 | 1.87E+04 | −0.87 | 0.31 | −0.47 | −0.38 |
| mmu-miR-376a-3p | yes | yes | NO | yes | 1.16 | 1.26E+02 | 1.08E+02 | −0.27 | 0.89 | −0.90 | −0.32 |
| mmu-miR-143-5p | yes | yes | NO | yes | 1.15 | 1.76E+03 | 1.20E+03 | −0.77 | 0.38 | −0.79 | −0.11 |
| mmu-miR-24-3p | yes | yes | yes | yes | 1.14 | 9.37E+04 | 7.44E+04 | −0.71 | 0.43 | −1.12 | 0.02 |
| mmu-let-7a-5p | yes | yes | NO | yes | 1.06 | 1.29E+05 | 1.04E+05 | −0.88 | 0.19 | −0.83 | −0.49 |
| mmu-miR-214-3p | yes | yes | NO | yes | 1.06 | 6.83E+03 | 3.94E+03 | −1.05 | 0.01 | −0.84 | −0.43 |
| mmu-miR-324-5p | yes | yes | NO | yes | 1.05 | 1.20E+03 | 9.42E+02 | −0.80 | 0.25 | −0.84 | −0.07 |
| mmu-miR-93-3p | yes | yes | NO | yes | 1.02 | 2.32E+02 | 1.80E+02 | −0.95 | 0.07 | −1.45 | −0.35 |
| mmu-miR-676-3p | yes | yes | NO | yes | 1.02 | 6.90E+02 | 4.52E+02 | −1.00 | 0.02 | −1.97 | −0.24 |
| mmu-miR-29a-5p | yes | yes | yes | yes | 0.99 | 1.80E+02 | 1.10E+02 | −0.84 | 0.15 | −0.78 | 0.16 |
| mmu-miR-455-3p | yes | yes | yes | yes | 0.97 | 8.27E+01 | 9.37E+01 | 0.42 | −0.55 | 0.28 | −0.35 |
| mmu-miR-296-5p | yes | yes | yes | yes | 0.96 | 2.71E+02 | 2.27E+02 | −0.60 | 0.37 | −0.79 | 0.16 |
| mmu-miR-129-2-3p | yes | NO | NO | yes | 0.93 | 2.76E+02 | 3.16E+02 | 0.21 | −0.72 | −1.46 | 0.11 |
| mmu-miR-491-5p | yes | yes | NO | yes | 0.92 | 8.17E+01 | 7.10E+01 | −0.73 | 0.18 | −2.61 | −0.27 |
| mmu-miR-145a-5p | yes | yes | NO | yes | 0.89 | 1.46E+04 | 1.11E+04 | −0.63 | 0.26 | −0.64 | −0.03 |
| mmu-miR-142a-3p | yes | NO | NO | yes | 0.81 | 2.17E+02 | 2.39E+02 | 0.43 | −0.38 | −2.35 | 0.46 |
| mmu-miR-1198-5p | yes | yes | NO | yes | 0.80 | 6.12E+02 | 5.43E+02 | −0.61 | 0.19 | −1.13 | −0.20 |
| mmu-miR-30c-2-3p | yes | yes | NO | yes | 0.80 | 1.72E+02 | 1.43E+02 | −0.74 | 0.06 | −0.99 | −0.49 |
| mmu-miR-23b-3p | yes | yes | NO | yes | 0.78 | 9.48E+03 | 7.21E+03 | −0.67 | 0.12 | −1.42 | −0.09 |
| mmu-miR-136-5p | yes | yes | NO | yes | 0.77 | 7.37E+01 | 6.17E+01 | −0.24 | 0.54 | −0.72 | −0.49 |
| mmu-miR-140-5p | yes | NO | NO | yes | 0.69 | 3.18E+02 | 3.56E+02 | 0.20 | −0.49 | −1.03 | 0.14 |
| mmu-miR-669o-3p | yes | yes | yes | yes | 0.67 | 1.42E+03 | 9.69E+02 | −0.59 | 0.08 | −0.53 | 0.26 |
| mmu-miR-669a-3p | yes | yes | yes | yes | 0.67 | 1.57E+04 | 1.07E+04 | −0.59 | 0.08 | −0.53 | 0.26 |
| mmu-miR-486a-5p | yes | NO | yes | yes | 0.66 | 2.03E+02 | 2.30E+02 | 0.23 | −0.43 | −1.07 | −0.29 |
| mmu-miR-449a-5p | yes | yes | yes | yes | 0.66 | 1.79E+02 | 1.34E+02 | −0.56 | 0.10 | −1.22 | 0.26 |
| mmu-miR-218-5p | yes | yes | NO | yes | 0.66 | 6.32E+04 | 4.67E+04 | −0.50 | 0.16 | −1.01 | −0.03 |
| mmu-miR-1298-5p | yes | yes | yes | yes | 0.65 | 7.30E+01 | 6.53E+01 | −0.23 | 0.42 | −1.22 | 0.20 |
| mmu-miR-92a-3p | yes | yes | NO | yes | 0.57 | 1.77E+04 | 1.48E+04 | −0.47 | 0.10 | −1.41 | −0.13 |
| mmu-miR-193a-5p | yes | yes | yes | yes | 0.55 | 2.66E+02 | 2.41E+02 | −0.32 | 0.23 | −1.47 | 0.00 |
| mmu-miR-345-3p | yes | NO | yes | yes | 0.52 | 1.75E+02 | 1.70E+02 | −0.04 | 0.48 | 0.53 | 0.24 |
| mmu-miR-674-3p | yes | yes | NO | yes | 0.52 | 5.63E+02 | 4.71E+02 | −0.38 | 0.13 | −0.66 | −0.03 |
| mmu-miR-497a-5p | yes | yes | yes | yes | 0.52 | 1.38E+02 | 1.32E+02 | −0.08 | 0.44 | −0.56 | 0.10 |
| mmu-miR-150-5p | yes | NO | NO | yes | 0.51 | 5.93E+01 | 7.70E+01 | 0.46 | −0.05 | −0.80 | 0.39 |
| mmu-miR-423-5p | yes | yes | yes | yes | 0.51 | 2.20E+03 | 2.10E+03 | −0.20 | 0.31 | −0.68 | 0.04 |
| mmu-let-7b-5p | yes | yes | NO | yes | 0.49 | 3.83E+04 | 3.24E+04 | −0.31 | 0.18 | −0.90 | −0.05 |
| mmu-miR-669c-5p | yes | yes | NO | yes | 0.48 | 7.69E+02 | 6.16E+02 | −0.41 | 0.07 | −0.87 | −0.06 |
| mmu-miR-669d-5p | yes | yes | yes | yes | 0.47 | 6.67E+01 | 5.40E+01 | −0.28 | 0.19 | −0.17 | 0.10 |
| mmu-miR-369-3p | yes | yes | NO | yes | 0.26 | 2.00E+02 | 1.92E+02 | −0.10 | 0.17 | −1.35 | −0.14 |
| mmu-miR-484 | yes | NO | yes | yes | 0.21 | 1.98E+03 | 2.04E+03 | 0.09 | −0.12 | −1.31 | −0.15 |
| mmu-miR-92b-3p | yes | yes | yes | yes | 0.18 | 2.48E+02 | 2.35E+02 | −0.11 | 0.07 | −0.74 | 0.25 |
| mmu-let-7e-5p | yes | yes | NO | yes | 0.16 | 1.08E+05 | 1.02E+05 | −0.13 | 0.03 | −1.24 | −0.21 |
| mmu-miR-129-5p | yes | NO | NO | yes | 0.03 | 1.78E+03 | 1.81E+03 | 0.03 | 0.00 | −1.61 | 0.09 |

[symbol]

indicates data missing or illegible when filed

TABLE 7
The 37 miRNAs of the “Candidate miRNA” Subset

| | RTV | ISRIB | Sal | | Abs Z | | | | | | |
|---|---|---|---|---|---|---|---|---|---|---|---|
| | Direc- | Direc- | Direc- | | Score | | | | | | |
| | tion | tion | tion | WT or | Sum | WT | Het | | | | |
| Mature | Opposite | Same as | Opposite | Het | (Geno + | Mean | Mean | HetvWT | RTVvHet | ISRIBvWT | SALvHet |
| miRNA | dE? | dE? | ISRIB? | [symbol] >60 | RTV) | [symbol] | [symbol] | Z Score | Z Score | Z Score | Z Score |
| mmu-miR-135a-5p | yes | yes | yes | yes | 10.43 | 1.47E+03 | 2.86E+02 | −2.37 | 8.06 | −1.21 | 0.71 |
| mmu-miR-182-5p | yes | yes | yes | yes | 7.27 | 1.19E+04 | 4.87E+03 | −1.94 | 5.33 | −0.64 | 0.01 |
| mmu-miR-542-5p | yes | yes | yes | yes | 7.20 | 8.10E+01 | 4.63E+01 | −5.20 | 2.00 | −0.40 | 0.39 |
| mmu-miR-298-5p | yes | yes | yes | yes | 6.72 | 2.53E+03 | 1.54E+03 | −3.12 | 3.59 | −0.18 | 0.16 |
| mmu-miR-183-5p | yes | yes | NO | yes | 5.55 | 6.18E+03 | 2.33E+03 | −2.14 | 3.42 | −0.62 | −0.06 |
| mmu-miR-296-3p | yes | yes | yes | yes | 5.10 | 6.46E+02 | 4.32E+02 | −2.02 | 3.08 | −0.34 | 0.05 |
| mmu-miR-96-5p | yes | yes | yes | yes | 5.01 | 3.00E+03 | 1.09E+03 | −1.91 | 3.10 | −0.64 | 0.13 |
| mmu-miR-344d-3p | yes | yes | NO | yes | 4.51 | 3.52E+02 | 7.00E+01 | −2.53 | 1.98 | −1.60 | −1.00 |
| mmu-miR-5121 | yes | yes | yes | yes | 4.47 | 1.23E+02 | 5.37E+01 | −1.82 | 2.65 | −0.20 | 0.22 |
| mmu-miR-140-3p | yes | yes | yes | yes | 4.39 | 1.73E+03 | 1.20E+03 | −1.85 | 2.54 | −1.40 | 0.50 |
| mmu-miR-344-3p | yes | yes | yes | yes | 4.24 | 2.61E+02 | 8.87E+01 | −2.95 | 1.30 | −1.22 | 0.44 |
| mmu-miR-187-3p | yes | yes | yes | yes | 3.96 | 2.84E+02 | 1.64E+02 | −2.83 | 1.13 | −1.00 | 0.30 |
| mmu-miR-130b-3p | yes | yes | yes | yes | 3.92 | 6.47E+02 | 2.37E+02 | −2.42 | 1.50 | −1.94 | 0.32 |
| mmu-miR-125b-1-3p | yes | yes | NO | yes | 3.85 | 4.16E+02 | 2.68E+02 | −2.83 | 1.02 | −2.45 | −0.41 |
| mmu-miR-34a-5p | yes | yes | NO | yes | 3.80 | 2.52E+04 | 1.50E+04 | −2.42 | 1.38 | −1.51 | −0.03 |
| mmu-miR-532-5p | yes | yes | NO | yes | 3.77 | 6.22E+03 | 2.95E+03 | −3.24 | 0.52 | −1.84 | −0.06 |
| mmu-miR-148a-3p | yes | yes | NO | yes | 3.69 | 4.29E+03 | 2.76E+03 | −1.84 | 1.86 | −2.39 | −0.02 |
| mmu-miR-3535 | yes | yes | NO | yes | 3.67 | 7.16E+02 | 4.16E+02 | −2.24 | 1.43 | −0.98 | −0.66 |
| mmu-miR-362-5p | yes | yes | NO | yes | 3.62 | 5.71E+02 | 2.38E+02 | −2.79 | 0.83 | −1.98 | −0.10 |
| mmu-miR-192-5p | yes | yes | yes | yes | 3.38 | 8.32E+02 | 4.07E+02 | −2.85 | 0.54 | −1.40 | 0.09 |
| mmu-miR-34c-5p | yes | yes | yes | yes | 3.36 | 9.36E+03 | 5.77E+03 | −2.03 | 1.33 | −1.49 | 0.07 |
| mmu-miR-1291 | yes | yes | NO | yes | 3.34 | 2.77E+02 | 9.43E+01 | −1.43 | 1.91 | −0.30 | −0.06 |
| mmu-miR-30h-5p | yes | yes | yes | yes | 3.25 | 2.06E+03 | 1.04E+03 | −2.02 | 1.23 | −0.80 | 0.15 |
| mmu-miR-362-3p | yes | yes | yes | yes | 3.23 | 7.78E+02 | 1.70E+02 | −2.18 | 1.05 | −1.63 | 0.47 |
| mmu-miR-671-5p | yes | yes | yes | yes | 3.19 | 1.52E+03 | 1.05E+03 | −1.43 | 1.76 | −1.04 | 0.26 |
| mmu-miR-31-5p | yes | yes | NO | yes | 3.05 | 3.03E+04 | 1.68E+04 | −1.64 | 1.41 | −0.48 | −0.14 |
| mmu-miR-22-3p | yes | yes | yes | yes | 2.70 | 2.04E+04 | 1.33E+04 | −1.44 | 1.26 | −1.17 | 0.17 |
| mmu-miR-199b-3p | yes | yes | NO | yes | 2.61 | 1.15E+05 | 6.65E+04 | −1.44 | 1.17 | −0.84 | −0.10 |
| mmu-miR-199a-3p | yes | yes | NO | yes | 2.60 | 2.30E+05 | 1.27E+05 | −1.53 | 1.07 | −0.84 | −0.10 |
| mmu-miR-30e-5p | yes | yes | yes | yes | 2.59 | 1.36E+04 | 8.25E+03 | −1.22 | 1.38 | −0.70 | 0.42 |
| mmu-miR-30c-5p | yes | yes | yes | yes | 2.58 | 1.93E+04 | 1.02E+04 | −1.51 | 1.07 | −0.84 | 0.07 |
| mmu-miR-30a-5p | yes | yes | NO | yes | 2.50 | 3.72E+04 | 2.44E+04 | −1.53 | 0.97 | −1.43 | −0.09 |
| mmu-miR-93-5p | yes | yes | NO | yes | 2.43 | 4.94E+04 | 3.19E+04 | −2.13 | 0.30 | −1.12 | −0.01 |
| mmu-miR-19b-3p | yes | yes | yes | yes | 2.38 | 4.66E+04 | 1.74E+04 | −1.70 | 0.68 | −0.73 | 0.17 |
| mmu-let-7i-5p | yes | yes | NO | yes | 2.35 | 6.62E+04 | 4.53E+04 | −1.38 | 0.97 | −0.95 | −0.06 |
| mmu-miR-103-3p | yes | yes | NO | yes | 2.29 | 4.19E+04 | 2.69E+04 | −1.81 | 0.48 | −0.84 | −0.10 |
| mmu-let-7f-5p | yes | yes | NO | yes | 2.22 | 1.53E+05 | 9.57E+04 | −1.51 | 0.71 | −1.16 | −0.04 |
| mmu-miR-26b-Sp | yes | yes | yes | yes | 2.08 | 2.96E+04 | 1.64E+04 | −1.35 | 0.73 | −1.11 | 0.05 |

[symbol]

indicates data missing or illegible when filed

### Example 5

### Validating the RNAseq Data Using RT-qPCR

EVs isolated from WT and Tor1A^{dGAG/+} mouse embryonic fibroblast were treated for 24 hours with either vehicle or RTV. The same 6 clones used in the experiments described above were used here. miRNAs were isolated and 11 specific miRNAs were measured using TaqMan Advanced miRNA probes. 9 of the 11 specific miRNAs were experimental while 2 of the 11 specific miRNAs were “housekeeping”. The goal of this experiment was to identify concordance of the miRNA differential expression between the original RNAseq analysis (described above) and the RT-qPCR approach. While mmu-miR-182-5p is not 100% conserved in humans, the same sequence (hsa-miR-182-5p) in humans is UUUGGCAAUGGUAGAACUCACACU (SEQ ID NO:270). Similarly, while mmu-miR-542-5p is not 100% conserved in humans, the same sequence (hsa-miR-542-5P) in humans is UCGGGGAUCAUCAUGUCACGAGA (SEQ ID NO:271). See Table 8 for the results of this validation experiment.

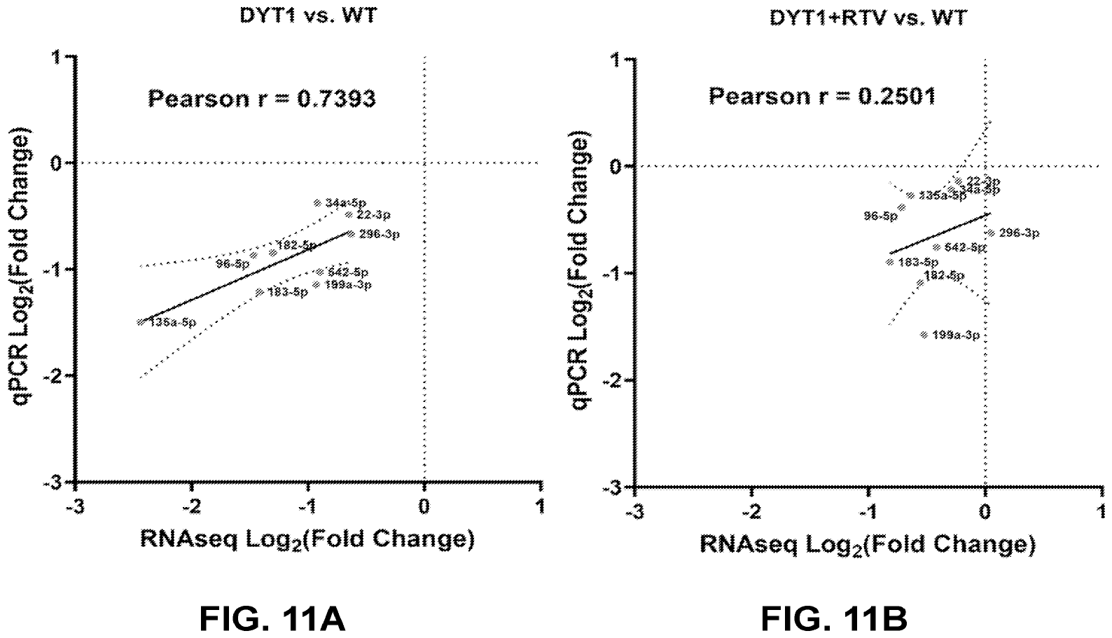

As FIG. 11A-11B show, there was a strong correlation between the RNAseq analysis (i.e., absolute quantification by unique molecular identifies/barcodes) and the RT-qPCR validation (i.e., relative quantification by averaging two housekeeping miRNAs) experiments. Here, FIG. 11A shows the correlation of DYT1 v. WT cells (Pearson r=0.79323) while FIG. 11B shows the correlation of DYT1 cells treated with RTV and WT cells (Pearson r=0.2501).

TABLE 8
The 11 miRNAs Validated by RT−qPCR

| | | | | | | | | | Abs Z | | | RNAseq |
|---|---|---|---|---|---|---|---|---|---|---|---|---|
| | | | 100% | | RTV | [symbol] | [symbol] | WT or | Score | | RT− | |
| | | | Mouse/ | | Direction | Direction | Direction | Het | Sum | | qPCR | [symbol] |
| | Human | Mouse Mature | Human | | Opposite | Same as | Opposite | UMI > | (Geno + | WT Mean | Log2 | Log2 |
| Mature miRNA | miRNA | Sequence | Homology | [symbol] | dE? | dE? | [symbol] | 60 | RTV) | UMIs | FC | FC |
| mmu−miR−135a−5p | hsa−miR−135a−5p | [symbol] | yes | TopZ | yes | yes | yes | yes | 10.43 | 1.47E−03 | −1.50 | −2.36 |
| | | [symbol] (SEQ ID NO: 1) | | | | | | | | | | |
| mmu−miR−182−5p | hsa−miR−182−5p | [symbol] | NO | TopZ | yes | yes | yes | yes | 7.27 | 1.19E−04 | −0.85 | −1.29 |
| | | [symbol] (SEQ ID NO: 2) | | | | | | | | | | |
| mmu−miR−542−5p | hsa−miR−542−5p | [symbol] | NO | TopZ | yes | yes | yes | yes | 7.20 | 8.10E−01 | −1.03 | −0.81 |
| | | [symbol] (SEQ ID NO: 3) | | | | | | | | | | |
| mmu−miR−183−5p | hsa−miR−183−5p | [symbol] | yes | TopZ | yes | yes | NO | yes | 5.55 | 6.18E−03 | −1.21 | −1.40 |
| | | [symbol] (SEQ ID NO: 4) | | | | | | | | | | |
| mmu−miR−296−3p | hsa−miR−296−3p | [symbol] | yes | TopZ | yes | yes | yes | yes | 5.10 | 6.46E−02 | −0.67 | −0.58 |
| | | [symbol] (SEQ ID NO: 5) | | | | | | | | | | |
| mmu−miR−96−5p | hsa−miR−96−5p | [symbol] | yes | TopZ | yes | yes | Ves | yes | 5.01 | 3.00E−03 | −0.87 | −1.46 |
| | | [symbol] (SEQ ID NO: 6) | | | | | | | | | | |
| mmu−miR−34a−5p | hsa−miR−34a−5p | [symbol] | yes | TopZ | yes | yes | NO | yes | 3.80 | 2.52E−04 | −0.38 | −0.74 |
| | | [symbol] (SEQ ID NO: 7) | | | | | | | | | | |
| mmu−miR−22−3p | hsu−miR−22−3p | [symbol] | yes | Top | yes | yes | yes | yes | 2.70 | 2.04E−04 | −0.49 | −0.62 |
| | | [symbol] (SEQ ID NO: 8) | | Abundance | | | | | | | | |
| mmu−miR−199a−3p | hsa−miR−199a−3p | [symbol] | yes | Top | yes | yes | NO | yes | 2.60 | 2.30E−05 | −1.14 | −0.86 |
| | | [symbol] (SEQ ID NO: 9) | | Abundance | | | | | | | | |
| mmu−miR−125b−5p | hsa−miR−125b−5p | [symbol] | yes | House− | NO | yes | NO | yes | 0.21 | 6.86E−05 | NA | −0.05 |
| | | [symbol] (SEQ ID NO: 10) | | keeping | | | | | | | | |
| mmu−let−7c−5p | hsa−let−7c−5p | [symbol] | yes | House− | yes | yes | NO | yes | 0.16 | 1.08E−05 | NA | −0.09 |
| | | [symbol] (SEQ ID NO: 11) | | keeping | | | | | | | | |

[symbol]

indicates data missing or illegible when filed

### Example 6

### Completion of Proteomics Targeted Assay—Parallel Reaction Monitoring

The development of the assay involved the synthesis of 258 stable isotope-labeled (SIL) peptides corresponding to 98 total proteins (of which 78 are protein markers and 20 are EV or plasma control markers). Peptides were selected based on performance in the original proteomics dataset and based on homology between mouse and human amino acid sequences using the Peptide Atlas database. JPT SpikeTides™ SILs were synthesized by JPT Peptide Technologies GmbH. This population contained protein biomarkers for a dystonia (n=78) as well as EV and plasma internal controls (n=20). The QC and characterization of the SIL peptides using mass spectrometry. The SIL peptide mixture was run to identify individual peptide behavior. The best precursor ion for each peptide was selected as a “target” for isolation into a collision cell and the instrument recalibrated to detect target SILs within defined retention time windows Then, the SIL peptides were run again to validate the targeted performance. To optimize performance for different biosample matrices with varying proteomic backgrounds, representative samples were generated to identify optimal retention times for each biosample type. As a positive control for validation of murine candidate biomarkers previously detected, a pooled conditioned media sample was generated from the six MEF clones used in Cohort 2. To identify performance on homologous human proteins, a pooled conditioned media sample was generated from hiPSC-derived neural progenitor cells (NPCs). Then, parallel reaction monitoring (PRM) was performed on EVs isolated from these conditioned media samples. PRM spectra were then analyzed using Skyline to identify peptides that were detected in MEF or NPC EVs (MacLean B, et al. (2010) Bioinformatics. 26(7): 966-968).

Table 9 shows the 258 SIL peptides corresponding to the 78 protein biomarkers (Table 10) and 20 control markers (Tables 13 and 14) for the PRM analysis. Table 10 shows the 78 protein biomarkers for the PRM analysis. Table 11 shows the homologous proteins having more than 1 human peptide in the PRM panel while Table 12 shows those homologous proteins having no human peptides in the PRM panel. Table 13 shows the EV control markers for the PRM analysis.

Table 14 shows the plasma control markers for the PRM analysis. Table 15 shows the successful detection of murine candidate biomarker proteins/peptides and controls in EVs derived from human iPSC-derived NPCs.

TABLE 9
The 258 SIL Peptides of the Parallel Reaction Monitoring Experiments

| | | | | | Avg. | | | |
|---|---|---|---|---|---|---|---|---|
| | | | | | Intensity | | | |
| | | | | SEQ | in MEF | Detected | Detected | Full |
| | ID | Gene | | ID | Discovery | in | in [symbol] | Protein |
| Accession | (Mouse) | Name | Peptide | NO | Dataset | NPC Pool | [symbol] | Names |
| O35598 | ADA10 | Adam10 | FSLCSIR | 12 | 1.56E−06 | present | present | [symbol] |
| | | | | | | | | [symbol] |
| | | | | | | | | [symbol] |
| O35598 | ADA10 | Adam10 | LVDADGPLAR | 13 | 2.88E−06 | present | present | [symbol] |
| | | | | | | | | [symbol] |
| | | | | | | | | [symbol] |
| O35598 | ADA10 | Adam10 | TITLQPGSPCNDFR | 14 | 1.86E−06 | present | present | [symbol] |
| | | | | | | | | [symbol] |
| | | | | | | | | [symbol] |
| P45376 | ALDR | [symbol] | MPTLGLGTWK | 15 | 8.33E−05 | absent | present | [symbol] |
| | | | | | | | | [symbol] |
| | | | | | | | | [symbol] |
| | | | | | | | | [symbol] |
| P45376 | ALDR | [symbol] | [symbol] | 16 | 7.98E−05 | present | present | [symbol] |
| | | | | | | | | [symbol] |
| | | | | | | | | [symbol] |
| | | | | | | | | [symbol] |
| P45376 | ALDR | [symbol] | SPPGQVTEAVK | 17 | 6.70E−05 | present | present | [symbol] |
| | | | | | | | | [symbol] |
| | | | | | | | | [symbol] |
| | | | | | | | | [symbol] |
| [symbol] | [symbol] | Alb | [symbol] | 18 | [symbol] | absent | absent | [symbol] |
| P47738 | ALDH2 | Aldh2 | GYFIQPTVFGDVK | 19 | 1.34E−05 | absent | present | [symbol] |
| | | | | | | | | [symbol] |
| P47738 | ALDH2 | Aldh2 | [symbol] | 20 | 4.00E−04 | absent | absent | [symbol] |
| | | | | | | | | [symbol] |
| P10107 | ANXA1 | Anxa1 | [symbol] | 21 | [symbol] | absent | present | [symbol] |
| | | | | | | | | [symbol] |
| | | | | | | | | [symbol] |
| P10107 | ANXA1 | Anxa1 | [symbol] | 22 | 4.42E−06 | present | present | [symbol] |
| | | | | | | | | [symbol] |
| | | | | | | | | [symbol] |
| P10107 | ANAX1 | Anxa1 | [symbol] | 23 | 5.73E−06 | present | present | [symbol] |
| | | | | | | | | [symbol] |
| | | | | | | | | [symbol] |
| P07356 | ANXA2 | Anxa2 | [symbol] | 24 | 5.64E−06 | absent | present | [symbol] |
| | | | | | | | | [symbol] |
| | | | | | | | | [symbol] |
| P07356 | ANXA2 | Anxa2 | [symbol] | 25 | 4.65E−05 | absent | absent | [symbol] |
| | | | | | | | | [symbol] |
| | | | | | | | | [symbol] |
| P48036 | ANXA5 | Anxa5 | NFATSLYSMIK | 26 | 8.30E−06 | present | present | [symbol] |
| | | | | | | | | [symbol] |
| | | | | | | | | [symbol] |
| | | | | | | | | [symbol] |
| | | | | | | | | [symbol] |
| P48036 | ANXA5 | Anxa5 | SEIDLFNIR | 27 | 1.06E−07 | present | present | [symbol] |
| | | | | | | | | [symbol] |
| | | | | | | | | [symbol] |
| | | | | | | | | [symbol] |
| | | | | | | | | [symbol] |
| P48036 | ANXA5 | Anxa5 | TPEELSAIK | 28 | 1.44E−07 | absent | present | [symbol] |
| | | | | | | | | [symbol] |
| | | | | | | | | [symbol] |
| | | | | | | | | [symbol] |
| | | | | | | | | [symbol] |
| E9Q414 | APOB | Apob | [symbol] | 29 | 3.30E−06 | absent | present | [symbol] |
| | | | | | | | | [symbol] |
| E9Q414 | APOB | Apob | QGFFPDSVNK | 30 | 3.36E−06 | absent | absent | [symbol] |
| | | | | | | | | [symbol] |
| E9Q414 | APOB | Apob | YENYELTLK | 31 | 1.86E−06 | absent | present | [symbol] |
| | | | | | | | | [symbol] |
| Q99KN1 | ARRDI | [symbol] | DTSPVVASILQK | 32 | 1.31E−05 | absent | absent | [symbol] |
| Q99KN1 | ARRDI | [symbol] | LGAPLPFR | 33 | 8.57E−06 | present | present | [symbol] |
| Q99KN1 | ARRDI | [symbol] | TIAEVEGTGNK | 34 | 1.49E−06 | absent | present | [symbol] |
| [symbol] | [symbol] | [symbol] | FSTFAGFLLFETK | 35 | 2.25E−06 | present | present | [symbol] |
| | | | | | | | | [symbol] |
| | | | | | | | | [symbol] |
| [symbol] | [symbol] | [symbol] | [symbol] | 36 | 1.62E−07 | present | present | [symbol] |
| | | | | | | | | [symbol] |
| | | | | | | | | [symbol] |
| [symbol] | [symbol] | [symbol] | [symbol] | 37 | 4.67E−04 | absent | absent | [symbol] |
| | | | | | | | | [symbol] |
| | | | | | | | | [symbol] |
| P01029 | CO4B | C4b | [symbol] | 38 | 3.47E−06 | absent | absent | [symbol] |
| | | | | | | | | [symbol] |
| | | | | | | | | [symbol] |
| P01029 | CO4B | C4b | VFALDQK | 39 | 2.09E−07 | present | present | [symbol] |
| | | | | | | | | [symbol] |
| | | | | | | | | [symbol] |
| P35564 | [symbol] | Canx | [symbol] | 40 | 2.47E−05 | absent | present | Calnexin |
| P35564 | [symbol] | Canx | GLVLMSR | 41 | 4.04E−05 | absent | present | Calnexin |
| P35564 | [symbol] | Canx | [symbol] | 42 | 4.06E−05 | absent | present | Caltexin |
| P41731 | CD63 | Cd63 | [symbol] | 43 | 3.31E−05 | absent | present | [symbol] |
| P41731 | CD63 | Cd63 | SFQQQMQNYLK | 44 | 7.85E−06 | absent | present | [symbol] |
| P35762 | [symbol] | Cd81 | [symbol] | 45 | 1.27E−06 | absent | absent | [symbol] |
| | | | | | | | | [symbol] |
| P35762 | [symbol] | Cd81 | [symbol] | 46 | 3.85E−06 | absent | present | [symbol] |
| | | | AK | | | | | [symbol] |
| P40240 | CD9 | Cd9 | ELQIFYK | 47 | 5.47E−06 | absent | absent | [symbol] |
| Q9DAW9 | CNN3 | Cnn3 | [symbol] | 48 | 4.94E−05 | present | present | [symbol] |
| Q9DAW9 | CNN3 | Cnn3 | GASQAGMLAPGTR | 49 | 2.96E−05 | absent | present | [symbol] |
| Q9DAW9 | CNN3 | Cnn3 | [symbol] | 50 | 2.82E−05 | present | present | [symbol] |
| Q6ZQ08 | CNOT1 | Cnot1 | [symbol] | 51 | 1.65E−05 | absent | absent | [symbol] |
| | | | | | | | | [symbol] |
| Q6ZQ08 | CNOT1 | Cnot1 | [symbol] | 52 | 1.99E−05 | absent | present | [symbol] |
| | | | | | | | | [symbol] |
| Q6ZQ08 | CNOT1 | Cnot1 | NLILSAFPR | 53 | 1.81E−05 | absent | present | [symbol] |
| | | | | | | | | [symbol] |
| O88207 | CO5A1 | [symbol] | [symbol] | 54 | 2.33E−06 | present | present | [symbol] |
| O88207 | CO5A1 | [symbol] | RPLGTQQNPAR | 55 | 4.69E−06 | present | present | [symbol] |
| O88207 | CO5A1 | [symbol] | [symbol] | 56 | 2.79E−06 | present | present | [symbol] |
| O55029 | COPB2 | Copb2 | IWDYQNK | 57 | 1.12E−06 | absent | present | [symbol] |
| O55029 | [symbol] | Copb3 | TYLPSQVSR | 58 | 1.64E−06 | present | present | [symbol] |
| O55029 | [symbol] | Copb3 | VLPTIPK | 59 | 1.58E−06 | present | present | [symbol] |
| Q61147 | CERU | Cp | DIFTGLIGPMK | 60 | 2.64E−05 | present | present | [symbol] |
| Q61147 | CERU | Cp | NMATRPYSIHAHGNK | 61 | 1.15E−06 | present | present | [symbol] |
| P23927 | CRYAB | DCTN2 | APSWIDTGLSEMR | 62 | 1.49E−05 | absent | absent | [symbol] |
| P23927 | CRYAB | DCTN2 | [symbol] | 63 | 1.90E−05 | absent | absent | [symbol] |
| P97792 | CXAR | DCTN2 | APQSPTLAPAK | 64 | 2.89E−05 | absent | absent | [symbol] |
| P97792 | CXAR | DCTN2 | VAAPNLSR | 65 | 3.92E−05 | present | present | [symbol] |
| P97792 | CXAR | DCTN2 | VGSDQCMLR | 66 | 4.08E−05 | absent | present | [symbol] |
| Q922B2 | SYDC | DCTN2 | [symbol] | 67 | 1.27E−06 | present | present | [symbol] |
| | | | | | | | | [symbol] |
| Q922B2 | SYDC | DCTN2 | IYVISLAEPR | 68 | 1.61E−06 | present | present | [symbol] |
| | | | | | | | | [symbol] |
| Q922B2 | SYDC | DCTN2 | [symbol] | 69 | 1.18E−06 | absent | present | [symbol] |
| | | | | | | | | [symbol] |
| Q99KJ8 | DCTN2 | DCTN2 | [symbol] | 70 | 1.55E−05 | present | present | [symbol] |
| | | | | | | | | [symbol] |
| | | | | | | | | [symbol] |
| Q99KJ8 | DCTN2 | DCTN2 | LLLQLEATK | 71 | 1.51E−05 | present | present | [symbol] |
| | | | | | | | | [symbol] |
| | | | | | | | | [symbol] |
| Q99KJ8 | DCTN2 | DCTN2 | VSALDLAVLDQVEAR | 72 | 1.65E−05 | present | present | [symbol] |
| | | | | | | | | [symbol] |
| | | | | | | | | [symbol] |
| Q9Z1N5 | DX39E | Ddx39b | [symbol] | 73 | 2.08E−05 | absent | present | [symbol] |
| | | | | | | | | [symbol] |
| | | | | | | | | [symbol] |
| Q9Z1N5 | DX39E | Ddx39b | [symbol] | 74 | 1.84E−05 | absent | present | [symbol] |
| | | | | | | | | [symbol] |
| | | | | | | | | [symbol] |
| Q62167 | DDX3X | Ddx3x | [symbol] | 75 | 9.46E−05 | absent | present | [symbol] |
| | | | | | | | | [symbol] |
| | | | | | | | | [symbol] |
| | | | | | | | | [symbol] |
| Q62167 | DDX3X | Ddx3x | HAIPIK | 76 | 1.11E−06 | absent | absent | [symbol] |
| | | | | | | | | [symbol] |
| | | | | | | | | [symbol] |
| | | | | | | | | [symbol] |
| Q62167 | DDX3X | Ddx3x | SPILVATAVAAR | 77 | 1.29E−06 | present | present | [symbol] |
| | | | | | | | | [symbol] |
| | | | | | | | | [symbol] |
| | | | | | | | | [symbol] |
| Q61656 | DDX5 | Ddx5 | LLQLVLDR | 78 | 8.60E−05 | absent | present | [symbol] |
| | | | | | | | | [symbol] |
| | | | | | | | | [symbol] |
| Q61656 | DDX5 | Ddx5 | [symbol] | 79 | 6.57E−05 | absent | present | [symbol] |
| | | | | | | | | [symbol] |
| | | | | | | | | [symbol] |
| Q61656 | DDX5 | Ddx5 | [symbol] | 80 | 4.41E−05 | absent | present | [symbol] |
| | | | | | | | | [symbol] |
| | | | | | | | | [symbol] |
| O35286 | [symbol] | [symbol] | EAMNDPLLER | 81 | 1.14E−06 | absent | present | [symbol] |
| | | | | | | | | [symbol] |
| O35286 | [symbol] | [symbol] | [symbol] | 82 | 1.11E−06 | absent | present | [symbol] |
| | | | | | | | | [symbol] |
| O35286 | [symbol] | [symbol] | [symbol] | 83 | 1.24E−06 | absent | present | [symbol] |
| | | | | | | | | [symbol] |
| O70133 | DHX9 | Dhx9 | DVVLAYPEVR | 84 | 6.44E−05 | absent | present | [symbol] |
| | | | | | | | | [symbol] |
| | | | | | | | | [symbol] |
| O70133 | DHX9 | Dhx9 | [symbol] | 85 | 9.06E−05 | absent | present | [symbol] |
| | | | | | | | | [symbol] |
| | | | | | | | | [symbol] |
| O70133 | DHX9 | Dhx9 | [symbol] | 86 | 5.35E−05 | absent | present | [symbol] |
| | | | | | | | | [symbol] |
| | | | | | | | | [symbol] |
| Q9D1M4 | MCA3 | Ecflel | AMVQQWLEFR | 87 | 3.99E−05 | absent | present | [symbol] |
| | | | | | | | | [symbol] |
| Q9D1M4 | MCA3 | [symbol] | DLNSYLEDK | 88 | 1.83E−05 | absent | absent | [symbol] |
| | | | | | | | | [symbol] |
| Q9D1M4 | MCAR | [symbol] | [symbol] | 89 | 3.59E−05 | absent | present | [symbol] |
| | | | | | | | | [symbol] |
| Q8BPB5 | FBLN3 | Efemp1 | CVNHYGGYLCLPK | 90 | 1.02E−06 | absent | present | [symbol] |
| | | | | | | | | [symbol] |
| Q8BPB5 | FBLN3 | Efemp1 | [symbol] | 91 | 8.55E−05 | absent | present | [symbol] |
| | | | | | | | | [symbol] |
| Q8BPB5 | FBLN3 | Efemp1 | [symbol] | 92 | 1.07E−06 | absent | present | [symbol] |
| | | | | | | | | [symbol] |
| [symbol] | ESYT1 | [symbol] | ALTLGALTLPLAR | 93 | 1.80E−05 | absent | present | [symbol] |
| | | | | | | | | [symbol] |
| [symbol] | ESYT1 | [symbol] | [symbol] | 94 | 2.668−05 | absent | present | [symbol] |
| | | | | | | | | [symbol] |
| [symbol] | ESYT1 | [symbol] | [symbol] | 95 | 1.13E−05 | absent | absent | [symbol] |
| | | | | | | | | [symbol] |
| O35382 | EXOC4 | Exoc4 | [symbol] | 96 | 7.00E−04 | absent | absent | [symbol] |
| O35382 | EXOC4 | Exoc4 | [symbol] | 97 | 7.04E−04 | present | present | [symbol] |
| O35382 | EXOC4 | Exoc4 | STTQVADSAYQR | 98 | 1.19E−05 | absent | present | [symbol] |
| E9PV24 | [symbol] | Fga | [symbol] | 99 | 1.10E−05 | absent | absent | [symbol] |
| | | | | | | | | [symbol] |
| E9PV24 | [symbol] | Fga | [symbol] | 100 | 3.04E−05 | absent | absent | [symbol] |
| | | | | | | | | [symbol] |
| Q8VCM7 | [symbol] | [symbol] | IDGSLDFK | 101 | 7.54E−05 | present | absent | [symbol] |
| Q60634 | FLOT2 | flot2 | [symbol] | 102 | 2.03E−05 | absent | absent | [symbol] |
| | | | | | | | | [symbol] |
| Q60634 | FLOT2 | Flot2 | NVVLQTLEGHLR | 103 | 8.18E−05 | present | present | [symbol] |
| | | | | | | | | [symbol] |
| Q60634 | FLOT2 | flot2 | QVLLAQAFAEK | 104 | 3.03E−05 | present | present | [symbol] |
| | | | | | | | | [symbol] |
| P09528 | [symbol] | [symbol] | [symbol] | 105 | 1.47E−05 | present | absent | [symbol] |
| | | | | | | | | [symbol] |
| P09528 | [symbol] | [symbol] | [symbol] | 106 | 1.02E−05 | absent | present | [symbol] |
| | | | | | | | | [symbol] |
| P56959 | FUS | Fus | [symbol] | 107 | 2.28E−05 | present | present | [symbol] |
| P56959 | FUS | Fus | [symbol] | 108 | 7.99E−04 | present | present | [symbol] |
| P56959 | FUS | Fus | [symbol] | 109 | 6.57E−04 | absent | present | [symbol] |
| O09131 | GSTO1 | Gsto1 | FCPFAQR | 110 | 5.61E−05 | absent | present | [symbol] |
| | | | | | | | | [symbol] |
| | | | | | | | | [symbol] |
| | | | | | | | | [symbol] |
| | | | | | | | | [symbol] |
| O09131 | GSTO1 | Gato1 | [symbol] | 111 | 1.04E−06 | absent | present | [symbol] |
| | | | | | | | | [symbol] |
| | | | | | | | | [symbol] |
| | | | | | | | | [symbol] |
| | | | | | | | | [symbol] |
| O09131 | GSTO1 | Gsto1 | VPPLIASFVR | 112 | 7.58E−05 | absent | present | [symbol] |
| | | | | | | | | [symbol] |
| | | | | | | | | [symbol] |
| | | | | | | | | [symbol] |
| | | | | | | | | [symbol] |
| DSYXG0 | [symbol] | [symbol] | ITGLSTIDFR | 113 | 3.96E−05 | present | present | [symbol] |
| D3YXG0 | [symbol] | [symbol] | [symbol] | 114 | 3.71E−05 | absent | present | [symbol] |
| D3YXG0 | [symbol] | [symbol] | QVTLECK | 115 | 9.47E−05 | absent | present | [symbol] |
| Q9Z2X1 | HNRPF | [symbol] | [symbol] | 116 | 3.92E−05 | absent | present | [symbol] |
| | | | | | | | | [symbol] |
| | | | | | | | | [symbol] |
| P35737 | [symbol] | [symbol] | GLPWSCSADEVQR | 117 | 2.54E−05 | present | present | [symbol] |
| | | | | | | | | [symbol] |
| | | | | | | | | [symbol] |
| O35737 | [symbol] | [symbol] | [symbol] | 118 | 1.85E−05 | absent | present | [symbol] |
| | | | | | | | | [symbol] |
| | | | | | | | | [symbol] |
| O35737 | [symbol] | [symbol] | [symbol] | 119 | 2.36E−05 | present | present | [symbol] |
| | | | | | | | | [symbol] |
| | | | | | | | | [symbol] |
| Q8R081 | [symbol] | [symbol] | [symbol] | 120 | 6.96E−05 | present | present | [symbol] |
| Q8R081 | [symbol] | [symbol] | [symbol] | 121 | 7.30E−05 | present | present | [symbol] |
| P07901 | HS90A | Hsp90 | [symbol] | 122 | 2.94E−06 | present | present | [symbol] |
| | | aa1 | | | | | | [symbol] |
| | | | | | | | | [symbol] |
| P07901 | HS90A | Hsp90 | [symbol] | 123 | 1.14E−07 | absent | present | [symbol] |
| | | aa1 | | | | | | [symbol] |
| | | | | | | | | [symbol] |
| P07901 | HS90A | Hsp90 | [symbol] | 124 | 7.64E−06 | absent | present | [symbol] |
| | | aa1 | | | | | | [symbol] |
| | | | | | | | | [symbol] |
| P11499 | HS90B | Hsp90 | ALLFIPR | 125 | 2.24E−07 | present | present | [symbol] |
| | | ab1 | | | | | | [symbol] |
| P11499 | HIS9OB | Hsp90 | [symbol] | 126 | 1.87E−07 | present | present | [symbol] |
| | | ab1 | | | | | | [symbol] |
| P11499 | HIS90B | Hsp90 | [symbol] | 127 | 1.41E−07 | present | present | [symbol] |
| | | ab1 | | | | | | [symbol] |
| P54071 | [symbol] | [symbol] | [symbol] | 128 | 1.98E−05 | absent | present | [symbol] |
| | | | | | | | | [symbol] |
| | | | | | | | | [symbol] |
| P54071 | [symbol] | [symbol] | [symbol] | 129 | 1.56E−05 | absent | present | [symbol] |
| | | | | | | | | [symbol] |
| | | | | | | | | [symbol] |
| P54071 | [symbol] | [symbol] | [symbol] | 130 | 1.95E−05 | absent | present | [symbol] |
| | | | | | | | | [symbol] |
| | | | | | | | | [symbol] |
| Q60751 | IGF1R | Igf1r | [symbol] | 131 | 1.68E−05 | absent | present | [symbol] |
| | | | | | | | | [symbol] |
| | | | | | | | | [symbol] |
| | | | | | | | | [symbol] |
| Q60751 | IGF1R | Igf1r | HSHALVSLSFLK | 132 | 1.97E−05 | absent | present | [symbol] |
| | | | | | | | | [symbol] |
| | | | | | | | | [symbol] |
| | | | | | | | | [symbol] |
| Q60751 | IGF1R | Igf1r | IGDFGMITR | 133 | 2.87E−05 | present | present | [symbol] |
| | | | | | | | | [symbol] |
| | | | | | | | | [symbol] |
| | | | | | | | | [symbol] |
| O55023 | IMPA1 | Impa1 | IVLSSMEK | 134 | 2.43E−05 | absent | present | [symbol] |
| | | | | | | | | [symbol] |
| | | | | | | | | [symbol] |
| | | | | | | | | [symbol] |
| O55023 | IMPA1 | Impa1 | SLLVTELGSSR | 135 | 3.47E−05 | present | present | [symbol] |
| | | | | | | | | [symbol] |
| | | | | | | | | [symbol] |
| | | | | | | | | [symbol] |
| Q8K2V6 | [symbol] | [symbol] | [symbol] | 136 | 1.58E−05 | absent | present | [symbol] |
| Q8K2V6 | [symbol] | [symbol] | [symbol] | 137 | 5.70E−04 | absent | absent | [symbol] |
| Q8K2V6 | [symbol] | [symbol] | [symbol] | 138 | 2.24E−05 | absent | present | [symbol] |
| Q9EPL8 | IPO7 | Ipo7 | [symbol] | 139 | 1.31E−06 | absent | present | [symbol] |
| Q9EPL8 | IPO7 | Ipo7 | [symbol] | 140 | 1.58E−06 | present | present | [symbol] |
| Q9EPL8 | IPO7 | Ipo7 | [symbol] | 141 | 1.56E−06 | present | present | [symbol] |
| Q54890 | ITB3 | [symbol] | [symbol] | 142 | 5.44E−05 | absent | absent | [symbol] |
| | | | | | | | | [symbol] |
| Q54890 | ITB3 | [symbol] | [symbol] | 143 | 7.94E−04 | absent | present | [symbol] |
| | | | | | | | | [symbol] |
| Q54890 | ITB3 | [symbol] | [symbol] | 144 | 4.40E−05 | absent | present | [symbol] |
| | | | | | | | | [symbol] |
| O89051 | ITM2B | [symbol] | VTFNSALAQK | 145 | 2.17E−05 | absent | absent | [symbol] |
| | | | | | | | | [symbol] |
| | | | | | | | | [symbol] |
| | | | | | | | | [symbol] |
| | | | | | | | | [symbol] |
| O89051 | ITM2B | [symbol] | YQTIEENIK | 146 | 2.60E−05 | absent | absent | [symbol] |
| | | | | | | | | [symbol] |
| | | | | | | | | [symbol] |
| | | | | | | | | [symbol] |
| | | | | | | | | [symbol] |
| Q9D892 | ITPA | Itpa | [symbol] | 147 | 3.31E−05 | absent | present | [symbol] |
| | | | | | | | | [symbol] |
| | | | | | | | | [symbol] |
| | | | | | | | | [symbol] |
| | | | | | | | | [symbol] |
| O35343 | IMA3 | Kpna4 | [symbol] | 148 | 3.15E−05 | absent | absent | [symbol] |
| | | | | | | | | [symbol] |
| O35343 | IMA3 | Kpna4 | [symbol] | 149 | 6.87E−04 | absent | present | [symbol] |
| | | | | | | | | [symbol] |
| O35343 | IMA3 | Kpna4 | [symbol] | 150 | 5.95E−04 | absent | absent | [symbol] |
| | | | | | | | | [symbol] |
| Q9WVG5 | [symbol] | [symbol] | [symbol] | 151 | [symbol] | absent | present | [symbol] |
| | | | | | | | | [symbol] |
| Q9WVG5 | [symbol] | [symbol] | NYLSQPSNPSR | 152 | 1.26E−05 | absent | present | [symbol] |
| | | | | | | | | [symbol] |
| Q9WVG5 | [symbol] | [symbol] | [symbol] | 153 | 1.46E−05 | absent | absent | [symbol] |
| | | | | | | | | [symbol] |
| P48678 | LMNA | Lmna | [symbol] | 154 | 4.96E−05 | absent | present | [symbol] |
| P48678 | LMNA | Lmna | LADALQELR | 155 | 7.33E−05 | absent | present | [symbol] |
| P48678 | LMNA | Lmna | [symbol] | 156 | 5.33E−05 | absent | present | [symbol] |
| Q8CG19 | LTBP1 | Ltbp1 | CPLPGTAAFK | 157 | 1.17E−06 | present | present | [symbol] |
| | | | | | | | | [symbol] |
| | | | | | | | | [symbol] |
| Q8CG19 | LTBP1 | Ltbp1 | [symbol] | 158 | 2.68E−06 | present | present | [symbol] |
| | | | | | | | | [symbol] |
| | | | | | | | | [symbol] |
| Q8CG19 | LTBP1 | Ltbp1 | VVFTPSICK | 159 | 2.27E−06 | present | present | [symbol] |
| | | | | | | | | [symbol] |
| | | | | | | | | [symbol] |
| P97310 | MCM2 | Mcm2 | [symbol] | 160 | 7.50E−05 | absent | present | [symbol] |
| | | | | | | | | [symbol] |
| | | | | | | | | [symbol] |
| P97310 | MCM2 | Mcm2 | [symbol] | 161 | 7.43E−05 | absent | present | [symbol] |
| | | | | | | | | [symbol] |
| | | | | | | | | [symbol] |
| P97310 | MCM2 | Mcm2 | VAVGELTDEDVK | 162 | 5.72E−05 | absent | present | [symbol] |
| | | | | | | | | [symbol] |
| | | | | | | | | [symbol] |
| PL2025 | MK | Mdk | [symbol] | 163 | 1.13E−05 | absent | present | [symbol] |
| | | | | | | | | [symbol] |
| PL2025 | MK | Mdk | [symbol] | 164 | 1.95E−05 | present | present | [symbol] |
| | | | | | | | | [symbol] |
| Q9CQ65 | MTAP | Mtap | [symbol] | 165 | 1.10E−06 | absent | present | [symbol] |
| | | | | | | | | [symbol] |
| | | | | | | | | [symbol] |
| Q9CQ65 | MTAP | Mtap | [symbol] | 166 | 1.02E−06 | absent | present | [symbol] |
| | | | | | | | | [symbol] |
| | | | | | | | | [symbol] |
| Q9CQ65 | MTAP | Mtap | [symbol] | 167 | 1.12E−06 | absent | present | [symbol] |
| | | | | | | | | [symbol] |
| | | | | | | | | [symbol] |
| Q9JMH9 | MY18A | Myo18a | IISNLFLGR | 168 | 2.74E−05 | absent | present | [symbol] |
| | | | | | | | | [symbol] |
| | | | | | | | | [symbol] |
| Q9JMH9 | MY18A | Myo18a | LTAELQDTK | 169 | 1.75E−05 | absent | absent | [symbol] |
| | | | | | | | | [symbol] |
| | | | | | | | | [symbol] |
| Q9JMH9 | MY18A | Myo18a | [symbol] | 170 | 1.73E−05 | absent | present | [symbol] |
| | | | | | | | | [symbol] |
| | | | | | | | | [symbol] |
| Q78ZA7 | [symbol] | [symbol] | [symbol] | 171 | 1.57E−06 | absent | present | [symbol] |
| Q78ZA7 | [symbol] | [symbol] | [symbol] | 172 | 4.67E−05 | absent | present | [symbol] |
| Q99J45 | NRBP | Nrbp1 | [symbol] | 173 | 1.80E−05 | absent | present | [symbol] |
| | | | | | | | | [symbol] |
| Q99J45 | NRBP | Nrbp1 | [symbol] | 174 | 1.52E−05 | present | present | [symbol] |
| | | | | | | | | [symbol] |
| Q99J45 | NRBP | Nrbp1 | LTSLLEETLNK | 175 | 7.77E−04 | absent | absent | [symbol] |
| | | | | | | | | [symbol] |
| Q7TQ13 | [symbol] | [symbol] | [symbol] | 176 | 7.80E−05 | absent | present | [symbol] |
| | | | | | | | | [symbol] |
| | | | | | | | | [symbol] |
| | | | | | | | | [symbol] |
| Q7TQ13 | [symbol] | [symbol] | [symbol] | 177 | 3.75E−05 | absent | absent | [symbol] |
| | | | | | | | | [symbol] |
| | | | | | | | | [symbol] |
| | | | | | | | | [symbol] |
| Q7TQ13 | [symbol] | [symbol] | LLTSGYLQR | 178 | 5.68E−05 | absent | absent | [symbol] |
| | | | | | | | | [symbol] |
| | | | | | | | | [symbol] |
| | | | | | | | | [symbol] |
| [symbol] | [symbol] | [symbol] | ELPELLQR | 179 | 5.68E−06 | present | present | [symbol] |
| | | | | | | | | [symbol] |
| | | | | | | | | [symbol] |
| [symbol] | [symbol] | [symbol] | LALASLGYEK | 180 | 6.77E−06 | present | present | [symbol] |
| | | | | | | | | [symbol] |
| | | | | | | | | [symbol] |
| [symbol] | [symbol] | [symbol] | NIQVSHQEFSK | 181 | 6.56E−06 | present | present | [symbol] |
| | | | | | | | | [symbol] |
| | | | | | | | | [symbol] |
| P08003 | PDIA4 | Pdia4 | [symbol] | 182 | 2.18E−05 | absent | present | [symbol] |
| | | | | | | | | [symbol] |
| P08003 | PDIA4 | Pdia4 | [symbol] | 183 | 5.48E−05 | absent | present | [symbol] |
| | | | | | | | | [symbol] |
| P08003 | PDIA4 | Pdia4 | [symbol] | 184 | 3.58E−05 | present | present | [symbol] |
| | | | | | | | | [symbol] |
| P22005 | PENK | Penk | SPQLEDEAK | 185 | 1.96E−05 | absent | present | [symbol] |
| | | | | | | | | [symbol] |
| | | | | | | | | [symbol] |
| | | | | | | | | [symbol] |
| P22005 | PENK | Penk | VGRPEWWMDYQK | 186 | 1.47E−05 | absent | present | [symbol] |
| | | | | | | | | [symbol] |
| | | | | | | | | [symbol] |
| | | | | | | | | [symbol] |
| P27612 | PLAP | Plaa | [symbol] | 187 | 1.15E−05 | absent | present | [symbol] |
| P27612 | PLAP | Plaa | IGDVVGSSGANQQTSGK | 188 | 9.56E−04 | absent | present | [symbol] |
| P27612 | PLAP | Plaa | ILPEQGLMLTGSADK | 189 | 1.55E−05 | absent | present | [symbol] |
| O35405 | PLD3 | [symbol] | ALLNVVDSAR | 190 | 2.03E−05 | absent | present | [symbol] |
| | | | | | | | | [symbol] |
| | | | | | | | | [symbol] |
| | | | | | | | | [symbol] |
| Q9QUR6 | PPCE | Prep | ELPDVLER | 191 | 1.05E−06 | absent | present | [symbol] |
| | | | | | | | | [symbol] |
| Q9QUR6 | PPCE | Prep | FIATLQYIVGR | 192 | 4.28E−05 | absent | present | [symbol] |
| | | | | | | | | [symbol] |
| Q9QUR6 | PPCE | Prep | [symbol] | 193 | 7.49E−05 | absent | present | [symbol] |
| | | | | | | | | [symbol] |
| Q99PV0 | [symbol] | [symbol] | ANPALYVLR | 194 | 9.40E−06 | absent | present | [symbol] |
| Q99PV0 | [symbol] | [symbol] | IDLTLLNR | 195 | 1.15E−06 | absent | present | [symbol] |
| Q99PV0 | [symbol] | [symbol] | TAEEVAALIR | 196 | 9.76E−05 | absent | present | [symbol] |
| Q3TNS7 | PSMD1 | Psmd1 | [symbol] | 197 | 7.75E−05 | absent | present | [symbol] |
| | | | | | | | | [symbol] |
| Q3TNS7 | PSMD1 | Psmd1 | [symbol] | 198 | 1.97E−06 | absent | present | [symbol] |
| | | | | | | | | [symbol] |
| Q3TXS7 | PSMD1 | Psmdl | QDVYDLLK | 199 | [symbol] | absent | present | [symbol] |
| | | | | | | | | [symbol] |
| P42669 | PURA | Pura | [symbol] | 200 | 2.93E−05 | absent | present | [symbol] |
| | | | | | | | | [symbol] |
| P42669 | PURA | Pura | [symbol] | 201 | 6.21E−05 | absent | present | [symbol] |
| | | | | | | | | [symbol] |
| P42669 | PURA | [symbol] | VSFVKPTYR | 202 | 2.96E−05 | absent | present | [symbol] |
| | | | | | | | | [symbol] |
| Q8BML9 | SYQ | Qars1 | AINFNEGYAK | 203 | 3.29E−05 | absent | absent | [symbol] |
| | | | | | | | | [symbol] |
| Q8BML9 | SYQ | Qars1 | [symbol] | 204 | 5.06E−05 | present | present | [symbol] |
| | | | | | | | | [symbol] |
| Q8BML9 | SYQ | Qarsl | [symbol] | 205 | 3.81E−05 | absent | present | [symbol] |
| | | | | | | | | [symbol] |
| P35279 | RAB6A | Rab6a | [symbol] | 206 | 4.40E−05 | present | present | [symbol] |
| P35279 | RAB6A | Rab6a | TMYLEDR | 207 | 4.40E−05 | present | present | [symbol] |
| P35279 | RAB6A | Rab6a | VAAALPGMESTQDR | 208 | 2.88E−05 | present | present | [symbol] |
| Q60972 | RBBP4 | Rbbp4 | [symbol] | 209 | 4.74E−05 | absent | absent | [symbol] |
| | | | | | | | | [symbol] |
| | | | | | | | | [symbol] |
| | | | | | | | | [symbol] |
| | | | | | | | | [symbol] |
| P67984 | RL22 | Rpl22 | AGNLGGGVVTIER | 210 | 3.26E−06 | present | present | [symbol] |
| P67984 | RL22 | Rpl22 | [symbol] | 211 | 2.64E−06 | present | present | [symbol] |
| P50543 | S10AB | S100a1l | [symbol] | 212 | 1.89E−06 | absent | present | [symbol] |
| | | | | | | | | [symbol] |
| | | | | | | | | [symbol] |
| P50543 | S10AB | S100a11 | [symbol] | 213 | 1.48E−06 | absent | present | [symbol] |
| | | | | | | | | [symbol] |
| | | | | | | | | [symbol] |
| O35114 | [symbol] | [symbol] | [symbol] | 214 | 1.38E−05 | absent | absent | [symbol] |
| | | | | | | | | [symbol] |
| | | | | | | | | [symbol] |
| Q01405 | SC23A | Sec23a | AETEEGPDVLR | 215 | 7.94E−05 | absent | present | [symbol] |
| Q01405 | SC23A | Sec23a | [symbol] | 216 | 1.14E−06 | absent | present | [symbol] |
| Q01405 | SC23A | Sec23a | [symbol] | 217 | 3.62E−D6 | absent | present | [symbol] |
| Q3UPL0 | [symbol] | [symbol] | FASSPLR | 218 | 5.50E−05 | absent | present | [symbol] |
| | | | | | | | | [symbol] |
| Q3UPL0 | SC31A | Sec31a | LITAVVMK | 219 | 6.26E−05 | absent | present | [symbol] |
| | | | | | | | | [symbol] |
| Q3UPL0 | SC31A | Sec3la | [symbol] | 220 | 4.29E−05 | present | present | [symbol] |
| | | | | | | | | [symbol] |
| Q9CZN7 | GLYM | [symbol] | [symbol] | 221 | 3.13E−05 | absent | present | [symbol] |
| | | | | | | | | [symbol] |
| Q9CZN7 | GLYM | [symbol] | [symbol] | 222 | 4.05E−05 | absent | present | [symbol] |
| | | | | | | | | [symbol] |
| Q9CZN7 | GLYM | [symbol] | VIPSPFK | 223 | 2.78E−05 | absent | present | [symbol] |
| | | | | | | | | [symbol] |
| Q6X893 | CTL1 | Slc44a1 | FAEALITFYSDNSVLHR | 224 | 2.51E−05 | absent | present | [symbol] |
| | | | | | | | | [symbol] |
| Q6X893 | CTL1 | Slc44al | LPVPASAPIPFFIR | 225 | 8.71E−05 | present | present | [symbol] |
| | | | | | | | | [symbol] |
| Q6X893 | CTL1 | Slc44al | NLPFTPILASVNR | 226 | 4.19E−05 | absent | present | [symbol] |
| | | | | | | | | [symbol] |
| P10923 | OSTP | Spp1 | [symbol] | 227 | [symbol] | absent | present | [symbol] |
| | | | | | | | | [symbol] |
| | | | | | | | | [symbol] |
| P10923 | OSTP | Spp1 | GDSLAYGLR | 228 | 7.58E−05 | absent | present | [symbol] |
| | | | | | | | | [symbol] |
| | | | | | | | | [symbol] |
| P10923 | OSTP | Spp1 | [symbol] | 229 | 9.12E−05 | present | present | [symbol] |
| | | | | | | | | [symbol] |
| | | | | | | | | [symbol] |
| P16546 | SPTN1 | Sptaa1 | DLSSVQTLLTK | 230 | 6.03E−05 | absent | present | [symbol] |
| | | | | | | | | [symbol] |
| P16546 | SPTN1 | Sptaa1 | LDENSAFLQFNWK | 231 | 6.23E−05 | absent | present | [symbol] |
| | | | | | | | | [symbol] |
| P16546 | SPTN1 | Sptaa1 | [symbol] | 232 | 7.22E−05 | absent | present | [symbol] |
| | | | | | | | | [symbol] |
| Q62261 | SPTB2 | Sptbn1 | VAVVNQIAR | 233 | 7.81E−05 | absent | present | [symbol] |
| | | | | | | | | [symbol] |
| Q62261 | SPTB2 | Sptbn1 | VLDNAIETEK | 234 | 7.91E−05 | absent | present | [symbol] |
| | | | | | | | | [symbol] |
| Q62261 | SPTB2 | Sptbn1 | VQAVVAVAR | 235 | 7.64E−05 | absent | present | [symbol] |
| | | | | | | | | [symbol] |
| Q8BL97 | SRSF7 | Srs17 | AFSYYGPLR | 236 | 5.70E−06 | absent | present | [symbol] |
| | | | | | | | | [symbol] |
| Q8BL97 | SRSF7 | Srs17 | RPFDPNDRCYECGEK | 237 | 2.42E−05 | absent | present | [symbol] |
| | | | | | | | | [symbol] |
| Q8BL97 | SRSF7 | Srs17 | VELSTGMPR | 238 | 1.87E−06 | absent | present | [symbol] |
| | | | | | | | | [symbol] |
| P32067 | LA | Ssh | [symbol] | 239 | 6.41E−05 | absent | present | [symbol] |
| | | | | | | | | [symbol] |
| P32067 | LA | Ssh | [symbol] | 240 | 2.52E−05 | absent | present | [symbol] |
| | | | | | | | | [symbol] |
| P32067 | LA | Ssb | NANNGNLLLR | 241 | 3.61E−05 | absent | present | [symbol] |
| | | | | | | | | [symbol] |
| Q9CYR0 | SSBP | Ssbp1 | DVAYQYVK | 242 | 1.09E−05 | absent | present | [symbol] |
| | | | | | | | | [symbol] |
| Q9CYR0 | SSBP | Ssbp1 | NPVTIFSLATNEMWR | 243 | 3.90E−04 | absent | present | [symbol] |
| | | | | | | | | [symbol] |
| Q9CYR0 | SSBP | Ssbp1 | SGDSEVYQMGDVSQK | 244 | 1.01E−05 | absent | present | [symbol] |
| | | | | | | | | [symbol] |
| A2AVA0 | SVEP1 | Svep1 | FAAGSVVSFK | 245 | 3.29E−05 | absent | present | [symbol] |
| | | | | | | | | [symbol] |
| A2AVA0 | SVEP1 | Svep1 | GAFQQAAQILR | 246 | 4.72E−05 | absent | present | [symbol] |
| | | | | | | | | [symbol] |
| A2AVA0 | SVEP1 | Svep1 | VAIVTFSSK | 247 | 2.97E−05 | absent | present | [symbol] |
| | | | | | | | | [symbol] |
| Q9D0R2 | SYTC | Tars1 | AILGSVER | 248 | 1.29E−06 | present | present | [symbol] |
| | | | | | | | | [symbol] |
| | | | | | | | | [symbol] |
| Q9D0R2 | SYTC | Tars1 | NFLSGALTGLTR | 249 | 9.45E−05 | present | present | [symbol] |
| | | | | | | | | [symbol] |
| | | | | | | | | [symbol] |
| Q9D0R2 | SYTC | Tars1 | VNTPITTVYR | 250 | 1.24E−06 | present | present | [symbol] |
| | | | | | | | | [symbol] |
| | | | | | | | | [symbol] |
| P62996 | TRA2R | Tra2b | GFAFVYFENVDDAK | 251 | 1.11E−05 | present | present | [symbol] |
| | | | | | | | | [symbol] |
| | | | | | | | | [symbol] |
| P62996 | TRA2B | Tra2b | YGPLADVSIVYDAQSR | 252 | 5.45E−05 | absent | present | [symbol] |
| | | | | | | | | [symbol] |
| | | | | | | | | [symbol] |
| Q61187 | TS101 | Tsg101 | ASLISANSDK | 253 | 9.80E−05 | present | present | [symbol] |
| | | | | | | | | [symbol] |
| Q61187 | TS101 | Tsg101 | DGTISEDTIR | 254 | 1.06E−06 | present | present | [symbol] |
| | | | | | | | | [symbol] |
| Q61187 | TS101 | Tsg101 | [symbol] | 255 | 1.75E−06 | absent | present | [symbol] |
| | | | | | | | | [symbol] |
| Q8C878 | UBA3 | Uba3 | [symbol] | 256 | 5.61E−04 | absent | absent | [symbol] |
| | | | | | | | | [symbol] |
| | | | | | | | | [symbol] |
| | | | | | | | | [symbol] |
| Q8C878 | UBA3 | Uba3 | SPAITATLEGK | 257 | 2.78E−05 | absent | present | [symbol] |
| | | | | | | | | [symbol] |
| | | | | | | | | [symbol] |
| | | | | | | | | [symbol] |
| P61089 | UBE2N | Ube2n | ICLDILK | 258 | 1.25E−05 | present | present | [symbol] |
| | | | | | | | | [symbol] |
| | | | | | | | | [symbol] |
| | | | | | | | | [symbol] |
| P61089 | UBE2N | Ube2n | LELFLPEEYPMAAPK | 259 | 2.00E−05 | absent | present | [symbol] |
| | | | | | | | | [symbol] |
| | | | | | | | | [symbol] |
| | | | | | | | | [symbol] |
| P61089 | UBE2N | Ube2n | TNEAQAIETAR | 260 | 5.41E−05 | present | present | [symbol] |
| | | | | | | | | [symbol] |
| | | | | | | | | [symbol] |
| | | | | | | | | [symbol] |
| Q9EPU0 | RENT1 | Upf1 | AGLSQSLFER | 261 | 3.02E−06 | absent | present | [symbol] |
| | | | | | | | | [symbol] |
| | | | | | | | | [symbol] |
| Q9EPU0 | RENT1 | Upf1 | [symbol] | 262 | 2.28E−05 | absent | present | [symbol] |
| | | | | | | | | [symbol] |
| | | | | | | | | [symbol] |
| Q9EPU0 | RENT1 | Upf1 | NVFLLGFIPAK | 263 | 2a.77E−05 | absent | present | [symbol] |
| | | | | | | | | [symbol] |
| | | | | | | | | [symbol] |
| Q9Z1Z0 | USO1 | Cso1 | [symbol] | 264 | 6.19E−04 | absent | present | [symbol] |
| | | | | | | | | [symbol] |
| Q9Z1Z0 | USO1 | Cso1 | TNNSNQNFFK | 265 | 9.13E−05 | absent | present | [symbol] |
| | | | | | | | | [symbol] |
| Q9Z1Z0 | USO1 | Cso1 | [symbol] | 266 | 7.34E−04 | absent | present | [symbol] |
| | | | | | | | | [symbol] |
| Q9WV55 | VAPA | Vapa | EAKPDELMDSK | 267 | 3.24E−05 | absent | absent | [symbol] |
| | | | | | | | | [symbol] |
| | | | | | | | | [symbol] |
| Q9WV55 | VAPA | Vapa | [symbol] | 268 | 1.01E−05 | absent | absent | [symbol] |
| | | | | | | | | [symbol] |
| | | | | | | | | [symbol] |
| Q9WV55 | VAPA | Vapa | VAHSDKPGSTSAVSFR | 269 | 1.11E−05 | absent | absent | [symbol] |
| | | | | | | | | [symbol] |
| | | | | | | | | [symbol] |

[symbol]

indicates data missing or illegible when filed

TABLE 10
The 78 Protein Biomarkers of the PRM Experiments

| | | | RTV | ISRIB |
|---|---|---|---|---|
| | | | Direction | Direction |
| | | | Opposite | Name as |
| Accession | GN | Description | dE? | dE? |
| P54071 | Idh2 | Isocitrate dehydrogenase [NAPP], mitochondrial | yes | yes |
| P47738 | Aldh2 | Aldehyde dehydrogenase, mitochondrial | yes | yes |
| Q9D892 | Itpa | Inosine triphosphate pyrophosphatase | yes | yes |
| Q9D1M4 | [symbol] | Eukaryotic translation [symbol] epsilon-1 | yes | yes |
| Q60972 | [symbol] | Histone-binding protein RBBP4 | yes | yes |
| Q89051 | Itm2b | Integral membrane protein 2B | yes | yes |
| Q9Z1N5 | Ddx39b | Spliceosome RNA helicase Ddx34b | yes | yes |
| Q8C878 | [symbol] | NEDD8-activating enzyme [symbol] subunit | yes | yes |
| Q35382 | Exoc4 | [symbol] complex component 4 | yes | yes |
| Q35343 | Kpna4 | Importin subunit alpha-3 | yes | yes |
| Q99KJ8 | [symbol] | Dynactin subunit 2 | yes | yes |
| Q8K2V6 | Ipo11 | Importin-11 | yes | yes |
| Q01405 | [symbol] | Protein transport protein [symbol] 23A | yes | yes |
| Q8BML9 | Qars | Glutamine--tRNA ligase | yes | yes |
| P09528 | Fth1 | Ferritin heavy chain | yes | yes |
| P27612 | Plaa | Phospholipase A-2-activating protein | yes | yes |
| Q6ZQ08 | Cnot1 | CCR4-NOT transcription complex subunit 1 | yes | yes |
| Q9CYR0 | Ssbp1 | Single-stranded DNA-binding protein, mitochondrial | yes | yes |
| Q922B2 | [symbol] | Aspartate--tRNA ligase, [symbol] | yes | yes |
| Q9Z2X1 | [symbol] | Heterogeneous nuclear ribonucleoprotein E | yes | yes |
| Q9CZN7 | Shrnt2 | Serine hydroxymethyltransferase, mitochondrial | yes | yes |
| P10923 | Spp1 | Osteopontin | yes | yes |
| Q99PV0 | Prpf8 | Pre-mRNA-processing-splicing factor N | yes | yes |
| Q7TQ [symbol] | Otub1 | Ubiquitin [symbol] OTUB1 | yes | yes |
| Q9WVG5 | Lipg | Endothelial lipase | yes | yes |
| Q9D0R2 | Tars | Threonine--tRNA ligase, cytoplasmic | yes | yes |
| Q55029 | Coph2 | Coatomer subunit beta′ | yes | yes |
| Q8R081 | Hnrnpl | Heterogeneous nuclear [symbol] L | yes | yes |
| Q9 [symbol] | Upf1 | Regulator of nonsense transcripts 1 | yes | yes |
| Q70133 | Dhx9 | ATP-dependent RNA helicase A | yes | yes |
| Q9QUR6 | Prep | Prolyl endopeptidase | yes | yes |
| P45376 | Akr1b1 | Aldose reductase | yes | yes |
| Q3 [symbol] | Sec31a | Protein transport protein Sec31A | yes | yes |
| Q8BPB5 | Efemp1 | EGF-containing fibulin-like ECM protein 1 | yes | yes |
| Q9CQ65 | Mtap | S-methyl-5′-thioadenosine phosphorylase | yes | yes |
| Q61656 | Ddx5 | Probable ATP-dependent RNA helicase DDX5 | yes | yes |
| Q3TXS7 | Psmd1 | 26S proteasome non-ATPase regulatory subunit 1 | yes | yes |
| P97310 | Mem2 | DNA replication licensing factor MCM2 | yes | yes |
| P48678 | L [symbol] | Prelamin-A C | yes | yes |
| Q35286 | Dhx15 | Pre-mRNA-splicing factor ATP-dependent RNA helicase DHX15 | yes | yes |
| Q62261 | Spthn1 | [symbol] beta chain, non-erythrocytic 1 | yes | yes |
| Q8CG19 | L [symbol] | Latent-transforming GF β-binding protein 1 | yes | yes |
| P16546 | Sptan1 | Spectrin alpha [symbol] non-erythrocyte I | yes | yes |
| Q88207 | Col5A1 | Collagen alpha-1(V) [symbol] | yes | yes |
| Q62167 | Ddx3x | ATP-dependent RNA helicase DDX3X | yes | yes |
| Q78ZA7 | Nap114 | Nucleosome assembly protein 1-like 4 | yes | NO |
| P97792 | Cxadr | Coxsackievirus and adenovirus receptor homolog | yes | NO |
| P56959 | Fus | RNA-binding protein FUS | yes | NO |
| Q55023 | [symbol] | Inositol monophosphatase 1 | yes | NO |
| Q9DAW9 | Cnn3 | Calponin-3 | yes | NO |
| Q9Z1Z0 | Usol | General vesicular transport factor p1115 | yes | NO |
| D3YXG0 | Hmen1 | Hemicentin-1 | yes | NO |
| Q99 [symbol] | Nrbp1 | Nuclear receptor binding protein | yes | NO |
| Q9WV55 | Vapa | Vesicle associated membrane protein-associated protein A | yes | NO |
| P61089 | Ube2n | Ubiquitin-conjugating enzyme [symbol] | yes | NO |
| Q35737 | H [symbol] | Heterogeneous nuclear ribonucleoprotein H | yes | NO |
| P50543 | S100a11 | Protein S100-A11 | yes | NO |
| Q09131 | Gsto1 | Glutathione S-transferase omega-1 | yes | NO |
| P12025 | Mdk | [symbol] | yes | NO |
| Q9JM [symbol] | Myo18a | Unconventional myosin-XVIIIa | yes | NO |
| P67984 | Rpl22 | 60S ribosomal protein L22 | yes | NO |
| Q35405 | Pld3 | Phospholipase D3 | yes | NO |
| Q9 [symbol] | C1qtnf3 | Complement C1q TSF--related protein 3 | yes | NO |
| P35279 | Rab6a | Ras-related protein Rab-6A | yes | NO |
| P62996 | Tra2b | Transformer-2 protein homolog beta | yes | NO |
| P42669 | Pura | Transcriptional [symbol] protein Pur-alpha | yes | NO |
| P08003 | Pdia4 | Protein disulfide-isomerase A4 | yes | NO |
| Q8BL97 | Srsf7 | Serine arginine-rich splicing factor 7 | yes | NO |
| P32067 | Ssb | Lupus La protein homolog | yes | NO |
| Q9EPL8 | Ipo7 | Importin-7 | yes | NO |
| P23927 | Cryab | Alpha-crystallin B chain | yes | NO |
| Q54890 | Itgb3 | Integrin beta-3 | yes | NO |
| Q60751 | Igf1r | Insulin-like growth factor 1 receptor | yes | NO |
| Q3 [symbol] | [symbol] | Extended synaptotagmin-1 | yes | NO |
| P22005 | Penk | Proenkephalin-A | yes | NO |
| Q35114 | Scarb2 | Lysosome membrane protein 2 | yes | NO |
| Q6X893 | Slc44a1 | Choline transporter-like protein 1 | yes | NO |
| A2AVA0 | S [symbol] | Sushi, von Willebrand factor type A, EGF and pentraxin | yes | NO |
| | | domain-containing protein 1 | | |

| | | | Abs Z | | | | | |
|---|---|---|---|---|---|---|---|---|
| | Sal | | Score | | | | | |
| | Direction | In | Sum | | | | | |
| | Opposite | Human | (Geno + | WT Mean | HetvWT | RTVvHet | ISRIBvWT | SALvHet |
| Accession | ISRIB? | Plasma | RTV) | Abundance | Z Score | Z Score | Z Score | Z Score |
| P54071 | yes | yes | 26.73 | 1.52E+06 | −3.12 | 23.61 | −2.01 | 0.46 |
| P47738 | yes | yes | 25.29 | 2.43E+05 | −7.08 | 18.21 | −7.25 | 0.22 |
| Q9D892 | yes | yes | 23.68 | 6.45E+05 | −10.88 | 12.80 | −1.25 | 4.47 |
| Q9D1M4 | yes | yes | 20.67 | 3.08E+06 | 3.77 | 16.90 | −3.65 | 7.47 |
| Q60972 | yes | yes | 18.10 | 3.99E+06 | −4.22 | 13.88 | −1.73 | 0.61 |
| Q89051 | yes | yes | 17.16 | 1.16E+06 | −5.63 | 11.53 | −2.13 | 1.23 |
| Q9Z1N5 | yes | yes | 16.28 | 4.68E+05 | −2.49 | 13.74 | −1.12 | 8.14 |
| Q8C878 | yes | yes | 14.97 | 9.06E+05 | −4.95 | 10.01 | −1.68 | 1.17 |
| Q35382 | yes | yes | 14.59 | 6.18E+05 | −6.89 | 7.70 | −1.51 | 0.87 |
| Q35343 | NO | yes | 14.28 | 2.73E+06 | −11.90 | 2.38 | −3.18 | −3.56 |
| Q99KJ8 | yes | yes | 13.94 | 2.16E+06 | 8.43 | 5.51 | 0.53 | 1.58 |
| Q8K2V6 | yes | yes | 13.53 | 2.07E+06 | −3.78 | 9.75 | −1.97 | 0.76 |
| Q01405 | NO | yes | 13.20 | 1.78E+07 | −5.06 | 8.14 | −0.95 | −0.14 |
| Q8BML9 | yes | yes | 13.17 | 9.15E+06 | −5.96 | 7.21 | −2.14 | 1.62 |
| P09528 | yes | yes | 12.58 | 2.27E+06 | −12.27 | 0.31 | −3.46 | 0.34 |
| P27612 | yes | yes | 12.45 | 2.10E+06 | −3.77 | 8.69 | −2.39 | 3.71 |
| Q6ZQ08 | yes | yes | 12.30 | 4.31E+06 | −6.76 | 5.54 | −2.56 | 0.71 |
| Q9CYR0 | yes | yes | 12.21 | 5.61E+05 | −8.91 | 3.30 | −1.49 | 0.33 |
| Q922B2 | yes | yes | 12.17 | 1.17E+07 | −2.85 | 9.32 | −1.13 | 1.39 |
| Q9Z2X1 | yes | yes | 12.02 | 9.49E+05 | −3.98 | 8.07 | −0.18 | 3.85 |
| Q9CZN7 | yes | yes | 11.93 | 4.69E+06 | −2.95 | 8.98 | −1.44 | 6.36 |
| P10923 | yes | yes | 11.35 | 6.30E+06 | 4.42 | −6.93 | 2.67 | −4.91 |
| Q99PV0 | yes | yes | 11.34 | 4.64E+07 | −3.39 | 7.95 | −0.01 | 2.09 |
| Q7TQ [symbol] | yes | yes | 11.30 | 4.71E+06 | −5.45 | 5.84 | 2.26 | 4.33 |
| Q9WVG5 | NO | yes | 11.27 | 1.58E+06 | −8.39 | 2.88 | −2.47 | −0.75 |
| Q9D0R2 | yes | yes | 9.50 | 1.92E+07 | −3.21 | 6.28 | −1.16 | 1.45 |
| Q55029 | NO | yes | 9.40 | 3.42E+07 | −3.26 | 6.13 | −1.11 | −0.12 |
| Q8R081 | yes | yes | 9.12 | 6.14E+06 | −2.95 | 6.17 | −0.33 | 1.08 |
| Q9 [symbol] | NO | yes | 9.02 | 6.08E+06 | −4.78 | 4.24 | −1.84 | −0.64 |
| Q70133 | yes | yes | 9.00 | 6.51E+06 | −2.77 | 6.23 | −1.27 | 0.08 |
| Q9QUR6 | yes | yes | 8.56 | 9.03E+06 | −3.15 | 5.40 | −1.11 | 0.60 |
| P45376 | yes | yes | 8.44 | 6.34E+06 | −3.12 | 5.32 | −0.22 | 3.31 |
| Q3 [symbol] | yes | yes | 8.41 | 5.92E+06 | −2.91 | 5.50 | −1.34 | 0.61 |
| Q8BPB5 | yes | yes | 8.34 | 7.02E+06 | 3.74 | −4.60 | 0.07 | −3.65 |
| Q9CQ65 | yes | yes | 8.12 | 6.29E+06 | −4.62 | 3.50 | −0.85 | 1.73 |
| Q61656 | yes | yes | 8.05 | 8.31E+06 | −3.57 | 4.48 | −0.92 | 2.12 |
| Q3TXS7 | yes | yes | 7.84 | 1.94E+07 | −3.50 | 4.33 | −0.65 | 1.22 |
| P97310 | NO | yes | 7.66 | 1.28E+07 | −3.70 | 3.96 | −2.10 | −0.30 |
| P48678 | yes | yes | 7.44 | 1.46E+07 | −3.47 | 3.47 | −0.19 | 2.54 |
| Q35286 | yes | yes | 7.27 | 2.27E+07 | −2.69 | 4.59 | −1.19 | 1.73 |
| Q62261 | yes | yes | 6.23 | 3.52E+07 | −2.95 | 3.27 | −0.82 | 1.78 |
| Q8CG19 | yes | yes | 6.03 | 3.35E+07 | 2.96 | −3.08 | 2.17 | −1.21 |
| P16546 | yes | yes | 6.02 | 2.31E+07 | −2.81 | 3.22 | −0.40 | 0.14 |
| Q88207 | NO | yes | 5.55 | 2.07E+07 | 4.66 | −1.39 | 0.06 | 0.43 |
| Q62167 | yes | yes | 5.09 | 2.01E+07 | −2.85 | 2.24 | −1.08 | 0.67 |
| Q78ZA7 | yes | yes | 50.77 | 2.29E+06 | −45.04 | 5.74 | 2.43 | −2.26 |
| P97792 | yes | yes | 16.73 | 4.16E+06 | −12.85 | 3.88 | 1.72 | −0.13 |
| P56959 | NO | yes | 14.88 | 1.18E+06 | −2.94 | 11.94 | 0.85 | 2.28 |
| Q55023 | NO | yes | 14.51 | 7.37E+05 | −3.62 | 10.89 | 1.73 | 3.95 |
| Q9DAW9 | NO | yes | 13.28 | 1.75E+06 | −8.50 | 4.78 | 2.76 | 1.46 |
| Q9Z1Z0 | yes | yes | 12.14 | 1.66E+06 | 3.02 | 4.12 | 2.44 | 0.59 |
| D3YXG0 | yes | yes | 11.51 | 9.50E+06 | 7.93 | −3.58 | −2.25 | 0.86 |
| Q99 [symbol] | yes | yes | 9.54 | 5.42E+05 | −3.20 | 6.35 | 0.18 | −2.12 |
| Q9WV55 | NO | yes | 8.93 | 7.68E+05 | −7.02 | 1.91 | 3.18 | 0.62 |
| P61089 | NO | yes | 8.62 | 1.06E+06 | −2.60 | 6.02 | 0.41 | 8.07 |
| Q35737 | NO | yes | 8.31 | 1.18E+06 | −4.08 | 4.23 | 2.47 | 0.44 |
| P50543 | yes | yes | 8.18 | 5.73E+06 | −3.69 | 4.49 | 0.33 | −0.12 |
| Q09131 | NO | yes | 8.09 | 6.66E+06 | −4.63 | 3.45 | 0.04 | 2.11 |
| P12025 | NO | yes | 7.88 | 6.99E+05 | −4.24 | 3.64 | 1.33 | 0.59 |
| Q9JM [symbol] | yes | yes | 7.78 | 3.49E+06 | −1.87 | 4.91 | 2.27 | −1.53 |
| P67984 | yes | yes | 7.66 | 7.30E+06 | −6.07 | 1.59 | 2.38 | −0.27 |
| Q35405 | NO | yes | 7.47 | 5.59E+05 | −6.86 | 0.61 | 1.18 | 0.57 |
| Q9 [symbol] | NO | yes | 6.95 | 1.65E+07 | 3.71 | −3.24 | −0.18 | −1.10 |
| P35279 | NO | yes | 6.80 | 1.61E+06 | −3.66 | 3.14 | 0.44 | 1.92 |
| P62996 | NO | yes | 6.63 | 1.51E+06 | −3.35 | 3.28 | 0.54 | 3.10 |
| P42669 | yes | yes | 6.04 | 2.78E+06 | −3.68 | 2.36 | 0.52 | −1.14 |
| P08003 | NO | yes | 5.92 | 4.54E+06 | −3.67 | 2.25 | 1.37 | 1.08 |
| Q8BL97 | yes | yes | 5.72 | 1.94E+07 | −2.81 | 2.91 | 0.13 | −0.89 |
| P32067 | NO | yes | 5.45 | 4.28E+06 | −2.06 | 2.79 | 0.41 | 0.09 |
| Q9EPL8 | yes | yes | 5.25 | 2.54E+07 | −3.01 | 2.25 | 0.30 | −0.95 |
| P23927 | NO | yes | 5.18 | 4.11E+05 | −4.08 | 1.11 | 0.39 | 3.46 |
| Q54890 | NO | yes | 4.87 | 1.79E+06 | −2.91 | 1.96 | 0.14 | 2.90 |
| Q60751 | NO | yes | 4.86 | 3.32E+06 | −4.23 | 0.63 | 0.75 | 0.24 |
| Q3 [symbol] | NO | yes | 4.61 | 1.52E+06 | −3.36 | 1.25 | 0.18 | −0.64 |
| P22005 | NO | yes | 4.31 | 1.35E+06 | 2.02 | −2.14 | −0.11 | −2.09 |
| Q35114 | NO | yes | 3.91 | 5.51E+05 | −3.66 | 0.24 | 0.09 | 1.13 |
| Q6X893 | NO | yes | 3.67 | 3.01E+06 | −2.83 | 0.84 | 0.80 | 1.03 |
| A2AVA0 | NO | yes | 2.64 | 2.02E+06 | 2.25 | −0.40 | −0.50 | −1.18 |

[symbol]

indicates data missing or illegible when filed

TABLE 11
The Homologous Proteins Having 1+ Human Peptides in the PRM Panel

| Accession | GN | Description |
|---|---|---|
| O35598 | Adam10 | Disintegrin and metalloproteinase domain-containing protein 10 |
| P45376 | Akr1b1 | Aldose reductase |
| P47738 | Aldh2 | Aldehyde dehydrogenase, mitochondrial |
| P10107 | Anxa1 | Annexin A1 |
| P07356 | Anxa2 | Annexin A2 |
| P48036 | Anxa5 | Annexin A5 |
| E9Q414 | Apob | Apolipoprotein B-100 |
| Q99KN1 | Arrdc1 | Arrestin domain-containing protein 1 |
| Q9ES30 | C1qtnf3 | Complement C1q tumor necrosis factor-related protein 3 |
| P01029 | C4b | Complement C4-B |
| P35564 | Canx | Calnexin |
| Q9DAW9 | Cnn3 | Calponin-3 |
| Q6ZQ08 | Cnot1 | CCR4-NOT transcription complex subunit 1 |
| O88207 | Col5a1 | Collagen alpha-1(V) chain |
| O55029 | Copb2 | Coatomer subunit beta' |
| Q61147 | Cp | Ceruloplasmin |
| P23927 | Cryab | Alpha-crystallin B chain |
| P97792 | Cxadr | Coxsackievirus and adenovirus receptor homolog |
| Q922B2 | Dars | Aspartate--tRNA ligase, cytoplasmic |
| Q99KJ8 | Dctn2 | Dynactin subunit 2 |
| Q9Z1N5 | Ddx39b | Spliceosome RNA helicase Ddx39b |
| Q62167 | Ddx3x | ATP-dependent RNA helicase DDX3X |
| Q61656 | Ddx5 | Probable ATP-dependent RNA helicase DDX5 |
| O35286 | Dhx15 | Pre-mRNA-splicing factor ATP-dependent RNA helicase DHX15 |
| Q9D1M4 | Eef1e1 | Eukaryotic translation elongation factor 1 epsilon-1 |
| Q8BPB5 | Efemp1 | EGF-containing fibulin-like extracellular matrix protein 1 |
| Q3U7R1 | Esyt1 | Extended synaptotagmin-1 |
| O35382 | Exoc4 | Exocyst complex component 4 |
| Q60634 | Flot2 | Flotillin-2 |
| P09528 | Fth1 | Ferritin heavy chain |
| P56959 | Fus | RNA-binding protein FUS |
| O09131 | Gsto1 | Glutathione S-transferase omega-1 |
| D3YXG0 | Hmcn1 | Hemicentin-1 |
| Q9Z2X1 | Hnrnpf | Heterogeneous nuclear ribonucleoprotein F |
| O35737 | Hnrnph1 | Heterogeneous nuclear ribonucleoprotein H |
| Q8R081 | Hnrnpl | Heterogeneous nuclear ribonucleoprotein L |
| P11499 | Hsp90ab1 | Heat shock protein HSP 90-beta |
| P54071 | Idh2 | Isocitrate dehydrogenase [NADP], mitochondrial |
| Q60751 | Igflr | Insulin-like growth factor 1 receptor |
| O55023 | Impa1 | Inositol monophosphatase 1 |
| Q8K2V6 | Ipo11 | Importin-11 |
| Q9EPL8 | Ipo7 | Importin-7 |
| O54890 | Itgb3 | Integrin beta-3 |
| O89051 | Itm2b | Integral membrane protein 2B |
| O35343 | Kpna4 | Importin subunit alpha-3 |
| P48678 | Lmna | Prelamin-A/C |
| Q8CG19 | Ltbp1 | Latent-transforming growth factor beta-binding protein 1 |
| P97310 | Mcm2 | DNA replication licensing factor MCM2 |
| P12025 | Mdk | Midkine |
| Q9CQ65 | Mtap | S-methyl-5′-thioadenosine phosphorylase |
| Q9JMH9 | Myo18a | Unconventional myosin-XVIIIa |
| Q99J45 | Nrbp1 | Nuclear receptor-binding protein |
| Q7TQ13 | Otub1 | Ubiquitin thioesterase OTUB1 |
| Q9WU78 | Pdcd6ip | Programmed cell death 6-interacting protein |
| P08003 | Pdia4 | Protein disulfide-isomerase A4 |
| P22005 | Penk | Proenkephalin-A |
| P27612 | Plaa | Phospholipase A-2-activating protein |
| Q9QUR6 | Prep | Prolyl endopeptidase |
| Q99PV0 | Prpf8 | Pre-mRNA-processing-splicing factor 8 |
| Q3TXS7 | Psmd1 | 26S proteasome non-ATPase regulatory subunit 1 |
| P42669 | Pura | Transcriptional activator protein Pur-alpha |
| Q8BML9 | Qars | Glutamine--tRNA ligase |
| P35279 | Rab6a | Ras-related protein Rab-6A |
| Q60972 | Rbbp4 | Histone-binding protein RBBP4 |
| P67984 | Rp122 | 60S ribosomal protein L22 |
| P50543 | S100a11 | Protein S100-A11 |
| Q01405 | Sec23a | Protein transport protein Sec23A |
| Q3UPL0 | Sec31a | Protein transport protein Sec31A |
| Q9CZN7 | Shmt2 | Serine hydroxymethyltransferase, mitochondrial |
| Q6X893 | Slc44a1 | Choline transporter-like protein 1 |
| P16546 | Sptan1 | Spectrin alpha chain, non-erythrocytic 1 |
| Q62261 | Sptbn1 | Spectrin beta chain, non-erythrocytic 1 |
| Q8BL97 | Srsf7 | Serine/arginine-rich splicing factor 7 |
| P32067 | Ssb | Lupus La protein homolog |
| Q9CYR0 | Ssbp1 | Single-stranded DNA-binding protein, mitochondrial |
| A2AVA0 | Svep1 | Sushi, von Willebrand factor type A, EGF and pentraxin domain- |
| | | containing protein 1 |
| Q9D0R2 | Tars | Threonine--tRNA ligase, cytoplasmic |
| P62996 | Tra2b | Transformer-2 protein homolog beta |
| Q61187 | Tsg101 | Tumor susceptibility gene 101 protein |
| Q8C878 | Uba3 | NEDD8-activating enzyme E1 catalytic subunit |
| P61089 | Ube2n | Ubiquitin-conjugating enzyme E2 N |
| Q9EPU0 | Upf1 | Regulator of nonsense transcripts 1 |
| Q9Z1Z0 | Uso1 | General vesicular transport factor p115 |
| Q9WV55 | Vapa | Vesicle-associated membrane protein-associated protein A |

TABLE 12
The Homologous Protein Having No
Human Peptides in the PRM Panel

| Accession | GN | Description |
|---|---|---|
| P07724 | Alb | Serum albumin |
| P41731 | Cd63 | CD63 antigen |
| P35762 | Cd81 | CD81 antigen |
| P40240 | Cd9 | CD9 antigen |
| O70133 | Dhx9 | ATP-dependent RNA helicase A |
| E9PV24 | Fga | Fibrinogen alpha chain |
| Q8VCM7 | Fgg | Fibrinogen gamma chain |
| P07901 | Hsp90aa1 | Heat shock protein HSP 90-alpha |
| Q9D892 | Itpa | Inosine triphosphate pyrophosphatase |
| Q9WVG5 | Lipg | Endothelial lipase |
| Q78ZA7 | Nap1l4 | Nucleosome assembly protein 1-like 4 |
| O35405 | Pld3 | Phospholipase D3 |
| O35114 | Scarb2 | Lysosome membrane protein 2 |
| P10923 | Spp1 | Osteopontin |

TABLE 13
The 20 EV Control Markers for the PRM Experiments
EV Markers

| | | | WT Mean | Geno | Geno |
|---|---|---|---|---|---|
| Accession | GN | Description | Abundance | FC | Pval |
| P11499 | Hsp90ab1 | Heat shock protein HSP 90-beta OS [symbol] Mus musculus OX [symbol] 10090 | 1.78E−08 | −1.18 | 0.551 |
| | | GN [symbol] Hsp90ab1 PE [symbol] 1 SV [symbol] 3 | | | |
| Q9WU78 | Pdcd6ip | Programmed cell death 6-interacting protein OS-Mus musculus | 1.44E−08 | 1.06 | 0.828 |
| | | OX [symbol] 10090 GN [symbol] Pdcd6ip PE [symbol] 1 SV [symbol] 3 | | | |
| P48036 | Anxa5 | Annexin A5 OS [symbol] Mus musculus OX [symbol] 10090 GN [symbol] Anxa5 PE [symbol] 1 | 9.81E−07 | −1.31 | 0.296 |
| | | SV [symbol] 1 | | | |
| P10107 | Anxa1 | Annexin A1 OS [symbol] Mus musculus OX [symbol] 10090 GN [symbol] Anxa1 PE [symbol] 1 | 5.83E−07 | −1.18 | 0.344 |
| | | SV [symbol] 2 | | | |
| P07356 | Anxa2 | Annexin A2 OS-Mus musculus OX-10090 GN [symbol] Anxa2 PE-1 | 4.75E−07 | −1.3 | 0.496 |
| | | SV [symbol] 2 | | | |
| P35762 | Cd81 | CD81 antigen OS Mus musculus OX [symbol] 10090 GN [symbol] Cd81 PE [symbol] 1 | 4.12E−07 | −1.23 | 0.541 |
| | | SV [symbol] 2 | | | |
| P07901 | Hsp90aa1 | Heat shock protein HSP 90-alpha OS-Mus musculus | 2.38E−07 | −1.25 | 0.474 |
| | | OX [symbol] 10090 GN [symbol] Hsp90aa1 PE [symbol] 1 SV [symbol] 4 | | | |
| O35598 | Adam10 | Disintegrin and metalloproteinase domain-containing protein 10 | 2.03F−07 | −1.19 | 0.525 |
| | | OS [symbol] Mus musculus OX [symbol] 10090 GN [symbol] Adam10 PE [symbol] 1 SV [symbol] 2 | | | |
| P40240 | Cd9 | CD9 antigen OS [symbol] Mus musculus OX [symbol] 10090 GN [symbol] Cd9 PE [symbol] 1 | 1.94E−07 | −1.03 | 0.982 |
| | | SV-2 | | | |
| P41731 | Cd63 | CD63 antigen OS-Mus musculus OX-10090 GN-Cd63 PE-1 | 1.57E−07 | −1.09 | 0.819 |
| | | SV [symbol] 2 | | | |
| Q61187 | Tsg101 | Tumor susceptibility gene 101 protein OS [symbol] Mus musculus | 1.24E−07 | 1.04 | 0.942 |
| | | OX-10090 GN-Tsg101 PE-1 SV-2 | | | |
| Q99KN1 | Arrde1 | Arrestin domain-containing protein 1 OS Mus musculus | 7.50E−06 | 1.27 | 0.617 |
| | | OX [symbol] 10090 GN [symbol] PE [symbol] 1 SV [symbol] 2 | | | |
| P35564 | Canx | Calnexin OS-Mus musculus OX-10090 GN-Canx PE-1 SV-1 | 2.97E−06 | −1.92 | 0.034 |
| Q60634 | Flot2 | Flotillin-2 OS [symbol] Mus musculus OX [symbol] 10090 GN [symbol] Flot2 PE [symbol] 1 | 2.62E−06 | −1.17 | 0.506 |
| | | SV [symbol] 2 | | | |

[symbol]

indicates data missing or illegible when filed

TABLE 14
The 6 Plasma Control Markers for the PRM Experiments
Plasma Markers

| | | | WT Mean | Geno | Geno |
|---|---|---|---|---|---|
| Accession | GN | Description | Abundance | FC | Pval |
| P07724 | Alb | Serum albumin OS [symbol] Mus musculus OX [symbol] 10090 GN [symbol] Alb PE [symbol] 1 | 6.12E−08 | 1.74 | 0.194 |
| | | SV [symbol] 3 | | | |
| P01029 | C4b | Complement C4-B OS [symbol] Mus musculus OX [symbol] 10090 GN [symbol] C4b | 3.87E−07 | 1.76 | 0.272 |
| | | PE-1 SV-3 | | | |
| E9Q414 | Apob | Apolipoprotein B-100 OS [symbol] Mus musculus OX [symbol] 10090 | 9.16E−06 | 1.88 | 0.208 |
| | | GN [symbol] Apob PE [symbol] 1 SV [symbol] 1 | | | |
| Q8VCM7 | Fgg | Fibrinogen gamma chain OS-Mus musculus OX-10090 | 3.68E−06 | 1.71 | 0.329 |
| | | GN [symbol] Fgg PE [symbol] 1 SV [symbol] 1 | | | |
| Q61147 | Cp | Ceruloplasmin OS-Mus musculus OX-10090 GN-Cp PE-1 | 1.41E−06 | 1.51 | 0.561 |
| | | SV [symbol] 2 | | | |
| E9PV24 | Fga | Fibrinogen alpha chain OS [symbol] Mus musculus OX [symbol] 10090 | 4.76E−05 | 1.84 | 0.093 |
| | | GN [symbol] Fga PE [symbol] 1 SV [symbol] 1 | | | |

[symbol]

indicates data missing or illegible when filed

TABLE 15
THE PRM Validation of Murine Candidate
Biomarkers in EVs Derived from hiPSCs

| | Total | | EV | Plasma |
|---|---|---|---|---|
| | Proteins (98) | Biomarkers (78) | Controls (14) | Controls (6) |
| Protein Total # | | | | |
| MEF EV | 89 | 73 | 13 | 3 |
| NPC EV | 43 | 31 | 9 | 3 |
| Protein Percent | | | | |
| MEF EV | 91% | 94% | 93% | 50% |
| NPC EV | 44% | 40% | 64% | 50% |
| | Total | | EV | Plasma |
| | Peptides (258) | Biomarkers (210) | Controls (37) | Controls (11) |
| Peptide Total # | | | | |
| MEF EV | 218 | 181 | 32 | 5 |
| NPC EV | 74 | 51 | 19 | 4 |
| Peptide Percent | | | | |
| MEF EV | 84% | 86% | 86% | 45% |
| NPC EV | 29% | 24% | 51% | 36% |

### Example 7

### Identification of Enriched Pathways

To determine whether eIF2alpha-dependent translational processes were altered in DYT1, broad proteomic differences were examined. Additional bioinformatic analyses for relationships among differentially expressed proteins were performed using the Metascape platform (Zhou Y, et al. (2019) Nat Commun. 10(1):1523) against a custom proteome background of the 1008 proteins overlapping between Cohort 1 and Cohort 2. To generate an appropriately sized list for pathway enrichment, a subset of 202 proteins shared between cohorts with a mean FC>2 and a Fisher's combined p-value<0.01 were identified. FIG. 4 shows the results of this analysis. Of note, several of the enriched pathways were associated with pathways previously associated with DYT1, such as nuclear envelope (Goodchild R E, et al. (2005) Neuron. 48:923-932), protein translation (Rittiner J E, et al. (2016) Neuron. 92:1238-1251) and NF-kappaB (Vaine C A, et al. (2017) Neurobiol Dis. 100:108-118).

1. A method of identifying a dystonia biomarker in a subject, the method comprising:
obtaining a biosample from a subject having a dystonia;
obtaining a biosample from a subject not having a dystonia;
determining the expression level of one or more proteins and/or miRNAs in both biosamples;
identifying those proteins and/or miRNAs that are differentially expressed in the biosample obtained from the subject having a dystonia when compared to the biosample from the subject not having a dystonia;
wherein those differentially expressed proteins and/or miRNAs are biomarkers of a dystonia.

2. The method of claim 1, wherein a determining the level of one or more proteins in one or both samples comprises using liquid chromatography with tandem mass spectrometry (LC-MS-MS).

3. The method of claim 1, wherein a determining the level of one or more miRNAs in one or both samples comprises using RNASeq or RT-qPCR.

4. The method of claim 1, wherein the biosample comprises extracellular vesicles.

5. The method of claim 1, wherein the one or more proteins comprise isocitrate dehydrogenase [NADP] (mitochondrial), aldehyde dehydrogenase (mitochondrial), inosine triphosphate pyrophosphatase, eukaryotic translation EF1 epsilon-1, histone-binding protein RBBP4, integral membrane protein 2B, spliceosome RNA helicase Ddx39b, NEDD8-activating enzyme E1 catalytic subunit, exocyst complex component 4, importin subunit alpha-3, dynactin subunit 2, importin-11, protein transport protein Sec23A, glutamine-tRNA ligase, ferritin heavy chain, phospholipase A-2-activating protein, CCR4-NOT transcription complex subunit 1, single-stranded DNA-binding protein, mitochondrial, aspartate-tRNA ligase, cytoplasmic, heterogeneous nuclear ribonucleoprotein F, hi stone H3.1, serine hydroxymethyltransferase, mitochondrial, osteopontin, pre-mRNA-processing-splicing factor 8, ubiquitin thioesterase OTUB1, endothelial lipase, threonine-tRNA ligase, cytoplasmic, coatomer subunit beta′, heterogeneous nuclear ribonucleoprotein L, Regulator of nonsense transcripts 1, ATP-dependent RNA helicase A, prolyl endopeptidase, aldose reductase, protein transport protein Sec31A, EGF-containing fibulin-like ECM protein 1, S-methyl-5′-thioadenosine phosphorylase, probable ATP-dependent RNA helicase DDX5, 26S proteasome non-ATPase regulatory subunit 1, DNA replication licensing factor MCM2, prelamin-A/C, Pre-mRNA-splicing factor ATP-dependent RNA helicase DHX15, spectrin beta chain, non-erythrocytic 1, latent-transforming growth factor beta-binding protein 1, spectrin alpha chain, non-erythrocytic 1, collagen alpha-1(V) chain, ATP-dependent RNA helicase DDX3X, and any combination thereof.

6. The method of claim 1, wherein the one or more miRNAs comprise miR-135a-5p, miR-182-5p, miR-542-5p, miR-298-5p, miR-183-5p, miR-296-3p, miR-96-5p, miR-344d-3p, miR-5121, miR-140-3p, miR-344-3p, miR-187-3p, miR-130b-3p, miR-125b-1-3p, miR-34a-5p, miR-532-5p, miR-148a-3p, miR-3535, miR-362-5p, miR-192-5p, miR-34c-5p, miR-1291, miR-30b-5p, miR-362-3p, miR-671-5p, miR-31-5p, miR-22-3p, miR-199b-3p, miR-199a-3p, miR-30e-5p, miR-30c-5p, miR-30a-5p, miR-93-5p, miR-19b-3p, let-7i-5p, miR-103-3p, let-7f-5p, miR-26b-5p, and any combination thereof.

7. The method of claim 1, wherein the dystonia comprises focal dystonia, blepharospasm, cervical dystonia, oromandibular dystonia, task-specific or occupational dystonia, spasmodic dysphonia, generalized dystonia, segmental dystonia, DYT1-related dystonia, DYT6-related dystonia, DYT28-related dystonia, dopa-responsive dystonia, myoclonic dystonia, X-linked dystonia-Parkinsonism, rapid-onset dystonia-Parkinsonism, paroxysmal dystonia choreoathetosis, paroxysmal kinesigenic dystonia, paroxysmal nonkinesigenic dyskinesia, paroxysmal exertion-induced dyskinesia, primary dystonia, acquired dystonia, tardive dyskinesia, or tardive dystonia.

8. The method of claim 1, further comprising treating the subject having a dystonia, wherein treating the subject comprises administering one or more agents that modulate the expression level of one or more differentially expressed proteins and/or miRNAs.

9. (canceled)

10. A method of treating a subject having a dystonia, the method comprising:
obtaining a biosample from a subject after treatment;
determining the expression level of one or more proteins and/or one or more miRNAs in the post-treatment biosample, wherein:
if the post-treatment expression level represents an improvement over a pre-treatment expression level of the one or more proteins and/or the one or more miRNAs, or
if the post-treatment expression level is within an acceptable range of a reference expression level,
then continuing to administer the treatment.

11. The method of claim 10, comprising obtaining a biosample from the subject prior to treatment and detecting the expression level of the one or more proteins and/or the one or more miRNAs in the pre-treatment biosample.

12. The method of claim 10, wherein determining the expression level of one or more proteins comprises using liquid chromatography with tandem mass spectrometry (LC-MS-MS).

13. The method of claim 10, wherein determining the level of one or more miRNAs in one or both samples comprises using RNASeq or RT-qPCR.

14. The method of claim 10, wherein the reference expression level comprises an expression level obtained from a biosample from a subject not having a dystonia or from biosamples of subjects not having a dystonia.

15. The method of claim 10, wherein the post-treatment expression level of the one or more proteins represents an improvement over a pre-treatment expression level when the post-treatment expression level is more similar to a reference expression level than to the pre-treatment expression level.

16. The method of claim 10, wherein the biosample comprises extracellular vesicles.

17. The method of claim 10, wherein the one or more proteins comprise isocitrate dehydrogenase [NADP] (mitochondrial), aldehyde dehydrogenase (mitochondrial), inosine triphosphate pyrophosphatase, eukaryotic translation EF1 epsilon-1, histone-binding protein RBBP4, integral membrane protein 2B, spliceosome RNA helicase Ddx39b, NEDD8-activating enzyme E1 catalytic subunit, exocyst complex component 4, importin subunit alpha-3, dynactin subunit 2, importin-11, protein transport protein Sec23A, glutamine-tRNA ligase, ferritin heavy chain, phospholipase A-2-activating protein, CCR4-NOT transcription complex subunit 1, single-stranded DNA-binding protein, mitochondrial, aspartate-tRNA ligase, cytoplasmic, heterogeneous nuclear ribonucleoprotein F, hi stone H3.1, serine hydroxymethyltransferase, mitochondrial, osteopontin, pre-mRNA-processing-splicing factor 8, ubiquitin thioesterase OTUB1, endothelial lipase, threonine-tRNA ligase, cytoplasmic, coatomer subunit beta′, heterogeneous nuclear ribonucleoprotein L, Regulator of nonsense transcripts 1, ATP-dependent RNA helicase A, prolyl endopeptidase, aldose reductase, protein transport protein Sec31A, EGF-containing fibulin-like ECM protein 1, S-methyl-5′-thioadenosine phosphorylase, probable ATP-dependent RNA helicase DDX5, 26S proteasome non-ATPase regulatory subunit 1, DNA replication licensing factor MCM2, prelamin-A/C, Pre-mRNA-splicing factor ATP-dependent RNA helicase DHX15, spectrin beta chain, non-erythrocytic 1, latent-transforming growth factor beta-binding protein 1, spectrin alpha chain, non-erythrocytic 1, collagen alpha-1(V) chain, ATP-dependent RNA helicase DDX3X, and any combination thereof.

18. The method of claim 10, wherein the one or more miRNAs comprise miR-135a-5p, miR-182-5p, miR-542-5p, miR-298-5p, miR-183-5p, miR-296-3p, miR-96-5p, miR-344d-3p, miR-5121, miR-140-3p, miR-344-3p, miR-187-3p, miR-130b-3p, miR-125b-1-3p, miR-34a-5p, miR-532-5p, miR-148a-3p, miR-3535, miR-362-5p, miR-192-5p, miR-34c-5p, miR-1291, miR-30b-5p, miR-362-3p, miR-671-5p, miR-31-5p, miR-22-3p, miR-199b-3p, miR-199a-3p, miR-30e-5p, miR-30c-5p, miR-30a-5p, miR-93-5p, miR-19b-3p, let-7i-5p, miR-103-3p, let-7f-5p, miR-26b-5p, and any combination thereof.

19. The method of claim 10, wherein the dystonia comprises focal dystonia, blepharospasm, cervical dystonia, oromandibular dystonia, task-specific or occupational dystonia, spasmodic dysphonia, generalized dystonia, segmental dystonia, DYT1-related dystonia, DYT6-related dystonia, DYT28-related dystonia, dopa-responsive dystonia, myoclonic dystonia, X-linked dystonia-Parkinsonism, rapid-onset dystonia-Parkinsonism, paroxysmal dystonia choreoathetosis, paroxysmal kinesigenic dystonia, paroxysmal nonkinesigenic dyskinesia, paroxysmal exertion-induced dyskinesia, primary dystonia, acquired dystonia, tardive dyskinesia, or tardive dystonia.

20. The method of claim 10, wherein the treatment comprises administering to the subject one or more agents that modulate the expression level of the one or more differentially expressed proteins and/or the one or more miRNAs.

21. The method of claim 20, wherein the one or more agents comprise ritonavir.

22.-32. (canceled)
